(19) United States
(12) Patent Application Publication
(10) Pub. No.: US 2004/0204368 A1
(43) Pub. Date: Oct. 14, 2004

(54) Oxadiazole derivative compounds and drugs containing these compounds as the active ingredient

(76) Inventors: Kazuyuki Ohmoto, Mishima-gun (JP); Kazuhito Kawabata, Mishima-gun (JP)

Correspondence Address: SUGHRUE MION, PLLC, 2100 PENNSYLVANIA AVENUE, N.W., SUITE 800, WASHINGTON, DC, 20037, US

(21) Appl. No.: 10/479,254

(22) PCT Filed: May 30, 2002
(86) PCT No.: PCT/JP02/05251

(30) Foreign Application Priority Data
May 31, 2001 (JP) 2001-163971

(51) Int. Cl. A61K 38/04; C07K 5/04

(52) U.S. Cl. 514/19; 560/41

(57) ABSTRACT

An oxadiazole derivative of formula (I) and a non-toxic salt thereof,

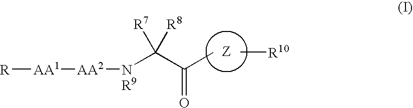

wherein all symbols have the same meaning as described in the specification. The compound of formula (I) has an inhibitory activity against cysteine protease and therefore it is useful as an agent for the prophylaxis and/or treatment of inflammatory diseases, diseases induced by apoptosis, diseases induced by disorders of immune responses, autoimmune diseases, diseases induced by decomposition of proteins which compose organism, shock, circulatory system disorders, blood coagulation system(s) disorders, malignant tumors, acquired immune deficiency syndrome (AIDS) and AIDS-related complex (ARC), parasitic diseases, nerve degeneration diseases, pulmonary disorders, bone resorption diseases, endocrinesthenia, etc.

### TECHNICAL FIELD

The present invention relates to an oxadiazole derivative.

Specifically, the present invention relates to:

1) an oxadiazole derivative of formula (I),

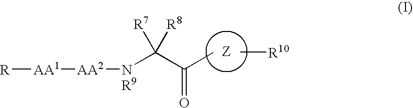

wherein all symbols have the same meanings as below, and a non-toxic salt thereof,

2) a method for the preparation thereof and

3) a pharmaceutical composition comprising the oxadiazole derivative and a non-toxic salt thereof as active ingredient.

### BACKGROUND ART

Cysteine protease is a generic name of proteases which have a cysteine residue in the activity center and catalyze protein degradation thereat. In animal cells, a large number of cysteine proteases are known; for example, cathepsin family, calpain family, caspase-1, etc. Cysteine protease exists in various kinds of cells extensively and plays a basic and essential role in the homeostasis, such as conversion (processing) of precursor protein into its active form and degradation of proteins which have become out of use, etc. Until now, its physiological effects are being vigorously studied, and as the studies progress and characteristics of the enzymes are revealed, cysteine protease came to be taken as a cause of really various kinds of diseases.

It is revealed that cathepsin S (See J. Immunol., 161, 2731 (1998)) and cathepsin L (See J. Exp. Med., 183, 1331 (1996)) play a role in processing of major histocompatibility antigen class-II in antigen presenting cells which play an important role in the early stage of immune responses. In an experimental inflammatory response model induced by antigens, a specific inhibitor of cathepsin S showed an inhibitory effect (see J. Clin. Invest., 101, 2351 (1998)). It is also reported that in a leishmania-infected immune response model cathepsin B inhibitor inhibited an immune response and by means of this effect it inhibited the proliferation of protozoans (See J. Immunol., 161, 2120 (1998)). In vitro, a result is given that a calpain inhibitor and a cysteine protease inhibitor E-64 inhibited apoptosis which is induced by stimuli on T cell receptors (see J. Exp. Med., 178, 1693 (1993)). Therefore, it is conceivable that cysteine protease is much concerned with the progress of immune responses.

It is speculated that caspase-1 or a cysteine protease similar thereto occupies an important position in the mechanism of cell death including apoptosis. Therefore it is expected for a cysteine protease inhibitor to be used as an agent for the prophylaxis and/or treatment of those diseases concerning apoptosis, such as infectious diseases, deterioration or sthenia of immune function and brain function, tumors, etc. Diseases concerning apoptosis are, acquired immune deficiency syndrome (AIDS), AIDS-related complex (ARC), adult T cell leukemia, hairy cell leukemia, spondylopathy, respiratory apparatus disorder, arthitis, HIV or HTLV-1 related diseases such as uveitis, virus-related diseases such as hepatitis C, cancer, collagenosis (systemic lupus erythematosus, rheumatoid arthritis, etc.), autoimmune diseases (ulcerative colitis, Sjögren's syndrome, primary biliary cirrhosis, spontaneous thrombocytopenic purpura, autoimmune hemolytic anemia, myasthenia gravis, insulin dependent (type I) diabetes, etc.), diseases accompanied by thrombocytopenia (osteomyelodysplasia syndrome, periodic thrombocytopenia, aplastic anemia, spontaneous thrombocytopenia, disseminated intravascular coagulation (DIC), etc.), hepatic diseases such as viral hepatitis (type C, A, B, F, etc.) or hepatitis medicamentosus and cirrhosis, dementia (Alzheimer's diseases, Alzheimer's senile dementia, etc.), cerebrovascular injury, nerve degeneration diseases, adult acute respiratory distress syndrome, infectious diseases, prostatomegaly, hysteromyoma, bronchial asthma, arteriosclerosis, all kinds of lusus naturae, nephropathy, senile cataract, chronic fatigue syndrome, myodystrophy, peripheral neuropathy, etc.

Moreover, caspase-1 is concerned with various inflammatory diseases and those diseases caused by immune disorders, by means of interleukin-1β (IL-1β) production. A lot of diseases are shown to be involved with caspase-1 including inflammatory diseases and autoimmune diseases listed below; inflammatory bowel diseases such as ulcerative colitis, insulin-dependent (type-I) diabetes, autoimmune thyroid diseases, infectious diseases, rejection of an organ transplantation, graft versus host diseases, psoriasis, periodontitis (above, see N. Eng. J. Med., 328, 106 (1993)), pancreatitis (see J. Interferon Cytokine Res., 17, 113 (1997)), hepatitis (see J. Leuko. Biol., 58, 90 (1995)), glomerulonephritis (see Kidney Int., 47, 1303 (1995)), endocarditis (see Infect. Immun., 64, 1638 (1996)), myocarditis (see Br. Heart J., 72, 561 (1995)), systemic lupus erythematosus (see Br. J. Rheumatol., 34, 107 (1995)), Hashimoto's diseases (see Autoimmunity, 16, 141(1993)), etc.), etc. Experimentally, it is reported that in liver injury model induced by lipopolysaccharide and D-galactosamine, a caspase-1 inhibitor depressed the symptoms, and it is expected that a caspase inhibitor shows an effect in sepsis, ischemic reperfusion and hepatitis gravis (see Am. J. Respir. Crit. Care Med., 159, 1308 (1999)).

It is also shown that cysteine protease is concerned with rheumatoid arthritis. IL-1β is shown to be concerned with this disease (see Arthritis Rheum., 39, 1092 (1996)), and in addition, as autoantibody toward calpastatin (endogenous calpain inhibitor) was found in the serum of the patients, it is considered that increase of calpain activity leads to the cause of diseases.

It is also known that cysteine protease causes a disease symptom by decomposing various proteins which compose the organism.

It is reported that cathepsin B plays a role in decomposing muscular protein in the chronic phase of sepsis (see J. Clin. Invest., 97, 1610 (1996)), and in decomposing muscular protein in myodystrophy model (see Biochem. J., 288, 643 (1992)). And it is also reported that calpain decomposes the myocyte cells protein of myodystrophy patients (see J. Biol. Chem., 270, 10909 (1995)).

In the ischemic reperfusion model, a result is given that calpain causes degeneration of brain tissues by means of degradation of protein kinase C-β (see J. Neurochem., 72, 2556 (1999)) and that a cathepsin B inhibitor inhibits nerve injury (see Eur. J. Neurosci., 10, 1723 (1998)).

In the brain ischemic model, it is known that the degradation of spectrin by calpain causes a damage and function disorder in the neurocyte (see Brain Res., 790, 1(1998)) and it is reported that an IL-1β receptor antagonist relieved the symptoms (see Brain Res. Bull., 29, 243 (1992)).

In myocardial ischemic model it is confirmed that cathepsin B activity increases in the lesion (see Biochem. Med. Metab. Biol., 45, 6 (1991)).

In the experiment utilizing ischemic liver injury model, it proved that necrosis and apoptosis of hepacyte were induced by means of protein-decomposing activity of calpain (see Gastroenterology, 116, 168 (1999)).

Besides, it is known that calpain causes cornea turbid in cataract by means of degradation of crystalline (see Biol. Chem., 268, 137 (1993)) and that in the lesion of contracted gut mucosa model it was confirmed that the activity of cathepsin B, H and L increased (see JPEN. J. Parenter. Enteral. Nutr., 19, 187 (1995)) and it is shown that cysteine protease is a cause of the diseases resulting from such protein degradation.

It has been revealed that cysteine protease is concerned with systemic disorders of organs and tissues by shock.

It is shown that IL-1β is concerned with septic shock and systemic inflammatory response syndrome (see Igakuno Ayumi, 169, 850 (1994)) and besides, it is reported that in endotoxin shock model induced by lipopolysaccharide, a calpain inhibitor prevented circulatory system disorder, disorders of liver and pancreas and acidosis by means of inhibitory effect of activation of nuclear factor KB (see Br. J. Pharmacol., 121, 695 (1997)).

Since it is reported that calpain is concerned with platelet coagulation process and a calpain inhibitor prevented the coagulation of platelets (see Am. J. Physiol., 259, C862 (1990)), it is conceivable that a cysteine protease inhibitor is useful for the disorder by blood coagulation. From the fact that calpain activity increased in the serum of the patients of purpura (thrombocytopenia) resulting from marrow transplantation, it is conceivable that calpain is concerned with the actual disease symptoms (see Bone Marrow Transplant., 24, 641 (1999)). Caspase-1 inhibitor inhibited the apoptosis of blood vessel endothelial cells, which is seen in the early phase of purpura (thrombocytopenia) and is thought to be important for the progression of the pathology afterwards (see Am. J. Hematol., 59, 279 (1998)), so it is expected that a cysteine protease inhibitor makes effect on purpura and hemolytic uremic syndrome.

The effect of cysteine protease and its inhibitor is being investigated in the field of cancer and metastasis of cancer.

Since the proliferations of pancreas cancer cells (see Cancer Res., 59, 4551 (1999)) and acute myeloid leukemia cells (see Clin. Lab. Haematol., 21, 173 (1999)) were inhibited by an inhibitor or receptor antagonist of caspase-1, it is expected that caspase-1 activity is essential for the process of proliferation of tumor cells, and that an inhibitor thereof is effective for these cancers. Cathepsin B activity increased in colon cancer metastasis model (see Clin. Exp. Metastasis, 16, 159 (1998)). Cathepsin K protein expression was recognized in human breast cancer cells and the relationship of cathepsin K and bone metastasis is shown (Cancer Res., 57, 5386 (1997)). Also, a calpain inhibitor inhibited migaration of the cells and it implied the possibility that calpain inhibition may inhibit metastasis of cancer (J. Biochem., 272, 32719 (1997)). From these, a cysteine protease inhibitor is presumed to show an inhibitory effect on the metastasis of various malignant tumors.

As to AIDS (see AIDS, 10, 1349 (1996)) and AIDS-related complex (ARC) (see Arch. Immunol. Ther. Exp. (Warsz), 41, 147 (1993)), it is shown that IL-1 is concerned with the progress of symptoms, so it is conceivable that cysteine protease inhibition leads to an effective therapy of AIDS and its complication.

Some parasites have cysteine protease activity in their body. Cysteine protease in the phagosome of malaria protozoan is an essential enzyme for supplying nutrition of the parasites. A result is given that the inhibitor of cysteine protease shows an inhibitory effect of the proliferation of the protozoan (see Blood, 87, 4448 (1996)). Thus, it is possible to apply the inhibitor of cysteine protease to malaria.

In Alzheimer-type dementia, it is said that adhesion of non-physiological protein called amyloid to brain is deeply involved with nervous function disorders. Cysteine protease has an activity of generating amyloid by decomposing its precursor protein. Clinically, it is shown that cathepsin B is an enzyme that possesses a processing activity of amyloid proteins in the brains of Alzheimer-type dementia patients (see Biochem. Biophys. Res. Commun., 177, 377 (1991)). Also, expressions of cathepsin B protein (see Virchows Arch. A. Pathol. Anat. Histpathol., 423, 185 (1993)), cathepsin S protein (see Am. J. Pathol., 146, 848 (1995)) and calpain protein (see Proc. Natl. Acad. Sci. USA, 90, 2628 (1993)) and increase of caspase-1 activity (see J. Neuropathol. Exp. Neurol., 58, 582 (1999)) were confirmed in the brain lesions. Besides, by the fact that calpain is concerned with the formation of paired helical filaments which accumulate in Alzheimer dementia patients and production of protein kinase C which stabilizes the protein by phosphorylation (see J. Neurochem., 66, 1539 (1996)) and by the knowledge that caspase is concerned with neurocyte death by 0 amyloid protein adhesion (see Exp. Cell Res., 234, 507 (1997)), it is implied that cysteine protease is concerned with the disease symptoms.

As to Huntington's chorea, cathepsin H activity increased in the patient's brain (see J. Neurol. Sci., 131, 65 (1995)), and the ratio of activated form of calpain increased (see J. Neurosci., 48, 181 (1997)). In Parkinson's diseases, the increase of expression of m-calpain was recognized in the mesencephalon of the patients (see Neuroscience, 73, 979 (1996)) and IL-1β protein was expressed in brain (see Neurosci. Let., 202, 17 (1995)). Therefore, it is speculated that cysteine protease is concerned with the genesis and progress of these diseases. Besides, in the central nervous system, spectrin degradation by calpain is found in the process of injury on neurocyte observed in the traumatic brain injury model. In spinal cord injured model it was recognized that in glia cells calpain messenger RNA increased and its activity increased in the lesion and the possibility was shown that calpain had much to do with the degeneration of myelin and actin after injury (see Brain Res., 816, 375 (1999)). And IL-1β was shown to be concerned with the genesis of multiple sclerosis (see Immunol. Today, 14, 260 (1993)). Therefore, it is conceivable that a cysteine protease inhibitor is promising as an agent for the treatment of these nerve-injuring diseases.

Normally, cathepsin S and cathepsin K do not exist in human arterial walls but it was confirmed that they expressed in arterial sclerosis lesion and they had an decomposing activity of alveolus elastica (see J. Clin. Invest., 102, 576 (1998)) and a calpain inhibitor and antisense of m-calpain inhibited the proliferation of human blood vessel smooth muscle cells and it is shown that m-calpain is concerned with the proliferation of smooth muscle (see Arteioscler. Thromb. Vssc. Biol., 18, 493 (1998)), so it is conceivable that a cysteine protease inhibitor is promising for the treatment of blood vessel lesion such as arteriosclerosis, restenosis after percutaneous transluminal coronary angioplasty (PTCA), etc.

It is reported that in liver, cathepsin B is activated in the process of injuring hepatocyte by bile acid (see J. Clin. Invest., 103, 137 (1999)) and so it is expected that a cysteine protease inhibitor is effective for cholestatic cirrhosis.

In lungs and respiratory system, it is shown that cathepsin S is an enzyme that plays a role in elastin degradation by alveolus macrophages (see J. Biol. Chem., 269, 11530 (1994)), so it is probable that cysteine protease is a cause of pulmonary emphysema. And it is also shown that lung injury (see J. Clin. Invest., 97, 963 (1996)), lung fibrosis (see Cytokine, 5, 57 (1993)) and bronchial asthma (see J. Immunol., 149, 3078 (1992)) are caused by production of IL-1β by caspase-1.

It is pointed out that cysteine protease is also concerned with diseases concerning bones and cartilages. Cathepsin K is specifically recognized in osteoclast and it has a decomposing activity against bone matrix (see J. Biol. Chem., 271, 12517 (1996)), so its inhibitor is expected to show an effect against osteoporosis, arthritis, rheumatoid arthritis, osteoarthritis, hypercalcemia and osteometastasis of cancer, where pathologic bone resorption is recognized. Since IL-1β is shown to be concerned with bone resorption and cartilage degradation, and a caspase-1 inhibitor and IL-1β receptor antagonist inhibit the bone resorption and symptoms of arthritis, a caspase-1 inhibitor and IL-1β receptor antagonist are expected to be effective for arthritis (see Cytokine, 8, 377 (1996)) and osteoporosis (J. Clin. Invest., 93, 1959 (1994)). And it is reported that IL-1β is also concerned with osteoarthritis (see Life Sci., 41, 1187 (1987)).

Cysteine protease is involved with production of various hormones. Since increase of messenger RNA of cathepsin S was recognized by stimuli of thytropin on thyroid epitheliocyte strains (see J. Biol. Chem., 267, 26038 (1992)), it is conceivable that a cysteine protease inhibitor is effective for hyperthyrodism.

Since quantity and activity of cathepsin B protein increased in the gingival sulcus liquid of periodontitis patients (see J. Clin. Periodontol., 25, 34 (1998)), it is pointed out that cysteine protease is concerned with periodontitis.

Therefore, it is expected that the compound that possesses the inhibitory activity of cysteine protease is useful as an agent for the prophylaxis and/or treatment of inflammatory diseases (periodontitis, arthritis, inflammatory bowel diseases, infectious diseases, pancreatitis, hepatitis, glomerulonephritis, endocarditis, myocarditis, etc.), diseases induced by apoptosis (graft versus host diseases, rejection of an organ transplantation, acquired immune deficiency syndrome (AIDS), AIDS-related complex (ARC), adult T cell leukemia, hairy cells leukemia, spondylopathy, disorders of respiratory apparatus, arthritis, HIV or HTLV-1 related diseases such as uveitis, virus-related diseases such as hepatitis C, cancer, collagenosis (systemic lupus erythematosus, rheumatoid arthritis, etc.), ulcerative colitis, Sjögren's syndrome, primary biliary cirrhosis, spontaneous thrombocytopenic purpura, autoimmune hemolytic anemia, myasthenia gravis, autoimmune diseases such as insulin dependent (type I) diabetes, diseases accompanying thrombocytopenia (osteomyelodysplasia syndrome, periodic thrombocytopenia, aplastic anemia, spontaneous thrombocytopenia, disseminated intravascular coagulation (DIC), etc.), hepatic diseases such as viral hepatitis (type A, B, C, F, etc.) or hepatitis medicamentosus and cirrhosis, dementia such as Alzheimer's diseases and Alzheimer's senile dementia, cerebrovascular injury, nerve degeneration diseases, adult acute respiratory distress syndrome, infectious diseases, prostatomegaly, hysteromyoma, bronchial asthma, arteriosclerosis, all kinds of lusus naturae, nephropathy, senile cataract, chronic fatigue syndrome, myodystrophy, peripheral neuropathy, etc.), diseases induced by disorders of immune response (graft versus host diseases, rejection of an organ transplantation, allergic diseases (bronchial asthma, atopic dermatitis, allergic rhinitis, pollinosis, diseases induced by house dusts, irritable pneumonia, food allergy, etc.), psoriasis, rheumatoid arthritis, etc.), autoimmune diseases (insulin-dependent (type I) diabetes, systemic lupus erythematosus, Hashimoto's diseases, multiple sclerosis, etc.), disease by degradation various proteins which compose the organism (myodystrophy, cataract, periodontitis, hepatocyte disease by bile acid such as cholestatic cirrhosis, etc.), decomposition of alveolus elastica such as pulmonary emphysema, ischemic diseases (brain ischemia, brain disorders (encephalopathy) by ischemic reperfusion, myocardial infarction, ischemic hepatopathy, etc.), shock (septic shock, systemic inflammatory response syndrome, endotoxin shock, acidosis, etc.), circulatory system disorders (arteriosclerosis, restenosis after percutaneous transluminal coronary angioplasty (PTCA), etc.)), blood coagulation disorders (thrombocytopenic purpura, hemolytic uremic syndrome, etc.), malignant tumor, acquired immune deficiency syndrome (AIDS) and AIDS-related complex (ARC), parasitic diseases such as malaria, nerve degenerative diseases (Alzheimer-type dementia, Huntington's chorea, Parkinson's diseases, multiple sclerosis, traumatic encephalopathy, traumatic spondylopathy, etc.), pulmopathy such as lung fibrosis, bone resorption diseases (osteoporosis, rheumatoid arthritis, arthritis, osteoarthritis, hypercalcemia, osteometastasis of cancer, etc.), endocrinesthenia such as hyperthyroidism.

On the other hand, what is the most important for inhibitors in inhibiting the activity of proteases is, the special reaction site which interacts with the amino acid residue that is the activity center of proteases. The surrounding structure of the reaction sites are represented by—P3P2P1-P1′P2′P3′—, centering peptide binding (P1-P1′) of the reaction site, and at P1 site there exist amino acid residues fitting the substance specificity of proteases which the inhibitors aim. Some reaction sites against cysteine proteases are known, for example, in the specification of WO99/54317, the followings are described;

P1 position against calpain I, II (norvaline, phenylalanine, etc.),

P1 position against calpain I (arginine, lysine, tyrosine, valine, etc.),

P1 position against papain (homophenylalanine, arginine, etc.),

P1 position against cathepsin B (homophenylalanine, phenylalanine, tyrosine, etc.),

P1 position against cathepsin S (valine, norleucine, phenylalanine, etc.),

P1 position against cathepsin L (homophenylalanine, lysine, etc.),

P1 position against cathepsin K (arginine, homophenylalanine, leucine, etc.),

P1 position against caspase (aspartic acid).

On the other hand, in the specification of WO 98/49190, it is disclosed that the compound of formula (A) or a pharmaceutically acceptable salt thereof has an inhibitory activity against cysteine proteases,

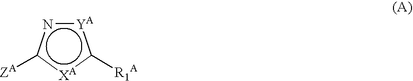

wherein Z^{A} is a cysteine protease binding moiety; X^{A} and Y^{A} are independently S, O or N, said N being optionally substituted with alkyl or alkenyl optionally substituted with 1-3 of halo, or (C5-C6)aryl, arylalkyl or arylalkenyl optionally comprising 1-3 heteroatoms selected from N, O and S, and optionally substituted with halo, cyano, nitro, haloalkyl, amino, aminoalkyl, dialkylamino, alkyl, alkenyl, alkynyl, alkoxy, haloalkoxy, carboxy, carboalkoxy, arylcarboxamide, alkylthio or haloalkylthio; R₁^{A} is alkyl or alkenyl optionally substituted with 1-3 halo or hydroxy; alkylamino, dialkylamino, alkyldialkylamino; or cycloalkyl, alkylcycloalkyl, (C5-C12)aryl, (C5-C12)arylalkyl or (C5-C12)arylalkenyl optionally comprising 1-4 heteroatoms selected from N, O and S, and optionally substituted with halo, cyano, nitro, haloalkyl, amino, aminodialkyl, dialkylamino, alkyl, alkenyl, alkynyl, alkoxy, haloalkyl, carboxyl, carboalkoxy, alkylcarboxamide, (C5-C6)aryl, —O—(C5-C6)aryl, arylcarboxamide, alkylthio or haloalkylthio; and at least one of Y or X is N.

### DISCLOSURE OF THE INVENTION

The present inventors have energetically investigated to find out such compounds that have cysteine protease inhibitory activity and found that the oxadiazole derivative of formula (I) of the present invention accomplishes the purpose:

The oxadiazole derivative of formula (I) of the present invention is not known at all as a cysteine protease inhibitor at all.

The present invention relates to

1) an oxadiazole derivative of formula (I),

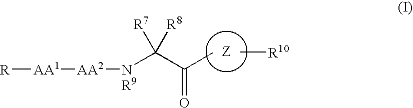

(i) hydrogen,

(ii) C1-8 alkyl,

(iii) CycA,

(iv) C1-8 alkyl substituted with a group selected from halo, CycA, nitro, CF₃ and cyano,

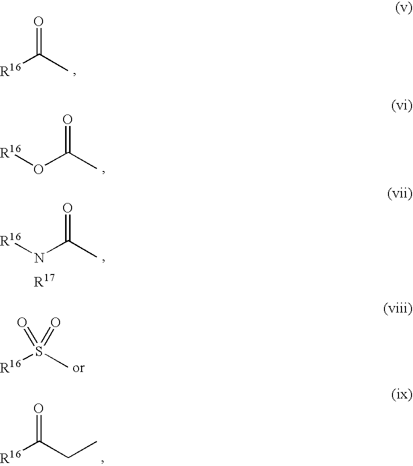

CycA is a mono-, bi- or tri-cyclic C3-15 carboring, or a mono-, bi- or tri-cyclic 3-15 membered heteroring comprising 1-4 of nitrogen, 1-2 of oxygen and/or 1 of sulfur,

R¹⁶ is

(1) C1-8 alkyl,

(2) C2-8 alkenyl,

(3) C2-8 alkynyl,

(4) CycA, or

(5) C1-8 alkyl, C2-8 alkenyl or C2-8 alkynyl substituted with 1-5 of halogen, nitro, trifluoromethyl, cyano, CycA, NR¹⁸R¹⁹, —OR¹⁸, —NHC(O) —CycA and —NHC(O)O—(C1-8 alkyl),

R¹⁷, R¹⁸ and R¹⁹ are each independently, hydrogen, C1-4 alkyl, CycA or C1-4 alkyl substituted with C1-4 alkyl,

AA¹ is

(i) a single bond, or

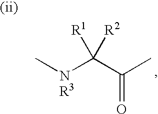

wherein R¹ and R² are the same or different to represent

(i) hydrogen,

(ii) C1-8 alkyl,

(iii) CycA or

(iv) C1-8 alkyl substituted with 1-5 of group selected from the following (1) to (8):

(1) —NR²¹R²²,

(2) —OR²³

(3) —SR²⁴,

(4) —COR²⁵,

(5) —NR²⁶CONR²¹R²²,

(6) guanidino,

(7) CycA,

(8) —NR²⁶SO₂R²¹; or

R¹ and R² are taken together to form C2-8 alkylene wherein one carbon atom may be replaced by oxygen, sulfur or —NR²⁰— and the alkylene may be substituted with —NR²¹R²² or —OR²³,

R²⁰ is hydrogen, C1-4 alkyl, —COO—(C1-4 alkyl), phenyl or C1-4 alkyl substituted with phenyl,

R²¹, R²², R²³, R²⁴ and R²⁶ are the same or different to represent hydrogen, C1-4 alkyl, phenyl or C1-4 alkyl substituted with phenyl,

R²⁵ is C1-4 alkyl, phenyl, —NR²¹R²² wherein all symbols have the same meaning as above, —OR²³ wherein R²³ is the same meaning as above, or C1-4 alkyl substituted with phenyl,

R³ is hydrogen, C1-8 alkyl, phenyl or C1-8 alkyl substituted with phenyl or

R³ is taken together with R¹ to form C2-6 alkylene wherein one carbon atom may be replaced by oxygen, sulfur or —NR²— and the alkylene may be substituted with —NR²¹R²² or —OR²³, or

when AA¹ is

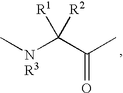

AA¹ and R may be taken together to form

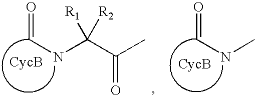

wherein

is a 5-12 membered mono- or bi-cyclic heteroring and the other symbols are the same meanings as above,

AA² is

(i) a single bond,

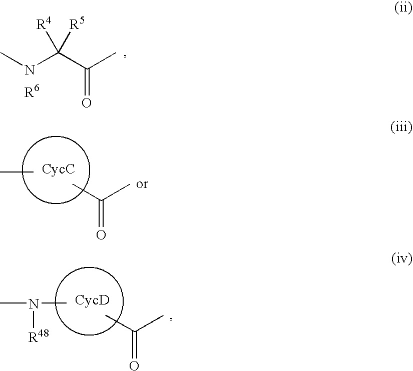

wherein R⁴ and R⁵ are the same or different to represent

(1) hydrogen,

(2) C1-8 alkyl,

(3) CycA or

(4) C1-8 alkyl substituted with 1-5 of group selected from the following (a) to (h):

(a) —NR⁴¹R⁴²,

(b) —OR⁴³,

(c) —SR⁴⁴,

(d) —COR⁴⁵,

(e) —NR⁴⁶CONR⁴¹R⁴²,

(f) guanidino,

(g) CycA,

(h) —NR⁴⁶SO₂R⁴′; or

R⁴ and R⁵ are taken together to form C2-8 alkylene wherein one carbon atom may be replaced by oxygen, sulfur or —NR⁴⁰— and the alkylene may be substituted with —NR⁴¹R⁴² or —OR⁴³,

R⁴⁰ is hydrogen, C1-4 alkyl, —COO-(C1-4 alkyl), phenyl or C1-4 alkyl substituted with phenyl,

R⁴¹, R⁴², R⁴³, R⁴⁴ and R⁴⁶ are the same or different to represent hydrogen, C1-4 alkyl, phenyl or C1-4 alkyl substituted with phenyl,

R⁴⁵ is C1-4 alkyl, phenyl, —NR⁴¹R⁴² wherein all symbols are the same meaning as above, —OR⁴³ wherein R⁴³ has the same meaning as above, or C1-4 alkyl substituted with phenyl,

R⁶ is hydrogen, C1-8 alkyl, phenyl or C1-8 alkyl substituted with phenyl or

R⁶ is taken together with R⁴ to form C2-6 alkylene wherein one carbon atom may be replaced by oxygen, sulfur or —NR⁴⁰— and the alkylene may be substituted with —NR⁴¹R⁴² or —OR⁴³

R⁴⁸ is hydrogen, C1-4 alkyl, phenyl or C1-4 alkyl substituted with phenyl or when AA¹ is a single bond, R⁴⁸ and R may be taken together to form C2-6 alkylene wherein one carbon atom may be replaced by oxygen, sulfur or —NR⁴⁷ wherein R⁴⁷ is hydrogen or C1-4 alkyl,

CycC is a 3-17 membered mono- or bi-cyclic heteroring, CycD is a C3-14 mono- or bi-cyclic carboring or a 3-14 membered mono- or bi-cyclic heteroring, or

AA² and AA¹ are taken together to form,

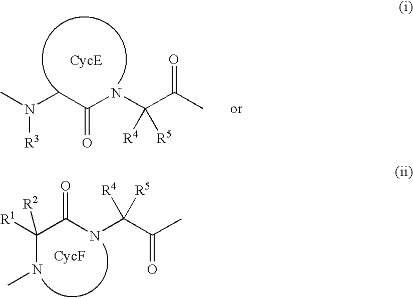

wherein CycE is a 4-18 membered mono- or bi-cyclic heteroring, CycF is a 5-8 membered monocyclic heteroring, and the other symbols have the same meanings as above,

R⁷ and R⁸ are the same or different to represent

(i) hydrogen,

(ii) C1-8 alkyl,

(iii) CycA or

(iv) C1-8 alkyl substituted with 1-5 of group selected from the following (1) to (8);

(1)—NR⁶¹R⁶²,

(2)—OR⁶³,

(3) —SR⁶⁴,

(4) —COR⁶⁵,

(5) —NR⁶⁶CONR⁶¹R⁶²,

(6) guanidino,

(7) CycA,

(8) —NR⁶⁶SO₂R⁶¹, or

R⁷ and R⁸ are taken together to form C2-8 alkylene wherein one carbon atom may be replaced by oxygen, sulfur or —NR⁶⁰— and the alkylene may be substituted with —NR⁶¹R⁶² or —OR⁶³,

R⁶⁰ is hydrogen, C1-4 alkyl, —COO—(C1-4 alkyl), phenyl or C1-4 alkyl substituted with phenyl,

R⁶¹, R⁶¹, R⁶³, R⁶⁴ and R⁶⁶ are the same or different to represent hydrogen, C1-4 alkyl, phenyl or C1-4 alkyl substituted with phenyl, R⁶⁵ is C1-4 alkyl, phenyl, —NR⁶¹R⁶² wherein all symbols are the same meanings as above, —OR⁶³ wherein R⁶³ is the same meaning as above, or C1-4 alkyl substituted with phenyl,

R⁹ is hydrogen, C1-8 alkyl, phenyl or C1-8 alkyl substituted with phenyl or

R⁹ is taken together with R⁷ to form C2-6 alkylene wherein one carbon atom may be replaced by oxygen, sulfur or —NR⁶⁰— and the alkylene may be substituted with —NR⁶¹R⁶² or —OR⁶³,

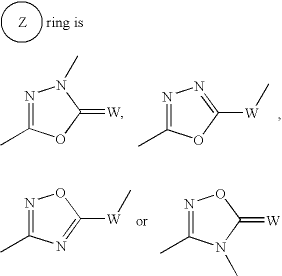

wherein W is oxygen or sulfur,

R¹⁰ is

(i) C1-8 alkyl,

(ii) C2-8 alkenyl,

(iii) CycA,

(iv) —COR⁷¹, or

(v) C1-8 alkyl substituted with 1-3 of CycA, guanidino, —COR⁷¹, —NR⁷²R⁷³, —OR⁷⁴, cyano, —P(O)(OR⁷⁸)₂ and —O—(C1-4 alkylene)-(C1-4 alkoxy),

wherein R⁷¹ is

(1) C1-4 alkyl,

(2) C1-4 alkoxy,

(3) CycA,

(4) —O-CycA,

(5) —NR⁷²R⁷³,

(6) C1-4 alkyl substituted with CycA,

(7) C1-4 alkoxy substituted with CycA or

(8) hydroxy,

R⁷² and R⁷³ are the same or different to represent

(1) hydrogen,

(2) C1-8 alkyl,

(3) C1-8 alkoxy,

(4) C2-8 acyl,

(5) C2-8 alkoxycarbonyl,

(6) CycA,

(7) —C(O)CycA,

(8) —SO₂CycA or

(9) C1-8 alkyl substituted with CycA, —C(O)CycA, —SO₂CycA, C1-8 alkoxy, C2-8 acyl or C2-8 alkoxycarbonyl, R⁷⁴ is

(1) hydrogen,

(2) C1-8 alkyl,

(3) CycA,

(4) C1-8 alkyl substituted with —SiR⁷⁵R⁷⁶R⁷⁷ wherein R⁷⁵, R⁷⁶ and R⁷⁷ are the same or different to represent C1-8 alkyl, phenyl or C1-8 alkyl substituted with phenyl, or

(5) C1-8 alkyl substituted with CycA,

R⁷⁸ is C1-8 alkyl, phenyl or C1-8 alkyl substituted with phenyl;

wherein CycA in R, R¹, R², R⁴, R⁵, R⁷, R⁸, R¹⁶ may be the same or different and CycA, CycB, CycC, CycD, CycE and CycF may be independently substituted with 1-5 of R²⁷;

R²⁷ is

(1) C1-8 alkyl,

(2) halo,

(3) —NR¹¹, R¹²,

(4) —OR¹³,

(5) —SR¹⁴,

(6) CycG,

(7) nitro,

(8) cyano,

(9) oxo,

(10) —COR¹⁵,

(11) —SO₂R¹⁵, or

(12) C1-8 alkyl substituted with 1-5 of the following (a) to (j):

(a) halo,

(b) —NR¹¹R¹²,

(c) —OR¹³,

(d) —SR¹⁴,

(e) CycG,

(f) nitro,

(g) cyano,

(h) —COR¹⁵, or

(j) —SO₂R¹⁵,

wherein R¹¹ and R¹² are the same or different to represent hydrogen, C1-4 alkyl, C1-4 alkoxy, —C(O)O—(C1-4 alkyl), CycG, or C1-4 alkyl substituted with CycG,

R¹³ and R¹⁴ are the same or different to represent hydrogen, C1-4 alkyl, trifluoromethyl, CycG, or C1-4 alkyl substituted with CycG,

CycG is a 5-8 membered mono- or bi-cyclic carboring or a 5-8 membered mono- or bi-cyclic heteroring,

R¹⁵ is C1-4 alkyl, CycG, —NR¹¹R¹² wherein all symbols have the same meanings as above, —OR¹³ wherein R¹³ has the same meaning as above, or C1-4 alkyl substituted with CycG, —NR¹¹R¹² wherein all symbols have the same meanings as above or —OR¹³ wherein R¹³ has the same meaning as above,

2) a method for the preparation thereof and

3) a pharmaceutical composition comprising the same as active ingredient.

In the compound of formula (I), in

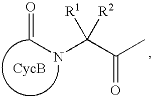

wherein AA¹ and R together form,

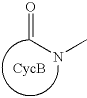

is a 5-12 membered heteroring comprising 1-3 of nitrogen, 1 of oxygen, and/or 1 of sulfur (this heteroring may be substituted with 1-5 of R²⁷).

And to describe

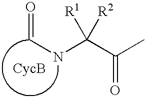

concretely, it is

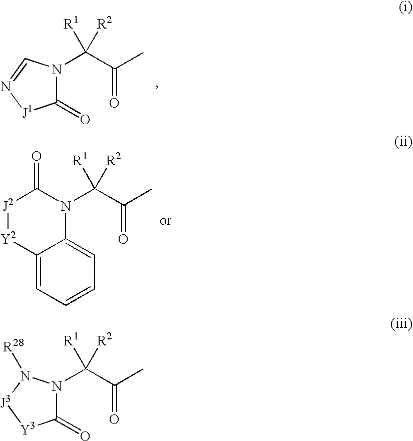

wherein J¹ is oxygen, sulfur, —NR²⁹— (wherein R²⁹ is hydrogen, C1-4 alkyl, CycA or C1-4 alkyl substituted with CycA), C1-3 alkylene or C2-3 alkenylene,

J² is a single bond or C1-2 alkylene,

Y² is —N═CH—, —CH═N— or C1-2 alkylene,

J³ is carbonyl or C1-3 alkylene,

Y³ is C1-3 alkylene, oxygen or —NR²⁹— wherein R²⁹ is the same meaning as above,

R²⁸ is hydrogen, C1-4 alkyl, CycA or C1-4 alkyl substituted with CycA, or

R²⁸ is taken together with R¹ to form C2-4 alkylene, and the other symbols have the same meaning as above and each ring may be substituted with 1-5 of R²⁷.

In the compound of formula (I), in

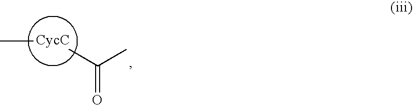

wherein AA² represents, CycC is a 3-17 membered heteroring which comprises 1-2 of nitrogen, 1 of oxygen and/or 1 of sulfur (this ring may be substituted with 1-5 of R²⁷).

And to describe

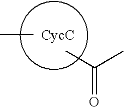

concretely,

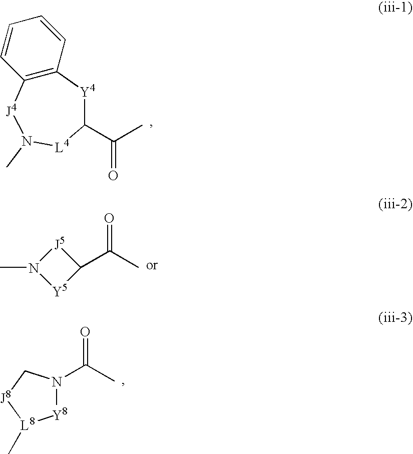

wherein J⁴, Y⁴ and L⁴ are the same or different to represent a single bond or C1-3 alkylene, wherein J⁴, Y⁴ and L⁴ do not represent a single bond at the same time, J⁵ is C1-6 alkylene,

Y⁵ is a single bond, C1-3 alkylene or —NR⁶⁷—, wherein R⁶⁷ is hydrogen, C1-4 alkyl, phenyl or C1-4 alkyl substituted with phenyl,

J⁸ is C1-5 alkylene, wherein one carbon atom may be replaced by oxygen,

Y⁸ is a single bond or C1-4 alkylene,

L⁸ is —N— or —CH—, and

the other symbols have the same meaning as above and each ring may be substituted with 1-5 of R)₇.

And in

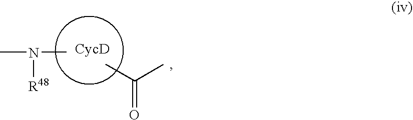

wherein AA² represents, CycD is a C3-14 mono- or bi-cyclic carboring or 3-14 membered heteroring which comprises 1-2 of nitrogen, 1 of oxygen and/or 1 of sulfur (this carboring and heteroring may be substituted with 1-5 of R²⁷).

And to describe

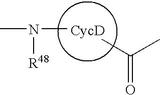

concretely, it is

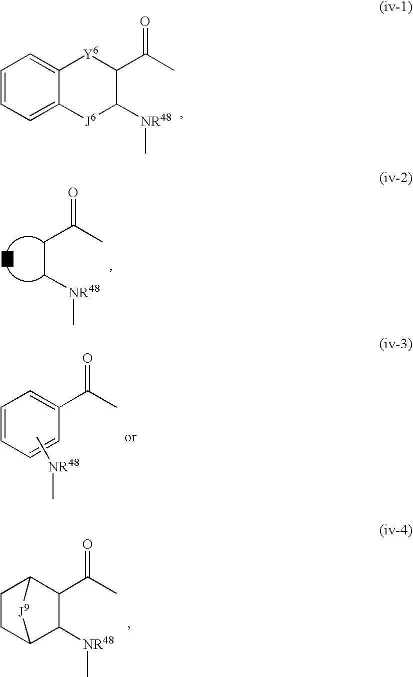

wherein J⁶ and Y⁶ are the same or different to represent a single bond or C1-3 alkylene, wherein J⁶ and Y⁶ do not represent a single bond at the same time,

J⁷ is C1-6 alkylene, wherein one carbon atom may be replaced by oxygen, sulfur or —NR⁶⁷, wherein R⁶⁷ has the same meaning as above,

J⁹ is C1-3 alkylene, oxygen, sulfur or —NR⁶⁷, wherein R⁶⁷ is the same meaning as above, and

the other symbols have the same meanings as above and each ring may be replaced by 1-5 of R²⁷.

In the compounds of the formula (I), in

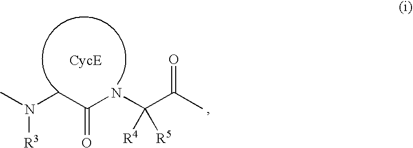

wherein AA¹ and AA² together form,

CycE is a 4-18 membered heteroring which comprises 1-2 of nitrogen, 1 of oxygen and/or 1 of —S(O)ₚ— (this heteroring may be substituted with 1-5 of R²⁷).

And to describe

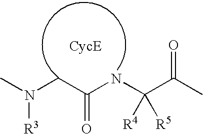

concretely, it is

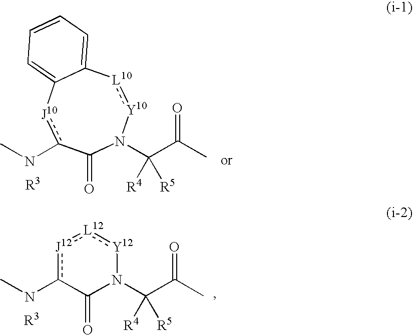

wherein [symbol] is a single bond or a double-bond,

J¹⁰ and Y¹⁰ are the same or different to represent a single bond or C1-3 alkylene,

L¹⁰ is a single bond, C1-3 alkylene, —NR⁵⁷—, wherein R⁵⁷ is hydrogen, C1-4 alkyl, phenyl or C1-4 alkyl substituted with phenyl, —N═, oxygen or —S(O)ₚ—, wherein p is 0 or an integer of 1 to 2,

J¹² and Y¹² are the same or different to represent a single bond or C1-3 alkylene,

L¹² is C1-3 alkylene, —NR⁵⁷-, wherein R⁵⁷ is the same meaning as above), —N═, ═N—, oxygen or —S(O)ₚ—, wherein p has the same meaning as above, and

the other symbols have the same meanings as above and each ring may be substituted with 1-5 of R²⁷.

And in

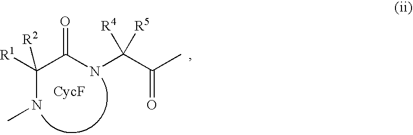

wherein AA¹ and AA² together form, CycF is a 5-8 membered heteroring comprising 2 of nitrogen.

And to describe

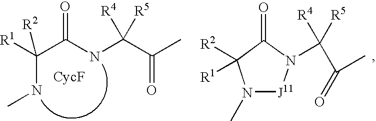

concretely, it is

wherein J¹¹ is carbonyl or C2-4 alkylene and the other symbols have the same meaning as above and the ring therein may be substituted with 1-5 of R²⁷.

In the present specification, C1-4 alkyl is methyl, ethyl, propyl, butyl and isomers thereof.

In the present specification, C1-8 alkyl is methyl, ethyl, propyl, butyl, pentyl, hexyl, heptyl, octyl and isomers thereof.

In the present specification, C1-4 alkoxy is methoxy, ethoxy, propoxy, butoxy and isomers thereof.

In the present specification, C1-8 alkoxy is methoxy, ethoxy, propoxy, butoxy, pentyloxy, hexyloxy, heptyloxy, oxtyloxy and isomers thereof.

In the present specification, C2-8 alkenyl is, ethyl, propyl, butyl, pentyl, hexyl, heptyl, octyl containing 1-3 of double bond and isomers thereof. For example, vinyl, propenyl, butenyl, hexenyl, hexadienyl, octadienyl, etc. are included.

In the present specification, C2-8 alkynyl is ethyl, propyl, butyl, pentyl, hexyl, heptyl, octyl containing 1-3 of triple bond and isomers thereof. For example, ethynyl, propynyl, butynyl, pentynyl, hexynyl, heptynyl, octynyl, etc. are included.

In the present specification, C1-4 alkyl substituted with phenyl is phenylmethyl, phenylethyl, phenylpropyl, phenylbutyl and isomers thereof.

In the present specification, C1-2 alkylene is, methylene, ethylene and isomers thereof.

In the present specification, C1-3 alkylene is, methylene, ethylene, trimethylene and isomers thereof.

In the present specification, C1-4 alkylene is methylene, ethylene, trimethylene, tetramethylene and isomers thereof.

In the present specification, C1-5 alkylene is methylene, ethylene, trimethylene, tetramethylene, pentamethylene and isomers thereof.

In the present specification, C1-6 alkylene is methylene, ethylene, trimethylene, tetramethylene, pentamethylene, hexamethylene and isomers thereof.

In the present specification, C2-4 alkylene is ethylene, trimethylene, tetramethylene and isomers thereof.

In the present specification, C2-6 alkylene is ethylene, trimethylene, tetramethylene, pentamethylene, hexamethylene and isomers thereof.

In the present specification, C2-8 alkylene is ethylene, trimethylene, tetramethylene, pentamethylene, hexamethylene, heptamethylene, octamethylene and isomers thereof.

In the present specification, C2-6 alkylene whose one carbon atom may be replaced by oxygen, sulfur, —NR²⁰, —NR⁴⁰— or —NR⁶⁰— is ethylene, trimethylene, tetramethylene, pentamethylene, hexamethylene and isomers thereof, wherein one carbon atom thereof may be replaced by oxygen, sulfur, —NR²⁰—, —NR⁴⁰—, or —NR⁶⁰—, for example, such groups are —CH₂—O—CH₂—, —CH₂—CH₂—O—CH₂—, —CH₂—CH₂—S—CH₂—, —CH₂—CH₂—NH—CH₂—, —CH₂—CH₂—O—CH₂—CH₂—, —CH₂—CH₂—S—CH₂—CH₂—, —CH₂—CH₂—NH—CH₂—CH₂—, —CH₂—CH₂—N(CH₃)—CH₂—CH₂—, etc.

In the present specification, C2-8 alkylene whose one carbon atom may be replaced by oxygen, sulfur, NR²⁰, —NR⁴⁰— or —NR⁶⁰— is ethylene, trimethylene, tetramethylene, pentamethylene, hexamethylene, heptamethylene, octamethylene and isomers thereof, wherein one carbon atom may be replaced by oxygen, sulfur, —NR²⁰—, —NR⁴⁰— or —NR⁶⁰—, for example, such groups are —CH₂—O—CH₂—, —CH₂—CH₂—O—CH₂—, —CH₂—CH₂—S—CH₂—, —CH₂—CH₂—NH—CH₂—, —CH₂—CH₂—O—CH₂—CH₂—, —CH₂—CH₂—S—CH₂—CH₂—, —CH₂—CH₂—NH—CH₂—CH₂—, —CH₂—CH₂—N(CH₃)—CH₂—CH₂—, etc.

In the present specification, C2-3 alkenylene means vinylene and allylene and isomers thereof.

In the present specification, halo means chlorine, fluorine, bromine and iodine atom.

In the present specification, mono- or bi-cyclic C5-10 carboring is mono- or bi-cyclic C5-10 carboaryl or partially or completely saturated one thereof. For example, cyclopentane, cyclohexane, cycloheptane, cyclopentene, cyclohexene, cyclopentadiene, cyclohexadiene, benzene, pentalene, indene, naphthalene, azulene, perhydropentalene, perhydroindene, perhydronaphthalene, perhydroazulene, adamantane ring, etc. are included.

In the present specification, mono-, bi- or tri-cyclic C3-15 carboring is mono-, bi- or tri-cyclic carboaryl or partially or completely saturated one thereof. For example, cyclopropane, cyclobutane, cyclopentane, cyclohexane, cycloheptane, cyclopentene, cyclohexene, cyclopentadiene, cyclohexadiene, benzene, pentalene, indene, naphthalene, azulene, fluorene, phenanthrene, anthracene, acenaphthylene, biphenylene, perhydropentalene, perhydroindene, perhydronaphthalene, perhydroazulene, perhydrofluorene, perhydrophenanthrene, perhydroanthracene, perhydroacenaphthylene, perhydrobiphenylene, adamantyl ring etc. are included.

In the present specification, mono- or bi-cyclic 5-10 membered heteroring comprising 1-4 of nitrogen, 1 of oxygen and/or sulfur is mono- or bi-cyclic 5-10 membered heteroaryl comprising 1-4 of nitrogen, 1 of oxygen and/or sulfur or partially or completely saturated one thereof.

Above 5-10 membered mono- or bi-cyclic heteroaryl comprising 1-4 of nitrogen, 1 of oxygen and/or 1 of sulfur is, for example, pyrrole, imidazole, triazole, tetrazole, pyrazole, pyridine, pyrazine, pyrimidine, pyridazine, azepine, diazepine, furan, pyrane, oxepine, thiophene, thiaine (thiopyrane), thiepine, oxazole, isooxazole, thiazole, isothiazole, oxadiazole, oxazine, oxadiazine, oxazepine, oxadiazepine, thiadiazole, thiazine, thiadiazine, thiazepine, thiadiazepine, indole, isoindole, benzofuran, isobenzofuran, benzothiophene, isobenzothiophene, indazole, quinoline, isoquinoline, phthalazine, naphthyridine, quinoxaline, quinazoline, cinnoline, benzoxazole, benzothiazole, benzoimidazole, etc.

Above partially or completely saturated mono- or bi-cyclic 5-10 membered heteroaryl comprising 1-4 of nitrogen, 1 of oxygen and/or 1 of sulfur is, for example, pyrroline, pyrrolidine, imidazoline, imidazolidine, triazoline, triazolidine, tetrazoline, tetrazolidine, pyrazoline, pyrazolidine, piperidine, piperazine, tetrahydropyridine, tetrahydropyrimidine, tetrahydropyridazine, dihydrofuran, tetrahydrofuran, dihydropyrane, tetrahydropyrane, dihydrothiophene, tetrahydrothiophene, dihydrothiaine (dihydrothiopyrane), tetrahydrothiaine (tetrahydrothiopyrane), oxazoline (dihydrooxazole), oxazolidine (tetrahydroxazole), dihydroisoxazole, tetrahydroisoxazole, oxadiazoline (dihydroxadiazole), oxadiazolidine (tetrahydroxadiazole), thiazoline (dihydrothiazole), thiazolidine (tetrahydrothiazole), dihydroisothiazole, tetrahydroisothiazole, morpholine, thiomorpholine, indoline, isoindoline, dihydrobenzofuran, perhydrobenzofuran, dihydroisobenzofuran, perhydroisobenzofuran, dihydrobenzothiophene, perhydrobenzothiophene, dihydroisobenzothiophene, perhydroisobenzothiophene, dihydroindazole, perhydroindazole, dihydroquinoline, tetrahydroquinoline, perhydroquinoline, dihydroisoquinoline, tetrahydroisoquinoline, perhydroisoquinoline, dihydrophthalazine, tetrahydrophthalazine, perhydrophthalazine, dihydronaphthyridine, tetrahydronaphthyridine, perhydronaphthyridine, dihydroquinoxaline, tetrahydroquinoxaline, perhydroquinoxaline, dihydroquinazoline, tetrahydroquinazoline, perhydroquinazoline, dihydrocinnoline, tetrahydrocinnoline, perhydrocinnoline, dihydrobenzoxazole, perhydrobenzoxazole, dihydrobenzothiazole, perhydrobenzothiazole, dihydrobenzoimidazole, perhydrobenzoimidazole, etc.

In the present specification, a 3-15 membered mono-, bi- or tri-cyclic heteroring comprising 1-4 of nitrogen, 1-2 of oxygen and/or 1 of sulfur is 3-15 membered mono-, bi- or tri-cyclic heteroaryl comprising 1-4 of nitrogen, 1-2 of oxygen and/or 1 of sulfur or partially or completely saturated one thereof.

Above 3-15 membered mono-, bi- or tri-cyclic heteroring comprising-1-4 of nitrogen, 1-2 of oxygen and/or 1 of sulfur is, for example, pyrrole, imidazole, triazole, tetrazole, pyrazole, pyridine, pyrazine, pyrimidine, pyridazine, azepine, diazepine, furan, pyrane, oxepine, oxazepine, thiophene, thiaine (thiopyrane), thiepine, oxazole, isoxazole, thiazole, isothiazole, oxadiazole, oxazine, oxadiazine, oxazepine, oxadiazepine, thiadiazole, thiazine, thiadiazine, thiazepine, thiadiazepine, indole, isoindole, benzofuran, isobenzofuran, benzothiophene, isobenzothiophene, indazole, quinoline, isoquinoline, phthalazine, naphthyridine, quinoxaline, quinazoline, cinnoline, benzoxazole, benzoxadiazole, benzothiazole, benzoimidazole, carbazole, acridine ring, etc.

Above partially or completely saturated mono-, bi- or tri-cyclic 5-15 membered heteroring comprising 1-4 of nitrogen, 1-2 of oxygen and/or 1 of sulfur is, aziridine, oxirane, azetidine, oxetane, thiirane, thietane, pyrroline, pyrrolidine, imidazoline, imidazolidine, triazoline, triazolidine, tetrazoline, tetrazolidine, pyrazoline, pyrazolidine, piperidine, piperazine, tetrahydropyridine, tetrahydropyrimidine, tetrahydropyridazine, dihydrofuran, tetrahydrofuran, dihydropyrane, tetrahydropyrane, dihydrothiophene, tetrahydrothiophene, dihydrothiaine (dihydrothiopyrane), tetrahydrothiaine (tetrahydrothiopyrane), oxazoline (dihydroxazole), oxazolidine (tetrahydroxazole), dihydroisoxazole, tetrahydroisoxazole, oxadiazoline (dihydroxadiazole), oxadiazolidine (tetrahydroxadiazole), thiazoline (dihydrothiazole), thiazolidine (tetrahydrothiazole), dihydroisothiazole, tetrahydroisothiazole, morpholine, thiomorpholine, indoline, isoindoline, dihydrobenzofuran, perhydrobenzofuran, dihydroisobenzofuran, perhydroisobenzofuran, dihydrobenzothiophene, perhydrobenzothiophene, dihydroisobenzothiophene, perhydroisobenzothiophene, dihydroindazole, perhydroindazole, dihydroquinoline, tetrahydroquinoline, perhydroquinoline, dihydroisoquinoline, tetrahydroisoquinoline, perhydroisoquinoline, dihydrophthalazine, tetrahydrophthalazine, perhydrophthalazine, dihydronaphthyridine, tetrahydronaphthyridine, perhydronaphthyridine, dihydroquinoxaline, tetrahydroquinoxaline, perhydroquinoxaline, dihydroquinazoline, tetrahydroquinazoline, perhydroquinazoline, dihydrocinnoline, tetrahydrocinnoline, perhydrocinnoline, dihydrobenzoxazole, perhydrobenzoxazole, dihydrobenzothiazole, perhydrobenzothiazole, dihydrobenzoimidazole, perhydrobenzoimidazole, benzoxazepine, benzoxadiazepine, benzothiazepine, benzothiadiazepine, benzazepine, benzodiazepine, indoloxazepine, indolotetrahydroxazepine, indoloxadiazepine, indolotetrahydroxadiazepine, indolothiazepine, indolotetrahydrothiazepine, indolothiadiazepine, indolotetrahydrothiadiazepine, indolazepine, indolotetrahydroazepine, indolodiazepine, indolotetrahydrodiazepine, benzofurazane, benzothiadiazole, benzotriazole, camphor, imidazothiazole, dihydrocarbazole, tetrahydrocarbazole, perhydrocarbazole, dihydroacridine, tetrahydroacridine, perhydroacridine, dioxolane, dioxane, dioxazine ring etc.

In the present specification, a 5-12 membered heteroring comprising 1-3 of nitrogen, 1 of oxygen and/or 1 of sulfur atom, i.e.

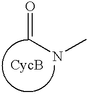

is, for example, a ring represented by

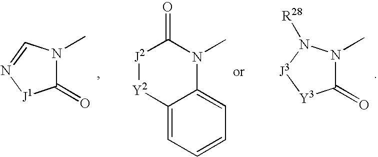

Specifically, 2-oxo-1,3,4-triazoline, 5-oxo-1,2,4-oxadiazoline, 5-oxo-1,2,4-thiadiazoline, 4-oxoimidazoline, 3,4-dihydro-4-oxopyrimidine, 3,4,5,6-tetrahydro-4-oxopyrimidine, 2-oxoindoline, 2-oxo-tetrahydroquinoline, 1,2-dihydro-2-oxoquinazoline, 1,2-dihydro-2-oxoquinoxaline, 3-oxopyrazolidine, perhydro-3-oxopyridazine, 2-oxo-1,3,4-oxadiazolidine, perhydro-2-oxo-1,3,4-oxadiazine, etc. are included.

In the specification, 3-17 membered heteroring comprising 1-2 of nitrogen, 1 of oxygen and/or 1 of sulfur represented by CycC is, for example, a ring represented by

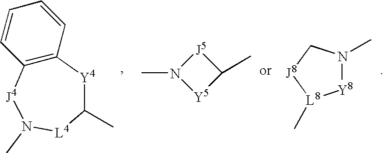

Specifically, pyrrolidine, imidazolidine, pyrazolidine, piperidine, piperazine, perhydropyrimidine, perhydropyridazine, thiazolidine, indoline, isoindoline, tetrahydroquinoline, tetrahydroisoquinoline, etc. are included.

In the specification, a C3-14 mono- or bi-cyclic carboring or 3-14 membered heteroring comprising 1-2 of nitrogen, 1 of oxygen, and/or 1 of sulfur represented by CycD is, for example, a ring represented by

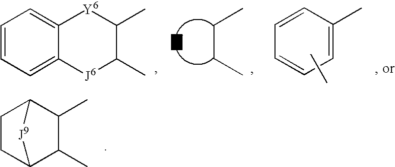

Specifically, cyclopentane, cyclohexane, cycloheptane, benzene, indan, tetrahydronaphthalene, oxorane, oxane, thiorane, thian, pyrrolidine, piperidine, bicyclo[2.2.1]heptane, bicyclo[2.2.2]octane, 7-azabicyclo[2.2.1]heptane, 7-oxobicyclo[2.2.1]heptane, 7-thiabicyclo[2.2.1]heptane, etc. are included.

In the specification, 4-18 membered heteroring comprising 1-2 of nitrogen, 1 of oxygen and/or 1 of —S(O)ₚ—, i.e. CycE is, for example, a ring represented by

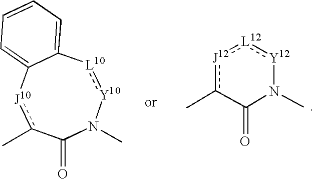

Specifically, 2-oxopyrrolidine, 2-oxopiperidine, 2-oxoperhydroazepine, 2-oxopiperazine, 3-oxomorpholine, 1,1-dioxo-3-isothiazolidine, 1,1-dioxo-3-isothiazine, 4-oxodiazepine, 2-oxoindoline, 2-oxo-tetrahydroquinoline, 1,1-dioxo-3-benzisothiazolidine, 1, 1-dioxo-3-benzisothiazine, etc. are included.

In the present invention, 5-8 membered heteroring which comprises 2 of nitrogen. i.e. CycF is, for example, a ring represented by

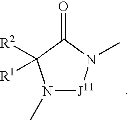

Specifically, 2,4-dioxoimidazolidine, 2-oxopiperazine, 2-oxoperhydrodiazepine substituted by R¹ and R² are included.

In the present invention, as may be easily understood by those skilled in the art, the symbol:

indicates that the substituent attached thereto is in front of the sheet (β-position) unless specified, indicates that the substituent attached thereto is behind the sheet (β-position) unless specified, and indicates that the substituent attached thereto is in β-position or α-position or a mixture thereof.

In the formula (I), all groups represented by R are preferable, and preferably,

R is

(i) hydrogen,

(ii) C1-8 alkyl,

(iii) CycA,

(iv) C1-8 alkyl substituted with a group selected from CycA and nitro,

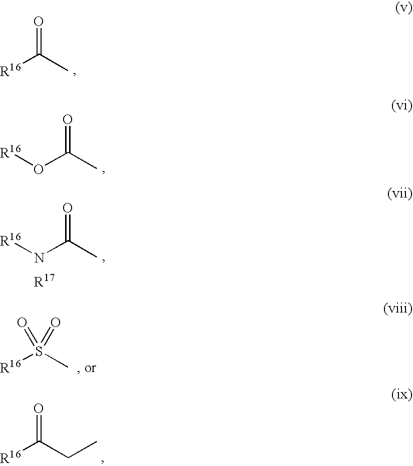

more preferably, C1-8 alkyl or C1-8 alkyl substituted with CycA or nitro.

R¹⁶ is all preferable, but more preferably, R¹⁶ is

[I] (1) C1-8 alkyl,

(2) C2-8 alkenyl,

(3) C2-8 alkynyl,

(4) CycA, or

(5) C1-8 alkyl substituted with a group selected from CycA or —NHC(O) —CycA,

(6) C2-8 alkenyl substituted with CycA or

(7) C2-8 alkynyl substituted with CycA,

wherein CycA may be substituted with 1-5 of R^{27a}, and

R^{27a} is

(1) C1-8 alkyl,

(2) halogen,

(3) —NR¹¹R¹²,

(4) —OR³,

(5) phenyl,

(6) nitro,

(7) CF₃,

(8) cyano,

(9) tetrazole,

(10) —SR¹⁴,

(11)—COR⁵,

(12) oxo or

(13) C1-8 alkyl substituted with 1-5 of group selected from the following (a) to (k): (a) halogen, (b)-NR¹¹R¹², (c) —OR³, (d) phenyl, (e) nitro, (f) CF₃, (g) cyano, (h) tetrazole, (j) —SR⁴, (k) —COR¹⁵; or

[II] (a) C1-8 alkyl, C2-8 alkenyl or C2-8 alkynyl substituted with a group selected from halogen, CF₃, nitro, cyano or NR¹⁸R¹⁹ or

(b) (1) CycA containing 1-5 of substituent R²⁷ or

(2) C1-8 alkyl, C2-8 alkenyl or C2-8 alkynyl substituted with CycA, which contains 1-5 of substituent R²⁷,

wherein at least one of R²⁷ described in (1) and (2) is selected from

(i) a C5-10 mono- or bi-cyclic carboring,

(ii) a 5-10 membered mono- or bi-cyclic heteroring,

(iii) —SO₂R¹⁵,

(iv) —OCF₃ or

(v) C1-8 alkyl substituted with 1-5 of the group selected from (a) halogen, (b) —NR¹¹R¹², (c) —OR³, (d) a C5-10 mono-or bi-cyclic carboring, (e) nitro, (f) CF₃, (g) cyano, (h) a 5-10 membered mono- or bi-cyclic heteroring, (j)-SR¹⁴, (k) —COR¹⁵, (1)—SO₂R¹⁵ and (m) —OCF₃ (at least one is a C5-10 mono-or bi-cyclic carboring, a 5-10 mono- or bi-cyclic heteroring, —SO₂R¹⁵ or —OCF₃)).

Particularly preferably,

[I] (1) C1-8 alkyl,

(2) C2-8 alkenyl,

(3) C2-8 alkynyl,

(4) CycA or

(5) C1-8 alkyl substituted with a group selected from CycA or —NHC(O)—CycA,

(6) C2-8 alkenyl substituted with CycA or

(7) C2-8 alkynyl substituted with CycA,

wherein CycA is a mono- or bi-cyclic C5-10 carboaryl which may be substituted with 1-5 of R²⁷ or partially or completely saturated one thereof, or mono- or bi-cyclic 5-10 membered heteroaryl comprising 1-2 of nitrogen, 1-2 of oxygen and/or 1 of sulfur atom, or partially or completely saturated one thereof or

[II] (a) C1-8 alkyl, C2-8 alkenyl or C2-8 alkynyl substituted with a group selected from halo, CF₃, nitro, cyano and NR¹⁸R¹⁹, or

(b) (1) CycA containing 1-5 of substituent R²⁷ or

(2) C1-8 alkyl, C2-8 alkenyl or C2-8 alkynyl substituted with CycA, which contains 1-5 of substituent R²⁷,

wherein at least one of R²⁷ described in (1) and (2) is selected-from

(i) a C5-10 mono- or bi-cyclic carboring,

(ii) a 5-10 membered mono- or bi-cyclic heteroring,

(iii)-SO₂R¹⁵,

(iv) —OCF₃ or

(v) C1-8 alkyl substituted with 1-5 of group selected from (a)-halogen, (b)-NR¹¹R¹², (c) —OR¹³, (d) a C5-10 mono- or bi-cyclic carboring, (e) nitro, (f) CF₃, (g) cyano, (h) a 5-10 membered mono- or bi-cyclic heteroring, (j) —SR¹⁴, (k) —COR¹⁵, (l) —SO₂R¹⁵ and (m) OCF₃, wherein at least one group is selected from a C5-10 mono- or bi-cyclic carboring or a 5-10 membered mono- or bi-cyclic heteroring, —SO₂R¹⁵ or OCF₃,

above CycA is C5-10 mono- or bi-cyclic carboaryl or partially or completely saturated one, or 5-10 membered mono- or bi-cyclic heteroaryl comprising 1-2 of nitrogen, 1-2 of oxygen and/or 1 of sulfur, or partially or completely saturated one thereof.

Particularly preferably,

[I] (1) C1-4 alkyl, (2) C2-4 alkenyl, (3) C2-4 alkynyl, (4) CycA or (5) C1-4 alkyl, C2-4 alkenyl or C2-4 alkynyl substituted with CycA which is preferably cyclopentane, cyclohexane, benzene, naphthalene, pyrrolidine, piperidine, piperazine, morpholine, pyrrole, furan, thiophene, pyridine, pyrimidine, pyrazine, pyridazine, indole, isoindole, quinoline, isoquinoline, quinazoline, quinoxaline, phthalazine, benzothiophene, benzofuran, benzoxazole, tetrahydroquinoline, tetrahydroquinazoline, tetrahydroquinoxaline, optionally substituted with 1-5 of R^{27a} or

[II] (a) C1-8 alkyl, C2-8 alkenyl or C2-8 alkynyl substituted with a group selected from halogen, CF₃, nitro, cyano or NR¹⁸R¹⁹ or

(b) (1) CycA which contains 1-5 of substituent R²⁷, or

(2) C1-8 alkyl, C2-8 alkenyl or C2-8 alkynyl substituted with CycA which contains 1-5 of substituent R²⁷,

wherein at least one of R²⁷ described in (1) and (2) is selected from

(i) a C5-10 mono- or bi-cyclic carboring,

(ii) a 5-10 membered mono- or bi-cyclic heteroring,

(iii)-SO₂R¹⁵,

(iv) —OCF₃, or

(v) C1-8 alkyl substituted with 1-5 of group selected from (a) halo, (b) —NR¹¹R¹², (c) —OR¹³, (d) a C5-10 mono- or bi-cyclic carboring, (e) nitro, (f) CF₃, (g) cyano, (h) a 5-10 membered mono- or bi-cyclic heteroring, (j) —SR¹⁴, (k) —COR¹⁵, (1)—SO₂R¹⁵-or (m) —OCF₃, wherein at least one group is selected from a C5-10 mono- or bi-cyclic carboring, a 5-10 membered mono- or bi-cyclic heteroring, —SO₂R¹⁵ or —OCF₃, and

CycA is preferably cyclopentane, cyclohexane, benzene, naphthalene, pyrrolidine, piperidine, piperazine, morpholine, pyrrole, furan, thiophene, pyridine, pyrimidine, pyrazine, pyridazine, indole, isoindole, quinoline, isoquinoline, quinazoline, quinoxaline, phthalazine, benzothiophene, benzofuran, benzoxadiazole, tetrahydroquinoline, tetrahydroquinazoline, or tetrahydroquinoxaline.

In the formula (I), AA¹ is preferably a single bond,

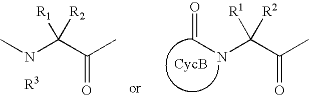

which is formed with R, and more preferably, AA¹ is a single bond or

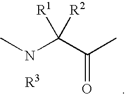

Any group represented by R¹ is preferable, and more preferably, R¹ is hydrogen, C1-8 alkyl, phenyl or C1-8 alkyl substituted with NH₂, C1-4 alkoxy, SH, SCH₃, phenyl, hydroxyphenyl, COOH, CONH₂, guanidino, imidazole or indole.

Particularly preferably, R¹ is hydrogen, C1-8 alkyl, phenyl or C1-8 alkyl substituted with C1-4 alkoxy or phenyl. Then, any group represented by R² is preferable, and hydrogen is particularly preferable.

And C3-6 alkylene which R¹ and R² together form is also preferable.

Any group represented by R³ is preferable, and more preferably R³ is hydrogen or C1-4 alkyl.

And C2-4 alkylene which R³ and R¹ together form is also preferable.

In the formula (I), AA² is all preferable, and more preferably, AA² is a single bond,

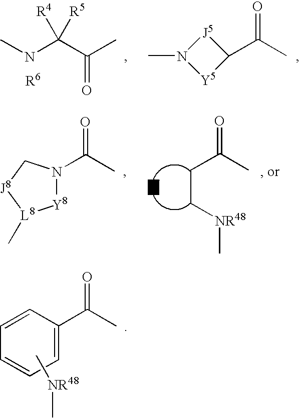

Particularly preferably, AA² is a single bond,

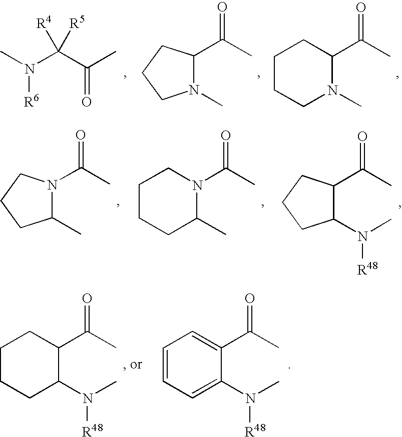

Any group represented by R⁴ is preferable, and more preferably, R⁴ is hydrogen, C1-8 alkyl, phenyl or C1-8 alkyl substituted with NH₂, C1-4 alkoxy, SH, SCH₃, phenyl, hydroxyphenyl, COOH, CONH₂, guanidino, imidazole or indole.

Particularly preferably, R⁴ is hydrogen, C1-8 alkyl, phenyl or C1-8 alkyl substituted with C1-4 alkoxy or phenyl. Then, any group represented by R⁵ is preferable, and hydrogen is particularly preferable.

And C3-6 alkylene which R⁴ and R⁵ together form is also preferable.

Any group represented by R⁶ is preferable, and more preferably R⁶ is hydrogen or C1-4 alkyl.

And C2-4 alkylene which R⁶ and R⁴ together form is also preferable.

R⁴⁸ is all preferable, but more preferably, R⁴⁸ is

[I] hydrogen, C1-4 alkyl, phenyl or C1-4 alkyl substituted with phenyl, or

[II] C2-6 alkylene, wherein one carbon atom may be replaced by oxygen, sulfur or —NR⁴⁷—, wherein R⁴⁷ is hydrogen or C1-4 alkyl to be formed together with R⁴, when AA¹ is a single bond.

Particularly preferably, R⁴¹ is

[I] hydrogen atom or C1-4 alkyl, or

[II] when AA¹ is a single bond, taken together with R to form tetramethylene, pentamethylene, —CH₂—CH₂—O—CH₂—CH₂—, —CH₂—CH₂—NH—CH₂—CH₂— or —CH₂—CH₂—N(CH₃)—CH₂—CH₂—.

In the formula (I), all the groups which AA¹ and AA² together form are preferable, and preferably, it is

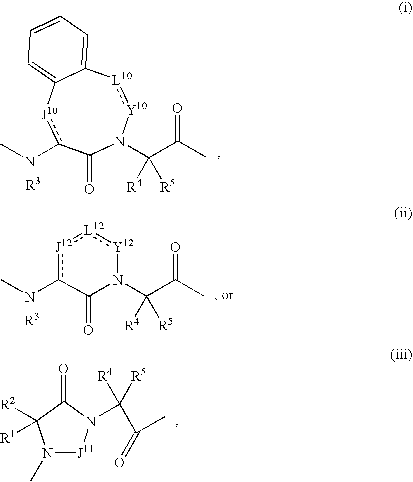

particularly preferably, it is

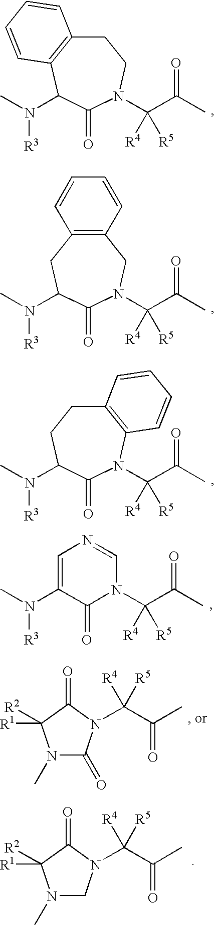

Any group represented by R⁷ is preferable. More preferably, R⁷ is hydrogen atom, C1-8 alkyl, phenyl, or C1-8 alkyl substituted with NH₂, C1-4 alkoxy, SH, SCH₃, phenyl, hydroxyphenyl, COOH, CONH₂, guanidino, imidazole or indole.

Particularly preferably, R⁷ is hydrogen, C1-8 alkyl, phenyl, or C1-8 alkyl substituted with C1-4 alkoxy or phenyl. Then, any group represented by R⁸ is preferable, but hydrogen is most preferable.

And C3-6 alkylene which R⁷ and R⁹ together form is also preferable.

Any group represented by R⁹ is preferable, but more preferably R⁹ is hydrogen or C1-4 alkyl.

And C2-4 alkylene which R⁹ and R⁷ together form is also preferable.

Any group represented by R¹⁰ is preferable, but more preferably R¹⁰ is C1-6 alkyl, CycA or C1-6 alkyl substituted with COR⁷′, NR⁷²R⁷³, hydroxy, OR⁷⁴ or CycA, more preferably C1-4 alkyl, or C1-4 alkyl substituted with phenyl, NR⁷²R⁷³ or C3-6 cycloalkyl.

In the present invention, in addition to the compounds described in the examples, the following compounds are preferable; the compounds of formula (Ia-1)

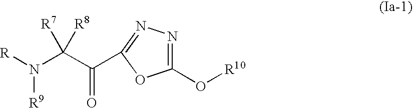

(wherein all symbols have the same meanings as above.), formula (lb-1)

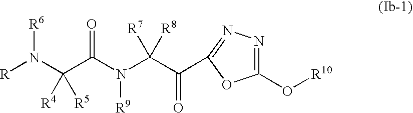

(wherein all symbols have the same meanings as above.), formula (Ic-1)

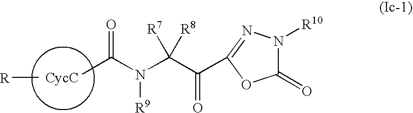

(wherein all symbols have the same meanings as above.), formula (Id-1)

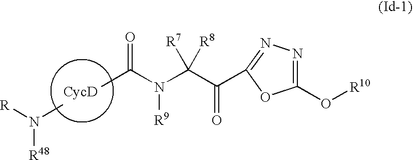

(wherein all symbols have the same meanings as above.), formula (Ie-1)

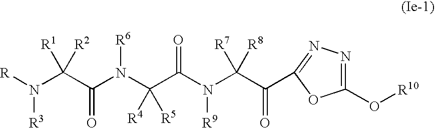

(wherein all symbols have the same meanings as above.), formula (If-1)

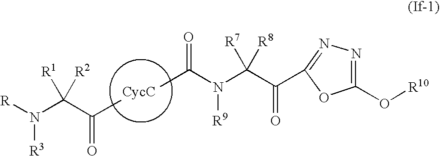

(wherein all symbols have the same meanings as above.), formula (Ig-1)

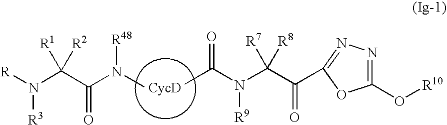

(wherein all symbols have the same meanings as above.), formula (Ih-1)

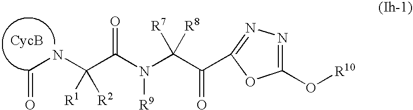

(wherein all symbols have the same meanings as above.), formula (Ii-1)

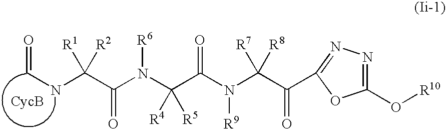

(wherein all symbols have the same meanings as above.), formula (Ij-1)

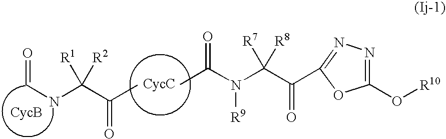

(wherein all symbols have the same meanings as above.), formula (Ik-31)

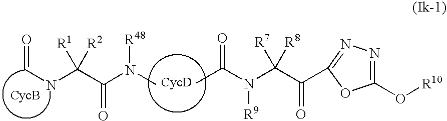

(wherein all symbols have the same meanings as above.), formula (Im-1)

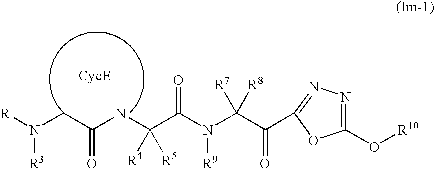

(wherein all symbols have the same meanings as above.), formula (In-1)

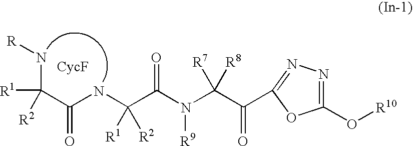

(wherein all symbols have the same meanings as above.), formula (Ia-2)

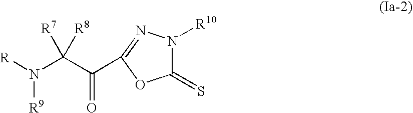

(wherein all symbols have the same meanings as above.), formula (Ib-2)

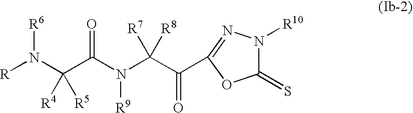

(wherein all symbols have the same meanings as above.), formula (Ic-2)

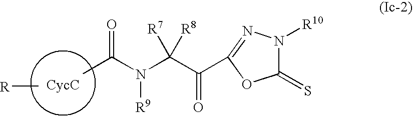

(wherein all symbols have the same meanings as above.), formula (Id-2)

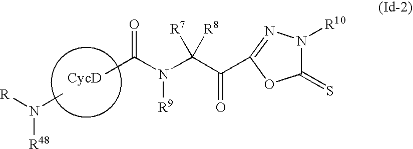

(wherein all symbols have the same meanings as above.), formula (Ie-2)

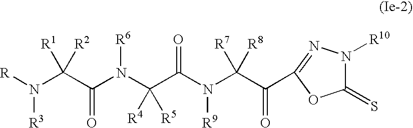

(wherein all symbols have the same meanings as above.), formula (If-2)

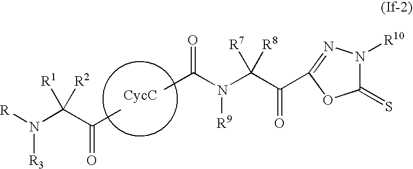

(wherein all symbols have the same meanings as above.), formula (Ig-2)

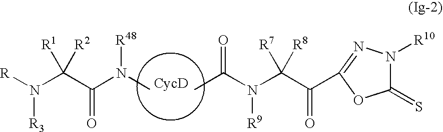

(wherein all symbols have the same meanings as above.), formula (Ih-2)

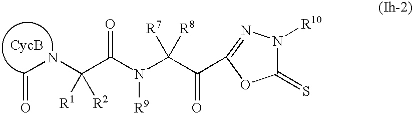

(wherein all symbols have the same meanings as above.), formula (Ii-2)

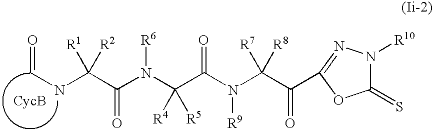

(wherein all symbols have the same meanings as above.), formula (Ij-2)

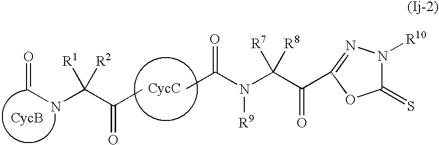

(wherein all symbols have the same meanings as above.), formula (Ik-2)

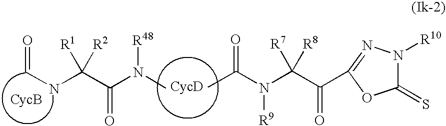

(wherein all symbols have the same meanings as above.), formula (Im-2)

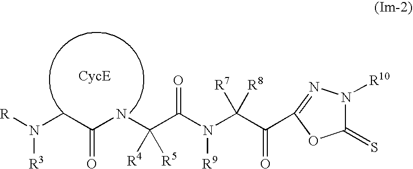

(wherein all symbols have the same meanings as above.), formula (In-2)

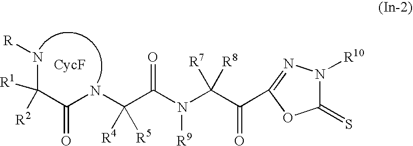

(wherein all symbols have the same meanings as above.), formula (Ia-3)

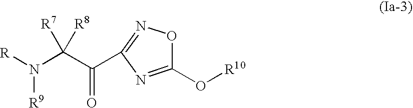

(wherein all symbols have the same meanings as above.), formula (Ib-3)

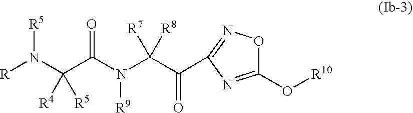

(wherein all symbols have the same meanings as above.), formula (Ic-3)

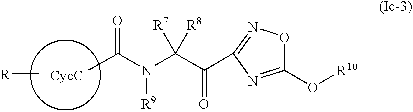

(wherein all symbols have the same meanings as above.), formula (Id-3)

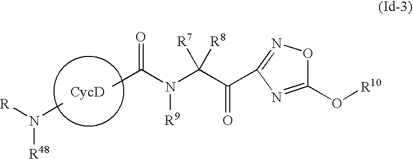

(wherein all symbols have the same meanings as above.), formula (Ie-3)

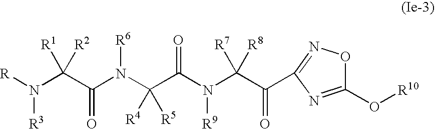

(wherein all symbols have the same meanings as above.), formula (If-3)

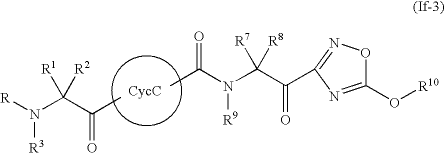

(wherein all symbols have the same meanings as above.), formula (Ig-3)

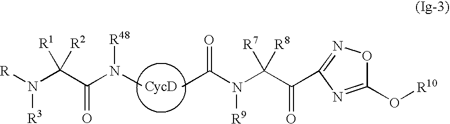

(wherein all symbols have the same meanings as above.), formula (Ih-3)

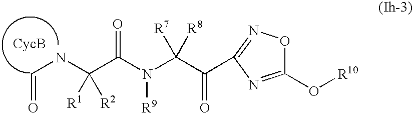

(wherein all symbols have the same meanings as above.), formula (Ii-3)

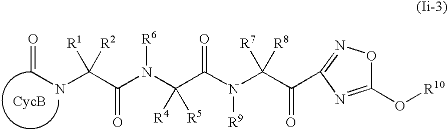

(wherein all symbols have the same meanings as above.), formula (Ij-3)

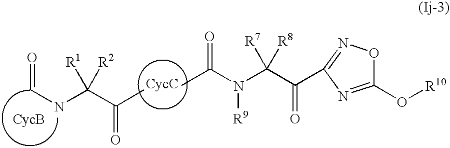

(wherein all symbols have the same meanings as above.), formula (Ik-3)

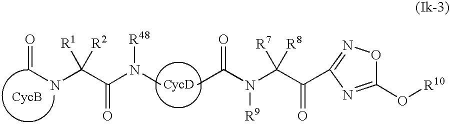

(wherein all symbols have the same meanings as above.), formula (Im-3)

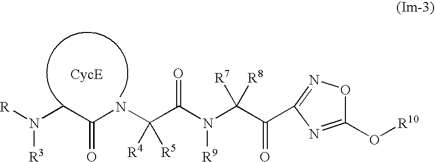

(wherein all symbols have the same meanings as above.), formula (In-3)

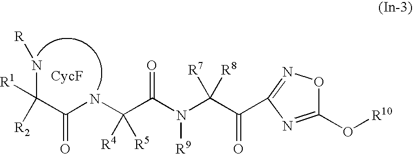

(wherein all symbols have the same meanings as above.), formula (Ia-4)

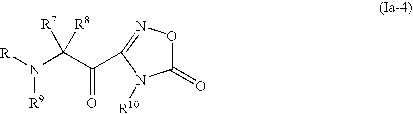

(wherein all symbols have the same meanings as above.), formula (Ib-4)

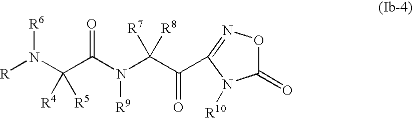

(wherein all symbols have the same meanings as above.), formula (Ic-4)

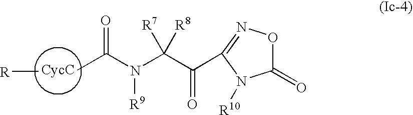

(wherein all symbols have the same meanings as above.), formula (Id-4)

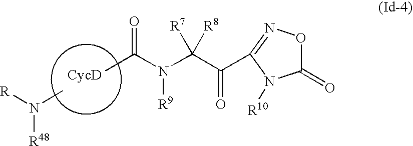

(wherein all symbols have the same meanings as above.), formula (Ie-4)

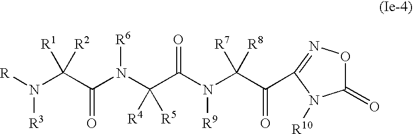

(wherein all symbols have the same meanings as above.), formula (If-4)

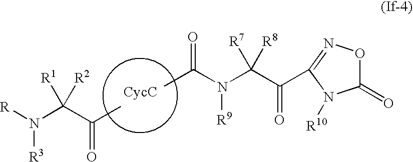

(wherein all symbols have the same meanings as above.), formula (Ig-4)

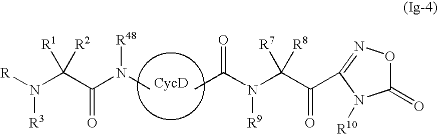

(wherein all symbols have the same meanings as above.), formula (Ih-4)

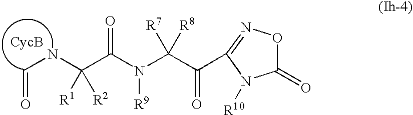

(wherein all symbols have the same meanings as above.), formula (Ii-4)

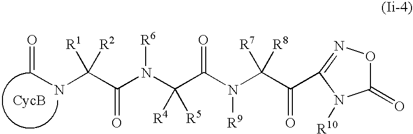

(wherein all symbols have the same meanings as above.), formula (Ij-4)

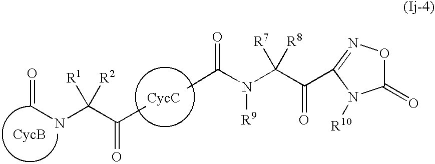

(wherein all symbols have the same meanings as above.), formula (Ik-4)

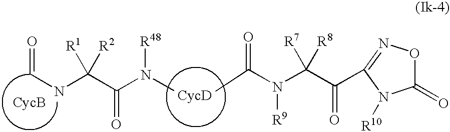

(wherein all symbols have the same meanings as above.), formula (Im-4)

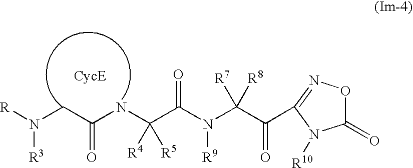

(wherein all symbols have the same meanings as above.), formula (In-4)

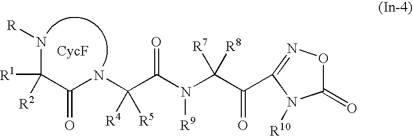

(wherein all symbols have the same meanings as above.), formula (Ia-5)

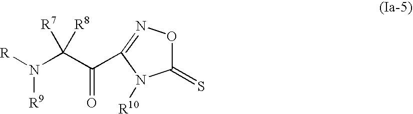

(wherein all symbols have the same meanings as above.), formula (Ib-5)

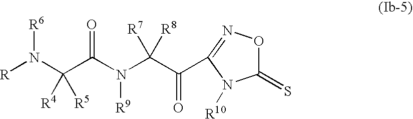

(wherein all symbols have the same meanings as above.), formula (Ic-5)

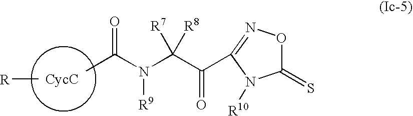

(wherein all symbols have the same meanings as above.), formula (Id-5)

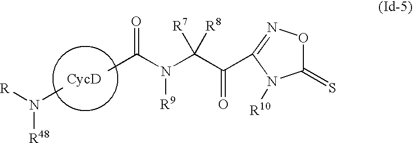

(wherein all symbols have the same meanings as above.), formula (Ie-5)

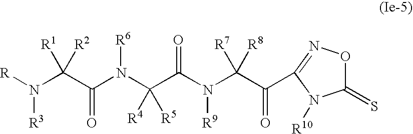

(wherein all symbols have the same meanings as above.), formula (If-5)

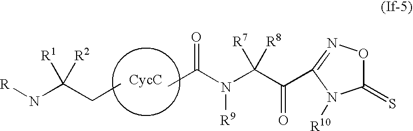

(wherein all symbols have the same meanings as above.), formula (Ig-5)

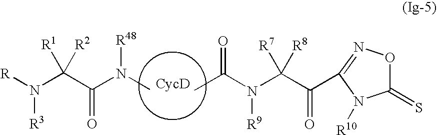

(wherein all symbols have the same meanings as above.), formula (Ih-5)

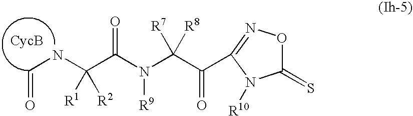

(wherein all symbols have the same meanings as above.), formula (Ii-5)

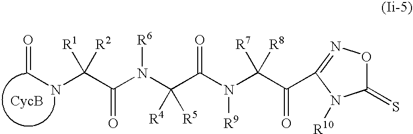

(wherein all symbols have the same meanings as above.), formula (Ij-5)

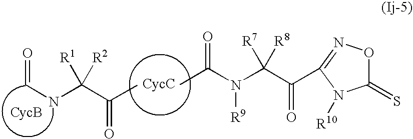

(wherein all symbols have the same meanings as above.), formula (Ik-5)

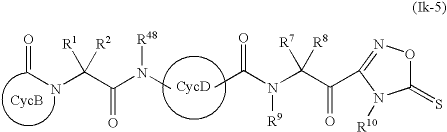

(wherein all symbols have the same meanings as above.), formula (Im-5)

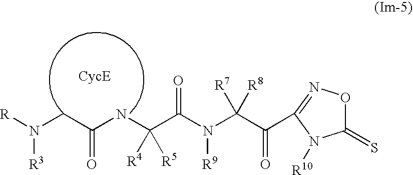

(wherein all symbols have the same meanings as above.), formula (In-5)

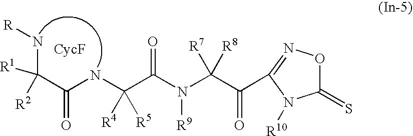

(wherein all symbols have the same meanings as above.) and non-toxic salts thereof.

Concretely, preferable are the compounds of the examples described below and the compounds shown in tables 1-20 and non-toxic salts thereof.

TABLE 1

| | |
|---|---|
| | (I-1A) |
| 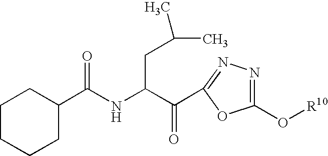 | |

| No. | R¹⁰ |
|---|---|
| 1 | 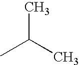 |
| 2 | 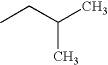 |
| 3 | 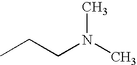 |
| 4 | 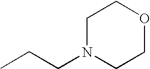 |
| 5 | 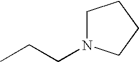 |
| 6 | 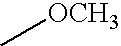 |
| 7 | 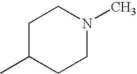 |
| 8 | 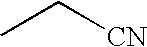 |
| 9 | 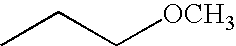 |
| 10 | 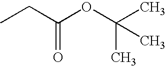 |
| 11 | 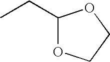 |
| 12 | 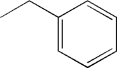 |
| 13 | 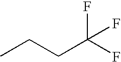 |

TABLE 2

| | |
|---|---|
| | (I-1B) |
| 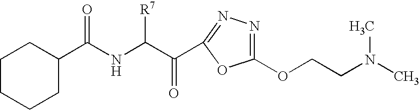 | |

| No. | R⁷ |
|---|---|
| 1 | 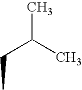 |
| 2 | 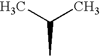 |
| 3 | 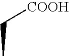 |
| 4 | 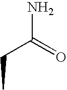 |
| 5 | 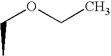 |
| 6 | 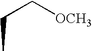 |
| 7 | 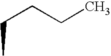 |
| 8 | 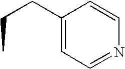 |
| 9 | 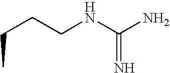 |
| 10 | 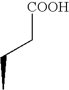 |
| 11 | 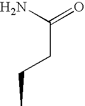 |
| 12 | 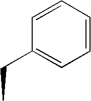 |
| 13 | 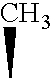 |
| 14 | 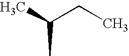 |
| 15 | 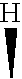 |
| 16 | 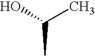 |
| 17 | 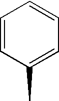 |
| 18 | 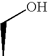 |
| 19 | 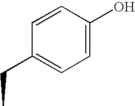 |
| 20 | 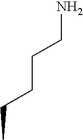 |
| 21 | 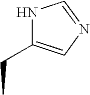 |

TABLE 3

| | |
|---|---|
| | (I-1C) |
| 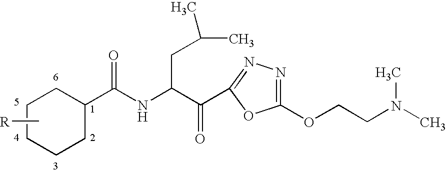 | |

| No. | R |
|---|---|
| 1 | 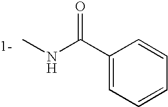 |
| 2 | 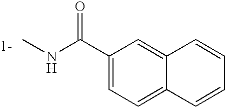 |
| 3 | 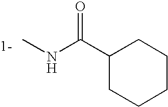 |
| 4 | 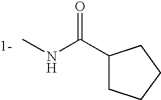 |
| 5 | 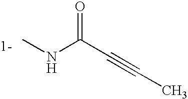 |
| 6 | 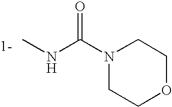 |
| 7 | 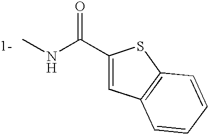 |
| 8 | 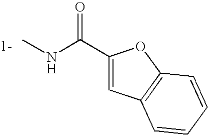 |
| 9 | 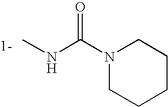 |
| 10 | 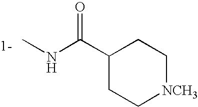 |
| 11 | 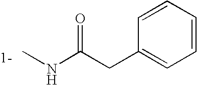 |
| 12 | 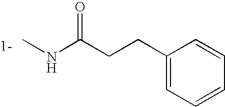 |
| 13 | 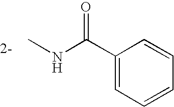 |
| 14 | 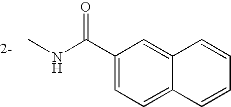 |
| 15 | 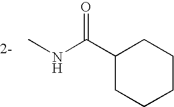 |
| 16 | 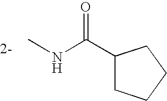 |
| 17 | 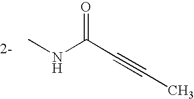 |
| 18 | 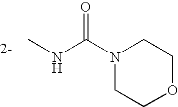 |

TABLE 4

| | |
|---|---|
| | (I-1D) |
| 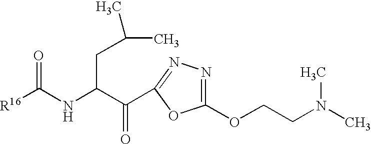 | |

| No. | R¹⁶ |
|---|---|
| 1 | 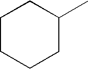 |
| 2 | 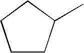 |
| 3 | 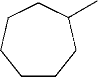 |
| 4 | 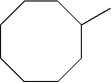 |
| 5 | 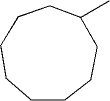 |
| 6 | 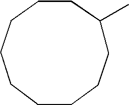 |
| 7 | 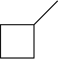 |
| 8 | 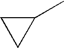 |
| 9 | 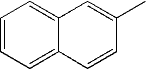 |
| 10 | 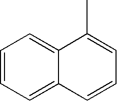 |
| 11 | 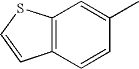 |
| 12 | 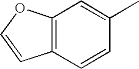 |
| 13 | 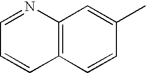 |
| 14 | 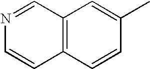 |
| 15 | 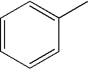 |
| 16 | 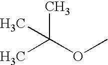 |
| 17 | 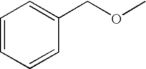 |
| 18 | 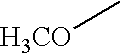 |
| 19 | 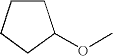 |
| 20 | 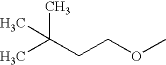 |

TABLE 5

| | |
|---|---|
| | (I-1E) |
| 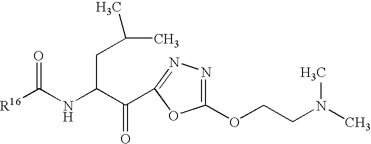 | |

| No. | R¹⁶ |
|---|---|
| 1 | 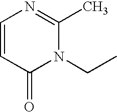 |
| 2 | 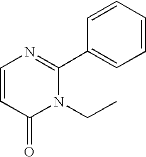 |
| 3 | 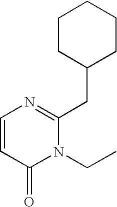 |
| 4 | 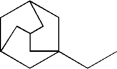 |
| 5 | 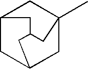 |
| 6 | 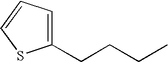 |
| 7 | 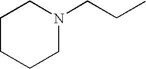 |
| 8 | 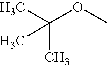 |
| 9 | 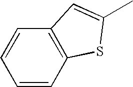 |
| 10 | 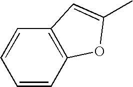 |
| 11 | 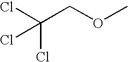 |

TABLE 6

| | |
|---|---|
| | (I-2A) |
| 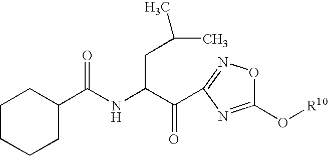 | |

| No. | R¹⁰ |
|---|---|
| 1 | 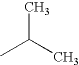 |
| 2 | 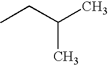 |
| 3 | 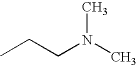 |
| 4 | 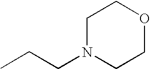 |
| 5 | 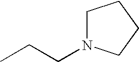 |
| 6 | 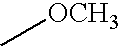 |
| 7 | 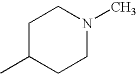 |
| 8 | 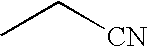 |
| 9 | 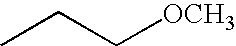 |
| 10 | 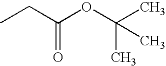 |
| 11 | 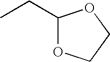 |
| 12 | 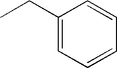 |
| 13 | 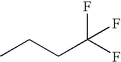 |

TABLE 7

| | |
|---|---|
| | (I-2B) |
| 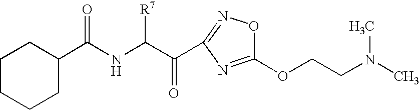 | |

| No. | R⁷ |
|---|---|
| 1 | 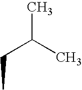 |
| 2 | 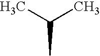 |
| 3 | 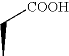 |
| 4 | 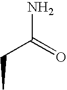 |
| 5 | 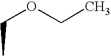 |
| 6 | 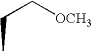 |
| 7 | 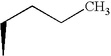 |
| 8 | 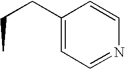 |
| 9 | 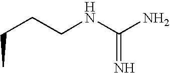 |
| 10 | 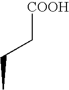 |
| 11 | 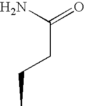 |
| 12 | 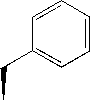 |
| 13 | 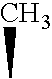 |
| 14 | 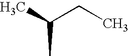 |
| 15 | 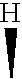 |
| 16 | 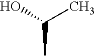 |
| 17 | 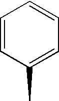 |
| 18 | 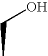 |
| 19 | 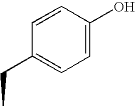 |
| 20 | 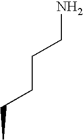 |
| 21 | 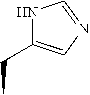 |

TABLE 8

| | |
|---|---|
| | (I-2C) |
| 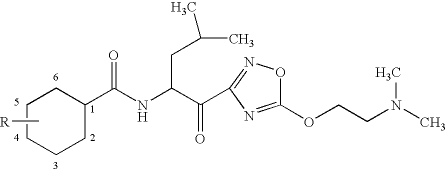 | |

| No. | R |
|---|---|
| 1 | 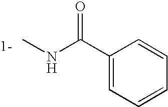 |
| 2 | 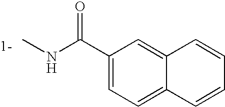 |
| 3 | 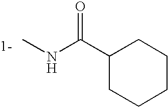 |
| 4 | 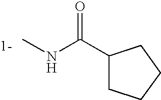 |
| 5 | 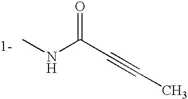 |
| 6 | 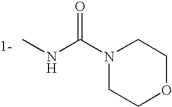 |
| 7 | 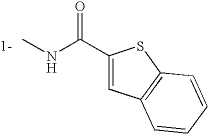 |
| 8 | 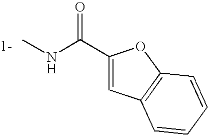 |
| 9 | 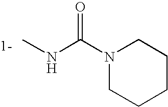 |
| 10 | 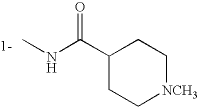 |
| 11 | 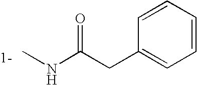 |
| 12 | 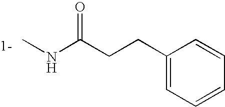 |
| 13 | 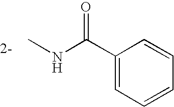 |
| 14 | 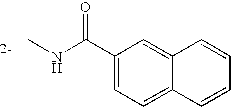 |
| 15 | 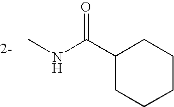 |
| 16 | 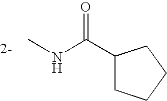 |
| 17 | 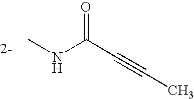 |
| 18 | 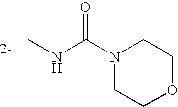 |

TABLE 9

| | |
|---|---|
| | (I-2D) |
| 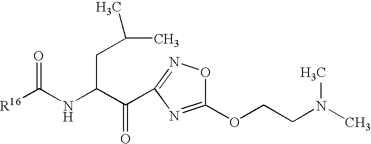 | |

| No. | R¹⁶ |
|---|---|
| 1 | 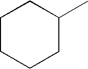 |
| 2 | 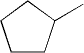 |
| 3 | 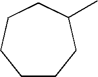 |
| 4 | 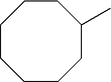 |
| 5 | 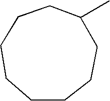 |
| 6 | 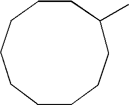 |
| 7 | 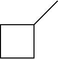 |
| 8 | 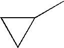 |
| 9 | 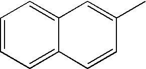 |
| 10 | 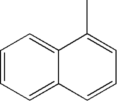 |
| 11 | 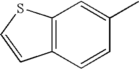 |
| 12 | 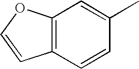 |
| 13 | 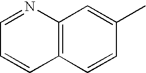 |
| 14 | 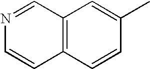 |
| 15 | 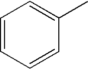 |
| 16 | 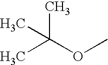 |
| 17 | 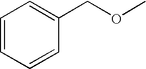 |
| 18 | 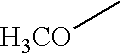 |
| 19 | 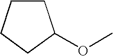 |
| 20 | 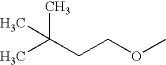 |

TABLE 10

| | |
|---|---|
| | (I-2E) |
| 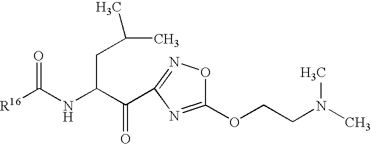 | |

| No. | R¹⁶ |
|---|---|
| 1 | 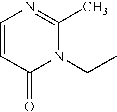 |
| 2 | 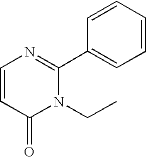 |
| 3 | 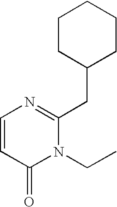 |
| 4 | 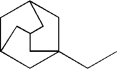 |
| 5 | 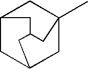 |
| 6 | 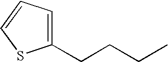 |
| 7 | 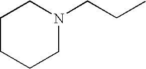 |
| 8 | 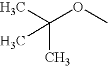 |
| 9 | 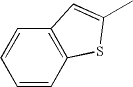 |
| 10 | 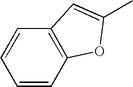 |
| 11 | 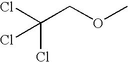 |

TABLE 11

| | |
|---|---|
| | (I-3A) |
| 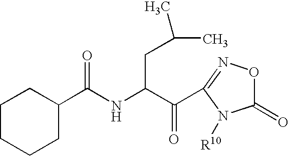 | |

| No. | R¹⁰ |
|---|---|
| 1 | 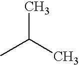 |
| 2 | 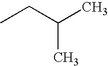 |
| 3 | 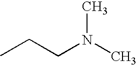 |
| 4 | 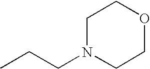 |
| 5 | 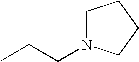 |
| 6 | 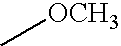 |
| 7 | 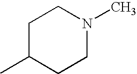 |
| 8 | 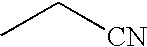 |
| 9 | 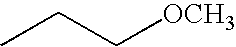 |
| 10 | 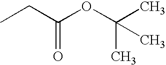 |
| 11 | 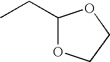 |
| 12 | 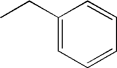 |
| 13 | 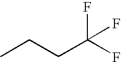 |

TABLE 12

| | |
|---|---|
| | (I-3B) |
| 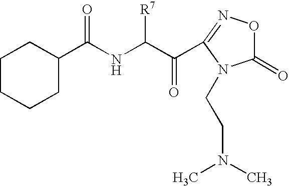 | |

| No. | R⁷ |
|---|---|
| 1 | 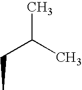 |
| 2 | 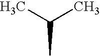 |
| 3 | 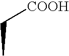 |
| 4 | 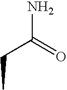 |
| 5 | 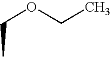 |
| 6 | 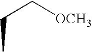 |
| 7 | 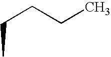 |
| 8 | 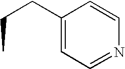 |
| 9 | 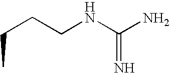 |
| 10 | 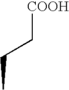 |
| 11 | 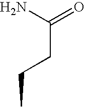 |
| 12 | 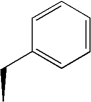 |
| 13 | 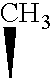 |
| 14 | 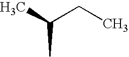 |
| 15 | 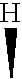 |
| 16 | 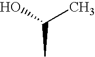 |
| 17 | 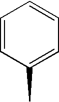 |
| 18 | 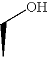 |
| 19 | 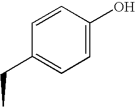 |
| 20 | 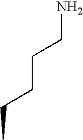 |
| 21 | 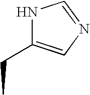 |

TABLE 13

| | |
|---|---|
| | (I-3C) |
| 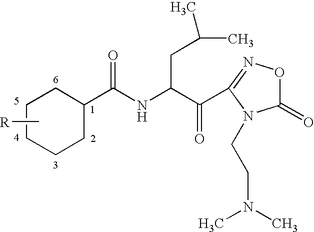 | |

| No. | R |
|---|---|
| 1 | 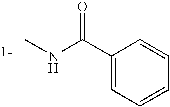 |
| 2 | 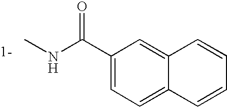 |
| 3 | 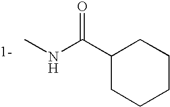 |
| 4 | 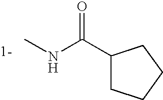 |
| 5 | 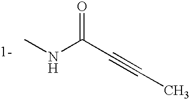 |
| 6 | 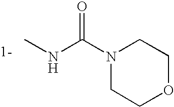 |
| 7 | 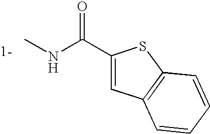 |
| 8 | 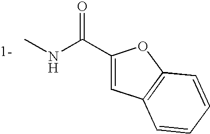 |
| 9 | 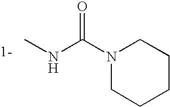 |
| 10 | 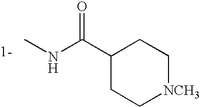 |
| 11 | 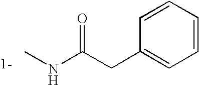 |
| 12 | 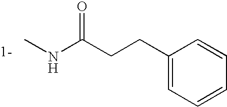 |
| 13 | 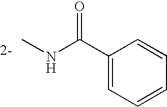 |
| 14 | 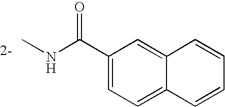 |
| 15 | 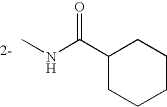 |
| 16 | 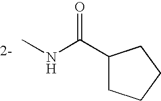 |
| 17 | 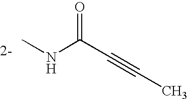 |
| 18 | 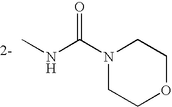 |

TABLE 14

| | |
|---|---|
| | (I-3D) |
| 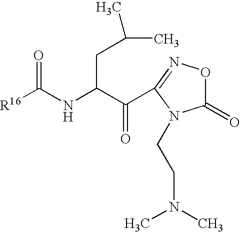 | |

| No. | R¹⁶ |
|---|---|
| 1 | 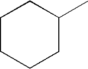 |
| 2 | 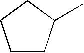 |
| 3 | 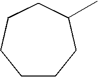 |
| 4 | 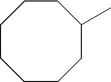 |
| 5 | 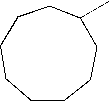 |
| 6 | 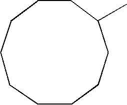 |
| 7 | 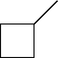 |
| 8 | 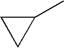 |
| 9 | 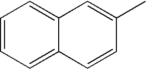 |
| 10 | 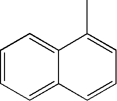 |
| 11 | 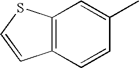 |
| 12 | 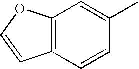 |
| 13 | 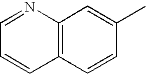 |
| 14 | 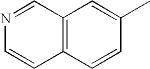 |
| 15 | 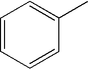 |
| 16 | 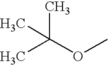 |
| 17 | 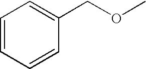 |
| 18 | 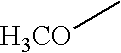 |
| 19 | 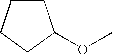 |
| 20 | 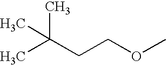 |

TABLE 15

| | |
|---|---|
| | (I-3E) |
| 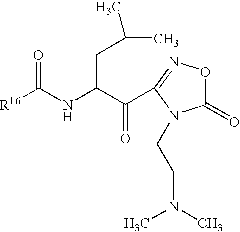 | |

| No. | R¹⁶ |
|---|---|
| 1 | 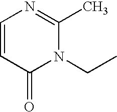 |
| 2 | 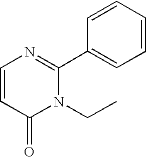 |
| 3 | 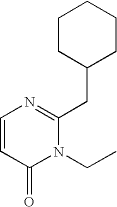 |
| 4 | 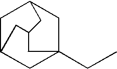 |
| 5 | 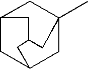 |
| 6 | 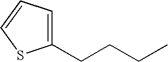 |
| 7 | 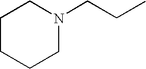 |
| 8 | 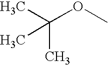 |
| 9 | 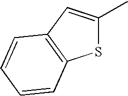 |
| 10 | 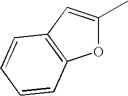 |
| 11 | 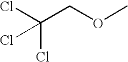 |

TABLE 16

| | |
|---|---|
| | (I-4A) |
| 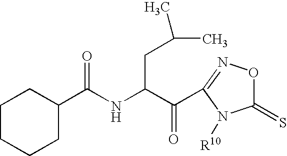 | |

| No. | R¹⁰ |
|---|---|
| 1 | 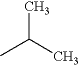 |
| 2 | 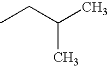 |
| 3 | 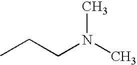 |
| 4 | 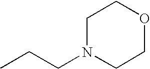 |
| 5 | 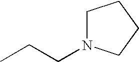 |
| 6 | 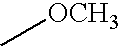 |
| 7 | 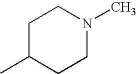 |
| 8 | 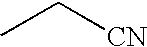 |
| 9 | 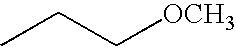 |
| 10 | 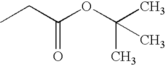 |
| 11 | 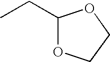 |
| 12 | 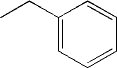 |
| 13 | 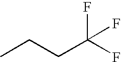 |

TABLE 17

| | |
|---|---|
| | (I-4B) |
| 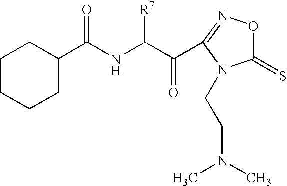 | |

| No. | R⁷ |
|---|---|
| 1 | 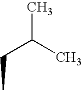 |
| 2 | 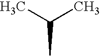 |
| 3 | 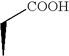 |
| 4 | 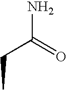 |
| 5 | 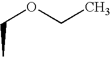 |
| 6 | 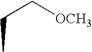 |
| 7 | 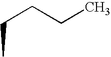 |
| 8 | 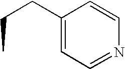 |
| 9 | 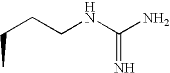 |
| 10 | 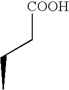 |
| 11 | 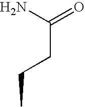 |
| 12 | 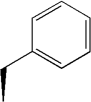 |
| 13 | 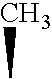 |
| 14 | 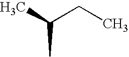 |
| 15 | 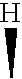 |
| 16 | 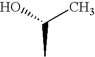 |
| 17 | 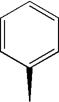 |
| 18 | 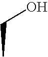 |
| 19 | 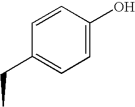 |
| 20 | 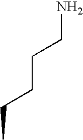 |
| 21 | 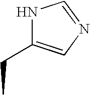 |

TABLE 18

| | |
|---|---|
| | (I-4C) |
| 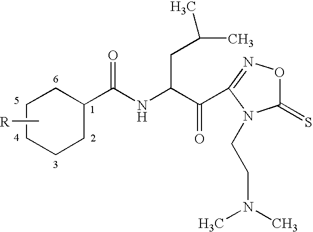 | |

| No. | R |
|---|---|
| 1 | 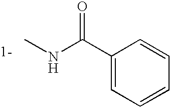 |
| 2 | 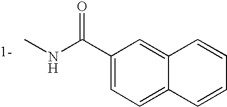 |
| 3 | 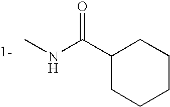 |
| 4 | 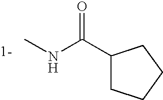 |
| 5 | 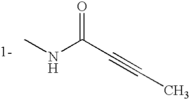 |
| 6 | 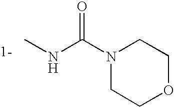 |
| 7 | 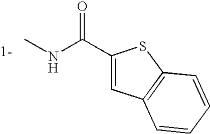 |
| 8 | 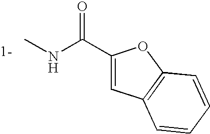 |
| 9 | 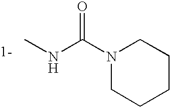 |
| 10 | 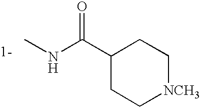 |
| 11 | 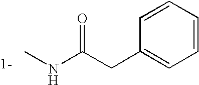 |
| 12 | 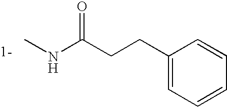 |
| 13 | 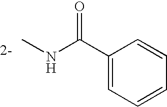 |
| 14 | 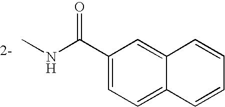 |
| 15 | 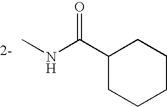 |
| 16 | 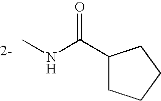 |
| 17 | 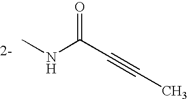 |
| 18 | 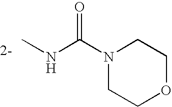 |

TABLE 19

| | |
|---|---|
| | (I-4D) |
| 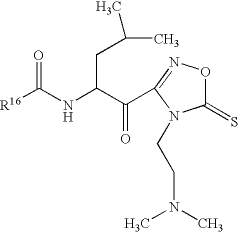 | |

| No. | R¹⁶ |
|---|---|
| 1 | 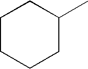 |
| 2 | 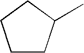 |
| 3 | 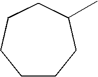 |
| 4 | 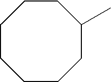 |
| 5 | 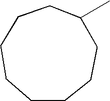 |
| 6 | 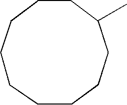 |
| 7 | 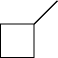 |
| 8 | 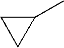 |
| 9 | 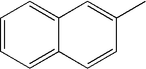 |
| 10 | 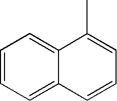 |
| 11 | 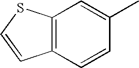 |
| 12 | 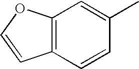 |
| 13 | 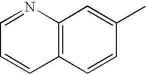 |
| 14 | 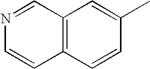 |
| 15 | 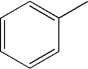 |
| 16 | 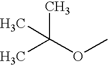 |
| 17 | 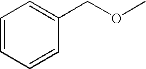 |
| 18 | 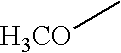 |
| 19 | 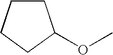 |
| 20 | 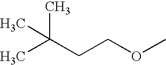 |

TABLE 20

| | |
|---|---|
| | (I-4E) |
| 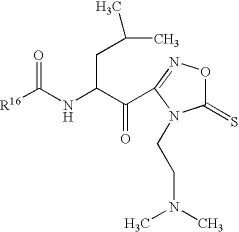 | |

| No. | R¹⁶ |
|---|---|
| 1 | 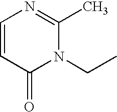 |
| 2 | 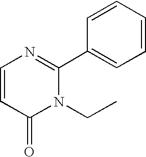 |
| 3 | 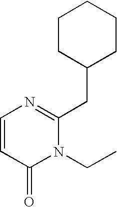 |
| 4 | 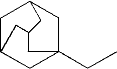 |
| 5 | 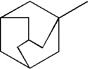 |
| 6 | 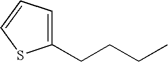 |
| 7 | 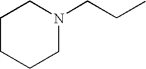 |
| 8 | 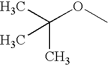 |
| 9 | 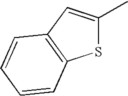 |
| 10 | 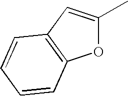 |
| 11 | 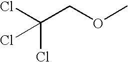 |

In the present invention, isomers are included unless specified. For example, alkyl, alkoxy, alkylthio, alkenyl, alkynyl and alkylene include straight and branched ones. Furthermore, the present invention includes isomers in double bond, ring, fused ring (E, Z, cis, trans), isomers by the presence of asymmetric carbon etc.(R, S, α, β, enantiomer, diastereomer), optical isomers having optical rotation (D, L, d, 1, +, −), polars by chromatography separation (more polar, less polar), equilibrium compound, a compound of arbitrary ratios of those and racemic mixture.

Salts:

The compounds of formula (I) of the present invention may be converted into corresponding non-toxic salts by conventional methods. Non-toxic salts include alkali metal salts, alkaline earth metal salts, amine salts, acid-addition salts and corresponding quaternary ammonium salts when the compound of formula (I) contains amino acid residues.

Non-toxic and water-soluble salts are preferable. Appropriate non-toxic salts include salts of alkali metals (potassium, sodium etc.), salts of alkaline-earth metals (calcium, magnesium, etc.), ammonium salts—and salts of pharmaceutically-acceptable organic amines (tetramethyl ammonium, triethylamine, methylamine, dimethylamine, cyclopentylamine, benzylamine, phenethylamine, piperidine, monoethanolamine, diethanolamine, tris(hydroxymethyl)amino methane, lysine, arginine, N-methyl-D-glucamine, etc.

Non-toxic, water-soluble acid-addition salts are preferable. Appropriate acid-addition salts are, inorganic salts such as hydrochloride, hydrobromide, sulfate, phosphate, nitrate, or organic salts such as acetate, trifluoroacetate, lactate, tartrate, oxalate, fumarate, malate, citrate, benzoate, methanesulfonate, ethanesulfonate, benzenesulfonate, toluenesulfonate, isethionate, glucuronate, gluconate.

The compounds of formula (I) of the present invention or a salt thereof may be converted into hydrate by a conventional method.

The compounds of formula (I) of the present invention may also be converted into N-oxide compounds by a conventional method.

Process for the Preparation of the Compounds of the Present Invention

(1) Among the compounds of formula (I):

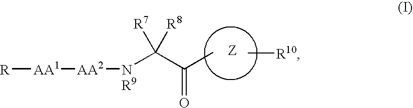

the compound wherein AA¹ and AA² represent a single bond at the same time and none of R, R⁷, R⁸ and R¹⁰ contains carboxy, hydroxy, amino, thiol, guanidino or phosphono, and R does not represent hydrogen, i.e. the compound of formula (IA-1)

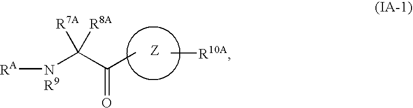

wherein R^{A}, R^{7A}, R^{8A} and R^{10A} have the same meanings as R, R⁷, R¹ and R¹⁰, with proviso that none of them contains carboxy, hydroxy, amino, thiol, guanidino or phosphono and R^{A} is not hydroge and the other symbols have the same meanings as above, may be prepared by subjecting to oxidation reaction a compound of formula (IIA-1)

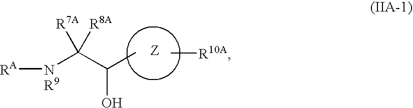

wherein all symbols have the same meaning as above.

This oxidation reaction is known, for example,

(1) a method of Swern oxidation,

(2) a method utilizing Dess-Martin reagent, and

(3) a method utilizing TEMPO reagent, etc. may be included.

To describe them concretely,

(1) the method of Swern oxidation is carried out, for example, in an inert organic solvent (chloroform, methylene chloride, etc.) by subjecting to a reaction oxalyl chloride and dimethylsulfoxide at −78° C. and then subjecting to a reaction the obtained solution with an alcohol compound, and then subjecting to a reaction with a tertiary amine such as at a temperature of −78 to 20° C.

(2) the method utilizing Dess-Martin reagent is carried out, for example, in an inert organic solvent (chloroform, dichloromethane, etc.) in the presence of Dess-Martin reagent (1,1,1-triacetoxy-1,1-dihydro-1,2-benzoiodoxol-3-(1H)-one) at a temperature of 0 to 40° C.

(3) the method utilizing TEMPO reagent is carried out, for example, in an inert organic solvent (chloroform, methylene chloride, etc.), in the presence of TEMPO reagent (2,2,6,6-tetramethyl-1-piperidinyloxy, free radical) at a temperature of 20 to 60° C.

These reactions of (1), (2) and (3) are desirably carried out under the atmosphere of an inert gas (argon, nitrogen, etc.) under anhydrous conditions.

The present invention further includes other oxidation reactions which oxidize alcohol to ketone easily and selectively. For example, Jones oxidation, oxidation by pyridinium chlorochromate (PCC), sulfur trioxide-pyridine complex or ones described in “Comprehensive Organic Transformations (Richard C. Larock, VCH Publishers, Inc., (1989) 604-614)” may be used.

(2) Among the compounds of formula (I), the compound wherein R is hydrogen and none of R⁷, R⁸ or R¹⁰ contains carboxy, hydroxy, amino, thiol, or guanidino group, i.e. the compound of formula (IB-1),

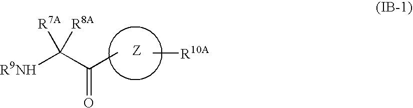

wherein all symbols have the same meaning as above, may be prepared by subjecting to a deprotection reaction of amino-protective group the compound among the compounds of formula (IA) prepared according to the above-described method, wherein R^{A} is a protective group for amino group, i.e. the compound of formula (IA-1-1),

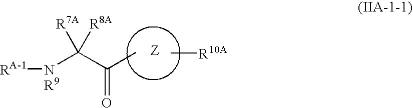

wherein R^{A-2} is a protective group of amino group, and the other symbols have the same meanings as above.

As protective groups for amino group, for example, benzyloxycarbonyl, t-butoxycarbonyl, trifluoroacetyl, 9-fluorenylmethoxycarbonyl may be included, and other groups that can be easily and selectively eliminated may also be used instead. For example, the groups described in T. W. Greene, Protective Groups in Organic Synthesis, Wiley, New York, 1991 may be used.

Deprotection reaction for protective groups of amino group is known, for example,

1) deprotection reaction under alkaline conditions,

2) deprotection reaction under acidic conditions,

3) deprotection reaction by hydration, etc. may be included.

To explain these methods concretely,

1) deprotection reaction under alkaline conditions is carried out, for example, in an organic solvent (methanol, tetrahydrofuran, dioxane, dimethylformamide, etc.) using a hydroxide of alkali metals (sodium hydroxide, potassium hydroxide, lithium hydroxide, etc.), hydroxide of alkaline earth metals (barium hydroxide, calcium hydroxide, etc.), organic amine (triethylamine, N-methylmorpholine, diisopropylethylamine, piperidine, etc.) or a quaternary ammonium salt (tetrabutyl ammonium fluoride etc.) or a solution thereof or a mixture thereof at a temperature of 0 to 40° C.;

2) deprotection reaction under acidic conditions is carried out, for example, in an organic solvent (methylene chloride, chloroform, dioxane, ethyl acetate, anisole, etc.), using organic acid (acetic acid, trifluoroacetic acid, methanesulfonic acid, etc.) or inorganic acid (hydrochloric acid, sulfuric acid, etc.) or a mixture thereof (hydrobromic acid/acetic acid, etc.) at a temperature of 0 to 100° C.;

3) deprotection reaction by hydration is, for example, carried out in a solvent (ethers (tetrahydrofuran, dioxane, dimethoxyethane, diethyl ether, etc.), alcohols (methanol, ethanol, etc.), benzenes (benzene, toluene, etc.), ketones (acetone, methyl ethyl ketone, etc.), nitriles such as acetonitrile, amides such as dimethylformamide, water, ethyl acetate, acetic acid or a mixture of more than two from above, etc.) in the presence of a catalyst (palladium-carbon, palladium black, palladium hydroxide, platinum oxide, Raney nickel, etc.) under the atmosphere of hydrogen of normal or suppressed pressure, or in the presence of ammonium formate at a temperature of 0 to 200° C.

As easily understood by those skilled in the art, the compounds of the present invention may be easily prepared by selecting these reactions.

(3) Among the compounds of formula (I), the compound wherein AA¹ and AA² represent a single bond at the same time, and at least one of R, R⁷, R⁸ and R¹⁰ contains carboxy, hydroxy, amino, thiol or guanidino, or R is hydrogen; i.e. the compound of formula (IC-1),

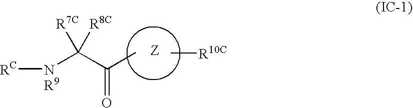

wherein R^{C}, R^{7C}, R^{8C} and R^{10C} have the same meanings as R, R⁷, R⁸ and R¹⁰, but at least one of them contains carboxy, hydroxy, amino, thiol, guanidino or phosphono, or R^{C} is hydrogen and the other symbols have the same meanings as above, may be prepared by subjecting to a deprotection reaction of a protective group of carboxy, hydroxy, amino, thiol, guanidino or phosphono, the compound among the compounds of formula (IA-1) prepared according to the above-described method, wherein at least one of R^{A}, R^{7A}, R^{8A} or R^{10A} contains a protected form of carboxy, hydroxy, amino, thiol, guanidino or phosphono, i.e. the compound of formula (IA-1-2),

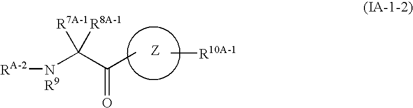

wherein R^{A-1}, R^{7A-1}, R^{1A-1} and R^{10A-1} have the same meanings as R^{A}, R^{7A}, R^{8A} and R^{10A} respectively, but at least one of R^{A-2}, R^{7A-1}, R^{8A-1} and R^{10A-1} contains a protected form of carboxy, hydroxy, amino, thiol, guanidino or phosphono, or R^{A-2} is a protective group of amino, and the other symbols have the same meanings as above, or the compound among the compound of formula (IB-1) prepared according to the above-described method, wherein at least one of R^{7A}, R^{8A} and R^{10A} contains a protected form of carboxy, hydroxy, amino, thiol, guanidino or phosphono; i.e. the compound of formula (IB-1-1),

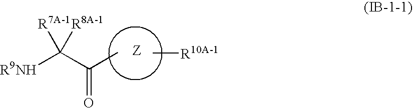

wherein all symbols have the same meanings as above.

Protective groups for carboxy include, for example, methyl, ethyl, t-butyl, benzyl.

Protective groups for hydroxy include, for example, methoxymethyl, 2-tetrahydropyranyl, t-butyldimethylsilyl, t-butyldiphenylsilyl, acetyl and benzyl.

Protective groups for amino include, the ones above specified.

Protective groups for thiol include, for example, benzyl, methoxybenzyl, methoxymethyl, 2-tetrahydropyranyl, diphenylmethyl, and acetyl.

Protective groups for guanidino include, for example, benzyloxycarbonyl, t-butoxycarbonyl, 9-fluorenylmethoxycarbonyl. Protective groups for carboxy, hydroxy, amino, thiol or guanidino are not limited to the above groups, but those groups which are eliminated easily and selectively are also allowed. For example, the ones described in T. W. Greene, Protective Groups in Organic Synthesis, Wiley, New York, 1991 are used.

Protective groups for phosphono include, for example, C1-2 alkyl, phenyl, benzyl, 2,2,2-trichloroethyl, cyanoethyl.

Deprotection reactions of the protective groups of carboxy, hydroxy, amino, thiol, guanidino or phosphono are well known, for example,

1) a deprotection reaction under alkaline conditions,

2) a deprotection reaction under acidic conditions,

3) a deprotection reaction by hydration,

4) a deprotection reaction of silyl-containing groups, etc. may be included.

The methods of 1), 2) and 3) are carried out by the methods described above.

4) A deprotection reaction of silyl-containing group is carried out, for example, in a water-miscible organic solvent (tetrahydrofuran, acetonitrile, etc.) using tetrabutylammonium fluoride at a temperature of 0 to 40° C.

Deprotection reaction of protective groups of phosphono is well-known, for example,

(a) Elimination of C1-2 alkyl is carried out by subjecting to a reaction in an organic solvent (chloroform etc.), using halogenated trimethylsilyl (e.g. trimethylsilyl chloride, trimethylsilyl bromide, trimethylsilyl iodide, etc.) as a reagent, in the presence or absence of alkali metal iodide (e.g. sodium iodide, potassium iodide, etc.) at a temperature of 0 to 40° C.

(b) Elimination of phenyl is carried out by subjecting to a reaction under atmosphere of hydrogen, in an organic solvent (methanol, ethanol, tetrahydrofuran, pyridine, acetic acid, etc.) or without a solvent, in the presence or absence of a catalyst (platinum oxide etc.) and an organic acid (acetic acid etc.) or inorganic acid (hydrochloric acid etc.) at a temperature of 0 to 50° C. for 24 hours to 3 days.

(c) Elimination of benzyl is carried out by subjecting to a reaction in an organic solvent (methanol, ethnanol, tetrahydrofuran, pyridine, acetic acid, etc.) in the presence or absence of a catalyst (palladium-carbon, palladium black, palladium hydroxide, etc.) at a temperature of 0 to 50° C.

(d) Elimination of 2,2,2-trichloroethyl is carried out in an organic solvent (methanol, ethanol, tetrahydrofuran, etc.) or without a solvent, using fine powder of zinc etc and an organic acid (acetic acid etc.) or an inorganic acid (hydrochloric acid etc.) at a temperature of 0 to −50° C.

(e) Elimination of cyanoethyl is carried out in a solvent (water, methanol, ethanol, tetrahydrofuran, pyridine, etc.) or without a solvent in the presence of a base (trimethylamine, dimethylamine, t-butylamine, etc.) at a temperature of 0 to 100° C.

As easily understood by those skilled in the art, the target compounds of the present invention may be easily prepared by selecting these reactions.

(4) Among the compound of formula (I), the compound wherein AA¹ and AA² do not represent a single bond at the same time, and none of R, AA¹, AA², R⁷, R⁸ or R¹⁰ include carboxy, hydroxy, amino, thiol, guanidino, or phosphono, i.e. the compound of formula (IA-2)

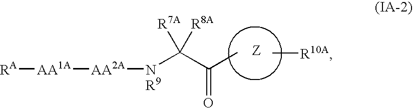

wherein AA^{1A} and AA^{2A} represent the same meanings as AA¹ and AA² respectively, with proviso that none of them includes carboxy, hydroxy, amino, thiol or guanidino, and AA^{1A} and AA^{2A} do not represent a single bond at the same time, and the other symbols have the same meanings as above, may be prepared according to the methods [I] and [2] described below.

[1] The compound of formula (IA-2) may be prepared by subjecting to an oxidation reaction the compound of formula (IIA-2)

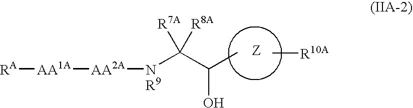

(wherein all symbols have the same meanings as above.). Oxidation reaction is carried out by the above-described method.

[2] The compound of formula (IA-2) may also be prepared by subjecting to amidation reaction the compound of formula (IB-1), prepared according to the above-described method and the compound of formula (X)

R^{A}—AA^{1A}—AA^{2A}—OH (X)

(wherein all symbols have the same meanings as above.).

Amidation reaction is known, for example,

1) a method using acid halide,

2) a method using mixed anhydride,

3) a method using a condensing agent (EDC, DCC, etc.), etc.

To explain these methods concretely,

1) the method using acid halide is carried out, for example, by subjecting to a reaction carboxylic acid and acid-halogenating agent (oxalyl chloride, thionyl chloride, etc.) in an organic solvent (chloroform, methylene chloride, diethyl ether, tetrahydrofuran, etc.) or without a solvent, between −20° C. and refluxing temperature, and then subjecting to a reaction thus obtained acid halide in the presence of tertiary amine (pyridine, triethylamine, dimethylaniline, dimethylaminopyridine, etc.) in an inert organic solvent (chloroform, methylene chloride, diethyl ether, tetrahydrofuran, etc.) at a temperature of 0 to 40° C.

And it may be carried out by subjecting to a reaction with acid halide in an organic solvent (dioxane, tetrahydrofuran, etc.) using an aqueous alkali solution (an aqueous solution of sodium bicarbonate or sodium hydroxide, etc.) at a temperature of 0 to 40° C.

2) The method using mixed anhydride is carried out, for example, by subjecting to a reaction in an organic solvent (chloroform, methylene chloride, diethyl ether, tetrahydrofuran, etc.) or without a solvent, in the presence of tertiary amine (pyridine, triethylamine, dimethylaniline, dimethylaminopyridine, etc.), carboxylic acid with acid halide (pivaloyl chloride, tosyl chloride, mesylchloride, etc.) or acid derivative (chloroethyl formate, chloroisobutyl formate, etc.) at a temperature of 0 to 40° C., and then subjecting to a reaction thus obtained mixed anhydride with amine in an organic solvent (chloroform, methylene chloride, diethyl ether, tetrahydrofuran, etc.) at a temperature of 0 to 40° C.

3) The method using a condensing agent is carried out, for example, in an organic solvent (chloroform, methylene chloride, dimethylformamide, diethyl ether, tetrahydrofuran, etc.) or without a solvent, in the presence or absence of a tertiary amine (pyridine, triethylamine, dimethylaniline, dimethylaminopyridine, etc.), using a condensing agent (1,3-dicychlorohexylcarbodiimide (DCC), 1-ethyl-3-[3-(dimethylamino)propyl]carbodiimide (EDC), 1,1′-carbonyldiimidazole (CDI), 2-chloro-1-methylpyridinium iodide, etc.) in the presence or absence of 1-hydroxybenzotriazole (1-HOBt), by subjecting to a reaction carboxylic acid and amine at a temperature of 0 to 40° C.

The reactions 1), 2) and 3) are desirably carried out under atmosphere of inert gas (argon, nitrogen, etc.) and anhydrous conditions.

(5) Among the compounds of formula (I), the compound wherein AA¹ and AA² do not represent a single bond at the same time, and at least one of R, AA¹, AA², R⁷, R⁸, R¹⁰ contains carboxy, hydroxy, amino, thiol, guanidino or phosphono, i.e. the compound of formula (IC-2),

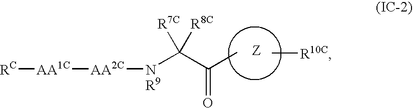

wherein R^{C}, AA^{1C}, AA^{2C}, R^{7C}, R^{8C} and R^{10C} have the same meaning as R, AA¹, AA², R⁷, R⁸ and R¹⁰ respectively, with proviso that AA^{1C} and AA^{2C} do not represent a single bond at the same time, and at least one of R^{C}, AA^{1C}, AA²C, R^{7C}, R^{8C} or R^{10C} represents a group which contains carboxy, hydroxy, amino, thiol, guanidino or phosphono or R^{C} is hydrogen, and the other symbols have the same meanings as above, may be prepared by subjecting to a deprotection reaction of protective groups of carboxy, hydroxy, amino, thiol, guanidino or phosphono, the compound among the compound of formula (IA-2), prepared by the above described method, wherein at least one of R, AA^{1C}, AA^{2C}, R^{7A}, R^{8A} or R^{10A} contains a protected form of carboxy, hydroxy, amino, thiol or guanidino, i.e. the compound of formula (IA-2-1),

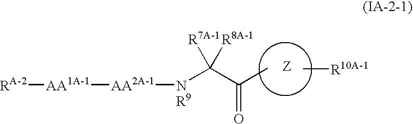

wherein R^{A-2}, AA^{1A-1}, AA^{2A-1} R^{7A-1} R^{8A-1} and R^{10A-1} have the same meanings as R^{A}, AA^{1A}, AA^{2A}, R^{7A}, R^{8A} and R^{10A}, with proviso that R^{A-2}, AA^{1A-1}, AA^{2A-1}, R^{7A-1}, R^{8A-1} and R^{10A-1} contain at least one protected carboxy, hydroxy, amino, thiol, guanidino or phosphono, or R^{A-2} is a protective group of amino, and the other symbols have the same meaning as above.

Deprotection reaction of protective groups of carboxy, hydroxy, amino, thiol, guanidino and phosphono may be carried out according to the above method.

And the compounds of formula (IIA-1) and (IIA-2) may be prepared according to the following reaction scheme 1.

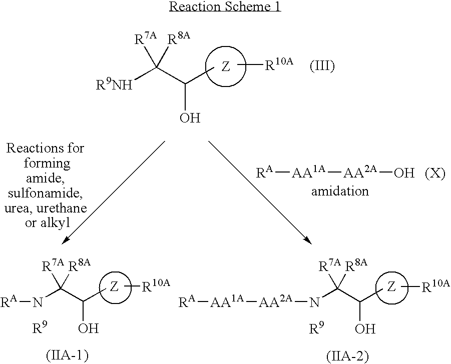

In the reaction scheme 1, all symbols have the same meanings as above.

In the compound of formula (III), the compound wherein Z is

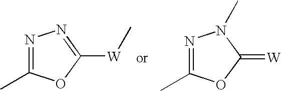

(wherein W has the same meaning as above.), i.e. the compound of formula (III-1)

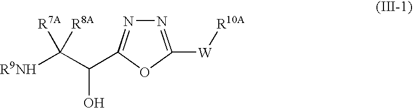

(wherein all symbols have the same meanings as above.) and the compound of formula (II-2)

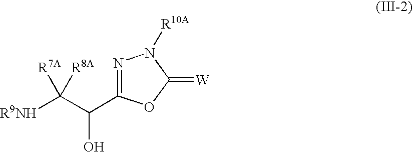

(wherein all symbols have the same meanings as above.) may be prepared according to the following reaction scheme 2.

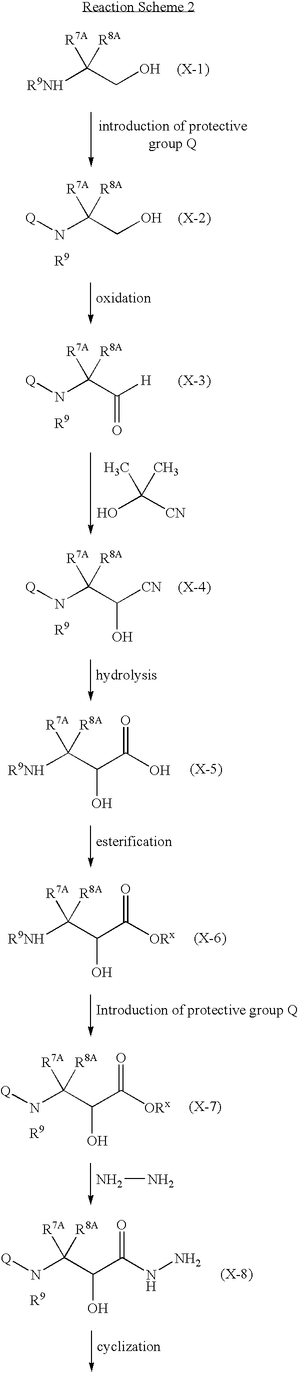

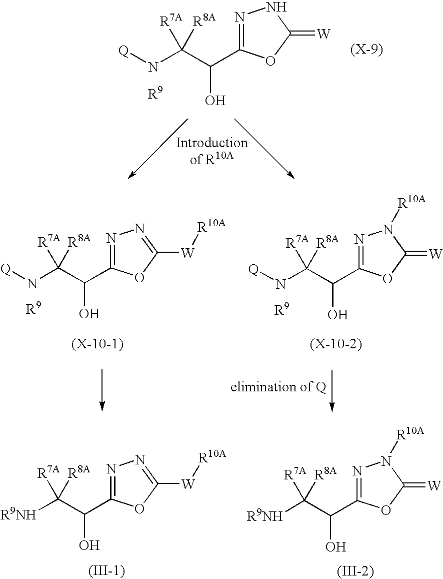

In reaction scheme 2, Q is t-butoxycarbonyl or benzyloxycarbonyl. Rx is methyl, ethyl or t-butyl, and the other symbols have the same meanings as above.

Among the compound of formula (III), the compound wherein Z ring is

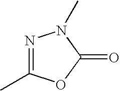

and R⁹ is hydrogen, i.e. the compound of formula (III-3)

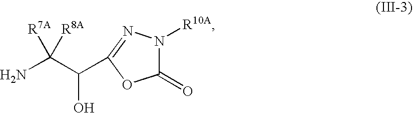

wherein all symbols have the same meanings as above, may be prepared according to the following reaction scheme 3.

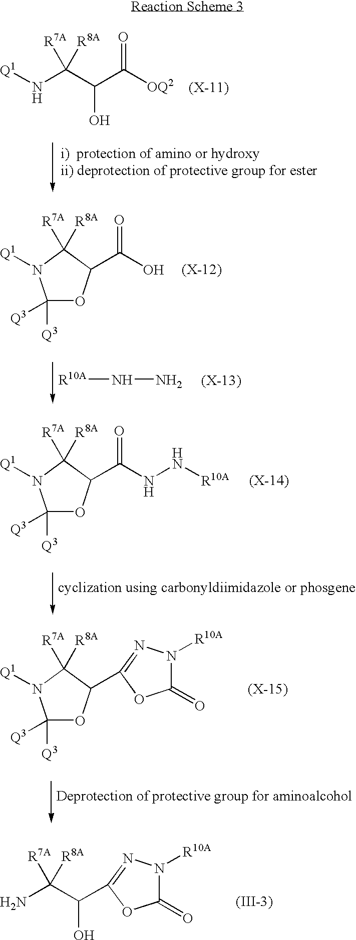

In the reaction scheme 3, Q1 is a protective group for amino (t-butoxycarbonyl etc.), Q² is a protective group for carboxy (methyl, ethyl, etc.),

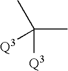

is a protective group for aminoalcohol (e.g. Q³ is methyl or ethyl.).

Among the compounds of formula (IIA-1) and (IIA-2), the compounds wherein Z is

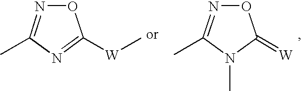

i.e. the compound of formula (IIA-1-A)

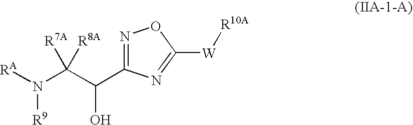

(wherein all symbols have the same meanings as above.), formula (IIA-1-B)

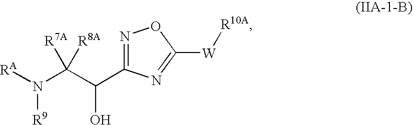

wherein all symbols have the same meanings as above, formula (IIA-2-A)

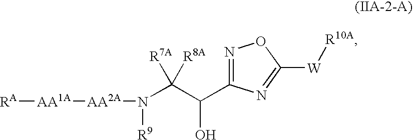

wherein all symbols have the same meanings as above, formula (IIA-2-B)

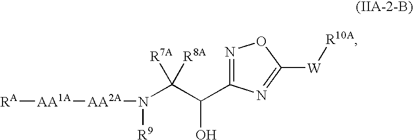

wherein all symbols have the same meanings as above, may be prepared according to the method described in the reaction schemes 4, 5 and 6.

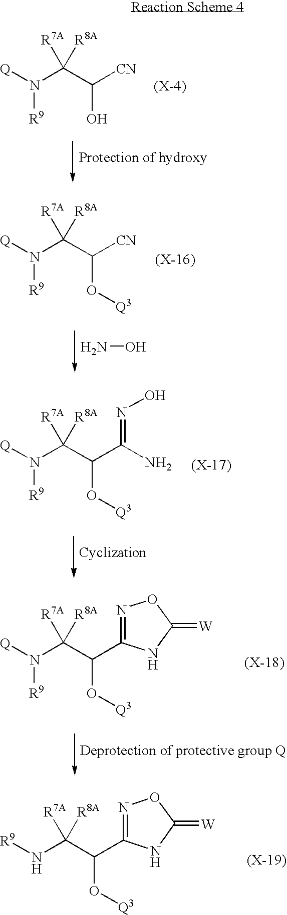

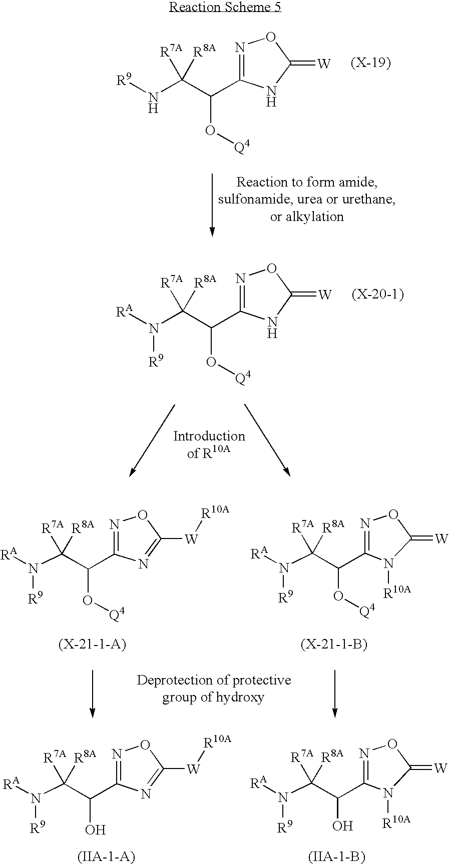

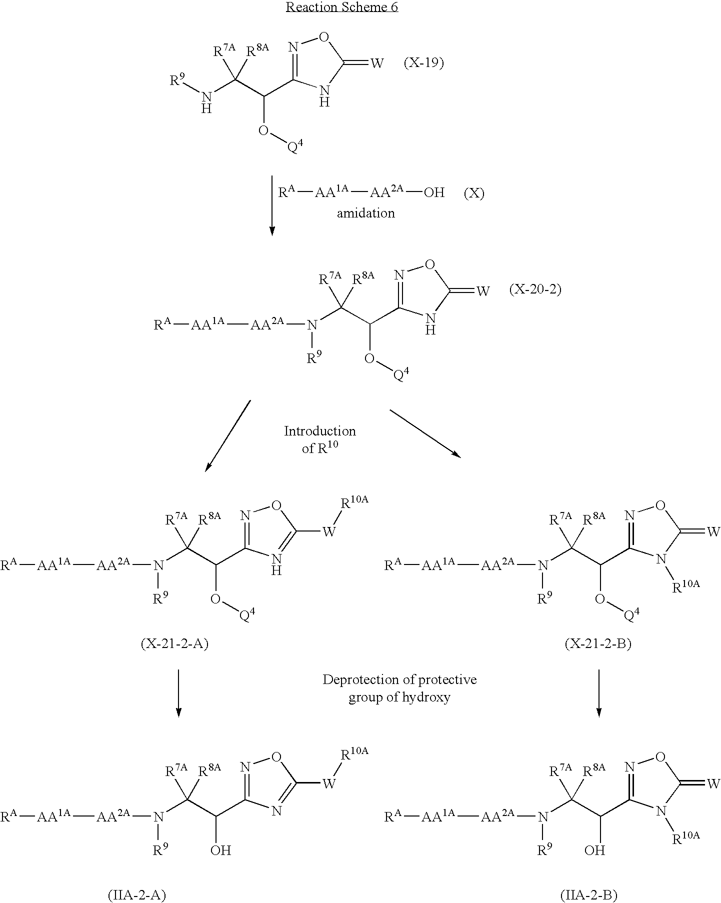

In the reaction schemes 4, 5 and 6, Q⁴ is a protective group for hydroxy (t-butyldimethylsilyl, trimethylsilyl, etc.) and the other symbols have the same meanings as above.

Among the compounds of formula (IIA-1) and (IIA-2), the compound wherein W is oxygen, i.e. the compound of formula (IIA-1-C)

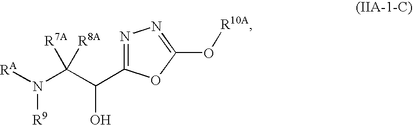

wherein all symbols have the same meanings as above, and (IIA-2-C)

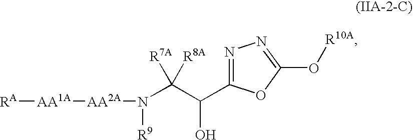

wherein all symbols have the same meanings as above, may also be prepared according to the method shown by the following reaction scheme 7.

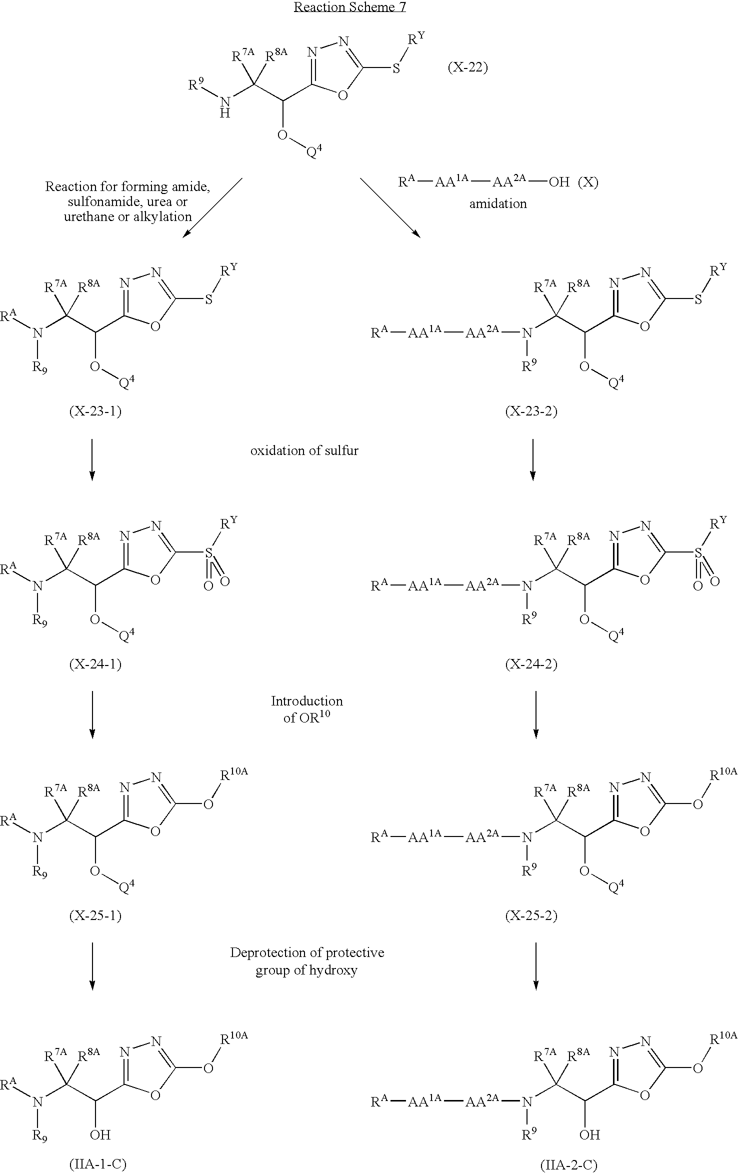

In the reaction scheme 7, Ry is lower alkyl such as methyl, ethyl, etc., and the other symbols have the same meanings as above.

The compounds of formula (X), (X-1) and (X-4), which are used as starting materials, are known per se or may be prepared according to the known methods. And the compound of formula (X-22) may be prepared by introducing a protective group Q⁴ to the compound of formula (III-1).

All reactions in the reaction schemes may be carried out by conventional methods. Other starting materials and agents in the present invention are known per se or may be prepared by conventional methods.

In each reaction of the present specification, reaction products may be purified by conventional techniques. For example, purification may be carried out by distillation under atmospheric or reduced pressure, by high performance liquid chromatography, thin layer chromatography or column chromatography using silica gel or magnesium silicate, by washing or by recrystallization, etc. Purification may be carried out after each reaction, or after a series of reactions.

Pharmacological Activity of the Compounds of the Present Invention:

It was confirmed by the following experiments that the compounds of the present invention of formula (I) have an inhibitory activity against cysteine protease.

(i) Measurement of Cathepsin K Inhibitory Activity

65 μL of Cathepsin K enzyme reaction buffer (50 mmol/L of 2-(N-morpholino)ethanesulfonate, 2 mmol/L of ethylene diamine tetraacetate (EDTA) and 4 mmol/L of dithio threitol (DTT) were mixed to adjust to pH 5.5), 5 μL of cysteine protease inhibitor solution of several concentrations, 20 μL of synthesized substrate (t-butyloxycarbonyl-L-alanyl-glycyl-L-prolyl-L-arginine-4-methyl-chromanyl-7-amide) solution of several concentrations and 10 μL of cathepsin K enzyme solution were mixed and the increase of fluorescence intensity when reacted at 37° C. was measured (Ex (excitation wavelength)=355 nm, Em (fluorescence wavelength)=460 nm). As to the substrate and the compound of the present invention, enzyme reactions were carried out in combination of several appropriate concentrations and Dixon plotting was prepared, to define the absolute value of X-coordinate of the intersection point of the graph as Ki value.

It was confirmed that the compound of the present invention of formula (I) had an inhibitory activity more than 50% at 10 μM. For example, the Ki value of inhibitory activity of the compounds of example 1 was 48 nM.

(ii) Measurement of Cathepsin B Inhibitory Activity 10 μL of Synthesized substrate (carbobenzoxy-L-arginyl-L-arginine-4-methyl-chromanyl-7-amide or carbo benzoxy-L-phenylalanyl-L-arginine-4-methyl-chromanyl-7-amide) solution of several concentrations, 10 μl of cysteine protease inhibitor solution of several concentrations, 70 μl of cathepsin B enzyme reaction buffer (mixture of 400 mmol/L in acetic acid, 4 mmol/L EDTA, 8 mmol/L DDT to adjust to pH 5.5) and 10 μl of cathepsin B enzyme solution were mixed and the increase of fluorescence intensity was measured (Ex=355 nm, Em=460 nm) when reacted at 37° C.

It was confirmed that the compound of the present invention of formula (I) had an inhibitory activity more than 50% at 10 μM.

(iii) Measurement of Cathepsin S Inhibitory Activity

10 μl of synthesized substrate (carbobenzoxy-L-leucyl-L-leucyl-L-arguinine-4-methyl-chromanyl-7-amide) solution and 5 μl of cysteine protease inhibitor solution of several concentrations, 75 μl of cathepsin S enzyme reaction buffer (100 mmol/L of sodium phosphate, 2 mmol/L of EDTA, 2 mmol/L of DTT were mixed to adjust to pH 6.5) and 10 μl of cathepsin S enzyme solution were mixed and the increase of fluorescence intensity was measured (Ex=355 nm, Em=460 nm) when reacted at 37° C.

It was confirmed that the compound of the present invention of formula (I) has an inhibitory effect more than 50% at 10 μM.

(iv) Measurement of Cathepsin L Inhibitory Activity

5 μl of Synthesized substrate (carbobenzoxy-L-phenylalanyl-L-arguine-4-methyl-chromanyl-7-amide or L-prolyl-L-phenylalanyl-L-arguinine-4-methyl-chromanyl-7-amide) solution and 5 μl of cysteine protease inhibitor solution of several concentrations, 80 μl of cathepsin L enzyme reaction buffer (400 mmol/L acetic acid, 4 mmol/L EDTA, 8 mmol/L DTT were mixed to adjust to pH 5.5) and 10 μl of cathepsin L enzyme solution were mixed and the increase of fluorescence intensity was measured (Ex=355 nm, Em=460 nm) when reacted at 37° C.

It was confirmed that the compound of the present invention of formula (I) had an inhibitory activity of more than 50% at 10 μM.

(v) Measurement of Calpain Inhibitory Activity

The activity was measured according to the method described in Calcium-depending protease, Seibutsukagaku-Jikkenhou (Biochemistry Experimental Method) Tanpakubunkaikouso (Protease) I, 57 (1993).

(vi) Measurement of Caspase-1 Inhibitory Activity

50 μl of caspase-1 enzyme reaction solution (20 mmol/L of 4-(2-hydroxyethyl)-1-piperazinethanesulfonate-sodium hydroxide buffer pH 7.4, 10 mmol/L of potassium chloride, 1.5 mmol/L of magnesium chloride, 0.1 mmol/L EDTA, 10% glycerol) and 50 μl of cysteine protease inhibitor solution of several concentrations, 50 μl of caspase-1 enzyme solution and 100 μl of synthesized substrate (acetyl-L-tyrosinyl-L-valinyl-L-alanyl-L-aspartic acid-4-methyl-chromanyl-7-amide)-solution of several concentrations were reacted at 37° C. and the fluorescence intensity was measured (Ex=355 nm, Em=460 nm).

(vii) Investigation in Bone Resorption Inhibitory Activity Using Mouse Calvaria Cultivation System

Mouse neonatal calvaria was cultured in D-minimum essential medium containing cysteine protease inhibitor (mixture of Penicillin G potassium (final concentration 100 U/ml), streptomycin sulfate (final concentration 0.1 mg/ml), bovine serum albumin (final concentration 0.1%), glutamine (final concentration 0.3 mg/ml) in D-minimal essential medium) with incitant (parathyroid hormone (PTH) or arotinoid) at 37° C. and the calcium concentration in the culture medium was measured.

(viii) Bone Resorption Pit Formation Test Using Rabbit Osteoclast Cells

Osteoclast cells collected from rabbit bones were sowed over slices of bovine cortical bone, dentine or teeth of toothed whale and were cultured at 37° C. in α-minimal essential medium containing final concentration 5% of fetal bovine serum and various concentrations of cysteine protease inhibitor. The pits formed on the slices by the osteoclast cells were observed and at the same time type-I collagen C-terminal telopeptide (CTx) concentration in culture medium was measured.

(ix) Investigation of Immune Reaction Inhibitory Effect Using Antigen-Sensitized Mouse Spleen Cells

Spleen cells were collected from mice sensitized by ovalbumin (OVA) several times. Inhibitory effect of cysteine protease inhibitors against immune response induced by OVA stimulus was investigated, using cytokine concentration and immunoglobulin concentration in culture solution as indicators.

(x) Investigation in Inhibitory Effect Against Bone Resorption Using the Rat PTH Hypercalcemia Model

The effect of cysteine protease inhibitor (compulsory oral administration, intraperitoneal administration) on bone resorption which was promoted by intravenous administration of parathyroid hormone (PTH) solution (30 μg/ml) was investigated in rats, using calcium concentration in blood as an indicator.

(xi) Studies on Bone Resorption Inhibitory Effect Using TPTx Rat PTHrP-Induced Hypercalcemia Model

The effect of cysteine protease inhibitor (compulsory oral administration, intraperitoneal administration) on bone resorption, promoted by subcutaneous administration of parathyroid hormone related peptide (PTHrP) to a fasting rat (thyroparathyroidectomized; TPTx) was investigated, using calcium concentration in blood as an indicator.

Toxicity:

The toxicity of the compounds of the present invention is very low and therefore it was confirmed that the compounds are safe for pharmaceutical use.

### INDUSTRIAL APPLICABILITY

Application to Pharmaceuticals:

The compound of formula (I) of the present invention has an inhibitory activity against cysteine proteases, and therefore it is useful as an agent for the prophylaxis and/or treatment of inflammatory diseases (periodontitis, arthritis, inflammatory bowel diseases, infectious diseases, pancreatitis, hepatitis, glomerulonephritis, endocarditis, myocarditis, etc.), diseases induced by apoptosis (graft versus host diseases, rejection of an organ transplantation, acquired immune deficiency syndrome (AIDS), AIDS-related complex (ARC), adult T cell leukemia, hairy cells leukemia, spondylopathy, disorders of respiratory apparatus, arthritis, HIV or HTLV-1 related diseases such as uveitis, virus-related diseases such as hepatitis C, cancer, collagenosis (systemic lupus erythematosus, rheumatoid arthritis, etc.), ulcerative colitis, Sjoegren's syndrome, primary biliary cirrhosis, spontaneous thrombocytopenic purpura, autoimmune hemolytic anemia, myasthenia gravis, autoimmune diseases such as insulin dependent (type I) diabetes, diseases accompanying thrombocytopenia (osteomyelodysplasia syndrome, periodic thrombocytopenia, aplastic anemia, spontaneous thrombocytopenia, disseminated intravascular coagulation (DIC), etc.), hepatic diseases such as viral hepatitis (type A, B, C, F, etc.) or hepatitis medicamentosa and cirrhosis, dementia such as Alzheimer's diseases and Alzheimer's senile dementia, cerebrovascular injury, nerve degeneration diseases, adult acute respiratory distress syndrome, infectious diseases, prostatomegaly, hysteromyoma, bronchial asthma, arteriosclerosis, all kinds of lusus naturae, nephropathy, senile cataract, chronic fatigue syndrome, myodystrophy, peripheral neuropathy, etc.), diseases induced by disorders of immune response (graft versus host diseases, rejection of an organ transplantation, allergic diseases (bronchial asthma, atopic dermatitis, allergic rhinitis, pollinosis, diseases induced by house dusts, irritable pneumonia, food allergy, etc.), psoriasis, rheumatoid arthritis, etc.), autoimmune diseases (insulin-dependent (type I) diabetes, systemic lupus erythematosus, Hashimoto's diseases, multiple sclerosis, etc.), disease by decomposing various proteins which compose the organism (myodystrophy, cataract, periodontitis, hepatocyte disease by bile acid such as cholestatic cirrhosis, etc.), decomposition of alveolus elastica such as pulmonary emphysema, ischemic diseases (brain ischemia, brain disorders by ischemic reperfusion, myocardial infarction, ischemic hepatopathy, etc.), shock (septic shock, systemic inflammation response syndrome, endotoxin shock, acidosis, etc.), circulatory system disorders (arteriosclerosis, restenosis after percutaneous transluminal coronary angioplasty (PTCA), etc.)), blood coagulation disorders (thrombocytopenic purpura, hemolytic uremic syndrome, etc.), malignant tumor, acquired immune deficiency syndrome (AIDS) and AIDS-related complex (ARC), parasitic diseases such as malaria, nerve degenerative diseases (Alzheimer-type dementia, Huntington's chorea, Parkinson's diseases, multiple sclerosis, traumatic encephalopathy, traumatic spondylopathy, etc.), pulmopathy such as fibroid lungs, bone resorption diseases (osteoporosis, rheumatoid arthritis, arthritis, osteoarthritis, hypercalcemia, osteometastasis of cancer etc.), endocrinesthenia such as hyperthyroidism.

For the purpose described above, the compounds of formula (I), of the present invention, non-toxic salts thereof, acid addition salts thereof or hydrates thereof may normally be administered systemically or locally, usually by oral or parenteral administration.

The doses to be administered are determined depending upon, e.g. age, body weight, symptom, the desired therapeutic effect, the route of administration, and the duration of the treatment. In the human adult, the doses per person at a time are generally from 1 to 1000 mg, by oral administration, up to several times per day, and from 1 to 100 mg, by parenteral administration (preferably intravenous administration), up to several times per day, or continuous administration for from 1 to 24 hours per day from vein.

As mentioned above, the doses to be used depend upon various conditions. Therefore, there are cases wherein doses lower or greater than the ranges specified above may be used.

The compounds of the present invention may be administered in the form of, e.g., solid compositions, liquid compositions or other compositions for oral administration, injections, liniments or suppositories for parenteral administration.

Solid compositions for oral administration include compressed tablets, pills, capsules, dispersible powders and granules.

Capsules include hard capsules and soft capsules.

In such solid compositions, one or more of the active compound(s) may be used as a dosage form, as is normal practice, to admix with excipient (e.g. lactose, mannitol, glucose, microcrystalline cellulose, starch), combining agents (hydroxypropyl cellulose, polyvinyl pyrrolidone or magnesium metasilicate aluminate), disintegrating agents (e.g. cellulose calcium glycolate), lubricating agents (e.g. magnesium stearate), stabilizing agents, agents to assist dissolution (e.g. glutamic acid or asparatic acid) and the like. The agents may, if desired, be coated with coating agents (e.g. sugar, gelatin, hydroxypropyl cellulose or hydroxypropylmethyl cellulose phthalate), or be coated with two or more films. Further, coating may include containment within capsules of absorbable materials such as gelatin.

Liquid compositions for oral administration include pharmaceutically acceptable solutions, suspensions, emulsions, syrups and elixirs. In such compositions, one or more of the active compound(s) are dissolved, suspended or emulsified in diluent commonly used (e.g. purified water, ethanol or mixture thereof). Furthermore, such liquid compositions may also comprise wetting agents or suspending agents, emulsifying agents, sweetening agents, flavoring agents, perfuming agents, preserving agents buffer agent etc.

Injections for parenteral administration include solutions, suspensions, emulsions and solids which are dissolved or suspended to use at a time to use. One or more of the active compound(s) in injections are dissolved, suspended and emulsified in a solvent. The solvents are, e.g., distilled water for injection, physiological salt solution, vegetable oil, propylene glycol, polyethylene glycol, alcohol such as ethanol or mixture thereof. Moreover the injections may also include stabilizing agents, agents to assist dissolution (e.g. glutamic acid, aspartic acid or POLYSORBATE80 (registered trade mark)), suspending agents, emulsifying agents, soothing agents, buffer agents, preserving agents, etc. They are sterilized in the last process or manufactured and prepared by sterile procedure. They may also be manufactured in the form of sterile solid compositions such as freeze-dried one and they may be sterilized or dissolved to use in sterile distilled water for injection or some other solvents immediately before use.

Other compositions for parenteral administration include liquids for external use, and ointment, endermic liniments, inhale, spray, suppositories for rectal administration and pessaries for vaginal administration which comprise one or more of the active compound(s) and are prescribed by methods known per se.

Spray compositions may comprise additional substances other than diluents: e.g. stabilizing agents (e.g. sodium sulfite hydride), isotonic buffers (e.g. sodium chloride, sodium citrate or citric acid). For preparation of such spray compositions, e.g., the method described in the U.S. Pat. No. 2,868,691 or No.3,095,355 may be used.

### BEST MODE FOR CARRYING OUT THE INVENTION

The following reference examples and Examples illustrate the present invention, but do not limit the present invention.

The solvents in the parentheses show the eluting or developing solvents and the ratios of the solvents used are by volume in chromatographic separations or TLC.

The solvents in the parentheses in NMR show the solvents used in measurement. In the chemical structures, TBS is t-butyldimethylsilyl, Boc is t-butoxycarbonyl and TsOH is tosyl acid.

### REFERENCE EXAMPLE 1

(2S)-2-(t-butoxycarbonylamino)-4-methylpentanol

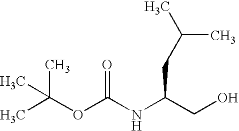

To a solution of (2S)-2-amino-4-methylpentanol ((L)-leucinol) (20 g) in tetrahydrofuran (THF; 1000 ml) was added di-t-butyl-dicarbonate (43 ml) at 0° C. and the mixture was stirred for 1.5 hours at room temperature. The reaction mixture was concentrated to give a crude compound of title compound having the following physical data.

TLC: Rf 0.50(chloroform:methanol=10:1); NMR (CDCl₃): δ 4.58 (br, 1H), 3.81-3.45 (m, 3H), 1.80-1.60 and 1.37-1.25 (each m, total 3H), 1.45 (s, 9H), 0.95-0.91 (m, 6H).

### REFERENCE EXAMPLE 2

(2S)-2-(t-butoxycarbonylamino)-4-methylpentanal

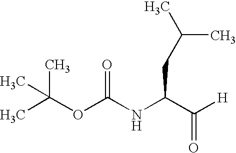

To a solution of the crude compound prepared in reference example 1 in dimethylsulfoxide (DMSO; 344 ml) were added triethylamine (72 ml) and sulfur trioxide-pyridine complex (82 g) in DMSO (280 ml) at 10° C. and the mixture was stirred for 1 hour. The reaction mixture was poured into ice-water and was extracted with ethyl acetate. The organic layer was washed with 10% aqueous solution of citric acid, water and a saturated aqueous solution of sodium chloride successively and was dried over anhydrous sodium sulfate and was concentrated to give the crude compound of the title compound having the following physical data.

TLC: Rf 0.45 (chloroform:methanol=10:1); NMR (CDCl₃): δ 9.59 (s, 1H), 4.91 (br, 1H), 4.12 (br, 1H), 1.80-1.60 and 1.40-1.30 (each m, total 3H), 1.46 (s, 9H), 1.00-0.87 (m, 6H).

### REFERENCE EXAMPLE 3

(3S)-3 (t-butoxycarbonylamino)-2-hydroxy-5-methylhexanenitrile

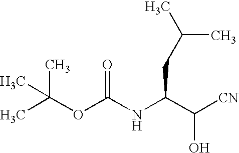

To a solution of the crude compound prepared in reference example 2 in methanol (180 ml) was added acetonecyanohydrine (19 ml) and potassium carbonate (4.7 g) and the mixture was stirred for 1 hour at room temperature. The reaction mixture was concentrated and the residue was extracted with ethyl acetate and water. The organic layer was washed with water and a saturated aqueous solution of sodium chloride, dried over anhydrous sodium sulfate and was concentrated. The residue was purified by column chromatography on silica gel (n-hexane:ethyl acetate=3:1) to give the title compound (33.6 g) having the following physical data.

TLC: Rf 0.40 (n-hexane:ethyl acetate=3:1); NMR (CDCl₃): δ 4.85-4.80 (m, 1H), 4.60-4.45 (m, 1H), 4.00-3.70 (m, 1H), 1.80-1.40 (m, 3H), 1.45 and 1.43 (each s, total 9H), 1.00-0.90 (m, 6H).

### REFERENCE EXAMPLE 4

(3S)-3-amino-2-hydroxy-5-methylhexanoic acid hydrochloride

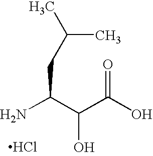

To the compound prepared in reference example 3 (33.6 g) was added a concentrated hydrochloric acid (300 ml) and the mixture was stirred for 5 hours at 80° C. The reaction mixture was concentrated to give a crude compound of the title compound having the following physical data.

TLC: Rf 0.30 (chloroform:methanol:water=6:4:1).

### REFERENCE EXAMPLE 5

methyl (3S)-3-amino-2-hydroxy-5-methylhexanoic acid hydrochloride

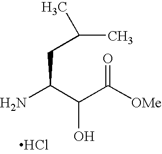

To methanol (1000 ml) was added thionyl chloride (92 ml) at −40° C. and the mixture was stirred for 10 minutes. The solution was dropped to a solution of the compound prepared in reference example 4 in methanol (250 ml) at −10° C. and the mixture was stirred at room temperature for 4 hours. The reaction mixture was concentrated to give the crude product of the title compound having the following physical data.

TLC: Rf 0.50(chloroform:methanol:water=6:4:1).

### REFERENCE EXAMPLE 6

Methyl (3S)-3-(t-butoxycarbonylamino)-2-hydroxy-5-methylhexanoic acid

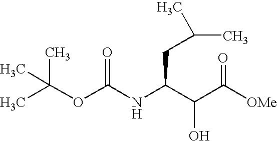

To a solution of methylene chloride (300 ml) of the crude compound prepared in reference example 5 (32 g) were added triethylamine (20 ml) and di-t-butyl dicarbonate (34 ml) at 0° C. and the mixture was stirred for 4 hours at room temperature. To the reaction mixture was added water and was extracted with ethyl acetate. The organic layer was washed with 10% aqueous solution of citric acid, a saturated aqueous solution of sodium bicarbonate, water and a saturated aqueous solution of sodium chloride, dried over anhydrous sodium sulfate and was concentrated. The residue was purified by column chromatography on silica gel (n-hexane:ethyl acetate=3:1) to give the title compound (28 g) having the following physical data.

TLC: Rf 0.40 and 0.35 (n-hexane:ethyl acetate=3:1); NMR (CD₃OD): δ 4.10-4.09 (m, 1H), 4.04-3.95 and 3.93-3.85 (each m, total 1H), 3.72 and 3.70 (each s, total 3H), 1.70-1.08 (m, 3H), 1.43 and 1.40 (each s, total 9H), 0.98-0.82 (m, 6H).

### REFERENCE EXAMPLE 7

(3S)-3-t-butoxycarbonylarino-2-hydroxy-5-methylhexanoic acid hydrazide

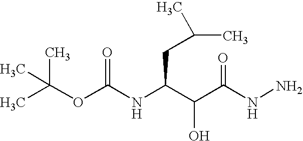

To hydrazine hydrate (99 ml) was added the compound prepared in reference example 6 (28 g) in methanol (110 ml) at 0° C. dropwise and the mixture was stirred for 1 hour at room temperature. To the reaction mixture was added water and was extracted with methylene chloride. The organic layer was washed with a saturated aqueous solution of sodium chloride and was dried over anhydrous sodium sulfate and was concentrated to give the title compound (21 g) having the following physical data.

TLC: Rf 0.40 (chloroform:methanol:water=9:1:0.1); NMR (CD₃OD): δ 4.10 (d, J=3.6 Hz, 0.5H), 4.00-3.90 (m, 1.5H), 1.70-1.30 (m, 3H), 1.43 and 1.41 (each s, total 9H), 0.95-0.88 (m, 6H).

### REFERENCE EXAMPLE 8

(2S)-2-(N-t-buxtoxycarbonylamino)-4-methyl-1-(2-thioxo-1,3,4-oxadiazolin-5-yl)pentanol

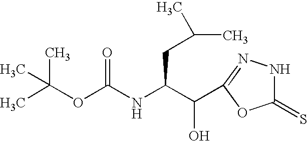

To a solution of the compound prepared in reference example 7 (3.0 g) in 95% ethanol (55 ml) were added potassium hydroxide (726 mg) and carbon disulfide (662 ml) and the mixture was stirred overnight at 90° C. The reaction mixture was cooled to room temperature and thereto was added cold 10% aqueous solution of citric acid and was extracted with ethyl acetate. The organic layer was washed with a saturated aqueous solution of sodium chloride and dried over anhydrous sodium sulfate and was extracted. The residue was purified by column chromatography on silica gel (ethyl acetate) and thereto was added a 10% aqueous solution of citric acid and was extracted with ethyl acetate. The organic layer was washed with a saturated aqueous solution of sodium chloride, dried over anhydrous sodium sulfate and was concentrated to give the title compound (3.1 g) having the following physical data.

TLC: Rf 0.31 (n-hexane:ethyl acetate=1: I); NMR (CDCl₃): δ 11.80 (br, 1H), 5.28 and 5.09 (each br, total 1H), 5.00-4.40 (m, 2H), 4.20-3.90 (m, 1H), 2.00-1.20 (m, 3H), 1.47 and 1.43 (each s, total 9H), 1.05-0.85 (m, 6H).

### REFERENCE EXAMPLE 9

(2S)-2-(N-t-butoxycarbonylamino)-4-methyl-1-[5-(2-dimethylaminoethylthio)-1,3,4-oxadiazol-2-yl]pentanol

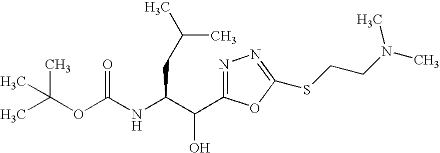

A solution of the compound prepared in reference example 8 (25.4 g), 2-chloroethyldimethylamine hydrochloride (12.7 g) and potassium carbonate (27.6 g) in N,N-dimethylformamide (DMF; 240 ml) was stirred for 13 hours at 50° C. The reaction mixture was poured into water and was extracted with ethyl acetate. The organic layer was washed with a saturated aqueous solution of sodium chloride and was dried over anhydrous magnesium sulfate and was concentrated. The residue was purified by column chromatography on silica gel (chloroform:methanol=100:2 to 4:1) to give the title compound (23.5 g) having the following physical data.

TLC: Rf 0.32(chloroform:methanol=9:1); NMR (CDCl₃): δ 4.91-4.70 (m, 2H), 4.23-4.10 and 4.07-3.92 (each m, total 1H), 3.44-3.34 (m, 2H), 2.76-2.67 (m, 2H), 2.30 (s, 6H), 1.80-1.20 (m, 3H), 1.45 and 1.39 (each s, total 9H), 1.00-0.91 (m, 6H).

### REFERENCE EXAMPLE 10

1-[5-(2-dimethylaminoethylthio)-1,3,4-oxadiazol-2-yl]-1-hydroxy-4-methyl-2-pentylamine bishydrochloride

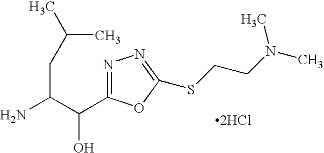

To a solution of the compound prepared in reference example 9 (1.53 g) in dioxane (8 ml) was added 4N hydrochloric acid-dioxane (16 ml) and the mixture was stirred for 1 hour at room temperature. The reaction mixture was concentrated to give a crude product of the title compound having the following physical data.

TLC: Rf 0.21 (chloroform:methanol:28% ammonia water=90:10:1); NMR (CDCl₃): δ 11.10-10.80 (br, 1H), 8.50-8.10 (br, 3H), 7.16 and 7.00 (each brd, J=5.1 Hz, total 1H), 5.17 and 5.01 (each brs, total 1H), 3.80-3.40 (m, 5H), 2.80 (s, 6H), 1.83-1.32 (m, 3H), 0.86 and 0.85 (each d, J=6.3 Hz, each 3H).

### REFERENCE EXAMPLE 11

cycloheptyl-N-[1-[5-(2-dimethylaminoethylthio)-1,3,4-oxadiazol-2-yl]-1-hydroxy-4-methyl-2-pentyl]carboxamide hydrochloride

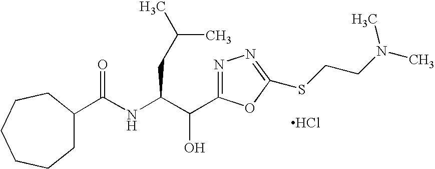

To a solution of the compound prepared in reference example 10 (1.42 g), cycloheptylcarboxylic acid (0.54 ml) and 1-hydroxybenzotriazole (725 mg) in DMF (15 ml) was added 1-ethyl-3-[3-(dimethylamino)propyl]carbodiimide hydrochloride (907 mg) and the mixture was stirred for 20 minutes. To the reaction mixture was added N-methylmorpholine (0.65 ml) and the mixture was stirred for 4 hours at room temperature. The organic layer was washed with a saturated aqueous solution of sodium bicarbonate and a saturated solution of sodium chloride successively, dried over anhydrous magnesium sulfate and was concentrated. The residue was purified by column chromatography on silica gel (chloroform:methanol=20:1) to give the title compound (128 mg) having the following physical data.

TLC: Rf 0.35 (chloroform:methanol:28% ammonia water=90:10:1); NMR (CDCl₃): δ 5.90-5.76 (m, 1H), 4.94-4.88 (m, 1H), 4.52-4.42 and 4.32-4.18 (each m, total 1H), 3.43-3.34 (m, 2H), 2.72 and 2.71 (each t, J=6.6 Hz, 2H), 2.34-2.14 (m, 1H), 2.30 (s, 6H), 1.92-1.31 (m, 15H), 0.94 and 0.92 (each d, J=6.3 Hz, total 6H).

### EXAMPLE 1

cycloheptyl-N-[1-[5-(2-dimethylaminoethylthio)-1,3,4-oxadiazol-2-yl]-4-methyl-1-oxo-2-pentyl]carboxamide hydrochloride

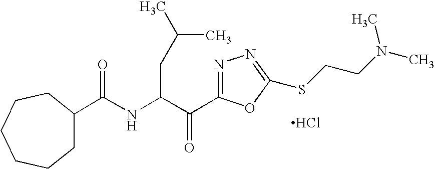

To a solution of Dess-Martin reagent (1,1,1-triacetoxy)-1,1-dihydro-1,2-benziodoxole-3(1H)-one; 1.72 g) in methylene chloride (15 ml) was added a solution of the compound prepared in reference examle 11 (835 mg) in methylene chloride (5 ml) at room temperature and the mixture was stirred for 2 hours. To the mixture was added a saturated aqueous solution of sodium thiosulfate (20 ml) and ethyl acetate and the mixture was stirred for 1 hour. The reaction mixture was poured into a saturated aqueous solution of sodium bicarbonate and the mixture was extracted with ethyl acetate, washed with a saturated sodium bicarbonate and a saturated aqueous solution of sodium chloride, dried over anhydrous magnesium sulfate and was concentrated. The residue was purified by column chromatography on silica gel (ethyl acetate:acetic acid:water=8:2:1) to give the compound of the present invention (407 mg) having the following physical data.

TLC: Rf 0.44 (chloroform:methanol=9:1); NMR (DMSO-d₆): δ 10.66-10.35 (br, 1H), 8.38 (d, J=6.3 Hz, 1H), 5.07-4.97 (m, 1H), 3.78-3.66 (m, 2H), 3.50 (t, J=7.2 Hz, 2H), 2.82 (brs, 6H), 2.45-2.30 (m, 1H), 1.86-1.30 (m, 15H), 0.91 and 0.90 (each d, J=6.0 Hz, each 3H).

### EXAMPLE 1(1)˜EXAMPLE 1(161)

By the same procedure as described in reference example 8→reference example 9→reference example 10→reference example 11→example 1 using corresponding compounds, optionally followed by converting to a corresponding salt by known methods, the compounds of the present invention having the following physical data were given.

### EXAMPLE 1(1)

1-benzoylaminocyclohexyl-N-[4-methyl-1-[5-(2-pyrrolidino ethylthio)-1,3,4-oxadiazol-2-yl]-1-oxo-2-pentyl]carboxamide

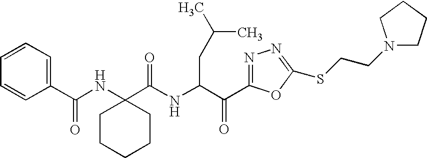

TLC: Rf 0.48 (chloroform:methanol=9:1); NMR (CDCl₃): δ 8.01 (d, J=6.6 Hz, 1H), 7.79-7.76 (m, 2H), 7.58-7.45 (m, 3H), 6.07 (s, 1H), 5.40-5.30 (m, 1H), 3.52-3.42 (m, 2H), 2.93 (t, J=6.9 Hz, 2H), 2.62 (br-s, 4H), 2.33-2.22 (m, 2H), 2.04-1.94 (m, 2H), 1.82-1.26 (m, 13H), 1.01 (d, J=6.0 Hz, 3H), 0.97 (d, J=6.0 Hz, 3H).

### EXAMPLE 1(2)

1-(4-morpholino-2-butynoylamino)cyclohexyl-N-[4-methyl-1-[5-(1-methylethylthio)-1,3,4-oxadiazol-2-yl]-oxo-2-pentyl]carboxamide

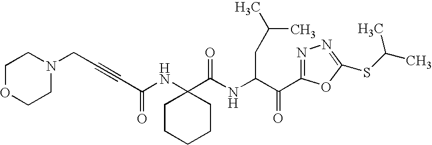

TLC: Rf 0.61 (chloroform:methanol=9:1); NMR (CDCl₃): δ 7.45 (brd, J=6.6 Hz, 1H), 5.76 (brs, 1H), 5.36 (m, 1H), 4.03 (septet, J=6.9 Hz, 1H), 3.76 (t, J=4.5 Hz, 4H), 3.43 (s, 2H), 2.60 (t, J=4.5 Hz, 4H), 2.10 (m, 2H), 1.95-1.20 (m, 11H), 1.53 (d, J=6.9 Hz, 6H), 1.01 (d, J=6.0 Hz, 3H), 0.98 (d, J=6.3 Hz, 3H).

### EXAMPLE 1(3)

cycloheptyl-N-[(2S)-1-[5-(1-methylethylthio)-1,3,4-oxadiazol-2-yl]-4-methyl-1-oxo-2-pentyl]carboxamide

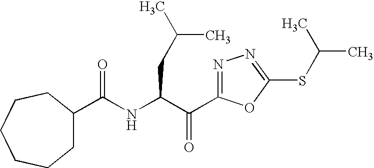

TLC: Rf 0.42 (ethyl acetate:n-hexane=1:1); NMR (CDCl₃): δ 5.95 (d, J=7.5 Hz, 1H), 5.42 (ddd, J=10.2, 7.5, 3.9 Hz, 1H), 4.11-3.96 (m, 1H), 2.38-2.24 (m, 1H), 1.96-1.38 (m, 15H), 1.53 (d, J=6.6 Hz, 6H), 1.03 and 0.97 (each d, J=6.3 Hz, each 3H).

### EXAMPLE 1(4)

1-(3-morpholinomethylbenzoylamino)cyclohexyl-N-[4-methyl-1-[5-(1-methylethylthio)-1,3,4-oxadiazol-2-yl]-1-oxo-2-pentyl]carboxamide

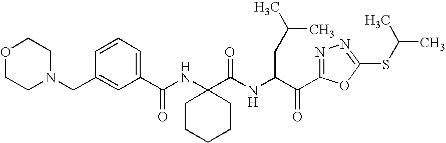

Free compound: TLC: Rf 0.56 (chloroform:methanol=9:1); NMR (CDCl₃): δ 8.00 (brd, J=6.6 Hz, 1H), 7.75 (brs, 1H), 7.65 (brd, J=7.8 Hz, 1H), 7.53 (brd, J=7.8 Hz, 1H), 7.42 (t, J=7.8 Hz, 1H), 6.09 (brs, 1H), 5.38 (m, 1H), 4.01 (septet, J=6.9 Hz, 1H), 3.72 (brt, J=4.5 Hz, 4H), 3.56 (s, 2H), 2.47 (m, 4H), 2.28 (m, 2H), 2.01 (m, 2H), 1.85-1.30 (m, 9H), 1.52 (d, J=6.9 Hz, 6H), 1.02 (d, J=6.3 Hz, 3H), 0.97 (d, J=6.3 Hz, 3H). Hydrochloride: TLC: Rf 0.56 (chloroform:methanol=9:1); NMR (DMSO-d₆): δ 11.01 (br, 1H), 8.13 (m, 2H), 7.85 (m, 2H), 7.75 (d, J=7.5 Hz, 1H), 7.54 (t, J=7.5 Hz, 1H), 4.98 (m, 1H),4.39 (s, 2H), 3.93 (m, 5H), 3.40-3.00 (m, 4H), 2.20-2.10 (m, 2H), 1.80-1.20 (m, 11H), 1.47 (d, J=6.9 Hz, 6H), 0.90-0.80 (m, 6H).

### EXAMPLE 1(5)

1-[(1R,2S)-2-(2-dimethylaminomethyl-4-fluorobenzoylamino)cyclohexyl]-N-[4-methyl-1-(5-methylthio-1,3,4-oxadiazol-2-yl)-1-oxo-2-pentyl]carboxamide hydrochloride

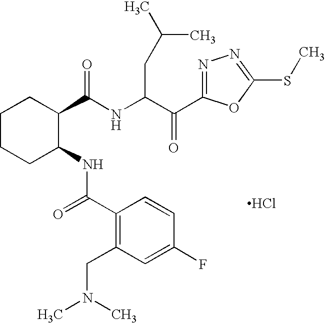

TLC: Rf 0.49 and 0.43 (chloroform:methanol=9:1); NMR (DMSO-d₆): δ 10.00-9.80 (broad, 1H), 8.66 and 8.57 (each brd, J=6.6 Hz, 1H), 8.47 and 8.32 (each brd, J=8.1 Hz, 1H), 7.65-7.52 (m, 2H), 7.45-7.21 (m, 1H), 5.00 (m, 1H), 4.42-4.20 (m, 3H), 2.80-2.65 (m, 10H), 2.00-1.20 (m, 11H), 0.89 and 0.87 (each d, J=6.3 Hz, 3H), 0.83 and 0.79 (each d, J=6.3 Hz, 3H).

### EXAMPLE 1(6)

1-[(1R,2S)-2-morpholinocyclohexyl]-N-[(2S)-4-methyl-1-[5-(1-methylethylthio)-1,3,4-oxadiazol-2-yl]-1-oxo-2-pentyl]carboxamide hydrochloride

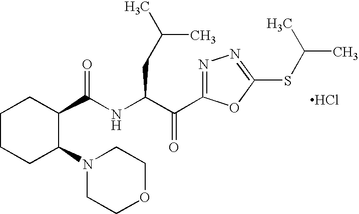

TLC: Rf 0.52 (chloroform:methanol=19:1); NMR (DMSO-d₆): δ 9.58 (br, 1H), 8.97 (d, J=6.3 Hz; 1H), 5.08-4.95 (m, 1H), 4.06-3.74 (m; 4H), 3.70-3.43 (m, 2H), 3.43-3.15 (m, 2H), 3.15-2.87 (m, 3H), 2.25-2.09 (m, 1H), 2.09-1.86 (m, 2H), 1.86-1.35 (m, 6H), 1.47 (d, J=6.9 Hz, 6H), 1.35-1.08 (m, 2H), 0.93 and 0.92 (each d, J=6.0 Hz, each 3H).

### EXAMPLE 1(7)

(2S)-N-[4-methyl-1-[5-(N-methylpiperidin-4-ylthio)-1,3,4-oxadiazol-2-yl]-1-oxo-2-pentyl]-2-benzyloxycarbonylamino-4-methylpentanamide

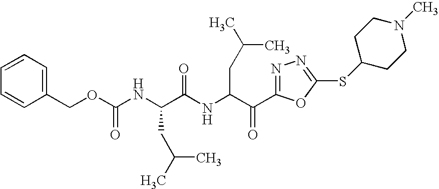

TLC: Rf 0.61 (chloroform:methanol:28% ammonia water=90:10:1); NMR (CDCl₃): δ 7.42-7.25 (m, 5H), 6.77-6.66 and 6.63-6.54 (each m, total 111), 5.48-5.37 (m, 1H), 5.20-5.00 (m, 3H), 4.36-4.12 (m, 1H), 3.93-3.80 (m, 1H), 2.89-2.74 (m, 2H), 2.35-2.14 and 2.00-1.41 (each m, total 12H), 2.31 (s, 3H), 1.07-0.80 (m, 12H).

### EXAMPLE 1(8)

cyclohexyl-N-[4-methyl-1-[5-(N-methylpiperidin-4-ylthio)-1,3,4-oxadiazol-2-yl]-1-oxo-2-pentyl]carboxamide

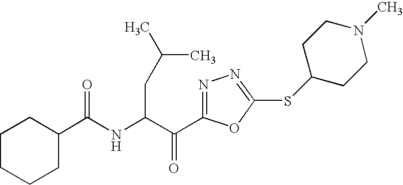

TLC: Rf 0.57 (chloroform:methanol:28% ammonia water=90:10:1); NMR (CDCl₃): δ 6.00 (brd, J=7.5 Hz, 1H), 5.47-5.38 (m, 1H), 3.94-3.80 (m, 1H), 2.90-2.74 (m, 2H), 2.36-1.17 (m, 20H), 2.31 (s, 3H), 1.03 and 0.97 (each d, J=6.0 Hz, each 3H).

### EXAMPLE 1(9)

1-[(1R,2S)-2-benzoylaminocyclohexyl]-N-[4-methyl 1-[5-(N-methylpiperdin-4 ylthio)-1,3,4-oxadiazol-2-yl]-1-oxo-2-pentyl]carboxamide

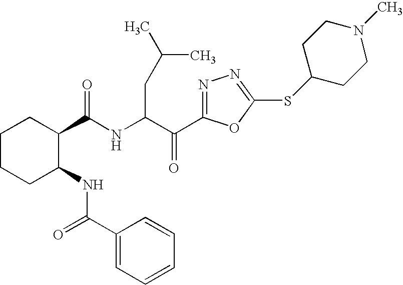

TLC: Rf 0.56 (chloroform:methanol:28% ammonia water=90:10:1); NMR (CDCl₃): δ 7.87-7.71 (m, 2H), 7.55-7.36 (m, 3H), 7.26-7.12 (m, 1H), 6.29 and 6.24 (each brd, J=7.5 Hz, total 1H), 5.49-5.33 (m, 1H), 4.45-4.26 (m, 1H), 3.94-3.79 (m, 1H), 2.91-2.72 (m, 3H), 2.35-1.40 (m, 17H), 2.30 (s, 3H), 1.02, 0.95, 0.91 and 0.85 (each d, J=6.3 Hz, total 6H).

### EXAMPLE 1(10)

1-benzoylaminocyclohexyl-N-[4-methyl-1-[5-(N-methylpiperidin-4-ylthio)-1,3,4-oxadiazol-2-yl]-1-oxo-2-pentyl]carboxamide

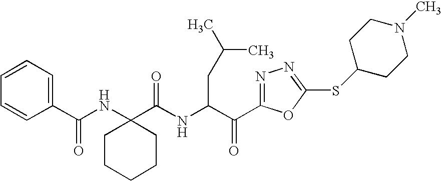

TLC: Rf 0.60 (chloroform:methanol:28% ammonia water=90: 10:1); NMR (CDCl₃): δ 8.03 (brd, J=6.9 Hz, 1H), 7.84-7.72 (m, 2H), 7.61-7.38 (m, 3H), 6.06 (s, 1H), 5.41-5.31 (m, 1H), 3.91-3.77 (m, 1H), 2.92-2.72 (m, 2H), 2.40-1.28 (m, 19H), 2.32 (s, 3H), 1.01, 0.98 and 0.97 (each d, J=6.0 Hz, total 6H).

### Example 1(11) cyclohexyl-N-[(2S)-1-[5-(1-methylethylthio)-1,3,4-oxadiazol-2-yl]-1-oxo-2-hexyl]carboxamide

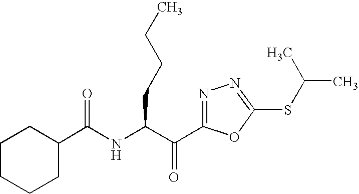

TLC: Rf 0.57 (ethyl acetate:n-hexane=1:2); NMR (CDCl₃): δ 6.10 (brd, J=7.5 Hz, 1H), 5.39 (ddd, J=8.7, 8.4, 5.1 Hz, 1H), 4.05 (septet, J=6.9 Hz, 1H), 2.22-1.20 (m, 22H), 0.90-0.85 (m, 3H).

### EXAMPLE 1(12)

20 cycloheptyl-N-[(2S)-1-[5-(1-methylethylthio)-1,3,4-oxadiazol-2-yl]-1-oxo-2-hexyl]carboxamide

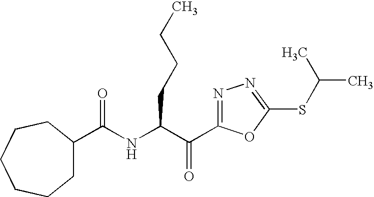

TLC: Rf 0.61 (ethyl acetate:n-hexane=1:2);NMR (CDCl₃): δ 6.02 (brd, J=7.2 Hz, 1H), 5.38 (ddd, J=8.4, 7.5, 4.8 Hz, 1H), 4.05 (septet, J=6.9 Hz, 1H), 2.35 (m, 1H), 2.14-1.35 (m, 24H), 0.90-0.85 (m, 3H).

### EXAMPLE 1(13)

cyclooctyl-N-[(2S)-4-methyl-1-[5-(1-methylethylthio)-1,3,4-oxadiazol-2-yl]-1-oxo-2-pentyl]carboxamide

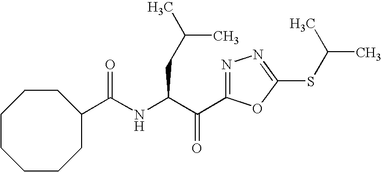

TLC: Rf 0.39 (ethyl acetate:n-hexane=1:3); NMR (CDCl₃): δ 5.94 (d, J=7.4 Hz, 1H), 5.42 (ddd, J=10.2, 7.4, 3.6 Hz, 1H), 4.11-3.96 (m, 1H), 2.42-2.29 (m, 1H), 1.90-1.40 (m, 23H), 1.03 and 0.98 (each d, J=6.3 Hz, each 3H).

### EXAMPLE 1(14)

1-morpholinocarbonylaminocyclohexyl-N-[1-[5-(2-dimethylaminoethylthio)-1,3,4-oxadiazol-2-yl]-4-methyl-1-oxo-2-pentyl]carboxamide

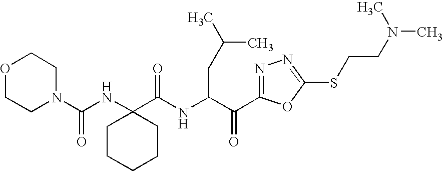

TLC: Rf 0.54 (chloroform:methanol:28% ammonia water=90:10:1); NMR (CDCl₃): δ 8.17 (brd, J=6.9 Hz, 1H), 5.35-5.25 (m, 1H), 4.42 (brs, 1H), 3.72 (t, J=4.8 Hz, 4H), 3.48 and 3.47 (each t, J=6.6 Hz, total 2H), 3.38 (t, J=4.8 Hz, 4H), 2.74 (t, J=6.6 Hz, 2H), 2.30 (s, 6H), 2.21-1.25 (m, 13H), 1.00 and 0.97 (each d, J=6.0 Hz, each 3H).

### EXAMPLE 1(15)

cyclohexyl-N-[1-[5-(2-dimethylaminoethylthio)-1,3,4-oxadiazol-2-yl]-1-oxo-2-hexyl]carboxamide

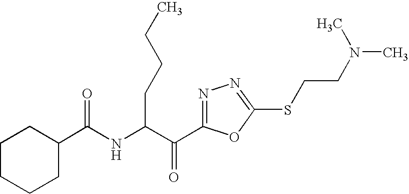

TLC: Rf 0.61 (methanol:chloroform:28% ammonia water=10:190:1); NMR (CDCl₃): δ 6.10 (brd, J=6.3 Hz, 1H), 5.38 (m, 1H), 3.50 (t, J=6.9 Hz, 2H), 2.75 (t, J=6.9 Hz, 2H), 2.31 (s, 6H), 2.20-1.20 (m, 17H), 0.95-0.82 (m, 3H).

### EXAMPLE 1(16)

cycloheptyl-N-[1-[5-(2-dimethylaminoethylthio)-1,3,4-oxadiazol-2-yl]-1-oxo-2-hexyl]carboxamide

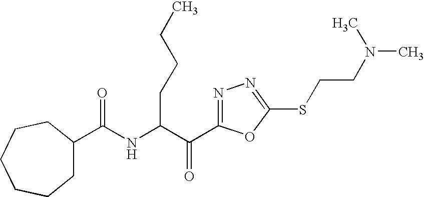

Free compound: TLC: Rf 0.62 (methanol:chloroform:28% ammonia water=10:190:1); NM (CDCl₃): δ 6.03 (brd, J=7.8 Hz, 1H), 5.39 (m, 1H), 3.50 (t, J=6.6 Hz, 2H), 2.75 (t, J=6.9 Hz, 2H), 2.31 (m, 7H), 2.20-1.20 (m, 18H), 1.00-0.80 (m, 3H). Hydrochloride: TLC: Rf 0.62 (chloroform:methanol:28% ammonia water 190:10:1); NMR (CDCl₃): δ 13.02 (br, 1H), 6.04 (brd, J=6.9 Hz, 1H), 5.34 (m, 1H), 3.90 (m, 2H), 3.55 (m, 2H), 2.93 (s, 6H), 2.31 (m, 1H), 2.20-1.20 (m, 18H), 1.00-0.80 (m, 3H).

### EXAMPLE 1(17)

cyclohexyl-N-[1-[5-(3-pyrrolidinopropylthio)-1,3,4-oxadiazol-2-yl]-4-methyl-1-oxo-2-pentyl]carboxamide

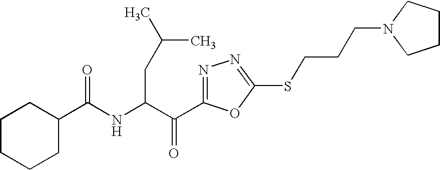

TLC: Rf 0.34 (chloroform:methanol:water=40:10:1); NMR (CDCl₃): δ 6.02 (d, J=7.2 Hz, 1H), 5.45-5.38 (m, 1H), 3.40 (t, J=7.2 Hz, 2H), 2.64 (t, J=7.2 Hz, 2H), 2.55 (br-s, 4H), 2.20-1.99 (m, 3H), 1.89-1.23 (m, 17H), 1.03 (d, J=6.3 Hz, 3H), 0.97 (d, J=6.3 Hz, 3H).

### EXAMPLE 1 (18)

1-[(1R,2S)-2-benzoylaminocyclohexyl]-N-[4-methyl-1-[5-(3-pyrrolidinopropylthio)-1,3,4-oxadiazol-2-yl]-1-oxo-2-pentyl]carboxamide

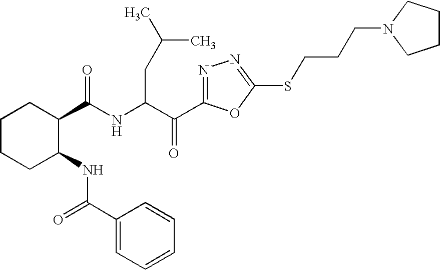

TLC: Rf 0.38 (chloroform:methanol:water=40:10:1); NMR (CDCl₃): δ 7.81-7.75 (m, 21H), 7.52-7.38 (m, 3H), 7.20 (d, J=8.1 Hz, 1H), 6.29 and 6.25 (each d, J=7.2 Hz, total 1H), 5.47-5.34 (m, 1H), 4.38-4.28 (m, 1H), 3.43-3.36 (m, 2H), 2.87-2.84 (m, 1H), 2.64 (t, J=6.9 Hz, 2H), 2.56 (br-s, 4H), 2.11-1.49 (m, 17H), 1.02, 0.95, 0.91, and 0.85 (each d, J=6.3 Hz, total 6H).

### EXAMPLE 1(19)

cyclohexyl-N-[1-[5-(3-morpholinopropylthio)-1,3,4-oxadiazol-2-yl]-4-methyl-1-oxo-2-pentyl]carboxamide

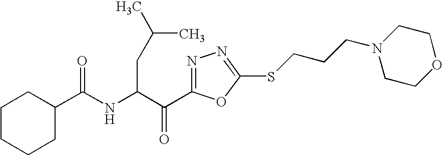

TLC: Rf 0.48 (ethyl acetate:methanol=9:1); NMR (CDCl₃): δ 6.00 (d, J=7.2 Hz, 1H), 5.45-5.38 (m, 1H), 3.71 (t, J=4.5 Hz, 4H), 3.48-3.33 (m, 2H), 2.51-2.45 (m, 6H), 2.21-2.11 (m, 1H), 2.09-1.98 (m, 2H), 1.89-1.15 (m, 13H), 1.03 (d, J=6.0 Hz, 3H), 0.96 (d, J=6.0 Hz, 3H).

### EXAMPLE 1(20)

1-[(1R,2S)-2-benzoylaminocyclohexyl]-N-[4-methyl-1-[5-(3-morpholinopropylthio)-1,3,4-oxadiazol-2-yl]-1-oxo-2-pentyl]carboxamide

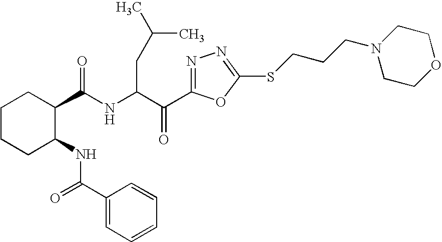

TLC: Rf 0.44 (ethyl acetate:methanol=9:1); NMR (CDCl₃): δ 7.81-7.75 (m, 2H), 7.52-7.39 (m, 3H), 7.24 and 7.18 (each d, J=8.4 Hz, total 1H), 6.29 and 6.23 (each d, J=7.2 Hz, total 1H), 5.47-5.35 (m, 1H), 4.37-4.31 (m, 1H), 3.71 (t, J=4.5 Hz, 4H), 3.42-3.33 (m, 2H), 2.89-2.83 (m, 1H), 2.51-2.46 (m, 6H), 2.07-1.50 (m, 13H), 1.01, 0.95, 0.91, and 0.85 (each d, J=6.0 Hz, total 6H).

### EXAMPLE 1(21)

cyclooctyl-N-[1-[5-(2-dimethylaminoethylthio)-1,3,4-oxadiazol-2-yl]-4-methyl-1-oxo-2-pentyl]carboxamide hydrochloride

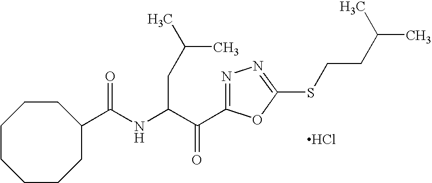

TLC: Rf 0.40 (ethyl acetate:methanol=8:2); NMR (CDCl₃): δ 13.1 (brs, 1H), 5.92 (brd, J=6.9 Hz, 1H), 5.40 (m, 1H), 3.94-3.86 (m, 2H), 3.58-3.48 (m, 2H), 2.95-2.91 (m, 6H), 2.42-2.35 (m, 1H), 1.90-1.45 (m, 17H), 1.03 (d, J=6.0 Hz, 3H), 0.99 (d, J=6.0 Hz, 3H).

### EXAMPLE 1(22)

cyclohexyl-N-[1-[5-(1-methylethylthio)-1,3,4-oxadiazol-2-yl]-4-methyl-1-oxo-2-pentyl]carboxamide

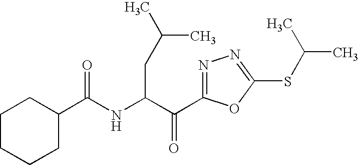

TLC: Rf 0.54 (n-hexane:ethyl acetate=7:3); NMR (CDCl₃): δ 6.02 (brd, J=7.8 Hz, 1H), 5.43 (m, 1H), 4.04 (septet, J=6.9 Hz, 1H), 2.17 (m, 1H), 1.90-1.18 (m, 13H), 1.54 (d, J=6.9 Hz, 6H), 1.03 (d, J=6.3 Hz, 3H), 0.97 (d, J=6.6 Hz, 3H).

### EXAMPLE 1(23)

N-[(2S)-4-methyl-1-[5-(1-methylethylthio)-1,3,4-oxadiazol-2-yl]-1-oxo-2-pentyl]-(3,4-dihydro-4-oxo-2-phenylpyrimidine-3-yl)acetamide

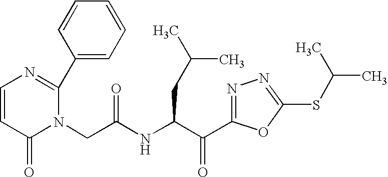

TLC: Rf 0.36 (ethyl acetate); NMR (CDCl₃): δ 8.01 (d, J=6.6 Hz, 1H), 7.60-7.42 (m, 5H), 6.67 (d, J=7.8 Hz, 1H), 6.51 (d, J=6.6 Hz, 1H), 5.54-5.44 (m, 1H), 4.62 and 4.54 (each d, J=15.3 Hz, each 1H), 4.13-3.96 (m, 1H), 1.90-1.48 (m, 3H), 1.55 (d, J=6.6 Hz, 6H), 1.04 and 0.96 (each d, J=6.0 Hz, each 3H).

### EXAMPLE 1(24)

N-cyclopentyloxycarbonyl-N-[4-methyl-1-[5-(1-methylethylthio)-1,3,4-oxadiazol-2-yl]-1-oxo-2-pentyl]amine

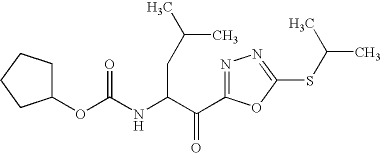

TLC: Rf 0.34 (ethyl acetate:n-hexane=1:4); NMR (CDCl₃): δ 5.31-5.03 (m, 3H), 4.05 (septet, J=6.6 Hz, 1H), 1.91-1.43 (m, 17H), 1.05 and 0.96 (each d, J=5.7 Hz, each 3H).

### EXAMPLE 1(25)

cyclohexyl-N-[1-[5-(3-dimethylaminopropylthio)-1,3,4-oxadiazol-2-yl]-1-oxo-2-hexyl]carboxamide hydrochloride

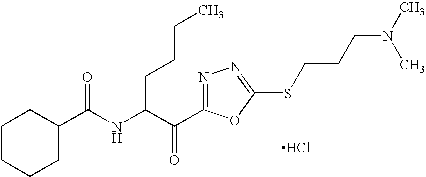

TLC: Rf 0.38 (ethyl acetate:acetic acid:water=3:1:1); NMR (CDCl₃): δ 6.10 (brd, J=7.2 Hz, 1H), 5.35 (m, 1H), 3.65-3.35 (m, 2H), 3.30-3.10 (m, 2H), 2.86 and 2.85 (each s, each 3H), 2.64-2.42 (m, 2H), 2.32 (m, 1H), 2.12-1.20 (m, 16H), 1.00-0.80 (m, 3H).

### EXAMPLE 1(26)

cycloheptyl-N-[1-[5-(3-dimethylaminopropylthio)-1,3,4-oxadiazol-2-yl]-1-oxo-2-hexyl]carboxamide hydrochloride

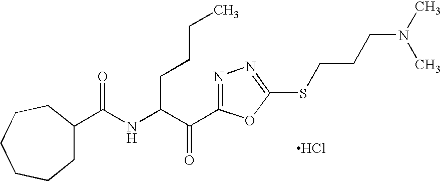

TLC: Rf 0.38 (ethyl acetate:acetic acid:water=3:1:1); NMR (CDCl₃): δ 12.60 (m, 1H), 6.12 (brd, J=7.2 Hz, 1H), 5.37 (m, 1H), 3.63-3.32 (m, 2H), 3.30-3.10 (m, 2H), 2.87 and 2.86 (each s, each 3H), 2.62-2.40 (m, 2H), 2.20 (m, 1H), 2.10-1.20 (m, 18H), 1.00-0.82 (m, 3H).

### EXAMPLE 1(27)

N-[(2S)-4-methyl-1-[5-(1-methylethylthio)-1,3,4-oxadiazol-2-yl]-1-oxo-2-pentyl]-5-methylhexanamide

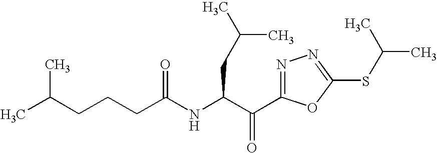

TLC: Rf 0.26 (ethyl acetate:n-hexane=1:3); NMR (CDCl₃): δ 6.01 (d, J=7.8 Hz, 1H), 5.45 (ddd, J=10.2, 7.8, 3.6 Hz, 1H), 4.11-3.96 (m, 1H), 2.22 (t, J=7.2 Hz, 2H), 1.87-1.45 (m, 6H), 1.54 (d, J=6.9 Hz, 6H), 1.24-1.13 (m, 2H), 1.03 and 0.97 (each d, J=6.3 Hz, each 3H), 0.87 (d, J=6.6 Hz, 6H).

### EXAMPLE 1(28)

N-[4-methyl-1-[5-(1-methylethylthio)-1,3,4-oxadiazol-2-yl]-1-oxo-2-pentyl]-4-cyclohexylbutanamide

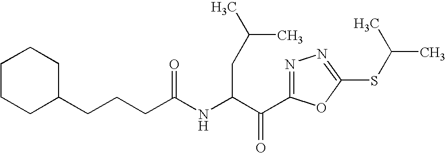

TLC: Rf 0.27 (ethyl acetate:n-hexane=1:3); NMR (CDCl₃): δ 6.00 (d, J=8.0 Hz, 1H), 5.44 (ddd, J=11.7, 8.0, 3.9 Hz, 1H), 4.11-3.95 (m, 1H), 2.22 (t, J=7.2 Hz, 2H), 1.87-1.46 (m, 10H), 1.54 (d, J=6.6 Hz, 6H), 1.29-1.08 (m, 6H), 1.03 and 0.97 (each d, J=6.3 Hz, each 3H), 0.94-0.76 (m, 2H).

### EXAMPLE 1(29)

(2S)-N-[(2S)-[4-methyl-1-(1-methylethylthio)-1,3,4-oxadiazol-5-yl]-1-oxo-2-pentyl]-2-(t-butoxycarbonylamino)-4-methylpentanamide

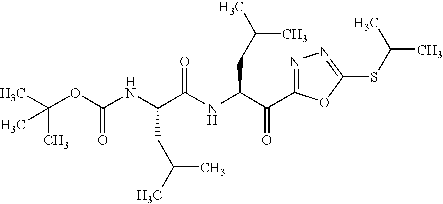

TLC: Rf 0.26 (ethyl acetate:n-hexane=1:3); NMR (CDCl₃): δ 6.74 (d, J=6.6 Hz, 114), 5.51-5.41 (m, 1H), 4.85 (d, J=6.9 Hz, 1H), 4.22-3.95 (m, 2H), 1.88-1.40 (m, 6H), 1.54 (d, J=6.9 Hz, 6H), 1.45 (s, 9H), 1.02, 0.95, 0.94 and 0.92 (each d, J=6.0 Hz, each 3H).

### EXAMPLE 1(30)

N-(2,2-dimethylpropyloxycarbonyl)-N-[4-methyl-2-(1-methylethylthio)-1,3,4-oxadiazol-5-yl-1-oxo-2-pentyl]amine

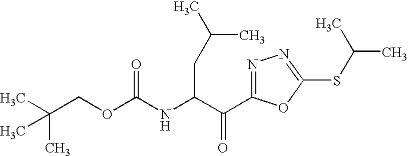

TLC: Rf 0.56 (ethyl acetate:n-hexane=1:1); NMR (CDCl₃): δ 5.31-5.20 (m, 2H), 4.05 (septet, J=6.9 Hz, 1H), 3.78 and 3.75 (each d, J=9.9 Hz, each 1H), 1.90-1.70 (m, 2H), 1.70-1.50 (m, 7H), 1.05 and 0.97 (each d, J=6.0 Hz, each 3H), 1.00-0.81 (m, 9H).

### EXAMPLE 1(31)

N-[4-methyl-1-[5-(1-methylethylthio)-1,3,4-oxadiazol-2-yl]-1-oxo-2-pentyl]cyclohexylacetamide

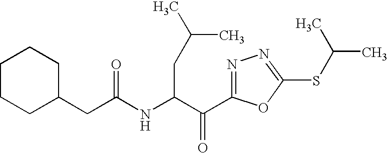

TLC: Rf 0.66 (ethyl acetate:n-hexane=1:3); NMR (CDCl₃): δ 5.96 (brd, J=8.1 Hz, 1H), 5.46 (ddd, J=10.5, 7.8, 3.6 Hz, 1H), 4.04 (septet, J=6.6 Hz, 1H), 2.10 (d, J=7.2 Hz, 2H), 1.90-1.43 (m, 10H), 1.54 (d, J=6.6 Hz, 6H), 1.40-0.90 (m, 4H), 1.04 and 0.97 (each d, J=6.3 Hz, each 3H).

### EXAMPLE 1(32)

1-(tetrahydropyran-4-yl)-N-[4-methyl-1-[5-(1-methylethylthio)-1,3,4-oxadiazol-2-yl]-1-oxo-2-pentyl]carboxamide

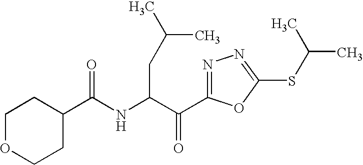

TLC: Rf 0.24 (ethyl acetate); NMR (CDCl₃): δ 6.05 (brd, J=7.8 Hz, 1H), 5.45 (ddd, =10.2, 7.8, 4.2 Hz, 1H), 4.10-3.98 (m, 3H), 3.43 (m, 2H), 2.42 (m, 1H), 1.90-1.50 (m, 13H), 1.04 and 0.97 (each d, J=6.3 Hz, each 3H).

### EXAMPLE 1(33)

1-cyclopropylcarbonylaminocyclohexyl-N-[(2S)-4-methyl-1-[5-(1-methylethylthio)-1,3,4-oxadiazol-2-yl]-1-oxo-2-pentyl]carboxamide

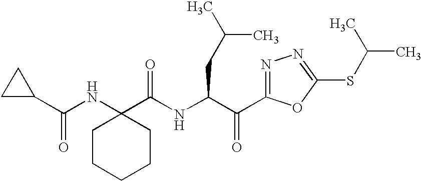

TLC: Rf 0.67 (ethyl acetate:n-hexane=1:1); NMR (CDCl₃): δ 7.97 (brd, J=6.6 Hz, 1H), 5.57 (brs, 1H), 5.33 (ddd, J=9.9, 6.6, 3.3 Hz, 1H), 4.03 (septet, J=6.6 Hz, 1H), 2.21-1.21 (m, 20H), 1.10-0.72 (m, 10H).

### EXAMPLE 1(34)

N-(2,2,2-trichloroethyloxycarbonyl)-N-[4-methyl-1-[2-(1-methylethylthio)-1,3,4-oxadiazol-5-yl]-1-oxo-2-pentyl]amine

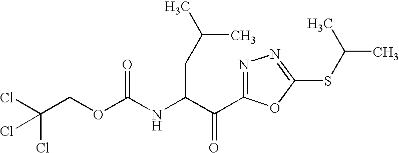

TLC: Rf 0.59 (ethyl acetate:n-hexane=1:3); NMR (CDCl₃): δ 5.59 (brd, J=8.1 Hz, 1H), 5.38-5.30 (m, 1H), 4.77 and 4.69 (each d, J=12.0 Hz, each 1H), 4.05 (septet, J=6.6 Hz, 1H), 1.90-1.50 (m, 3H), 1.55 (d, J=6.6 Hz, 6H), 1.07 and 0.98 (each d, J=6.3 Hz, each 3H).

### EXAMPLE 1(35)

N-(t-butoxycarbonyl)-N-[1-[5-(2-dimethylaminoethylthio)-1,3,4-oxadiazol-2-yl]-4-methyl-1-oxo-2-pentyl]amine hydrochloride

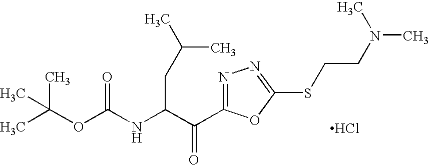

TLC: Rf 0.45 (methanol:chloroform=1:9); NMR (CDCl₃): δ 13.15 (br, 1H), 5.21 (m, 1H), 5.04 (brd, J=8.1 Hz, 1H), 3.92 (m, 2H), 3.51 (m, 2H), 2.93 (s, 6H), 1.90-1.40 (m, 12H), 1.05 and 0.98 (each d, J=6.3 Hz, each 3H).

### EXAMPLE 1(36)

cyclohexyl-N-[(2S)-1-[5-(2-t-butoxycarbonylaminoethylthio)-1,3,4-oxadiazol-2-yl]-4-methyl-1-oxo-2-pentyl]carboxamide

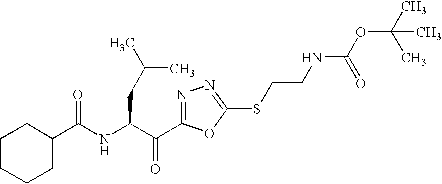

TLC: Rf 0.21 (n-hexane:ethyl acetate=7:3); NMR (DMSO-d₆): δ 8.35 (brd, J=6.3 Hz, 1H), 7.11 (m, 1H), 5.00 (m, 1H), 3.40-3.32 (m, 4H), 2.19 (m, 1H), 1.80-1.00 (m, 13H), 1.36 (s, 9H), 0.91 (d, J=6.6 Hz, 3H), 0.90 (d, J=6.3 Hz, 3H).

### EXAMPLE 1(37)

15 cyclononyl-N-[4-methyl-1-[5-(1-methylethylthio)-1,3,4-oxadiazol-2-yl]-1-oxo-2-pentyl]carboxamide

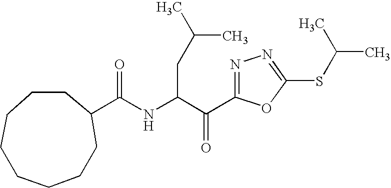

TLC: Rf 0.34 (ethyl acetate:n-hexane=1:3); NMR (CDCl₃): δ 5.95 (d, J=7.5 Hz, 1H), 5.45 (ddd, J=10.2, 7.5, 3.9 Hz, 1H), 4.12-3.96 (m, 1H), 2.46-2.33 (m, 1H), 1.89-1.36 (m, 19H), 1.53 (d, J=6.9 Hz, 6H), 1.03 and 0.98 (each d, J=6.3 Hz, each 3H).

### EXAMPLE 1(38)

N-[4-methyl-1-[5-(1-methylethylthio)-1,3,4-oxadiazol-2-yl]-1-oxo-2-pentyl]-2-propylpentanamide

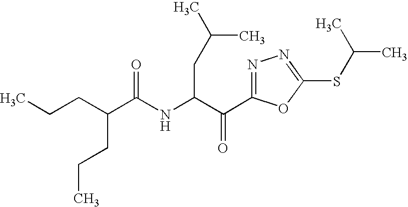

TLC: Rf 0.69 (ethyl acetate:n-hexane=1:2); NMR (CDCl₃): δ 5.96 (brd, J=7.2 Hz, 1H), 5.48 (ddd, J=10.2, 7.2, 3.9 Hz, 1H), 4.04 (septet, J=6.6 Hz, 1H), 2.14 (m, 1H), 1.85-1.18 (m, 17H), 1.04 and 0.98 (each d, J=6.3 Hz, each 3H), 0.90 and 0.88 (each t, J=6.9 Hz, each 3H).

### EXAMPLE 1(39)

N-[4-methyl-1-[5-(1-methylethylthio)-1,3,4-oxadiazol-2-yl]-1-oxo-2-pentyl]-6-phenylhexanamide

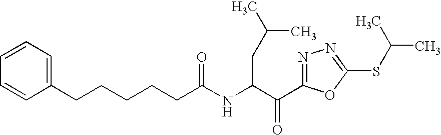

TLC: Rf 0.47 (ethyl acetate:n-hexane=1:2); NM (CDCl₃): δ 7.31-7.15 (m, 5H), 5.96 (brd, J=7.8 Hz, 1H), 5.45 (ddd, J=10.2, 7.8, 3.9 Hz, 1H), 4.04 (septet, J=6.6 Hz, 1H), 2.61 (t, J=7.8 Hz, 2H), 2.24 (t, J=7.8 Hz, 2H), 1.90-1.31 (m, 15H), 1.03 and 0.97 (each d, J=6.3 Hz, each 3H).

### EXAMPLE 1(40)

1-(2,2,3,3-tetramethylcyclopropyl)-N-[4-methyl-1-[5-(1-methylethylthio)-1,3,4-oxadiazol-2-yl]-1-oxo-2-pentyl]carboxamide

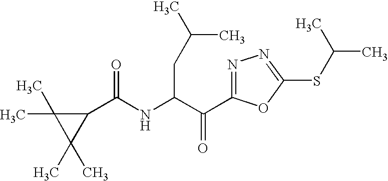

TLC: Rf 0.73 (ethyl acetate:n-hexane=1:2); NMR (CDCl₃): δ 5.92 (brd, J=7.5 Hz, 1H), 5.39 (ddd, J=9.9, 7.5, 3.0 Hz, 1H), 4.06 (septet, J=6.6 Hz, 1H), 1.90-1.50 (m, 9H), 1.23 (s, 3H), 1.17 (s, 9H), 1.03 and 0.96 (each d, J=6.0 Hz, each 3H), 0.95 (s, 1H).

### EXAMPLE 1(41)

N-[4-methyl-1-[5-(1-methylethylthio)-1,3,4-oxadiazol-2-yl]-1-oxo-2-pentyl]-4-(2-thienyl)butanamide

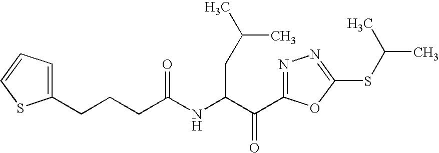

TLC: Rf 0.45 (ethyl acetate:n-hexane=1:2); NMR (CDCl₃): δ 7.13 (dd, J=5.1, 1.2 Hz, 1H), 6.92 (dd, J=5.1, 3.0 Hz, 1H), 6.81 (dd, J=3.01.2 Hz, 1H), 5.95 (brd, J=7.5 Hz, 1H), 5.46 (ddd, J=9.9, 7.5, 3.6 Hz, 1H), 4.04 (septet, J=6.3 Hz, 1H), 2.88 (t, J=7.5 Hz, 2H), 2.28 (t, J=6.6 Hz, 2H), 2.03 (m, 2H), 1.90-1.50 (m, 9H), 1.04 and 0.97 (each d, J=6.3 Hz, each 3H).

### EXAMPLE 1(42)

N-[4-methyl-1-[5-(1-methylethylthio)-1,3,4-oxadiazol-2-yl]-1-oxo-2-pentyl]-(tricyclo[3.3.1.1]decan-1-yl)acetamide

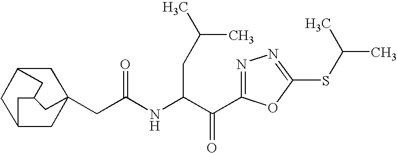

TLC: Rf 0.50 (ethyl acetate:n-hexane=1:2); NNM (CDCl₃): δ 5.87 (brd, J=7.2 Hz, 1H), 5.45 (ddd, J=9.9, 7.2, 3.6 Hz, 1H), 4.04 (septet, J=6.3 Hz, 1H), 2.05-1.92 (m, 5H), 1.90-1.51(m, 21H), 1.04 and 0.97 (each d, J=6.3 Hz, each 3H).

### EXAMPLE 1(43)

tricyclo[3.3.1.1]decan-1-yl-N-[4-methyl-1-[2-(1-methylethylthio)-1,3,4-oxadiazol-5-yl]-1-oxo-2-pentyl]carboxaniide

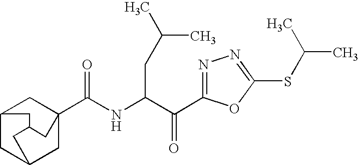

TLC: Rf 0.59 (ethyl acetate:n-hexane=1:2); NMR (CDCl₃): δ 6.19 (brd, J=7.5 Hz, 1H), 5.39 (ddd, J=9.6, 7.5, 3.9 Hz, 1H), 4.04 (septet, J=6.3 Hz, 1H), 2.04 (brs, 3H), 2.00-1.50 (m, 21H), 1.02 and 0.98 (each d, J=6.3 Hz, each 3H).

### EXAMPLE 1(44)

cyclohexyl-N-[(2S)-1-[5-(t-butoxycarbonylmethylthio)-1,3,4-oxadiazol-2-yl]-4-methyl-1-oxo-2-pentyl]carboxamide

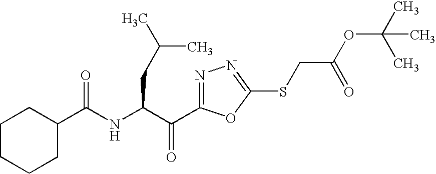

TLC: Rf 0.80 (n-hexane:ethyl acetate=1:1); NMR (CDCl₃): δ 6.01 (brd, J=7.5 Hz, 1H), 5.40 (m, 1H), 4.06 (s, 2H), 2.16 (m, 1H), 1.90-1.20 (m, 13H), 1.49 (s, 9H), 1.02 (d, J=6.3 Hz, 3H), 0.97 (d, J=6.3 Hz, 3H).

### EXAMPLE 1(45)

cyclohexyl-N-[(2S)-1-[5-(2-methoxyethylthio)-1,3,4-oxadiazol-2-yl]-4-methyl-1-oxo-2-pentyl]carboxamide

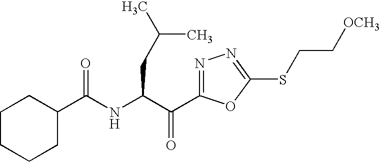

TLC: Rf 0.53 (n-hexane:ethyl acetate=1:1); NMR (CDCl₃): δ 6.00 (brd, J=6.9 Hz, 0.1H), 5.43 (m, 1H), 3.77 (t, J=6.0 Hz, 2H), 3.53 (m, 2H), 3.40 (s, 3H), 2.17 (m, 1H), 1.90-1.20 (m, 13H), 1.03 (d, J=6.0 Hz, 3H), 0.97 (d, J=6.3 Hz, 3H).

### EXAMPLE 1(46)

cyclohexyl-N-[(2S)-1-[5-(1,3-dioxolan-2-ylmethylthio)-1,3,4-oxadiazol-2-yl]-4-methyl-1-oxo-2-pentyl]carboxamide

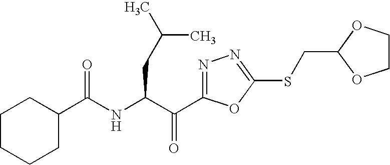

TLC: Rf 0.48 (n-hexane:ethyl acetate=1:1); NMR (CDCl₃): δ 6.01 (brd, J=7.8 Hz, 1H), 5.41 (m, 1H), 5.31 (t, J=3.6 Hz, 1H), 4.07-3.90 (m, 4H), 3.62 (d, J=3.6 Hz, 2H), 2.16 (m, 1H), 1.90-1.20 (m, 13H), 1.02 (d, J=6.0 Hz, 3H), 0.97 (d, J=6.3 Hz, 3H).

### EXAMPLE 1(47)

cycloheptyl-N-[(2S)-4-methyl-1-[5-(2-morpholinoethylthio)-1,3,4-oxadiazol-2-yl] 1-oxo-2-pentyl]carboxamide hydrochloride

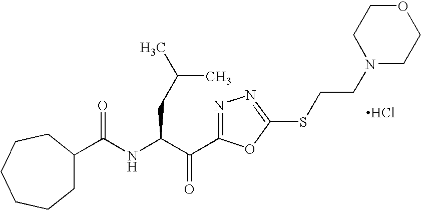

TLC: Rf 0.54 (ethyl acetate:methanol=9:1); NMR (DMSO-d₆): δ 11.2 (brs, 1H), 8.38 (brd, J=6.3 Hz, 1H), 5.02 (m, 1H), 4.00-3.00 (m, 12H), 2.37 (m, 1H), 1.80-1.30 (m, 15H), 0.91 (d, J=6.3 Hz, 3H), 0.90 (d, J=6.3 Hz, 3H).

### EXAMPLE 1(48)

cycloheptyl-N-[(2S)-1-(5-cyanomethylthio-1,3,4-oxadiazol-2-yl)-4-methyl-1-oxo-2-pentyl]carboxamide

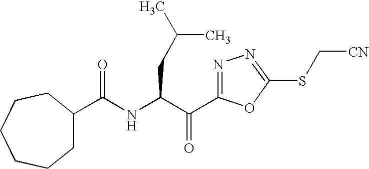

TLC: Rf 0.50 (n-hexane:ethyl acetate=:1:1); NMR (CDCl₃): δ 5.91 (brd, J=6.9 Hz, 1H), 5.36 (m, 1H), 4.19 and 4.09 (each d, J=17 Hz, each 1H), 2.32 (m, 1H), 1.90-1.40 (m, 15H), 1.04 (d, J=6.3 Hz, 3H), 1.00 (d, J=6.3 Hz, 3H).

### EXAMPLE 1(49)

cycloheptyl-N-[1-[5-(1-t-butoxycarbonyl-1-methylethylthio)-1,3,4-oxadiazol-2-yl]-4-methyl-1-oxo-2-pentyl]carboxamide

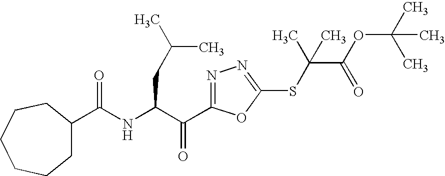

TLC: Rf 0.84 (n-hexane:ethyl acetate=1:1); NMR (CDCl₃): δ 5.95 (brd, J=8.1 Hz, 1H), 5.40 (m, 1H), 2.31 (m, 1H), 1.90-1.40 (m, 15H), 1.78 (s, 6H), 1.44 (s, 9H), 1.03 (d, J=6.3 Hz, 3H), 0.97 (d, J=6.3 Hz, 3H).

### EXAMPLE 1(50)

cycloheptyl-N-[1-[5-(2,4-dioxo-1,5,5-trimethylpyrrolidine-3-ylmethylthio)-1,3,4-oxadiazol-2-yl]-4-methyl-1-oxo-2-pentyl]carboxamide

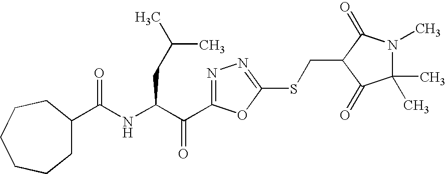

TLC: Rf 0.18 (n-hexane:ethyl acetate=1:1); NMR (CDCl₃): δ 5.98 (brd, J=7.8 Hz, 1H), 5.41 (m, 1H), 5.27 and 5.20 (each d, J=13 Hz, each 1H), 2.88 (s, 3H), 2.31 (m, 1H), 1.90-1.40 (m, 15H), 1.44 (s, 9H), 1.39 (s, 6H), 1.03 (d, J=6.3 Hz, 3H), 0.97 (d, J=6.3 Hz, 3H).

### EXAMPLE 1(51)

cycloheptyl-N-[(2S)-1-[5-[2-(2-methoxyethyloxy)ethylthio]-1,3,4-oxadiazol-2-yl]-4-methyl-1-oxo-2-pentyl]carboxamide

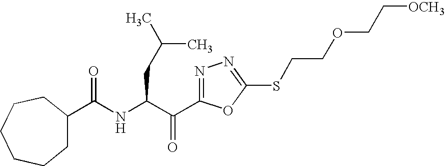

TLC: Rf 0.34 (n-hexane:ethyl acetate=1:1); NMR (CDCl₃): δ 5.94 (brd, J=7.8 Hz, 1H), 5.42 (m, 1H), 3.88 (t, J=6.0 Hz, 2H), 3.68-3.65 (m, 2H), 3.55-3.44 (m, 4H), 3.39 (s, 3), 2.32 (m, 1H), 1.90-1.40 (m, 15H), 1.03 (d, J=6.3 Hz, 3H), 0.97 (d, J=6.3 Hz, 3H).

### EXAMPLE 1(52)

1-phenoxycarbonylaminocyclohexyl-N-[1-[5-(2-dimethylaminoethylthio)-1,3,4-oxadiazol-2-yl]-4-methyl-1-oxo-2-pentyl]carboxamide hydrochloride

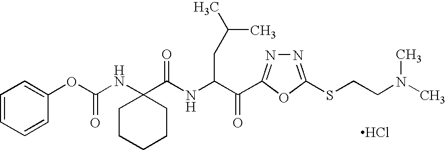

TLC: Rf 0.45 (methylene chloride:methanol:acetic acid=10:1:1); NMR (DMSO-d₆): δ 10.58-10.42 (br, 1H), 8.27 (d, J=7.2 Hz, 1H), 7.58 (s, 1H), 7.42-7.32 (m, 2H), 7.22-7.15 (m, 1H), 7.14-7.04 (m, 2H), 5.12-5.02 (m, 1H), 3.74-3.64 (m, 2H), 3.52-3.40 (m, 2H), δ 2.80 (s, 6H), 2.00-1.20 (m, 13H), 0.90-0.85 (m, 6H).

### EXAMPLE 1(53)

1-(t-butoxycarbonylamino)cyclopentyl-N-[(2S)-4-methyl-1-[5-(1-methylethylthio)-1,3,4-oxadiazol-2-yl]-1-oxo-2-pentyl]carboxamide

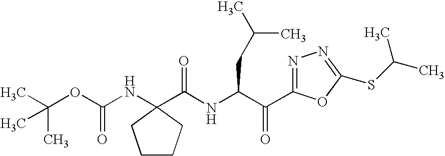

TLC: Rf 0.74 (n-hexane:ethyl acetate=1:1); NMR (CDCl₃): δ 7.45 (broad, 1H), 5.42 (m, 1H), 4.84 (brs, 1H), 4.03 (septet, J=6.9 Hz, 1H), 2.43-2.14 (m, 2H), 1.95-1.40 (m, 9H), 1.53 (d, J=6.9 Hz, 6H),1.45 (s, 9H), 1.03 (d, J=6.3 Hz, 3H), 0.96 (d, J=6.3 Hz, 3H).

### EXAMPLE 1(54)

1-(t-butoxycarbonylamino)cyclohexyl-N-[4-methyl-1-[5-(2-dimethylaminoethylthio)-1,3,4-oxadiazol-2-yl]-1-oxo-2-pentyl]carboxamide hydrochloride

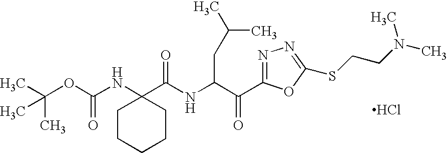

TLC: Rf 0.56 (methanol:chloroform=1:9); NMR (CDCl₃): δ 13.18 (br, 1H), 7.50 (br, 1H), 5.38 (m, 1H), 4.69 (brs, 1H), 3.88 (m, 2H), 3.52 (m, 2H), 2.93 (brs, 6H), 2.10-1.22 (m, 22H), 1.02 and 0.96 (each d, J=6.0 Hz, each 3H).

### EXAMPLE 1(55)

(indan-2-yl)-N-[1-[5-(1-methylethylthio)-1,3,4-oxadiazol-2-yl]-4-methyl-1-oxo-2-pentyl]carboxamide

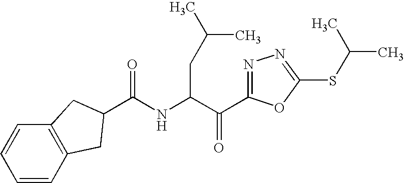

TLC: Rf 0.54 (ethyl acetate:n-hexane=1:2); NMR (CDCl₃): δ 7.24-7.13 (m, 4H), 6.07 (brd, J=7.8 Hz, 1H), 5.48 (ddd, J=9.9, 7.8, 3.9 Hz, 1H), 4.04 (septet, J=6.6 Hz, 1H), 3.32-3.15 (m, 5H), 1.90-1.50 (m, 3H), 1.54 (d, J=6.6 Hz, 6H), 1.03 and 0.97 (each d, J=6.0 Hz, each 3H).

### EXAMPLE 1(56)

1-cyclopropylcarbonylaminocyclohexyl-N-[4-methyl-1-[5-(2-dimethylaminoethylthio)-1,3,4-oxadiazol-2-yl]-1-oxo-2-pentyl]carboxamide hydrochloride

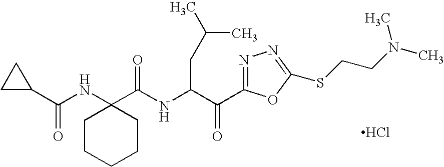

TLC: Rf 0.42 (ethyl acetate:acetic acid:water=3:3:1); NMR (CDCl₃): δ 13.10 (br, 1H), 8.05 (brd, J=6.0 Hz, 1H), 5.74 (brs, 1H), 5.28 (m, 1H), 3.85 and 3.52 (each m, each 2H), 2.93 (brs, 6H), 2.21-1.30 (m, 14H), 1.03-0.72 (m, 10H).

### EXAMPLE 1(57)

N-[4-methyl-1-[5-(1-methylethylthio)-1,3,4-oxadiazol-2-yl-1-oxo]-2-pentyl]-3-diethylaminopropanamide hydrochloride

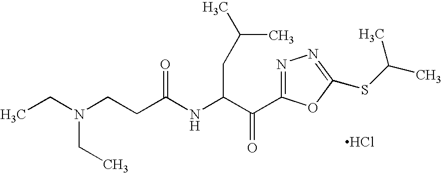

TLC: Rf 0.43 (methanol:chloroform:water=2:8:1); NMR (CDCl₃): δ 11.61 (br, 1H), 7.77 (brd, J=6.3 Hz, 1H), 5.38 (m, 1H), 4.04 (septet, J=6.9 Hz, 1H), 3.43-2.85 (m, 6H), 1.92-1.30 (m, 17H), 1.06-0.90 (m, 6H).

### EXAMPLE 1(58)

1-methylpiperidin-4-yl-N-[4-methyl-1-[5-(1-methylethylthio)-1,3,4-oxadiazol-2-yl]-1-oxo-2-pentyl]carboxamide hydrochloride

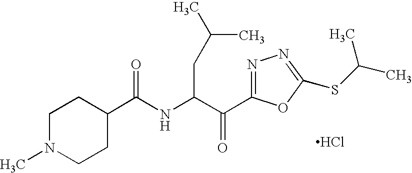

TLC: Rf 0.46 (ethyl acetate:acetic acid:water=3:1:1); NMR (CDCl₃): δ 11.60 (br, 1H), 7.10 (brd, J=6.8 Hz, 1H), 5.39 (m, 1H), 4.05 (septet, J=7.2 Hz, 1H), 3.70-1.20 (m, 21H), 1.04-0.80 (m, 6H).

### EXAMPLE 1(59)

N-[4-methyl-1-[5-(1-methylethylthio)-1,3,4-oxadiazol-2-yl]-1-oxo-2-pentyl]-1-piperidinopropanamide hydrochloride

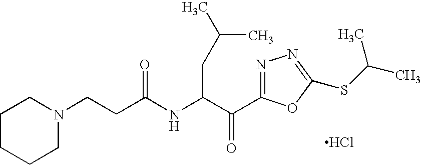

TLC: Rf 0.47 (ethyl acetate:acetic acid:water=3:1:1); NMR (CDCl₃): δ 11.50 (br, 1H), 7.83 (brd, J=6.0 Hz, 1H), 5.35 (m, 1H), 4.04 (septet, J=6.6 Hz, 1H), 3.70-1.30 (m, 23H), 1.10-0.95 (m, 6H).

### EXAMPLE 1(60)

N-[4-methyl-1-[5-(1-methylethylthio)-1,3,4-oxadiazol-2-yl]-1 oxo-2-pentyl]4-dimethylaminobutanamide hydrochloride

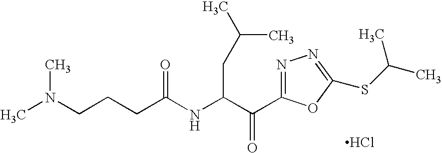

TLC: Rf 0.45 (ethyl acetate:acetic acid:water=3:1:1); NMR (CDCl₃): δ 11.70 (br, 1H), 8.79 (brd, J=5.4 Hz, 1H), 5.35 (m, 1H), 4.05 (septet, J=6.9 Hz, 1H), 3.50 and 3.10 (each m, each 1H), 3.01 and 2.81 (each m, each 3H), 2.90 and 2.40 (each m, each 1H), 2.30-1.40 (m, 5H), 1.54 (d, J=6.9 Hz, 6H), 1.05-0.90 (m, 6H).

### EXAMPLE 1(61)

N-[(2S)-4-methyl-1-[5-(1-methylethylthio)-1,3,4-oxadiazol-2-yl]-1-oxo-2-pentyl]-(3,4-dihydro-4-oxo-2-methylpyrimidine-3-yl)acetaniide

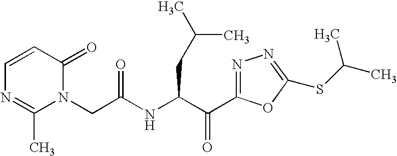

TLC: Rf 0.35 (chloroform:methanol=9:1); NMR (CDCl₃): δ 7.85 (d, J=6.6 Hz, 1H), 7.03 (d, J=7.4 Hz, 1H), 6.42 (d, J=6.6 Hz, 1H), 5.41 (ddd, J=10.2, 7.4, 3.9 Hz, 1H), 4.75 (s, 2H), 4.10-3.95 (m, 1H), 2.61 (s, 3H), 1.91-1.46 (m, 3H), 1.54 (d, J=6.9 Hz, 6H), 1.01 and 0.96 (each d, J=6.3 Hz, each 3H).

### EXAMPLE 1(62)

N-[(2S)-4-methyl-1-[5-(1-methylethylthio)-1,3,4-oxadiazol-2-yl]-1-oxo-2-pentyl]-(3,4-dihydro-4-oxo-2-cyclohexylmethylpyrimidine-3-yl)acetamide

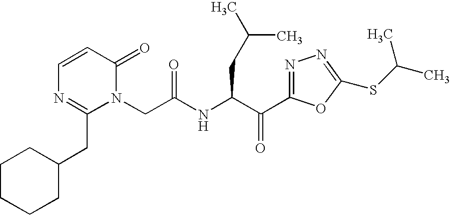

TLC: Rf 0.50 (chloroform:methanol=9:1); NMR (CDCl₃): δ 7.90 (d, J=6.6 Hz, 1H), 7.07 (d, J=7.1 Hz, 1H), 6.41 (d, J=6.6 Hz, 1H), 5.39 (ddd, J=10.4, 7.1, 3.6 Hz, 1H), 4.76 (s, 2H), 4.10-3.95 (m, 1H), 2.70 (d, J=7.2 Hz, 2H), 1.97-1.36 and 1.36-0.83 (each m, total 14H), 1.54 (d, J=6.9 Hz, 6H), 1.00 and 0.96 (each d, J=6.3 Hz, each 3H).

### EXAMPLE 1(63)

1-(benzo[b]thiophene-2-ylcarbonylaminocyclohexyl)-N-[4-methyl-1-[5-(1-methylethylthio)-1,3,4-oxadiazol-2-yl]-1-oxo-2-pentyl]carboxamide

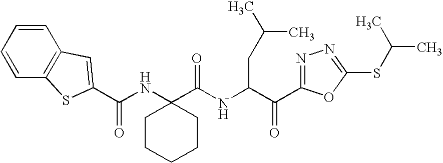

TLC: Rf 0.66 (n-hexane:ethyl acetate=1:1); NMR (CDCl₃): δ 7.92-7.85 (m, 3H), 7.81 (d, J=0.6 Hz, 1H), 7.50-7.40 (m, 2H), 6.07 (brs, 1H), 5.37 (m, 1H), 3.94 (septet, J=6.9 Hz, 1H), 2.35-2.22 (m, 2H), 2.08-1.95 (m, 2H), 1.88-1.25 (m, 9H), 1.50 (d, J=6.9 Hz, 3H), 1.48 (d, J=6.9 Hz, 3H), 1.01 (d, J=6.0 Hz, 3H), 0.97 (d, J=6.3 Hz, 3H).

### EXAMPLE 1(64)

-(benzo[b]thiophene-2-ylcarbonylamino)cyclohexyl-N-[4-methyl-1-[5-(2-dimethylaminoethylthio)-1,3,4-oxadiazol-2-yl]-1-oxo-2-pentyl]carboxamide hydrochloride

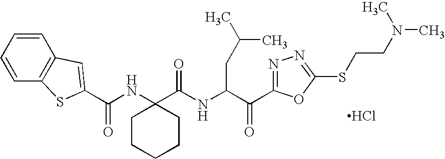

TLC: Rf 0.58 (ethyl acetate:acetic acid:water=3:1:1); NMR(DMSO-d₆): δ 10.38 (broad, 1H), 8.27 (s, 1H), 8.18 (brd, J=6.6 Hz, 1H), 8.08 (brs, 1H), 8.05-7.90 (m, 2H), 7.50-7.40 (m, 2H), 5.04 (m, 1H), 3.69 (m, 2H), 3.50 (m, 2H), 2.82 (s, 6H), 2.12 (m, 2H), 1.85-1.20 (m, 11H), 0.87 (d, J=6.0 Hz, 3H), 0.84 (d, J=6.0 Hz, 3H).

### EXAMPLE 1(65)

N-[(2S)-4-methyl-1-[5-(1-methylethylthio)-1,3,4-oxadiazol-2-yl]-1-oxo-2-pentyl]-4-(t-butoxycarbonyl amino)-4-methylpentanamide

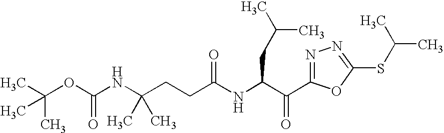

TLC: Rf 0.13 (ethyl acetate:n-hexane=1:3); NMR (CDCl₃): δ 6.44-6.15 (br, 1H), 5.50-5.36 (m, 1H), 4.52 (brs, 1H), 4.13-3.95 (m, 1H), 2.32-2.21 and 2.10-1.96 (each m, each 2H), 1.88-1.37 (m, 3H), 1.54 (d, J=6.9 Hz, 6H), 1.44 (s, 9H), 1.26 and 1.24 (each s, each 3H), 1.04 and 0.98 (each d, J=6.3 Hz, each 3H).

### EXAMPLE 1(66)

cycloheptyl-N-[(2S)-1-(5-dimethylaminocarbonylmethylthio-1,3,4-oxadiazol-2-yl)-4-methyl-1-oxo-2-pentyl]carboxamide

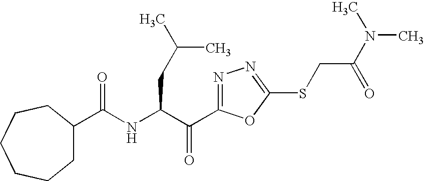

TLC: Rf 0.51 (ethyl acetate); NNM (CDCl₃): δ 5.93 (d, J=7.7 Hz, 1H), 5.43 (ddd, J=10.1, 7.7, 3.9 Hz, 1H), 4.41 (s, 2H), 3.14 and 3.03 (each s, each 3H), 2.38-2.24 (m, 1H), 1.97-1.36 (m, 15H), 1.03 and 0.97 (each d, J=6.3 Hz, each 3H).

### EXAMPLE 1(67)

N-[1-[5-(2-dimethylaminoethylthio)-1,3,4-oxadiazol-2-yl]-4-methyl-1-oxo-2-pentyl]acetamide hydrochloride

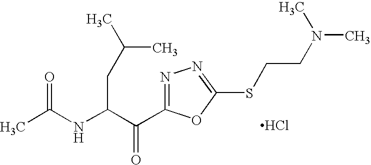

TLC: Rf 0.17 (ethyl acetate:methanol-9:1); NMR (CDCl₃): δ 6.02 (d, J=7.2 Hz, 1H), 5.49-5.36 (m, 1H), 3.95-3.83 and 3.58-3.43 (each m, each 2H), 2.91 (s, 6H), 2.05 (s, 3H), 1.86-1.48 (m, 3H), 1.04 and 0.99 (each d, J=6.0 Hz, each 3H).

### EXAMPLE 1(68)

cycloheptyl-N-[(2S)-4-methyl-1-[5-(2-methylpropylthio)-1,3,4-oxadiazol-2-yl]-1-oxo-2-pentyl]carboxamide

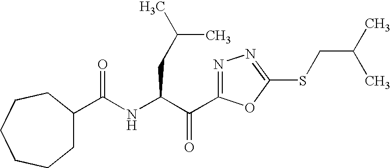

TLC: Rf 0.36 (ethyl acetate:n-hexane=1:3); NMR (CDCl₃): δ 5.95 (d, J=7.8 Hz, 1H), 5.41 (ddd, J=10.1, 7.8, 3.9 Hz, 1H), 3.26 and 3.21 (each dd, J=12.9, 6.9 Hz, each 1H), 2.37-2.25 (m, 1H), 2.19-2.02 (m, 1H), 1.96-1.36 (m, 15H), 1.08 (d, J=6.3 Hz, 6H), 1.03 and 0.97 (each d, J=6.0 Hz, each 3H).

### EXAMPLE 1(69)

N-benzyloxycarbonyl-N-[1-[5-(2-dimethylaminoethylthio)-1,3,4-oxadiazol-2-yl]-4-methyl-1-oxo-2-pentyl]amine hydrochloride

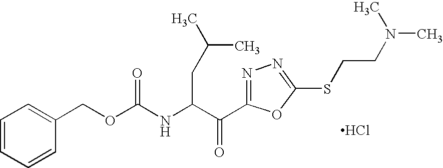

TLC: Rf 0.33 (ethyl acetate:methanol=9:1); NMR (CDCl₃): δ 7.45-7.23 (m, 5H), 5.43-5.20 (m, 2H), 5.10 (s, 2H), 3.95-3.84 and 3.60-3.45 (each m, each 2H), 2.93 (s, 6H), 1.89-1.47 (m, 3H), 1.05 and 0.97 (each d, J=5.9 Hz, each 3H).

### EXAMPLE 1(70)

1-(3-diethylaminopropanoylamino)cyclohexyl-N-[4-methyl-1-[5-(1-methylethylthio)-1,3,4-oxadiazol-2-yl]-1-oxo-2-pentyl]carboxamide hydrochloride

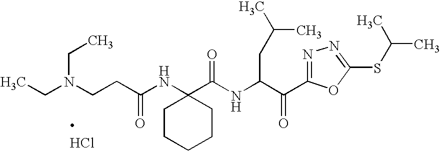

TLC: Rf 0.52 (ethyl acetate:acetic acid:water=3:1:1); NMR (DMSO-d₆): δ 10.12-9.85 (br, 1H), 8.25 (brd, J=6.6 Hz, 1H), 7.87 (s, 1H), 4.96-4.85 (m, 1H), 3.96 (sept, J=6.6 Hz, 1H), 3.24-3.14 (m, 2H), 3.14-3.01 (m, 4H), 2.70 (t, J=6.9 Hz, 2H), 1.97-1.05 (m, 13H), 1.46 (d, J=6.6 Hz, 6H), 1.21 (t, J=7.2 Hz, 6H), 0.88 (d, J=6.0 Hz, 6H).

### EXAMPLE 1(71)

cycloheptyl-N-[(2S)-4-methyl-1-oxo-1-[5-(pyridin-2-ylmethylthio)-1,3,4-oxadiazol-2-yl]-2-pentyl]carboxamide hydrochloride

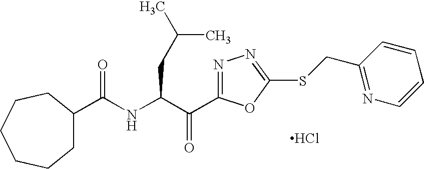

TLC: Rf 0.60 (ethyl acetate:n-hexane=2:1); NMR (CDCl₃): δ 8.71 (d, J=4.8 Hz, 1H), 8.30 (t, J=8.1 Hz, 1H), 8.21 (d, J=8.1 Hz, 1H), 7.86-7.74 (m, 1H), 5.92 (d, J=6.9 Hz, 1H), 5.45-5.33 (m, 1H), 5.12 (s, 2H), 2.40-2.23 (m, 1H), 1.96-1.33 (m, 15H), 1.03 and 0.98 (each d, J=6.0 Hz, each 3H).

### EXAMPLE 1(72)

cycloheptyl-N-[(2S)-4-methyl-1-oxo-1-[5-(pyridin-3-ylmethylthio)-1,3,4-oxadiazol-2-yl]-2-pentyl]carboxamide hydrochloride

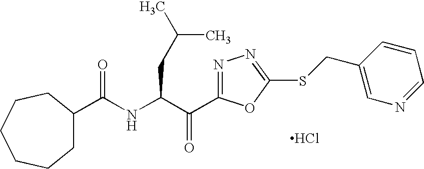

TLC: Rf 0.27 (ethyl acetate:n-hexane=2:1); NMR (CDCl₃): δ 9.17 (s, 1H), 8.72 (d, J=5.4 Hz, 1H), 8.62 (d, J=8.0 Hz, 1H), 7.88 (dd, J=8.0, 5.4 Hz, 1H), 6.00 (d, J=7.2 Hz, 1H), 5.40-5.26 (m, 1H), 4.75 (s, 2H), 2.40-2.25 (m, 1H), 2.00-1.32 (m, 15H), 1.02 and 0.98 (each d, J=6.0 Hz, each 3H).

### Example 1(73)

cycloheptyl-N-[(2S)-1-[5-(pyridin-4-ylmethylthio)-1,3,4-oxadiazol-2-yl]-4-methyl-1-oxo-2-pentyl]carboxamide hydrochloride

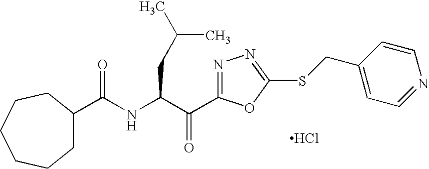

TLC: Rf 0.22 (ethyl acetate:n-hexane=2:1); NMR (CDCl₃): δ 8.77 (d, J=6.2 Hz, 2H), 8.10 (d, J=6.2 Hz, 2H), 5.97 (d, J=6.9 Hz, 1H), 5.39-5.24 (m, 1H), 4.70 (s, 2H), 2.40-2.23 (m, 1H), 1.97-1.35 (m, 15H), 1.01 and 0.98 (each d, J=6.3 Hz, each 3H).

### EXAMPLE 1(74)

cycloheptyl-N-[(2S)-1-[5-(1-dimethylaminocarbonyl-1-methylethylthio)-1,3,4-oxadiazol-2-yl]-4-methyl-1-oxo-2-pentyl]carboxamide

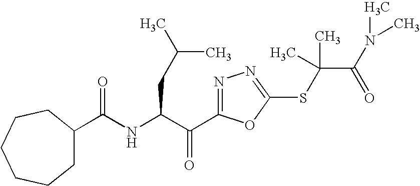

TLC: Rf 0.24 (ethyl acetate:n-hexane=1:1); NMR (DMSO-d₆): δ 8.37 (d, J=6.3 Hz, 1H), 5.02-4.90 (m, 1H), 3.04 (brs, 6H), 2.42-2.27 (m, 1H), 1.90-1.30 (m, 15H), 1.79 (s, 6H), 0.91 and 0.90 (each d, J=6.3 Hz, each 3H).

### EXAMPLE 1(75)

1-(4-dimethylaminomethylbenzoylamino)cyclohexyl-N-[4-methyl-1-[5-(2-dimethylaminoethylthio)-1,3,4-oxadiazol-2-yl]-1-oxo-2-pentyl]carboxamide bishydrochloride

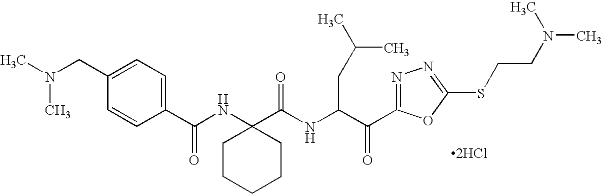

TLC: Rf 0.64 (methanol:chloroform:28% ammonia water=2:8:0.4); NMR (DMSO-d₆): δ 10.88 and 10.53 (br, each 1H), 8.13 (brd, J=6.9 Hz, 1H), 7.88 (d, J=8.1 Hz, 3H), 7.65 (d, J=8.1 Hz, 2H), 5.05 (m, 1H), 4.33 (s, 2H), 3.70 (m, 2H), 3.49 (m, 2H), 2.81 and 2.68 (each s, each 6H), 2.20-2.05 (m, 2H), 1.82-1.20 (m, 11H), 0.92-0.80 (m, 6H).

### EXAMPLE 1(76)

1-(3-dimethylaminomethylbenzoylamino)cyclohexyl-N-[1-[5-(2-dimethylaminoethylthio)-1,3,4-oxadiazol-2-yl]-4-methyl-1-oxo-2-pentyl]carboxamide bishydrochloride

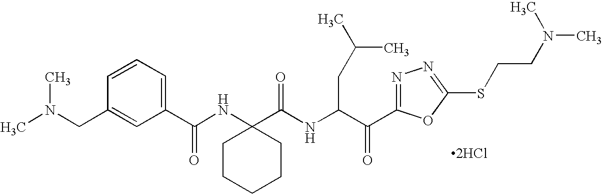

TLC: Rf 0.65 (methanol:chloroform:28% ammonia water=2:8:0.4); NMR (DMSO-d₆): δ 10.71 and 10.43 (br, each 1H), 8.16 (brd, J=6.7 Hz, 1H), 8.06 (brs, 1H), 7.88 (d, J=7.2 Hz, 1H), 7.85 (s, 1H), 7.70 (d, J=7.2 Hz, 1H), 7.55 (t, J=7.2 Hz, 1H), 5.02 (m, 1H), 4.34 (s, 2H), 3.70 (m, 2H), 3.51 (m, 2H), 2.81 and 2.71 (each s, each 6H), 2.20-2.03 (m, 2H), 1.90-1.20 (m, 11H), 0.90-0.80 (m, 6H).

### EXAMPLE 1(77)

cycloheptyl-N-[4-methyl-1-oxo-1-[5-(2-trimethylammonioethylthio)-1,3,4-oxadiazol-2-yl]-2-pentyl]carboxamide iodide

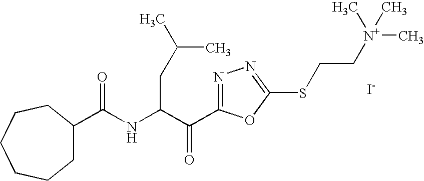

TLC: Rf 0.34 (ethyl acetate:acetic acid:water=3:1:1); NMR (CDCl₃): δ 6.38 (d, J=7.2 Hz, 111), 5.42-5.26 (m, 1H), 4.26-4.07 and 4.07-3.90 (each m, each 2H), 3.57 (s, 9H), 2.45-2.30 (m, 1H), 1.96-1.36 (m, 15H), 1.01 and 0.98 (each d, J=6.0 Hz, each 3H).

### EXAMPLE 1(78)

cycloheptyl-N-[(2S)-4-methyl-1-[5-(2,4-dioxo-1-methyl-1,3-imidazolidin-3-ylmethylthio)-1,3,4-oxadiazol-2-yl]-1-oxo-2-pentyl]carboxamide

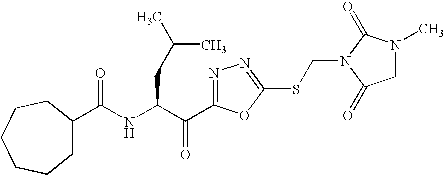

TLC: Rf 0.27 (n-hexane:ethyl acetate=1:2); NMR (CDCl₃): δ 5.98 (d, J=7.5 Hz, 1H), 5.39 (ddd, J=10.1, 7.5, 3.6 Hz, 1H), 5.30 and 5.23 (each d, J=13.2 Hz, each 1H), 3.93 (s, 2H), 3.01 (s, 3H), 2.39-2.25 (m, 1H), 1.96-1.37 (m, 15H), 1.03 and 0.98 (each d, J=6.0 Hz, each 3H).

### EXAMPLE 1(79)

1-benzoylaminocyclohexyl-N-[(2S)-4-methyl-1-[5-(2,4-dioxo-1-methyl-11,3-iniidazolidin-3-ylmethylthio)-1,3,4-oxadiazol-2-yl]-1-oxo-2-pentyl]carboxamide

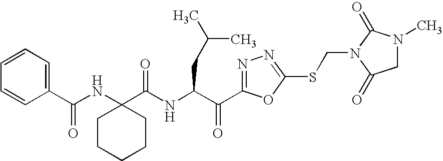

TLC: Rf 0.18 (n-hexane:ethyl acetate=1:2); NMR (CDCl₃): δ 8.10 (d, J=6.6 Hz, 1H), 7.84-7.70 (m, 2H), 7.60-7.39 (m, 3H), 6.12 (s, 1H), 5.35-5.23 (m, 1H), 5.25 and 5.18 (each d, J=13.2 Hz, each 1H), 3.90 (s, 2H), 3.00 (s, 3H), 2.40-2.15 (m, 2H), 2.10-1.20 (m, 11H), 1.01 and 0.98 (each d, J=6.0 Hz, each 3H).

### EXAMPLE 1(80)

cycloheptyl-N-[(2S)-4-methyl-1-[5-(1,2,4-oxadiazol-3-ylmethylthio)-1,3,4-oxadiazol-2-yl]-1-oxo-2-pentyl] carboxamide

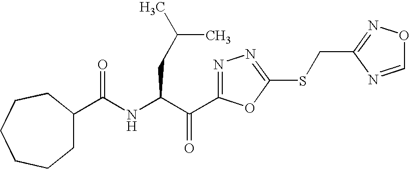

TLC: Rf 0.37 (n-hexane:ethyl acetate=1:1); NMR (CDCl₃): δ 8.74 (s, 1H), 5.94 (d, J=7.5 Hz, 1H), 5.39 (ddd, J=10.2, 7.5, 3.9 Hz, 1H), 4.70 (s, 2H), 2.39-2.25 (m, 1H), 1.96-1.35 (m, 15H), 1.02 and 0.98 (each d, J=6.3 Hz, each 3H).

### EXAMPLE 1(81)

1-benzoylaminocyclohexyl-N-[(2S)-4-methyl-1-[5-(2,4-dioxo-1,5,5-trimethyl-1,3-imidazolidin-3-ylmethylthio)-1,3,4-oxadiazol-2-yl]-1-oxo-2-pentyl]amide

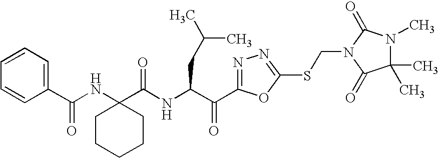

TLC: Rf 0.31 (n-hexane:ethyl acetate=1:2); NMR (DMSO-d₆): δ 8.05 (d, J=7.2 Hz, 1H), 7.82-7.80 (m, 3H), 7.53-7.44 (m, 2H), 5.14 (s, 2H), 5.05-4.97 (m, 1H), 2.77 (s, 3H), 2.18-2.05 (m, 2H), 1.93-1.17 (m, 11H), 1.28 (s, 6H), 0.87 (d, J=5.7 Hz, 3H), 0.84 (d, J=5.7 Hz, 3H).

### EXAMPLE 1(82)

cycloheptyl-N-[(2S)-4-methyl-1-[5-(2-pyrazolylethylthio)-1,3,4-oxadiazol-2-yl]-1-oxo-2-pentyl]carboxamide

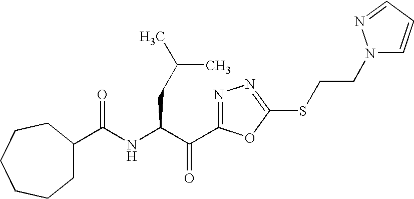

TLC: Rf 0.27 (n-hexane:ethyl acetate=1:1); NMR (CDCl₃): δ 7.55 (d, J=2.1 Hz, 1H), 7.44 (d, J=2.1 Hz, 1H), 6.26 (t, J=2.1 Hz, 1H), 5.94 (brd, J=7.5 Hz, 1H), 5.47-5.37 (m, 1H), 4.61 (t, J=6.3 Hz, 2H), 3.78 (t, J=6.3 Hz, 2H), 2.38-2.26 (m, 1H), 1.99-1.39 (m, 15H), 1.04 and 0.99 (each d, J=6.3 Hz, each 3H).

### EXAMPLE 1(83)

1-(N-methylpiperidin-4-ylcarbonylamino)cyclohexyl-N-[4-methyl-1-[5-(1-methylethylthio)-1,3,4-oxadiazol-2-yl]-1-oxo-2-pentyl]carboxamide hydrochloride

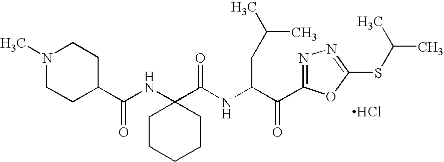

TLC: Rf 0.43(ethyl acetate:acetic acid:water=3:1:1); NMR (DMSO-d₆): δ 10.05-9.80(br, 1H), 8.21 (d, J=7.2 Hz, 1H), 7.65 (5, 1H), 5.00-4.88 (m, 1H), 3.96 (sept, J=6.6 Hz, 1H), 3.50-2.78 (m, 4H), 2.73 and 2.71 (each s, total 3H), 2.06-1.04 (m, 18H), 1.46 (d, J=6.6 Hz, 6H), 0.88 (d, J=5.7 Hz, 6H).

### EXAMPLE 1(84)

1-(3-piperidinopropanoylamino)cyclohexyl-N-[4-methyl-1-[5-(1-methylethylthio)-1,3,4-oxadiazol-2-yl]-1-oxo-2-pentyl]carboxamide hydrochloride

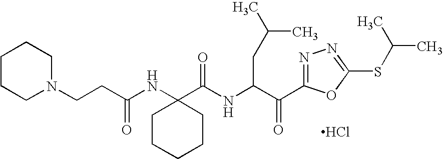

TLC: Rf 0.51(ethyl acetate:acetic acid:water-3:1:1); NMR (DMSO-d₆): δ 10.25-10.05 (br, 1H), 8.24 (d, J=6.9 Hz, 1H), 7.85 (s, 1H), 4.97-4.88 (m, 1H), 3.98 (sept, J=6.6 Hz, 1H), 3.25-3.12 (m, 2H), 2.90-2.64 (m, 4H), 2.00-1.93 (m, 2H), 1.93-1.05 (m, 19H), 1.46 (d, J=6.6 Hz, 6H), 0.92-0.80 (m, 6H).

### EXAMPLE 1(85)

1-(4-phenylbenzoylamino)cyclohexyl-N-[4-methyl-1-[5-(2-dimethylaminoethylthio)-1,3,4-oxadiazol-2-yl]-1-oxo-2-pentyl]carboxamide hydrochloride

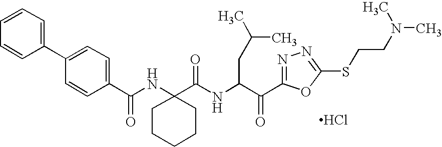

TLC: Rf 0.44 (chloroform:methanol=9:1); NMR (DMSO-d₆): δ 10.50 (br, 1H), 8.12 (brd, J=7.2 Hz, 1H), 7.90 (m, 3H), 7.70 (m, 4H), 7.52-7.40 (m, 3H), 5.03 (m, 1H), 3.80-3.60 (m, 4H), 2.82 (s, 6H), 2.22-2.05 (m, 2H), 1.90-1.20 (m, 11H), 0.90-0.80 (m, 6H).

### EXAMPLE 1(86)

1-(3-trifluoromethylbenzoylamino)cyclohexyl-N-[4-methyl-1-[5-(2-dimethylaminoethylthio)-1,3,4-oxadiazol-2-yl]-1-oxo-2-pentyl]carboxamide hydrochloride

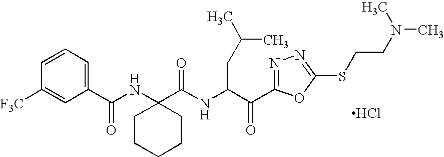

TLC: Rf 0.70 (chloroform:methanol=9:1); NMR (DMSO-d₆): δ 10.50 (br, 1H), 8.22-8.05 (m, 4H), 7.90 (d, J=7.5 Hz, 1H), 7.71 (t, J=7.5 Hz, 1H), 5.01 (m, 1H), 3.73-3.62 (m, 2H), 3.52-3.43 (m, 2H), 2.82 (s, 6H), 2.10 (m, 2H), 1.90-1.20 (m, 11H), 0.90-0.80 (m, 6H).

### EXAMPLE 1(87)

1-(4-trifluoromethyloxybenzoylamino)cyclohexyl-N-[4-methyl-1-[5-(2-dimethylaminoethylthio)-1,3,4-oxadiazol-2-yl]-1-oxo-2-pentyl]carboxamide hydrochloride

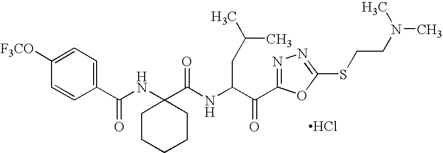

TLC: Rf 0.63 (chloroform:methanol=9:1); NMR (DMSO-d₆): δ 10.50 (br, 1H), 8.13 (brd, J=6.9 Hz, 1H), 7.95 (m, 3H), 7.46 (d, J=8.4 Hz, 2H), 5.01 (m, 11H), 3.73-3.62 (m, 2H), 3.52-3.43 (m, 2H), 2.82 (s, 6H), 2.10 (m, 2H), 1.90-1.20 (m, 11H), 0.90-0.80 (m, 6H).

### EXAMPLE 1(88)

cyclohexyl-N-[1-[5-(2-azetidinoethylthio)-1,3,4-oxadiazol-2-yl]-4-methyl-1-oxo-2-pentyl]carboxamide hydrochloride

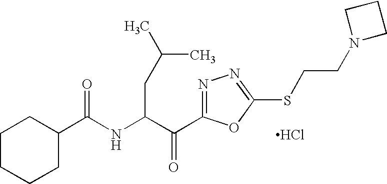

TLC: Rf 0.19 (ethyl acetate:methanol=4:1); NMR (CDCl₃): δ 13.36 (brs, 1H), 6.01 (brd, J=7.5 Hz, 1H), 5.48-5.38 (m, 1H), 4.55-4.39 (m, 2H), 4.10-3.88 (m, 2H), 3.78-3.50 (m, 4H), 2.91-2.71 and 2.54-2.37 (each m, total 2H), 2.25-2.13 (m, 1H), 2.00-1.10 (m, 13H), 1.04 and 1.00 (each d, J=6.0 Hz, each 3H).

### EXAMPLE 1(89)

1-(4-trifluoromethylbenzoylamino)cyclohexyl-N-[1-[5-(2-dimethylaminoethylthio)-1,3,4-oxadiazol-2-yl]-4-methyl-1-oxo-2-pentyl]carboxamide hydrochloride

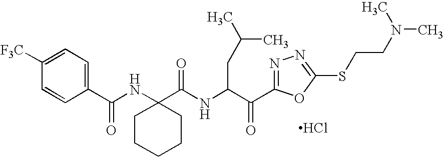

TLC: Rf 0.36 (methanol:chloroform=1:9); NMR (DMSO-d₆): δ 10.56 (br, 1H), 8.16 (brd, J=7.2 Hz, 1H), 8.07 (brs, 1H), 8.01 (d, J=7.8 Hz, 2H), 7.83 (d, J=7.8 Hz, 2H), 5.01 (m, 1H), 3.73 and 3.49 (each m, each 2H), 2.82 (s, 6H), 2.10 (m, 2H), 1.90-1.20 (m, 11H), 0.86 (d, J=6.0 Hz, 3H), 0.84 (d, J=6.0 Hz, 3H).

### EXAMPLE 1(90)

1-(2-trifluoromethylbenzoylamino)cyclohexyl-N-[1-[5-(2-dimethylaminoethylthio)-1,3,4-oxadiazol-2-yl]-4-methyl-1-oxo-2-pentyl]carboxamide hydrochloride

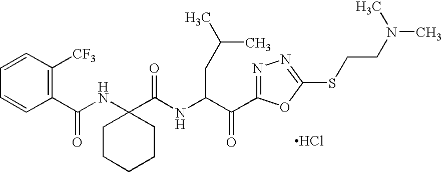

TLC: Rf 0.36 (methanol:chloroform=1:9); NMR (DMSO-d₆): δ 10.68 (br, 1H), 8.27 (brs, 1H), 8.14 (brd, J=6.9 Hz, 1H), 7.83-7.60 (m, 4H), 5.08 (q, J=6.9 Hz, 1H), 3.71 (t, J=7.5 Hz, 2H), 3.48 (m, 2H), 2.81 (s, 6H), 2.10 (m, 2H), 1.82-1.18 (m, 11H), 0.89 (d, J=6.0 Hz, 3H), 0.88 (d, J=6.0 Hz, 3H).

### EXAMPLE 1(91)

1-(3-trifluoromethyloxybenzoylamino)cyclohexyl-N-[1-[5-(2-dimethylaminoethylthio)-1,3,4-oxadiazol-2-yl]-4-methyl-1-oxo-2-pentyl]carboxamide hydrochloride

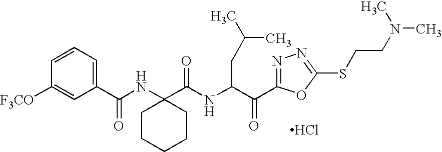

TLC: Rf 0.48 (methanol:chloroform=1:9); NMR (DMSO-d₆): δ 10.50 (br, 1H), 8.15 (brd, J=7.2 Hz, 1H), 8.00 (brs, 1H), 7.86 (brd, J=7.5 Hz, 1H), 7.76 (brs, 1H), 7.60 (t, J=7.5 Hz, 1H), 7.52 (d, J=7.5 Hz, 1H), 5.02 (m, 1H), 3.71 (m, 2H), 3.48 (t, J=7.5 Hz, 2H), 2.81 (s, 6H), 2.08 (m, 2H), 1.82-1.18 (m, 111H), 0.89-0.77 (m, 6H).

### EXAMPLE 1(92)

1-(4-fluorobenzoylamino)cyclohexyl-N-[1-[5-(2-dimethylaminoethylthio)-1,3,4-oxadiazol-2-yl]-4-methyl-1-oxo-2-pentyl]carboxamide hydrochloride

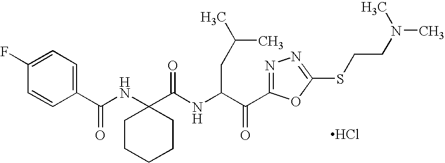

TLC: Rf 0.42 (methanol:chloroform=1:9); NMR (DMSO-d₆): δ 10.60 (br, 1H), 8.11 (brd, J=7.2 Hz, 1H), 7.91 (brs, 1H), 7.86 (dd, J=8.7, 3.3 Hz, 2H), 7.29 (t, J=8.7 Hz, 2H), 5.02 (m, 1H), 3.71 (m, 2H), 3.48 (m, 2H), 2.81 (s, 6H), 2.08 (m, 2H), 1.82-1.18 (m, 11H), 0.86 (d, J=6.0 Hz, 3H), 0.83 (d, J=6.0 Hz, 3H).

### EXAMPLE 1(93)

1-(3-fluorobenzoylamino)cyclohexyl-N-[1-[5-(2-dimethylaminoethylthio)-1,3,4-oxadiazol-2-yl]-4-methyl-1-oxo-2-pentyl]carboxamide hydrochloride

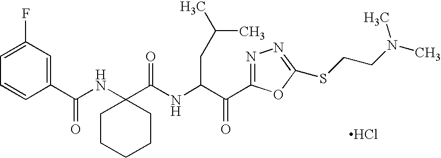

TLC: Rf 0.42 (methanol:chloroform=1:9); NMR (DMSO-d₆): δ 10.40 (br, 1H), 8.12 (brd, J=6.9 Hz, 1H), 7.91 (brs, 1H), 7.70-7.60 (m, 2H), 7.50 (dt, J=5.7, 7.8 Hz, 1H), 7.40 (dt, J=2.4, 7.8 Hz, 1H), 5.02 (m, 1H), 3.69 (m, 2H), 3.48 (m, 2H), 2.81 (s, 6H), 2.08 (m, 2H), 1.82-1.18 (m, 11H), 0.86 (d, J=6.0 Hz, 3H), 0.83 (d, J=6.0 Hz, 3H).

### EXAMPLE 1(94)

1-(2-fluorobenzoylamino)cyclohexyl-N-[1-[5-(2-dimethylaminoethylthio)-1,3,4-oxadiazol-2-yl]-4-methyl-1-oxo-2-pentyl]carboxamide hydrochloride

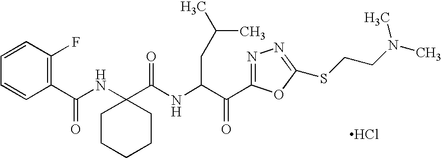

TLC: Rf 0.42 (methanol:chloroform=1:9); NMR (DMSO-d₆): δ 10.70 (br, 1H), 8.11 (brd, J=7.2 Hz, 1H), 7.83 and 7.81 (each brs, total 1H), 7.63 (t, J=7.2 Hz, 1H), 7.54 (m, 1H), 7.38-7.29 (m, 2H), 5.05 (m, 1H), 3.71 (t, J=8.1 Hz, 2H), 3.48 (t, J=8.1 Hz, 2H), 2.81 (s, 6H), 2.12 (br, 2H), 1.78-1.18 (m, 11H), 0.90 (d, J=6.0 Hz, 3H), 0.86 (d, J=6.0 Hz, 3H).

### EXAMPLE 1(95)

1-(3-methoxybenzoylamino)cyclohexyl-N-[1-[5-(2-dimethylaminoethylthio)-1,3,4-oxadiazol-2-yl]-4-methyl-1-oxo-2-pentyl]carboxamide hydrochloride

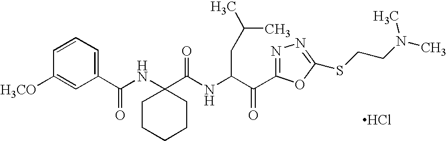

TLC: Rf 0.43 (methanol:chloroform=1:9); NMR (DMSO-d₆): δ 10.54 (br, 1H), 8.08 (brd, J=7.2 Hz, 1H), 7.78 (brs, 1H), 7.43-7.30 (m, 3H), 7.10 (m, 1H), 5.05 (m, 1H), 3.80 (s, 3H), 3.71 (m, 2H), 3.48 (t, J=7.2 Hz, 2H), 2.81 (s, 6H), 2.12 (br, 2H), 1.80-1.20 (m, 111H), 0.86 (d, J=6.0 Hz, 3H), 0.84 (d, J=6.0 Hz, 3H).

### EXAMPLE 1(96)

1-nicotinoylaminocyclohexyl-N-[1-[5-(2-dimethylaminoethylthio)-1,3,4-oxadiazol-2-yl]-4-methyl-1-oxo-2-pentyl]carboxamide bishydrochloride

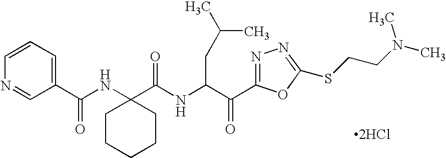

TLC: Rf 0.76 (methanol:chloroform=1:4); NMR (DMSO-d₆): δ 10.70 (br, 1H), 9.22 (s, 1H), 8.89 (d, J=5.4 Hz, 1H), 8.53 (brd, J=7.8 Hz, 1H), 8.38 (brs, 1H), 8.32 (d, J=6.9 Hz, 1H), 7.84 (dd, J=5.4, 6.9 Hz, 1H), 4.97 (m, 1H), 3.69 (m, 2H), 3.50 (m, 2H), 2.81 (s, 6H), 2.10 (br, 2H), 1.82-1.19 (m, 11H), 0.90-0.77 (m, 6H).

### EXAMPLE 1(97)

1-isonicotinoylaminocyclohexyl-N-[1-[5-(2-dimethylaminoethylthio)-1,3,4-oxadiazol-2-yl]-4-methyl-1-oxo-2-pentyl]carboxamide bishydrochloride

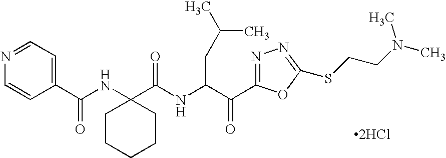

TLC: Rf 0.38 (methanol:chloroform=1:9); NMR (DMSO-d₆): δ 10.70 (br, 1H), 8.90 (d, J=5.4 Hz, 2H), 8.40 (brs, 1H), 8.29 (brd, J=6.9 Hz, 1H), 8.05 (d, J=5.4 Hz, 2H), 5.00 (m, 1H), 3.73 (m, 2H), 3.43 (m, 2H), 2.81 (s, 6H), 2.10 (br, 2H), 1.82-1.19 (m, 11H), 0.90-0.77 (m, 6H).

### EXAMPLE 1(98)

1-benzyloxymethylcyclohexyl-N-[(2S)-1-[5-(1-methylethylthio)-1,3,4-oxadiazol-2-yl]-4-methyl-1-oxo-2-pentyl]carboxamide

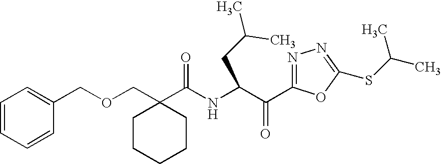

TLC: Rf 0.35 (n-hexane:ethyl acetate=4:1); NMR (CDCl₃): δ 7.36-7.26 (m, 5H), 7.15 (d, J=6.6 Hz, 1H), 5.39-5.30 (m, 1H), 4.61 (d, J=11.4 Hz, 1H), 4.53 (d, J=11.4 Hz, 1H), 4.07-3.98 (m, 1H), 3.52 (d, J=9.3 Hz, 1H), 3.49 (d, J=9.3 Hz, 1H), 2.01-1.85 (m, 2H), 1.89-1.26 (m, 11H), 1.53 (d, J=6.6 Hz, 6H), 0.95 (d, J=6.0 Hz, 3H), 0.86 (d, J=6.0 Hz, 3H).

### EXAMPLE 1(99)

1-benzyloxymethylcyclohexyl-N-[(2S)-1-[5-(2,4-dioxo-1,5,5-trimethylimidazolidin-3-ylmethylthio)-1,3,4-oxadiazol-2-yl]-4-methyl-1-oxo-2-pentyl]carboxamide

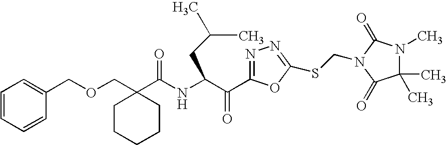

TLC: Rf 0.29 (n-hexane:ethyl acetate=1:1); NMR (CDCl₃): δ 7.36-7.26 (m, 5H), 7.19 (d, J=6.3 Hz, 1H), 5.36-5.29 (m, 1H), 5.27 (d, J=13.2 Hz, 1H), 5.20 (d, J=13.2 Hz, 1H), 4.61 (d, J=11.7 Hz, 1H), 4.53 (d, J=11.7 Hz, 1H), 3.52 (d, J=9.3 Hz, 1H), 3.46 (d, J=9.3 Hz, 1H), 2.88 (s, 3H), 2.00-1.85 (m, 2H), 1.91-1.32 (m, 11H), 1.39 (s, 6H), 0.95 (d, J=6.3 Hz, 3H), 0.86 (d, J=6.3 Hz, 3H).

### EXAMPLE 1(100)

1-benzyloxymethylcyclohexyl-N-[1-[5-(2-dimethylaminoethylthio)-1,3,4-oxadiazol-2-yl]-4-methyl-1-oxo-2-pentyl]carboxamide hydrochloride

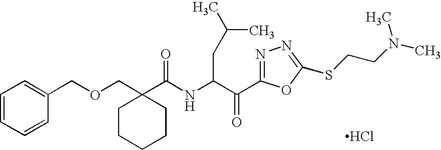

TLC: Rf 0.40 (ethyl acetate:methanol=9:1); NMR (DMSO-d₆): δ 8.06 (d, J=5.7 Hz, 1H), 7.35-7.24 (m, 5H), 5.11-5.04 (m, 1H), 4.41 (s, 2H), 3.71 (t, J=6.3 Hz, 2H), 3.53-3.35 (m, 4H), 2.81 (s, 6H), 2.08-1.92 (m, 2H), 1.74-1.14 (m, 11H), 0.87 (d, J=6.0 Hz, 3H), 0.86 (d, J=6.0 Hz, 3H).

### EXAMPLE 1(101)

cyclohexyl-N-[1-[5-[2-(methylphenylamino)ethylthio]-1,3,4-oxadiazol-2-yl]-4-methyl-1-oxo-2-pentyl]carboxamide hydrochloride

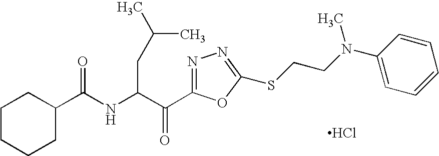

TLC: Rf 0.74 (n-hexane:ethyl acetate=1:1); NMR (DMSO-d₆): δ 8.38 (d, J=6.3 Hz, 1H), 7.24-7.14 (m, 2H), 6.83 (d, J=7.8 Hz, 2H), 6.67 (d, J=7.2 Hz, 1H), 5.06-4.96 (m, 1H), 3.78-3.66 (m, 2H), 3.52-3.40 (m, 2H), 2.94 (s, 3H), 2.28-2.12 (m, 1H), 1.80-1.48 and 1.38-1.07 (each m, total 13H), 0.92 and 0.91 (each d, J=6.0 Hz, total 6H).

### Example 1(102) 1-cyclohexylcarbonylaminocyclohexyl-N-[1-[5-(2-dimethylaminoethylthio)-1,3,4-oxadiazol-2-yl]-4-methyl-1-oxo-2-pentyl]carboxamide hydrochloride

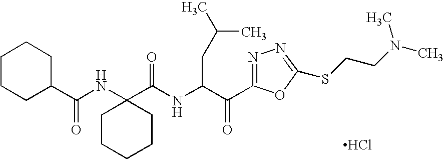

TLC: Rf 0.32 (ethyl acetate:methanol=8:2); NMR (DMSO-d₆): δ 10.35-10.15 (broad, 1H), 7.97 (brd, J=6.9 Hz, 1H), 7.35 (brs, 1H), 5.02 (m, 1H), 3.70 (m, 2H), 3.49 (m, 2H), 2.82 (s, 6H), 2.26 (m, 1H), 2.00-1.05 (m, 23H), 0.87 (d, J=6.3 Hz, 6H).

### EXAMPLE 1(103)

cyclohexyl-N-[1-[5-[2-(N-benzyl-N-methylamino)ethylthio]-1,3,4-oxadiazol-2-yl]-4-methyl-1-oxo-2-pentyl]carboxamide hydrochloride

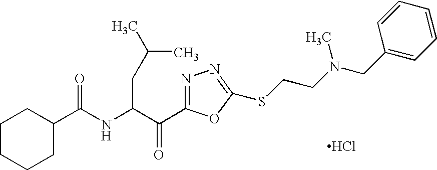

TLC: Rf 0.48 (n-hexane:ethyl acetate=1:1); NMR (DMSO-d₆): δ 10.95-10.70 (br, 1H), 8.39 (d, J=6.6 Hz, 1H), 7.66-7.37 (m, 5H), 5.08-4.97 (m, 1H), 4.47 (d, J=13.2 Hz, 1H), 4.30 (d, J=13.2 Hz, 1H), 3.90-3.66 (m, 2H), 3.63-3.40 (m, 2H), 2.74 (s, 3H), 2.29-2.12 (m, 1H), 1.83-1.48 and 1.37-1.06 (each m, total 13H), 0.91 and 0.90 (each d, J=6.3 Hz, total 6H).

### EXAMPLE 1(104)

1-(t-butoxycarbonylamino)cyclohexyl-N-[1-[5-(2-dimethylaminoethylthio)-1,3,4-oxadiazol-2-yl]-4-methyl-1-oxo-2-pentyl]carboxamide hydrochloride

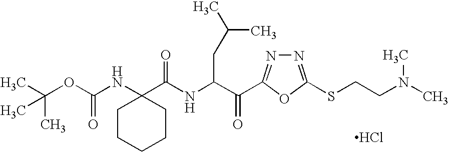

TLC: Rf 0.46 (chloroform:methanol=9:1); NMR (DMSO-d₆): δ 10.78 (br, 1H), 7.91 (brd, J=6.6 Hz, 1H), 6.65 (brs, 1H), 5.02 (m, 1H), 3.72 (m, 2H), 3.43 (m, 2H), 2.81 (s, 6H), 2.05 (br, 2H), 1.82-1.19 (m, 11H), 1.10 (s, 9H), 0.92 (d, J=6.0 Hz, 6H).

### EXAMPLE 1(105)

1-(3-phenylpropionylamino)cyclohexyl-N-[1-[5-(2-dimethylbaminoethylthio)-1,3,4-oxadiazol-2-yl]-4-methyl-1-oxo-2-pentyl]carboxamide hydrochloride

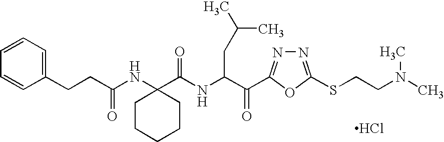

TLC: Rf 0.47(chloroform:methanol=9:1); NMR (DMSO-d₆): δ 10.20 (br, 1H), 8.07 (brd, J=6.9 Hz, 1H), 7.50 (brs, 1H), 7.30-7.10 (m, 5H), 4.98 (q, J=6.9 Hz, 1H), 3.72 (m, 2H), 3.50 (m, 2H), 2.84-2.70 (m, 8H), 2.50 (m, 2H), 2.00-1.83 (m, 2H), 1.76-1.05 (m, 11H), 0.92 (d, J=6.0 Hz, 6H).

### EXAMPLE 1(106)

1-benzylcarbonylaminocyclohexyl-N-[1-[5-(2-dimethylaminoethylthio)-1,3,4-oxadiazol-2-yl]-4-methyl-1-oxo-2-pentyl]carboxamide hydrochloride

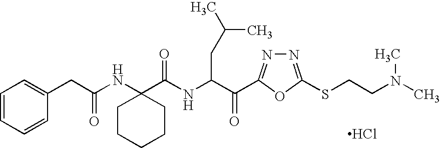

TLC: Rf 0.34 (chloroform:methanol:acetic acid=10:2:1); NMR (DMSO-d₆): δ 10.18 (br, 1H), 8.03 (brd, J=6.9 Hz, 1H), 7.74 (brs, 1H), 7.30-7.10 (m, 5H), 4.98 (m, 1H), 3.68 (t, J=7.8 Hz, 2H), 3.50 (m, 4H), 2.81 (s, 6H), 2.02-1.88(m, 2H), 1.70-1.10 (m, 11H), 0.90-0.78 (m, 6H).

### EXAMPLE 1(107)

1-cyclopentylcarbonylaminocyclohexyl-N-[1-[5-(2-dimethylaminoethylthio)-1,3,4-oxadiazol-2-yl]-4-methyl-1-oxo-2-pentyl]carboxamide hydrochloride

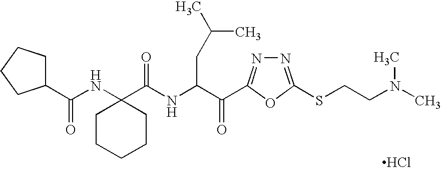

TLC: Rf 0.39 (chloroform:methanol=9:1); NMR (DMSO-d₆): δ 10.53 (br, 1H), 8.01 (brd, J=6.9 Hz, 1H), 7.42 (brs, 1H), 5.00 (m, 1H), 3.68 (m, 2H), 3.50 (m, 2H), 2.81 (s, 6H), 2.79 (m, 1H), 2.02-1.88 (m, 2H), 1.80-1.10 (m, 19H), 0.88 (d, J=6.0 Hz, 6H).

### EXAMPLE 1(108)

1-(2-thienylcarbonylamino)cyclohexyl-N-[1-[5-(2-dimethylaminoethylthio)-1,3,4-oxadiazol-2-yl]-4-methyl-1-oxo-2-pentyl]carboxamide hydrochloride

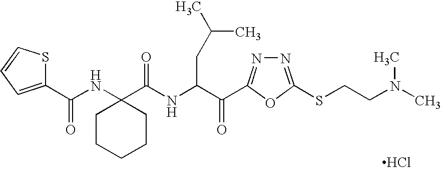

TLC: Rf 0.44 (chloroform:methanol=9:1); NMR (DMSO-d₆): δ 10.58 (br, 1H), 8.13 (brd, J=7.2 Hz, 1H), 7.89 (d, J=3.9 Hz, 1H), 7.79 (brs, 1H), 7.73 (d, J=4.8 Hz, 1H), 7.13 (dd, J=4.8, 3,9 Hz, 1H), 5.01 (m, 1H), 3.70 (m, 2H), 3.50 (m, 2H), 2.81 (s, 6H), 2.20-2.00 (m, 2H), 1.84-1.20 (m, 11H), 0.86 (d, J=6.0 Hz, 3H), 0.84 (d, J=6.0 Hz, 3H).

### EXAMPLE 1(109)

1-(4-methoxybenzoylamino)cyclohexyl-N-[1-[5-(2-dimethylaminoethylthio)-1,3,4-oxadiazol-2-yl]-4-methyl-1-oxo-2-pentyl]carboxamide hydrochloride

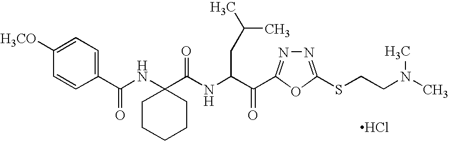

TLC: Rf 0.43 (chloroform:methanol=9:1); NMR (DMSO-d₆): δ 10.62 (br, 1H), 8.08 (brd, J=6.6 Hz, 1H), 7.78 (d, J=8.7 Hz, 2H), 7.63 (brs, 1H), 6.98 (d, J=8.7 Hz, 2H), 5.01 (m, 1H), 3.80 (s, 3H), 3.70 (m, 2H), 3.50 (m, 2H), 2.82 (s, 6H), 2.20-2.03 (m, 2H), 1.82-1.17 (m, 11H), 0.86 (d, J=6.0 Hz, 3H), 0.84 (d, J=6.0 Hz, 3H).

### EXAMPLE 1(110)

1-(2-furylcarbonylamino)cyclohexyl-N-[1-[5-(2-dimethylaminoethylthio)-1,3,4-oxadiazol-2-yl]-4-methyl-1-oxo-2-pentyl]carboxamide hydrochloride

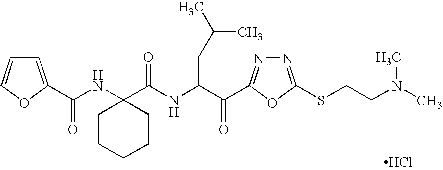

TLC: Rf 0.44 (chloroform:methanol=9:1); NMR (DMSO-d₆): δ 10.72 (br, 1H), 8.17 (brd, J=6.9 Hz, 1H), 7.85 (d, J=1.5 Hz, 1H), 7.51 (brs, 1H), 7.13 (d, J=3.6 Hz, 1H), 7.13 (d, J=3.6 Hz, 1H), 6.62 (dd, J=3.6, 1.5 Hz, 1H), 5.01 (m, 1H), 3.70 (m, 2H), 3.50 (m, 2H), 2.82 (s, 6H), 2.20-2.03 (m, 2H), 1.82-1.17 (m, 11H), 0.86 (d, J=6.0 Hz, 3H), 0.84 (d, J=6.0 Hz, 3H).

### EXAMPLE 1 (111)

1-(4-dimethylaminomethylbenzoylamino)cyclohexyl-N-[1-[5-(2,4-dioxo-1,5,5-trimethylimidazolidin-3-ylmethylthio)-1,3,4-oxadiazol-2-yl]-4-methyl-1-oxo-2-pentyl]carboxamide

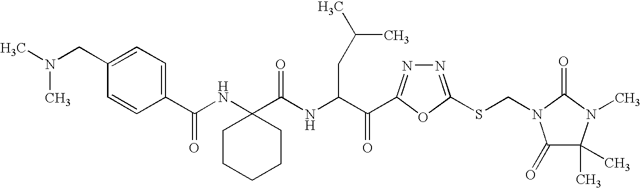

TLC: Rf 0.50 (chloroform:methanol:water=40:10:1); NMR (DMSO-d₆): δ 8.04 (d, J=6.9 Hz, 1H), 7.77 (d, J=7.8 Hz, 2H), 7.76 (s, 1H), 7.37 (d, J=7.8 Hz, 2H), 5.14 (s, 2H), 5.02-4.98 (m, 1H), 3.47 (s, 2H), 2.77 (s, 3H), 2.28-2.09 (m, 2H), 2.17 (s, 6H), 1.83-1.42 (m, 11H), 1.28 (s, 6H), 0.87 (d, J=5.4 Hz, 3H), 0.84 (d, J=5.4 Hz, 3H).

### EXAMPLE 1(112)

1-(3-methylbenzoylamino)cyclohexyl-N-[1-[5-(2-dimethylaminoethylthio)-1,3,4-oxadiazol-2-yl]-4-methyl-1-oxo-2-pentyl]carboxamide hydrochloride

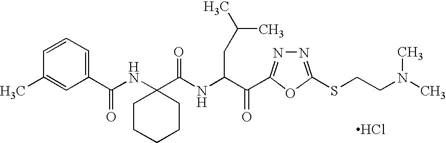

TLC: Rf 0.40 (methylene chloride:methanol:acetic acid=9:1:1); NMR (DMSO-d₆): δ 10.42-10.22 (br, 1H), 8.07 (d, J=6.9 Hz, 1H), 7.73 (s, 1H), 7.62 (s, 1H), 7.61-7.54 (m, 1H), 7.34 (d, J=4.8 Hz, 2H), 5.13-4.98 (m, 1H), 3.76-3.64 (m, 2H), 3.56-3.48 (m, 2H), 2.83 (s, 6H), 2.36 (s, 3H), 2.21-1.17 (m, 13H), 0.87 and 0.85 (each d, J=6.0 Hz, total 6H).

### EXAMPLE 1 (113)

1-(4-methylbenzoylamino)cyclohexyl-N-[1-[5-(2-dimethylaminoethylthio)-1,3,4-oxadiazol-2-yl]-4-methyl-1-oxo-2-pentyl]carboxamide hydrochloride

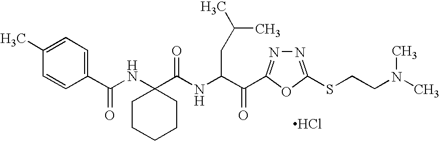

TLC: Rf 0.42 (methylene chloride:methanol:acetic acid=9:1:1); NMR (DMSO-d₆): δ 10.63-10.45 (br, 1H), 8.08 (d, J=7.2 Hz, 1H), 7.71 (d, J=8.1 Hz, 2H), 7.70 (s, 1H), 7.26 (d, J=8.1 Hz, 2H), 5.08-4.96 (m, 1H), 3.78-3.62 (m, 2H), 3.53-3.44 (m, 2H), 2.82 (s, 6H), 2.35 (s, 3H), 2.18-1.18 (m, 13H), 0.87 and 0.84 (each d, J=6.0 Hz, total 6H).

### EXAMPLE 1 (114)

cyclohexyl-N-[(2S)-1-[5-[2-(1-methyl-2,4-dioxoimidazolidin-3-yl)ethylthio]-1,3,4-oxadiazol-2-yl]-4-methyl-1-oxo-2-pentyl]carboxamide

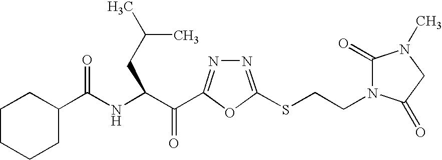

TLC: Rf 0.65 (ethyl acetate:methanol=9:1); NMR (CDCl₃): δ 6.02 (d, J=7.8 Hz, 1H), 5.46-5.39 (m, 1H), 4.02 (dt, J=2.7, 6.3 Hz, 2H), 3.89 (s, 2H), 3.60 (t, J=6.3 Hz, 2H), 3.00 (s, 3H), 2.17 (tt, J=11.4, 3.3 Hz, 1H), 1.89-1.23 (m, 13H), 1.03 (d, J=6.0 Hz, 3H), 0.97 (d, J=6.0 Hz, 3H).

### Example 1(115)

1-(t-butoxycarbonylamino)cyclohexyl-N-[4-methyl-1-[5-(2-morpholinoethylthio)-1,3,4-oxadiazol-2-yl]-1-oxo-2-pentyl]carboxaniide hydrochloride

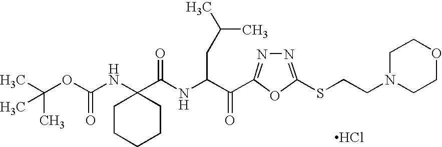

TLC: Rf 0.58 (ethyl acetate); NMR (DMSO-d₆): δ 11.2-10.6 (broad, 1H), 7.97 (brd, J=6.6 Hz, 1H), 6.54 (brs, 1H), 5.05 (m, 1H), 4.05-3.00 (m, 12H), 1.95-1.10 (m, 13H), 1.45 (s, 9H), 0.89 (d, J=6.0 Hz, 6H).

### EXAMPLE 1(116)

1-(t-butoxycarbonylamino)cyclohexyl-N-[1-[5-(3-dimethylaminopropylthio)-1,3,4-oxadiazol-2-yl]-4-methyl-1-oxo-2-pentyl]carboxamide hydrochloride

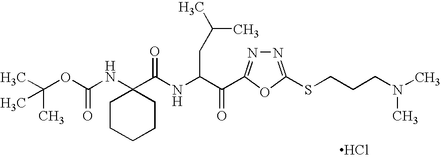

TLC: Rf 0.74 (chloroform:methanol:acetic acid=10:2:1); NMR (DMSO-d₆): δ 10.1-9.90 (broad, 1H), 7.95 (brd, J=6.6 Hz, 1H), 6.54 (brs, 1H), 5.05 (m, 1H), 3.42 (brt, J=6.6 Hz, 2H), 3.16 (m, 2H), 2.76 and 2.74 (s, 6H), 2.18 (m, 2H), 1.90-1.10 (m, 13H), 1.45 (s, 9H), 0.89 (d, J=6.0 Hz, 6H).

### EXAMPLE 1(117)

cyclohexyl-N-[1-[5-(2-dimethylaminoethylthio)-1,3,4-oxadiazol-2-yl]-4-methyl-1-oxo-2-pentyl]carboxamide hydrochloride

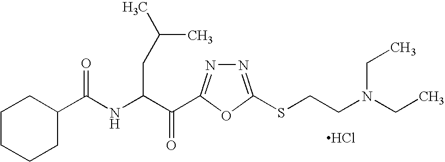

TLC: Rf 0.50 (ethyl acetate:methanol=9:1); NMR (DMSO-d₆): δ 8.40 (d, J=6.6 Hz, 1H), 5.05-4.98 (m, 1H), 3.73-3.68 (m, 2H), 3.49-3.44 (m, 2H), 3.20-3.14 (m, 4H), 2.29-2.13 (m, 1H), 1.83-1.47 (m, 7H), 1.38-1.07 (m, 12H), 0.91 (d, J=6.3 Hz, 3H1), 0.90 (d, J=6.3 Hz, 3H).

### EXAMPLE 1(118)

1-(4-morpholinomethylbenzoylamino)cyclohexyl-N-[1 [5-(2-dimethylaminoethylthio)-1,3,4-oxadiazol-2-yl]-4-methyl-1-oxo-2-pentyl]carboxamide bishydrochloride

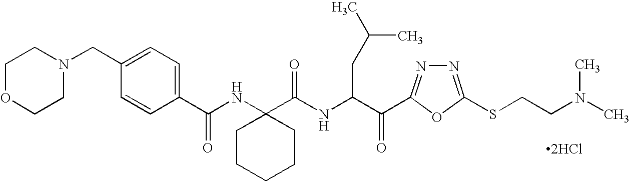

TLC: Rf 0.84 (methanol:chloroform:28% ammonia water=2:8:0.4); NMR (DMSO-d₆): δ 11.58 (br, 1H), 16.58 (br, 1H), 8.12 (brd, J=7.2 Hz, 111), 7.89 (brs, 1H), 7.87 (d, J=7.8 Hz, 2H), 7.71 (d, J=7.8 Hz, 2H), 5.01 (m, 1H), 4.28 (brs, 2H), 3.99-2.98 (m, 12H), 2.81 (s, 6H), 2.10 (br, 2H), 1.82-1.19 (m, 11111), 0.90-0.77 (m, 6H).

### Example 1(119)

1-(3-morpholinomethylbenzoylamino)cyclohexyl-N-[11-[5-(2-dimethylaminoethylthio)-1,3,4-oxadiazol-2-yl]-4-methyl-1-oxo-2-pentyl]carboxamide bishydrochloride

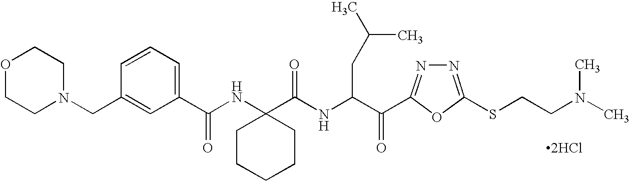

TLC: Rf 0.78 (methanol chloroform:28% ammonia water=2:8:0.4); NMR (DMSO-d₆): δ 11.58 (br, 1H), 10.70 (br, 1H), 8.20 (m, 2H), 7.85 (m, 2H), 7.75 (d, J=7.5 Hz, 111), 7.53 (t, J=7.5 Hz, 111), 5.01 (m, 1H), 4.40 (brs, 2H), 3.99-3.00 (m, 12H), 2.81 (s, 6H), 2.10 (br, 2H), 1.82-1.19 (m, 11H), 0.90-0.77 (m, 6H).

### EXAMPLE 1(120)

1-(2-butynyloxycarbonylamino)cyclohexyl-N-[1 [5-(2-dimethylaminoethylthio)1,3,4-oxadiazol-2-yl]-4-methyl-1-oxo-2-pentyl]carboxamide hydrochloride

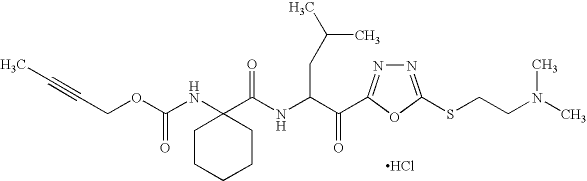

TLC: Rf 0.60 (methanol:chloroform=1:9); NMR (DMSO-d₆): δ 10.62 (br, 1H), 8.04 (d, J=6.9 Hz, 1H), 7.05 (brs, 1H), 4.98 (m, 1H), 4.50 (brs, 2H), 3.70 and 3.44 (each t, J=6.6 Hz, each 2H), 2.81 (s, 6H), 1.81 (s, 3H), 1.90-1.10 (m, 13H), 0.87 (d, J=6.0 Hz, 6H).

### EXAMPLE 1(121)

1-(3-butynyloxycarbonylamino)cyclohexyl-N-[1-[5-(2-dimethylaminoethylthio)-1,3,4-oxadiazol-2-yl]-4-methyl-1-oxo-2-pentyl]carboxamide hydrochloride

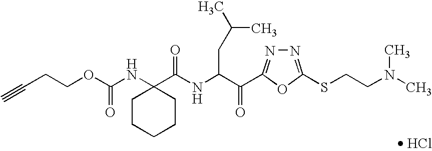

TLC: Rf 0.52 (methanol:chloroform=1:9); NMR (DMSO-d₆): δ 10.28 (br, 1H), 8.05 (brd, J=7.2 Hz, 1H), 7.02 (brs, 1H), 4.98 (m, 1H), 3.97 (t, J=6.6 Hz, 2H), 3.70 and 3.51 (each t, J=6.6 Hz, each 2H), 2.83 (brs, 1H), 2.81 (s, 6H), 2.43 (m, 2H), 1.90-1.10 (m, 13H), 0.87 (d, J=6.0 Hz, 6H).

### EXAMPLE 1(122)

1-methoxycarbonylaminocyclohexyl-N-[1-[5-(2-dimethylaminoethylthio)-1,3,4-oxadiazol-2-yl]-4-methyl-1-oxo-2-pentyl]carboxamide hydrochloride

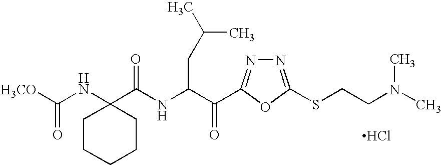

TLC: Rf 0.48 (methanol:chloroform=1:9); NMR (DMSO-d₆): δ 10.63 (br, 1H), 8.05 (brd, J=7.2 Hz, 1H), 6.90 (brs, 1H), 4.98 (m, 1H), 3.70 (t, J=6.6 Hz, 2H), 3.48 (m, 5H), 2.80 (s, 6H), 1.90-1.10 (m, 13H), 0.88 (d, J=6.0 Hz, 6H).

### EXAMPLE 1(123)

1-benzoylaminocyclohexyl-N-[1-[5-(2-diethylaminoethylthio)-1,3,4-oxadiazol-2-yl]-4-methyl-1-oxo-2-pentyl]carboxamide hydrochloride

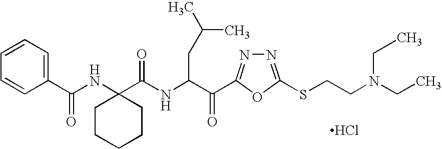

TLC: Rf 0.67 (chloroform:methanol:water=40:10:1); NMR (DMSO-d₆): δ 8.10 (d, J=7.2 Hz, 1H), 7.81 (d, J=6.9 Hz, 2H), 7.80 (s, 1H), 7.56-7.43 (m, 3H), 5.04-4.98 (m, 1H), 3.72-3.67 (m, 2H), 3.51-3.41 (m, 2H), 3.22-3.07 (m, 4H), 2.18-2.03 (m, 2H), 1.82-1.42 (m, 10H), 1.32-1.14 (m, 1H), 1.24 (t, J=7.2 Hz, 6H), 0.90-0.83 (m, 6H).

### EXAMPLE 1(124)

cyclohexyl-N-[1-[5-[2-(N-methoxy-N-methylamino)ethylthio]-1,3,4-oxadiazol-2-yl]-4-methyl-1-oxo-2-pentyl]carboxamide hydrochloride

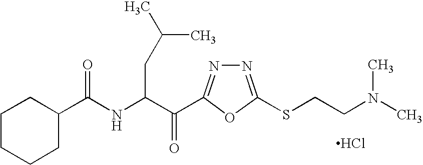

TLC: Rf 0.51 (n-hexane:ethyl acetate=1:1);NMR (DMSO-d₆): δ 8.37 (d, J=6.3 Hz, 1H), 5.04-4.94 (m, 1H), 3.51 (t, J=6.3 Hz, 2H), 3.44 (s, 3H), 3.00 (t, J=6.3 Hz, 2H), 2.56 (s, 3H), 2.28-2.12 (m, 1H), 1.80-1.47 and 1.34-1.08 (each m, total 13H), 0.91 and 0.90 (each d, J=6.3 Hz, total 6H).

### EXAMPLE 1(125)

cyclohexyl-N-[(2S)-1-[5-[2-(2,4-dioxoimidazolidin-3-yl)ethylthio]-1,3,4-oxadiazol-2-yl]-4-methyl-1-oxo-2-pentyl]carboxamide hydrochloride

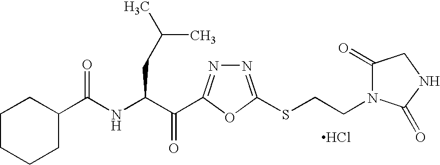

TLC: Rf 0.54 (chloroform:methanol=9:1); NMR (CDCl₃): δ 6.03 (brd, J=7.5 Hz, 1H), 5.69 (brs, 1H), 5.43-5.33 (m, 1H), 4.03 (t, J=5.7 Hz, 2H), 3.97 (s, 2H), 3.64-3.56 (m, 2H), 2.23-2.12 (m, 1H), 1.92-1.17 (m, 13H), 1.03 and 0.98 (each d, J=6.0 Hz, each 3H).

### EXAMPLE 1(126)

1-(4-chlorobenzoylamino)cyclohexyl-N-[1-[5-(2-dimethylaminoethylthio)-1,3,4-oxadiazol-2-yl]-4-methyl-1-oxo-2-pentyl]carboxamide hydrochloride

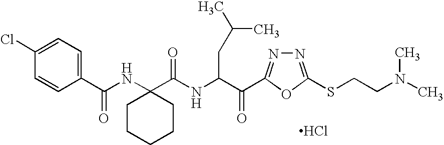

TLC: Rf 0.41 (methylene chloride:methanol:acetic acid=9:1:1); NMR (DMSO-d₆): δ 10.20-10.10 (br, 1H), 8.10 (d, J=7.2 Hz, 1H), 7.90 (s, 1H), 7.83 (d, J=8.4 Hz, 2H), 7.53 (d, J=8.4 Hz, 2H), 5.08-4.95 (m, 1H), 3.72-3.64 (m, 2H), 3.52-3.44 (m, 2H), 2.81 (s, 6H), 2.12-1.20 (m, 13H), 0.86 and 0.84 (each d, J=6.3 Hz, total 6H).

### EXAMPLE 1(127)

1-(2-trifluoromethyloxybenzoylamino)cyclohexyl-N-[1-[5-(2-dimethylaminoethylthio)-1,3,4-oxadiazol-2-yl]-4-methyl-1-oxo-2-pentyl]carboxamide hydrochloride

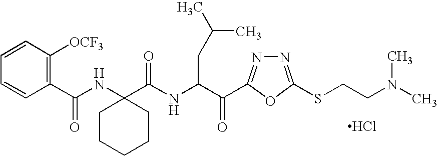

TLC: Rf 0.38 (methylene chloride:methanol:acetic acid=9:1:1); NMR (DMSO-d₆): δ 10.30-10.05 (br, 1H), 8.04 (d, J=6.9 Hz, 1H), 8.00 (s, 1H), 7.70-7.58 (m, 2H), 7.52-7.42 (m, 2H), 5.14-5.04 (m, 1H), 3.72-3.65 (m, 2H), 3.52-3.48 (m, 2H), 2.82 (s, 6H), 2.20-1.20 (m, 13H), 0.90 and 0.88 (each d, J=6.3 Hz, total 6H).

### EXAMPLE 1(128)

1-(1,3-benzodioxol-5-ylcarbonylamino)cyclohexyl-N-[1-[5-(2-dimethylaminoethylthio)-1,3,4-oxadiazol-2-yl]-4-methyl-1-oxo-2-pentyl]carboxamide hydrochloride

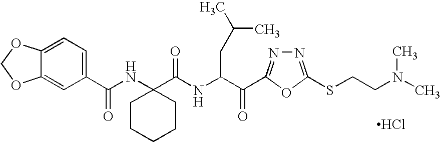

TLC: Rf 0.35 (methylene chloride:methanol:acetic acid=9:1:1); NMR (DMSO-d₆): δ 10.05-9.95 (br, 1H), 8.05 (d, J=6.9 Hz, 1H), 7.64 (s, 1H), 7.41-7.33 (m, 2H), 7.00-6.95 (m, 1H), 6.09 (s, 2H), 5.08-4.90 (m, 1H), 3.75-3.62 (m, 2H), 3.60-3.46 (m, 2H), 2.84 (s, 6H), 2.20-1.05 (m, 13H), 0.86 and 0.85 (each d, J=6.0 Hz, total 6H).

### EXAMPLE 1(129)

1-phenoxymethylcarbonylaminocyclohexyl-N-[1-[5-(2-dimethylaminoethylthio)-1,3,4-oxadiazol-2-yl]-4-methyl-1-oxo-2-pentyl]carboxamide hydrochloride

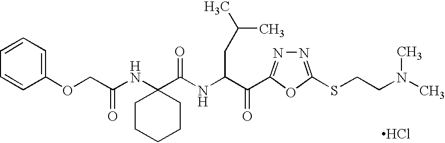

TLC: Rf 0.54 (methylene chloride:methanol:acetic acid=10:1:1); NMR (DMSO-d₆): δ 10.38-10.20 (br, 1H), 8.19 (d, J=6.3 Hz, 1H), 7.49 (s, 1H), 7.34-7.25 (m, 2H), 7.02-6.90 (m, 3H), 5.06-4.92 (m, 1H), 4.53 (s, 2H), 3.75-3.65 (m, 2H), 3.55-3.40 (m, 2H), 2.82 (s, 6H), 2.10-1.05 (m, 13H), 0.87 (d, J=5.7 Hz, 6H).

### EXAMPLE 1(130)

1-(2-chlorobenzoylamino)cyclohexyl-N-[1-[5-(2-dimethylaminoethylthio)-1,3,4-oxadiazol-2-yl]-4-methyl-1-oxo-2-pentyl]carboxamide hydrochloride

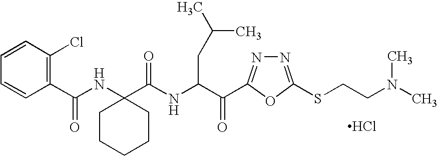

TLC: Rf 0.42(methylene chloride:methanol:acetic acid=10:1:1); NMR (DMSO-d₆): δ 10.18-10.02 (br, 1H),8.16 (s, 1H), 8.04 (d, J=7.2 Hz, 1H),7.58-7.38 (m, 4H),5.16-5.06 (m, 1H),3.74-3.64 (m, 2H), 3.54-3.44 (m, 2H), 2.81 (s, 6H), 2.18-1.15 (m, 13H), 0.91 and 0.89 (each d, J=6.0 Hz, total 6H).

### EXAMPLE 1(131)

1-(2-thienylmethylcarbonylamino)cyclohexyl-N-[1-[5-(2-dimethylaminoethylthio)-1,3,4-oxadiazol-2-yl]-4-methyl-1-oxo-2-pentyl]carboxamide hydrochloride

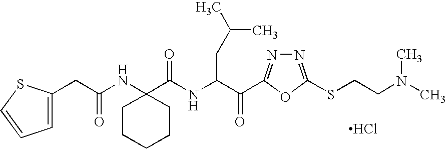

TLC: Rf 0.48 (chloroform:methanol:acetic acid=10:1:1); NMR (DMSO-d₆): δ 10.22-10.05 (br, 1H), 8.05 (d, J=6.9 Hz, 1H), 7.78 (s, 1H), 7.34 (dd, J=3.9, 1.2 Hz, 1H), 6.96-6.88 (m, 2H), 5.20-4.92 (m, 1H), 3.72 (s, 2H), 3.70-3.66 (m, 2H), 3.51-3.42 (m, 2H), 2.82 (s, 6H), 2.02-1.05 (m, 13H), 0.90-0.70 (m, 6H).

### EXAMPLE 1(132)

1-[4-(N-methoxy-N-methylaminomethyl)benzoylamino]cyclohexyl-N-[1-[5-(2-dimethylaminoethylthio)-1,3,4-oxadiazol-2-yl]-4-methyl-1-oxo-2-pentyl]carboxamide bishydrochloride

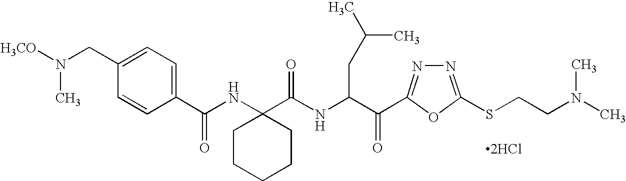

TLC: Rf 0.56 (chloroform:methanol=9:1); NMR (DMSO-d₆): δ 10.62 (br, 1H), 8.10 (brd, J=7.2 Hz, 1H), 7.78 (brs, 1H), 7.77 (d, J=8.4 Hz, 2H), 7.43 (d, J=8.4 Hz, 2H), 5.01 (m, 1H), 3.86 (s, 2H), 3.71 (m, 2H), 3.49 (m, 2H), 3.31 (s, 3H), 2.81 (s, 3H), 2.79 (s, 3H), 2.58 (s, 3H), 2.10 (m, 2H), 1.82-1.11 (m, 11H), 0.90-0.78 (m, 6H).

### EXAMPLE 1(133)

1-benzoylaminocyclohexyl-N-[(2S)-1-[5-[2-(2,4-dioxoimidazolidin-3-yl)ethylthio]-1,3,4-oxadiazol-2-yl]-4-methyl-1-oxo-2-pentyl]carboxamide

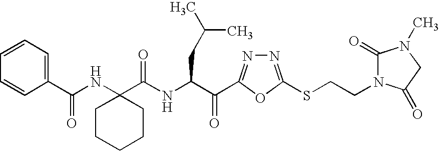

TLC: Rf 0.47 (ethyl acetate); NMR (CDCl₃): δ 8.20 (d, J=6.9 Hz, 1H), 7.79 (d, J=7.8 Hz, 2H), 7.55-7.44 (m, 3H), 6.20 (s, 1H), 5.27-5.20 (m, 1H), 3.97-3.82 (m, 2H), 3.87 (s, 2H), 3.52 (t, J=6.0 Hz, 2H), 2.98 (s, 3H), 2.37 (d, J=13.2 Hz, 1H), 2.24 (d, J=14.1 Hz, 1H), δ 2.01-1.37 (m, 11H), 1.02-0.98 (m, 6H).

### EXAMPLE 1(134)

cyclohexyl-N-[(2S)-1-[5-[2-(N-benzoyl-N-methylamino)ethylthio]-1,3,4-oxadiazol-2-yl]-4-methyl-1-oxo-2-pentyl]carboxamide

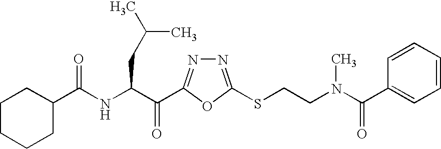

TLC: Rf 0.68(ethyl acetate); NMR (CDCl₃): δ 7.72-7.20 (brm, 5H), 5.99 (brd, J=6.6 Hz, 1H), 5.49-5.35 (m, 1H), 4.06-3.28 (brm, 4H), 3.09 (brs, 3H), 2.17 (tt, J=11.4, 3.3 Hz, 1H), 1.93-1.12 (m, 13H), 1.03 and 0.98 (each d, J=6.0 Hz, each 3H).

### EXAMPLE 1(135)

cyclohexyl-N-[(2S)-1-[5-[2-[N-methyl-N-(2-pyridylsulfonyl)amino]ethylthio]-1,3,4-oxadiazol-2-yl]-4-methyl-1-oxo-2-pentyl]carboxamide

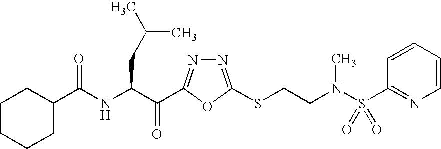

TLC: Rf 0.79 (chloroform:methanol=9:1); NMR (CDCl₃): δ 8.69 (d, J=4.8 Hz, 1H), 7.97 (d, J=7.5 Hz, 1H), 7.92 (dt, J=1.5, 7.5 Hz, 1H), 7.50 (ddd, J=7.5, 4.8, 1.5 Hz, 1H), 5.97 (brd, J=7.5 Hz, 1H), 5.46-5.36 (m, 1H), 3.79 (t, J=6.9 Hz, 2H), 3.66-3.49 (m, 2H), 3.03 (s, 3H), 2.23-2.10 (m, 1H), 1.93-1.12 (m, 13H), 1.02 and 0.97 (each d, J=6.3 Hz, each 3H).

### EXAMPLE 1(136)

1-[4-(N-methoxy-N-methylaminomethyl)benzoylamino]cyclohexyl-N-[1-[5-(1-methylethylthio)-1,3,4-oxadiazol-2-yl]-4-methyl-1-oxo-2-pentyl]carboxamide hydrochloride

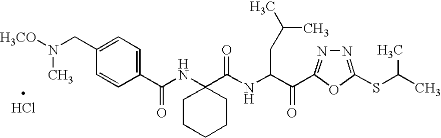

TLC: Rf 0.42 (n-hexane:ethyl acetate=1:1); NMR (DMSO-d₆): δ 8.04 (brd, J=6.9 Hz, 1H), 7.75 (brs, 1H), 7.76 (d, J=8.4 Hz, 2H), 7.40 (d, J=8.4 Hz, 2H), 4.98 (m, 1H), 3.89 (m, 1H), 3.81 (s, 2H), 3.28 (s, 3H), 2.56 (s, 3H), 2.10 (m, 2H), 1,82-1.11 (m, 11H), 1.45 (d, J=6.6 Hz, 6H), 0.90-0.78 (m, 6H).

### EXAMPLE 1(137)

cyclohexyl-N-[(2S)-1-[5-[2-[N-methyl-N-(N-oxidopyridin-2-ylsulfonyl)amino]ethylthio]-1,3,4-oxadiazol-2-yl]-4-methyl-1-oxo-2-pentyl]carboxamide

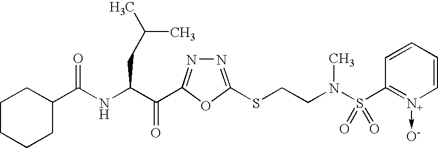

TLC: Rf 0.58 (chloroform:methanol=9:1); NMR (CDCl₃): δ 8.21 (d, J=6.3 Hz, 1H), 8.06 (dd, J=7.8, 2.1 Hz, 1H), 7.43 (dt, J=2.1, 6.3 Hz, 1H), 7.35 (dt, J=1.2, 7.8 Hz, 1H), 6.01 (brd, J=7.5 Hz, 1H), 5.45-5.34 (m, 1H), 3.91 (t, J=6.9 Hz, 2H), 3.54 (t, J=6.9 Hz, 2H), 3.09 (s, 3H), 2.17 (tt, J=11.7, 3.6 Hz, 1H), 1.93-1.13 (m, 13H), 1.03 and 0.97 (each d, J=6.3 Hz, each 3H).

### EXAMPLE 1(138)

(2S)-N-[2-methyl-1-[5-(2-dimethylaminoethylthio)-1,3,4-oxadiazol-2-yl]-1-oxo-2-propyl]-2-benzyloxycarbonylamino-4-methylpentanamide hydrochloride

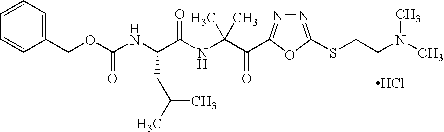

TLC: Rf 0.48 (chloroform:methanol=9:1); NMR (DMSO-d₆): δ 10.65 (br, 1H), 9.05 (brs, 1H), 7.40-7.20 (m, 6H), 4.97 (s, 2H), 3.99 (m, 1H), 3.63 (m, 2H), 3.43 (m, 2H), 2.79 (s, 6H), 1.45 (s, 6H), 1.60-1.11 (m, 3H), 0.85-0.77 (m, 6H).

### EXAMPLE 1(139)

cyclohexyl-N-[1-[5-[2-(N-ethyl-N-methylamino)ethylthio]-1,3,4-oxadiazol-2-yl]-4-methyl-1-oxo-2-pentyl]carboxamide hydrochloride

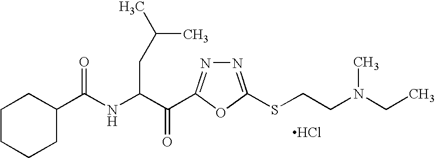

TLC: Rf 0.46 (ethyl acetate:methanol=9:1); NMR (DMSO-d₆): δ 8.38 (d, J=6.6 Hz, 1H), 5.06-4.99 (m, 1H), 3.73-3.69 (m, 2H), 3.60-3.05 (m, 4H), 2.80 and 2.78 (each s, total 3H), 2.28-2.12 (m, 1H), 1.80-1.50 (m, 8H), 1.35-1.07 (m, 8H), 0.91 (d, J=6.3 Hz, 3H), 0.90 (d, J=6.3 Hz, 3H).

### EXAMPLE 1(140)

1-(4-cyanobenzoylamino)cyclohexyl-N-[1-[5-(2-dimethylaminoethylthio)-1,3,4-oxadiazol-2-yl]-4-methyl-1-oxo-2-pentyl]carboxamide hydrochloride

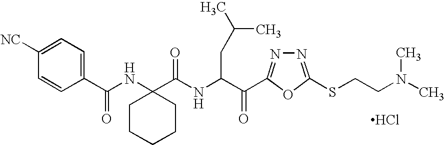

TLC: Rf 0.57(methylene chloride:methanol:acetic acid=10:1:1); NMR (DMSO-d₆): δ 10.35-10.25 (br, 1H), 8.15 (d, J=7.2 Hz, 11), 8.10 (s, 1H), 8.02-7.94 (m, 4H), 5.06-4.92 (m, 1H), 3.74-3.64 (m, 2H), 3.53-3.44 (m, 2H), 2.82 (s, 6H), 2.18-1.20 (m, 13H), 0.88-0.81 (m, 6H).

### EXAMPLE 2

cyclohexyl-N-[(2S)-1-[5-(2-aminoethylthio)-1,3,4-oxadiazol-2-yl]-4-methyl-1-oxo-2-pentyl]carboxamide hydrochloride

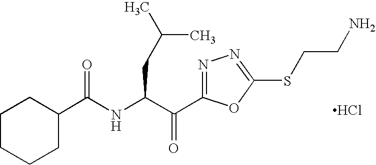

By the same procedure as described in reference example 10 using the compound prepared in example 1(36), the compound of the present invention having the following physical data was given.

TLC: Rf 0.76 (ethyl acetate:acetic acid:water=3:1:1); NMR (DMSO-d₆): δ 8.41 (brd, J=6.0 Hz, 1H), 8.30 (brs, 3H), 5.01 (m, 1H), 3.59 (t, J=6.9 Hz, 2H), 3.30-3.18 (m, 2H), 2.20 (m, 1H), 1.80-1.00 (m, 13H), 0.91 (d, J=6.3 Hz, 3H), 0.90 (d, J=6.3 Hz, 3H).

### EXAMPLE 2(1)-EXAMPLE 2(3)

By the same procedure as described in example 2 using corresponding compounds, the compounds of the present invention having the following physical data were given.

### EXAMPLE 2(1)

cycloheptyl-N-[(2S)-1-[5-(2-aminoethylthio)-1,3,4-oxadiazol-2-yl]-4-methyl-1-oxo-2-pentyl]carboxamide hydrochloride

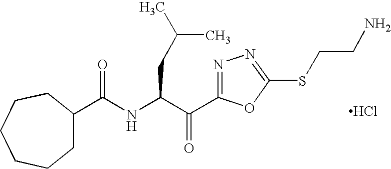

TLC: Rf 0.69 (ethyl acetate:acetic acid:water=3:1:1); NMR (DMSO-d₆): δ 8.40 (d, J=6.0 Hz, 1H), 8.28 (brs, 3H), 5.00 (m, 1H), 3.59 (t, J=6.9 Hz, 2H), 3.25 (m, 2H), 2.37 (m, 1H), 1.80-1.30 (m, 15H), 0.91 (d, J=6.6 Hz, 3H), 0.90 (d, J=6.3 Hz, 3H).

### EXAMPLE 2(2)

N-[(2S)-1-[5-(1-methylethylthio)-1,3,4-oxadiazol-2-yl]-4-methyl-1-oxo-2-pentyl]-4-amino-4-methylpentanamide hydrochloride

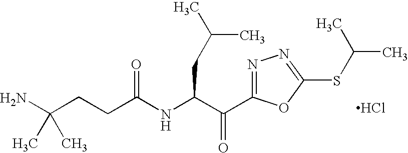

TLC: Rf 0.39 (ethyl acetate:acetic acid:water=3:1:1); NMR (DMSO-d₆): δ 8.64 (d, J=6.3 Hz, 1H), 8.27-7.87 (m, 3H), 5.14-5.01 (m, 1H), 4.06-3.89 (m, 1H), 2.30-2.18 and 1.82-1.38 (each m, total 7H), 1.47 (d, J=6.9 Hz, 6H), 1.19 (s, 6H), 0.91 (d, J=6.0 Hz, 6H).

### EXAMPLE 2(3)

N-[(2S)-4-methyl-1-[5-(1-methylethylthio)-1-oxo-1,3,4-oxadiazol-2-yl]-1-oxo-2-pentyl]-(2S)-2-amino-4-methylpentanamide hydrochloride

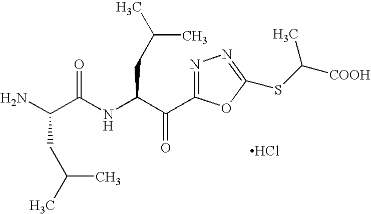

NM (DMSO-d₆): δ 9.11 (d, J=6.6 Hz, 1H), 8.45-8.13 (brs, 3H), 5.27-5.13 (m, 1H), 4.06-3.89 (m, 1H), 3.89-3.71 (m, 1H), 1.90-1.35 (m, 6H), 1.47 (d, J=6.6 Hz, 6H), 1.03-0.82 (m, 12H).

### EXAMPLE 3

cycloheptyl-N-[(2S)-1-[5-(1-carboxy-1-methylethylthio)-1,3,4-oxadiazol-2-yl]-4-methyl-1-oxo-2-pentyl]carboxamide

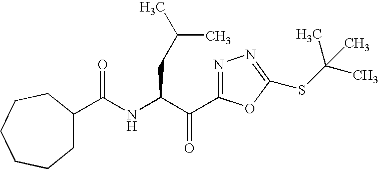

To a solution of the compound prepared in example 1(49) (200 mg) in methylene chloride (1 ml) was added trifluoroacetic acid (1 ml) and the mixture was stirred for 1 hour. The reaction mixture was concentrated and to the residue were added a saturated aqueous solution of sodium bicarbonate and ethyl acetate. The organic layer was extracted with a saturated aqueous solution of sodium bicarbonate three times. Combined aqueous layers were acidified with 2N hydrochloric acid and the mixture was extracted with ethyl acetate. The organic layer was washed with a saturated aqueous solution of sodium chloride, dried over anhydrous magnesium sulfate and concentrated to give the title compound (145 mg) having the following physical data.

TLC: Rf 0.22 (ethyl acetate:methanol=8:2); NMR (CDCl₃): δ 6.07 (brd, J=7.5 Hz, 1H), 5.36 (m, 1H), 2.33 (m, 1H), 1.95-1.40 (m, 15H), 1.83 and 1.82 (each s, each 3H), 1.02 (d, J=6.3 Hz, 3H), 0.98 (d, J=6.3 Hz, 3H).

### EXAMPLE 3(1)

cyclohexyl-N-[(2S)-1-(5-carboxymethylthio-1,3,4-oxadiazol-2-yl)-4-methyl-1-oxo-2-pentyl]carboxamide

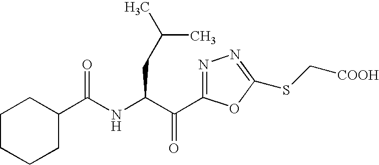

By the same procedure as described in example 3 using a corresponding compound, the compound of the present invention having the following physical data was given.

TLC: Rf 0.14 (ethyl acetate:methanol=8:2); NMR (DMSO-d₆): δ 13.2 (brs, 1H), 8.36 (brd, J=6.3 Hz, 1H), 4.98 (m, 1H), 4.24 (s, 2H), 2.18 (m, 1H), 1.78-1.00 (m, 13H), 0.90 (d, J=6.0 Hz, 3H), 0.89 (d, J=6.3 Hz, 3H).

### REFERENCE EXAMPLE 12

[(4S)-2,2-dimethyl-3-t-butoxycarbonyl-4-(2-methylpropyl)-1,3-oxazolidin-5-yl]carboxylic acid methyl ester

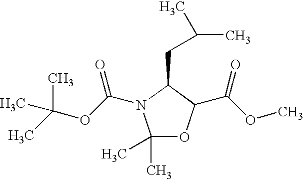

To a solution of the compound prepared in reference example 6 (4.40 g) in DMF (16 ml) was added methyl-2-propenyl ether (4.60 ml) was added dl-camphorsulfonic acid (186 mg) and the mixture was stirred overnight at room temperature. The residue was purified by column chromatography on silica gel (n-hexane:ethyl acetate=10:1 to 4:1) to give the title compound (3.90 g) having the following physical data.

TLC: Rf 0.52 and 0.48(n-hexane:ethyl acetate=2:1); NMR (CDCl₃): δ 4.61 and 4.44-4.12 (d and m, J=5.1 Hz, total 211), 3.78 (s, 3H), 1.70-1.30 (m, 9H), 1.48 and 1.47 (each s, total 9H), 1.05-0.87 (m, 6H).

### REFERENCE EXAMPLE 13

[(4S)-2,2-dimethyl-3-(t-butoxycarbonyl)-4-(2-methylpropyl)-1,3-oxazolidin-5-yl]carboxylic acid

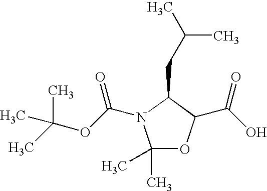

To a solution of the compound prepared in reference example 12 (3.84 g) in ethanol (12 ml)-water (6 ml) was added lithium hydroxide monohydrate (559 mg) and the mixture was stirred for 2 hours at room temperature. To the mixture was added n-hexane and extracted. To the aqueous layer was added 1N hydrochloric acid and was extracted with ethyl acetate. The extract was washed with a saturated aqueous solution of sodium chloride, dried over anhydrous magnesium sulfate and was concentrated to give a crude title compound (3.82 g) having the following physical data.

NMR (CDCl₃): δ 4.45-4.22 (m, 2H), 1.85-1.30 (m, 3H), 1.66 and 1.58 (each s, total 6H), 1.48 (s, 9H), 1.04-0.85 (m, 6H).

### REFERENCE EXAMPLE 14

N-[(4S)-3-(t-butoxycarbonyl)-2,2-dimethyl-4-(2-methylpropyl)-1,3-oxazolidin-5-ylcarbonyl]-N′-phenylhydrazide

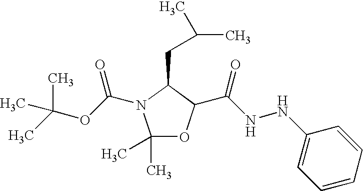

To a solution of the compound prepared in reference example 13 (3.81 g) in DMF (60 ml) were added 1-hydroxybenzotriazole (2.22 g), phenylhydrazine (1.79 ml) and 1-ethyl-3-[3-(dimethylamino)propyl]carbodiimide hydrochloride (2.78 g) at 0° C. successively, and the mixture was stirred for 3 hours. The reaction mixture was poured into 0.5N hydrochloric acid and was extracted with ethyl acetate. The extract was washed with a saturated aqueous solution of sodium bicarbonate and a saturated aqueous solution of sodium chloride and was extracted with ethyl acetate. The extract was washed with a saturated aqueous solution of sodium bicarbonate and a saturated aqueous solution of sodium chloride successively, dried over anhydrous magnesium sulfate and concentrated to give a crude title compound (4.39 g) having the following physical data.

TLC: Rf 0.44, 0.29 (n-hexane:ethyl acetate=2:1); NMR (CDCl₃): δ 8.28-8.16 (m, 1H), 7.28-7.19 (m, 2H), 6.97-6.81 (m, 3H), 4.63 and 4.39 (each d, J=5.1 and 2.4 Hz), 4.42-4.30 (m, I), 1.76-1.30 (m, 9H), 1.48 and 1.47 (each s, total 9H), 1.01-0.85 (m, 6H).

### REFERENCE EXAMPLE 15

[(4S)-3-(t-butoxycarbonyl)-2,2-dimethyl-4-(2-methylpropyl)-1,3-oxazolidin-5-yl]-3-phenyl-2-oxo-1,3,4-oxadiazoline

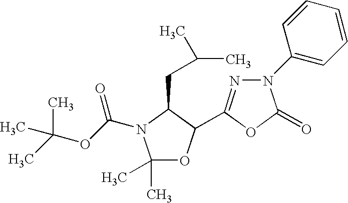

To a solution of the compound prepared in reference example 14 (4.36 g) in THF (110 ml) were added triethylamine (4.64 ml) and 1,1-carbonyldiimidazole(9.00 g) successively and the mixture was refluxed overnight. The extract was washed with a saturated aqueous solution of sodium bicarbonate and a saturated aqueous solution of sodium chloride successively, dried over anhydrous magnesium sulfate and was concentrated to give a crude title compound (3.32 g) having the following physical data.

TLC: Rf 0.45, 0.39 (n-hexane:ethyl acetate=4:1); NMR (CDCl₃): δ 7.89-7.81 (m, 2H), 7.49-7.41 (m, 2H), 7.32-7.24 (m, 1H), 5.05 (d, J=8.7 Hz, 0.5H), 4.81 (d, J=2.1 Hz, 0.5H), 4.60-4.15 (br, 1H), 1.90-1.30 (m, 9H), 1.50 (s, 9H), 0.99, 0.93 and 0.86 (each d, J=6.0 Hz, total 6H).

### REFERENCE EXAMPLE 16

(2S)-2-amino-4-methyl-1-(2-oxo-3-phenyl-1,3,4-oxadiazolin-5-yl)pentanol tosylate

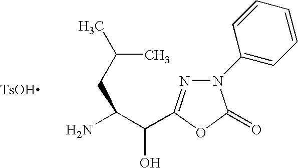

To a solution of the compound prepared in reference example 15 (1.26 g) in ethanol (30 ml) was added toluenesulfonic acid monohydrate (800 mg) and the mixture was refluxed for 3 hours. The reaction mixture was concentrated. The residue was washed with diethyl ether and dried to give a crude title compound (1.24 g) having the following physical data.

TLC: Rf 0.22 (chloroform:methanol=9:1); NMR (CDCl₃): δ 8.12-7.87-(br, 13H), 7.81-7.73 (m, 2H), 7.56-7.44 (m, 4H), 7.36-7.29 (m, 1H), 7.10 (d, J=8.7 Hz, 2H), 4.85 and 4.79 (each d, J=4.2 Hz, total 1H), 3.54-3.35 (br, 1H), 2.27 (s, 3H), 1.84-1.68 (m, 1H), 1.64-1.44 (m, 2H), 1.00-0.82 (m, 6H).

### EXAMPLE 4

1-[(1R,2S)-2-(4-dimethylaminomethylbenzoylamino)cyclohexyl]-N-[(2S)-4-methyl-1-oxo-1-[2-oxo-3-phenyl-1,3,4-oxadiazolidin-5-yl]-2-pentyl]carboxamide hydrochloride

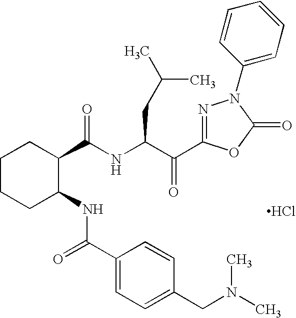

By the same procedure as described in reference example 11→example 1 using the compound reference example 16 in place of the compound prepared in reference example 10 and using (1R,2S)-2-(4-dimethylaminomethylbenzoyl amino)cyclohexylcarboxylic acid, the compound of the present invention having the following physical data was given.

TLC: Rf 0.32 (chloroform:methanol=9:1); NMR (CDCl₃): δ 7.91-7.82 (m, 4H), 7.69 (d, J=8.7 Hz, 2H), 7.54-7.41 (m, 3H), 7.39-7.32 (m, 1H), 6.26 (d, J=7.5 Hz, 1H), 5.49-5.40 (m, 1H), 4.39-4.25 (m, 1H), 4.17 (s, 2H), 2.89-2.79 (m, 1H), 2.74 (s, 6H), 2.11-1.37 (m, 11H), 0.99 and 0.93 (each d, J=6.0 Hz, each 3H).

### EXAMPLE 4(1)

cyclohexyl-N-[(2S)-4-methyl-1-oxo-1-[2-oxo-3-phenyl-1,3,4-oxadiazolin-5-yl]-2-pentyl]carboxamide

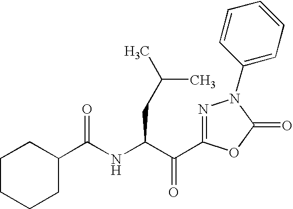

By the same procedure as described in example 4 using a corresponding compound, the compound of the present invention having the following physical data was given.

TLC: Rf 0.54 (ethyl acetate:n-hexane=1:2); NMR (CDCl₃): δ 7.92-7.84 (m, 2H), 7.54-7.45 (m, 2H), 7.39-7.31 (m, 1H), 5.91 (d, J=7.8 Hz, 1H), 5.49 (ddd, J=9.8, 7.8, 3.9 Hz, 1H), 2.25-2.10 (m, 1H), 1.94-1.15 (m, 13H), 1.06 and 1.00 (each d, J=6.0 Hz, each 3H).

### REFERENCE EXAMPLE 17

(2S)-2-(N-t-butoxycarbonylamino)-4-methyl-1-(2-oxo-1,3,4-oxadiazolin-5-yl)pentanol

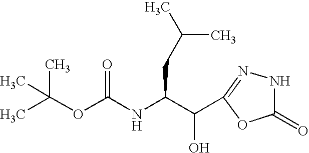

To a solution of the compound prepared in reference example 7 (20 g) and 1,1-carbonyldiimidazole (14 g) in THF (400 ml) was added triethylamine (12 ml) at 0° C. and the mixture was stirred for 5 hours at room temperature. To the reaction mixture was added 10% aqueous solution of citric acid and was extracted with ethyl acetate. The organic layer was washed with a saturated aqueous solution of sodium bicarbonate, water and a saturated sodium chloride successively, dried over anhydrous sodium sulfate and was concentrated. The residue was purified by column chromatography on silica gel (chloroform:methanol=20:1) to give the title compound (17 g) having the following physical data.

TLC: Rf 0.50 and 0.45 (chloroform:methanol=10:1); NMR (CDCl₃): δ 4.87 and 4.80 (each brd, each J=9.3 Hz, total 1H), 4.604.50 (m, 1H), 4.10-3.90 (m, 1H), 1.80-1.30 (m, 3H), 1.45 and 1.41 (each s, total 9H), 1.00-0.80 (m, 6H).

### REFERENCE EXAMPLE 18

(2S)-1-[3-cyclopropylmethyl-2-oxo-1,3,4-oxadiazolin-5-yl]-4-methyl-2-(t-butoxycarbonylamino)pentanol

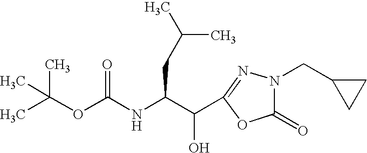

To a solution of the compound prepared in reference example 17 (13.4 g) in DMF (1L) was added potassium carbonate (8.28 g) and the mixture was stirred. Thereto was added cyclopropylmethyl iodide (4.27 ml) dropwise and the mixture was stirred for 80 minutes at 0° C. To the reaction mixture were added n-hexane and ethyl acetate and the mixture was poured into ice water. The organic layer was washed with water and a saturated aqueous solution of sodium chloride successively, dried over anhydrous magnesium sulfate and was concentrated. The residue was purified by column chromatography on silica gel (n-hexane:ethyl acetate=1:1) to give the title compound (8.98 g) having the following physical data.

TLC: Rf 0.79 (n-hexane:ethyl acetate=3:7); NMR (DMSO-d₆): δ 6.72 and 6.58 (each brd, J=9.0 Hz, total 1H), 6.12 and 5.92 (each d, J=6.0 Hz, total 1H), 4.38 and 4.07 (each m, total 1H), 3.79-3.60 (m, 1H), 3.79-3.60 (m, 1H), 3.55-3.40 (m, 2H), 1.65-1.00 (m, 4H), 1.34 and 1.29 (each s, total 9H), 0.52-0.45 (m, 2H), 1.35-1.28 (m, 2H).

### REFERENCE EXAMPLE 19

(2S)-1-(3-cyclopropylmethyl-2-oxo-1,3,4-oxadiazolin-5-yl)-4-methyl-2-aminopentanol hydrochloride

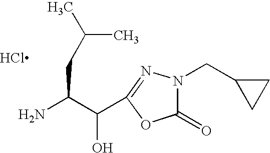

By the same procedure as described in reference example 4 using the compound prepared in reference example 18, the title compound having the following physical data was given.

TLC: Rf 0.15 and 0.10 (chloroform:methanol:water=9:1:0.1); NMR (DMSO-d₆): δ 8.40-8.10 (brd, 3H), 7.00 and 6.81 (each d, J=5.4 Hz, total 1H), 4.83 and 4.66 (each t, J=5.4 Hz, total 1H), 3.60-3.40 (m, 3H), 1.80-1.02 (m, 4H), 0.89-0.83 (m, 6H), 1.55-1.45 (m, 2H), 1.38-1.30 (m, 2H).

### REFERENCE EXAMPLE 20

1-[(1R,2S)-2-(t-butoxycarbonylamino)cyclohexyl]-N-[(2S)-1-[3-cyclopropylmethyl-2-oxo-1,3,4-oxadiazolin-5-yl]-1-hydroxy-4-methyl-2-pentyl]carboxamide

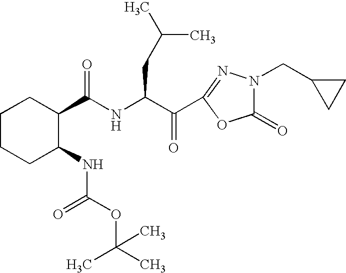

A solution of the compound prepared in reference example 19 (7.50 g), (1R,2S)-2-t-butoxycarbonylaminocyclohexane carboxylic acid (6.12 g) and 1-hydroxybenzotriazole (3.86 g) in DMF (50 ml) was cooled to 0° C. and thereto was added N-methylmorpholine (3.32 ml) and thereto 1-ethyl-3-[3-(dimethylamino)propyl]carbodiimide hydrochloride (5.80 g) and the mixture was stirred for 5 hours. Thereto was added N,N-dimethylethylenediamine (0.5 ml) and the mixture was stirred for 1 hour. To the reaction mixture was added water, and was extracted with ethyl acetate. The organic layer was washed with 0.5N hydrochloric acid, a saturated aqueous solution of sodium bicarbonate and a saturated aqueous solution of sodium chloride, dried over anhydrous magnesium sulfate and was concentrated to give the title compound (11.5 g) having the following physical data.

TLC: Rf 0.45(n-hexane:ethyl acetate=1:1); NMR (CDCl₃): δ 7.65 and 7.59 (each brd, J=9.0 Hz, total 1H), 6.13 and 5.98 (each d, J=4.8 Hz, total 1H), 6.05-5.90 (m, 1H), 4.40 and 4.06 (each m, total 1H), 4.09-4.00 (m, total 1H), 3.72-3.54 (m, 1H), 3.53-3.43 (m, 2H), 2.50-2.38 (m, 1H), 1.90-1.00 (m, 12H), 1.34 (s, 9H), 0.84 and 0.78 (each d, J=6.0 Hz, each 3H), 0.52-0.42 (m, 2H), 0.35-0.25 (m, 2H).

### EXAMPLE 5

1-[(1R,2S)-2-(t-butoxycarbonylamino)cyclohexyl]-N-[(2S)-1-[3-cyclopropylmethyl-2-oxo-1,3,4-oxadiazolin-5-yl]-4-methyl-1-oxo-2-pentyl]carboxamide

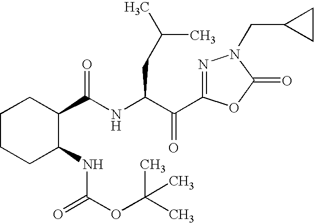

By the same procedure as described in example 1 using the compound prepared in reference example 20, the compound of the present invention having the following physical data was given.

TLC: Rf 0.68 (n-hexane:ethyl acetate=1:1); NMR (CDCl₃): δ 6.11 (brd, J=7.5 Hz, 1H), 5.35 (m, 1H), 5.19 (brd, J=5.0 Hz, 1H), 3.83 (m, 1H), 3.77 (dd, J=15, 7.2 Hz, 1H), 3.63 (dd, J=15, 7.5 Hz, 1H), 2.69 (brq, J=5.1 Hz, 1H), 2.00-1.35 (m, 11H), 1.43 (s, 9H), 1.23 (m, 1H), 1.02 (d, J=6.0 Hz, 3H), 0.97 (d, J=6.3 Hz, 3H). 0.68-0.60 (m, 2H), 0.45-0.40 (m, 2H).

### EXAMPLE 5(1)-EXAMPLE 5(3)

By the same procedure as described in example 5 using a corresponding compound, the compounds of the present invention having the following physical data were given,

### EXAMPLE 5(1)

1-[(1R,2S)-2-[4-dimethylamino(2-butynoyl)amino]cyclohexyl]-N-[1-[3-cyclopropylmethyl-2-oxo-1,3,4-oxadiazolin-5-yl]-4-methyl-1-oxo-2-pentyl]carboxamide

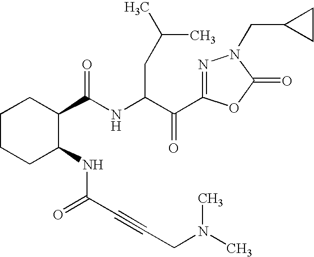

TLC: Rf 0.48 (chloroform:methanol=9:1); NMR (CDCl₃): δ 6.78 and 6.73 (each brd, J=9.0 Hz, 1H), 6.11 and 6.08 (each brd, J=7.5 Hz, 1H), 5.36 and 5.30 (each m, 1H), 4.22-4.10 (m, 1H), 3.78 (dd, J=14, 7.2 Hz, 1H), 3.64 (dd, J=14, 7.2 Hz, 1H), 3.36 (s, 2H), 2.78-2.75 (m, 1H), 2.32 and 2.31 (each s, 6H), 2.00-1.35 (m, 11H), 1.35-1.18 (m, 1H), 1.03 (d, J=6.0 Hz, 3H), 0.99 and 0.98 (each d, J=6.3 Hz, 3H), 0.68-0.60 (m, 2H), 0.45-0.40 (m, 2H).

### EXAMPLE 5(2)

1-[(1R,2S)-2-[4-morpholino(2-butynoyl)amino]cyclohexyl]-N-[1-[3-cyclopropylmethyl-2-oxo-1,3,4-oxadiazolin-5-yl]-4-methyl-1-oxo-2-pentyl]carboxamide

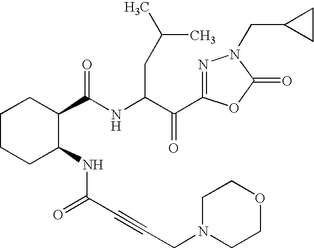

TLC: Rf 0.50 (chloroform:methanol=9:1); NMR (CDCl₃): δ 6.79 and 6.76 (each brd, J=9.0 Hz, 1H), 6.05 (brd, J=7.5 Hz, 1H), 5.35 and 5.30 (each m, 1H), 4.20-4.10 (m, 1H), 3.79 and 3.78 (each dd, J=14, 7.2 Hz, 1H), 3.74 (t, J=4.5 Hz, 4H), 3.64 (dd, J=14, 7.5 Hz, 1H), 3.40 and 3.38 (each s, 2H), 2.80-2.72 (m, 1H), 2.57 (t, J=4.5 Hz, 4H), 2.00-1.35 (m, 11H), 1.35-1.17 (m, 1H), 1.03 (d, J=6.0 Hz, 3H), 0.99 and 0.98 (each d, J=6.0 Hz, 3H), 0.70-0.60 (m, 2H), 0.50-0.38 (m, 2H).

### EXAMPLE 5(3)

1-[(1R,2S)-2-(2-butynoylamino)cyclohexyl]-N-[(2S)-1-[3-cyclopropylmethyl-2-oxo-1,3,4-oxadiazolin-5-yl]-4-methyl-1-oxo-2-pentyl]carboxamide

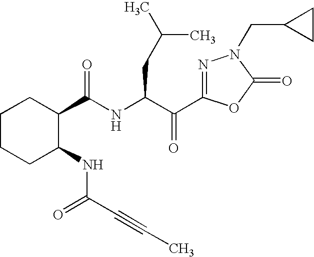

TLC: Rf 0.30 (n-hexane:ethyl acetate=1:1); NMR (CDCl₃): δ 6.61 (brd, J=8.7 Hz, 11H), 6.10 (brd, J=7.5 Hz, 1H), 5.35 (each m, 1H), 4.15 (m, 1H), 3.78 (dd, J=14, 7.2 Hz, 1H), 3.64 (dd, J=14, 7.5 Hz, 1H), 2.75 (m, 1H), 2.00-1.35 (m, 11H), 1.93 (s, 3H), 1.27 (m, 1H), 1.03 (d, J=6.0 Hz, 3H), 0.98 (d, J=6.3 Hz, 3H), 0.65-0.60 (m, 2H), 0.48-0.40 (m, 2H).

### EXAMPLE 5(4)

cyclohexyl-N-[(2S)-1-[3-(2-dimethylaminoethyl)-2-oxo-1,3,4-oxadiazolin-5-yl]-4-methyl-1-oxo-2-pentyl]carboxamide hydrochloride

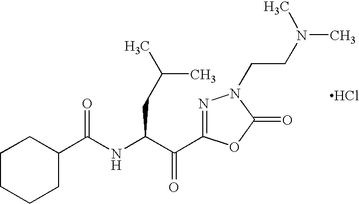

TLC: Rf 0.62 (ethyl acetate:acetic acid:water=3:1:1); NMR (DMSO-d₆): δ 10.3-9.90 (broad, 1H), 8.25 (brd, J=6.6 Hz, 1H), 4.96 (m, 1H), 4.23 (t, J=6.0 Hz, 2H), 3.43 (m, 2H), 2.83 (brs, 6H), 2.20 (m, 1H), 1.80-1.05 (m, 13H), 0.90 (d, J=6.3 Hz, 3H), 0.89 (d, J=6.3 Hz, 3H).

### REFERENCE EXAMPLE 21

(3S)-3-(t-butoxycarbonylamino)-5-methylhexanenitrile

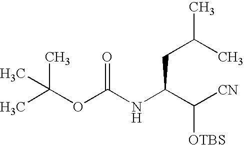

To a solution of the compound prepared in reference example 3 (2.04 g) in DMF (15 ml) were added imidazole (1.26 g) and t-butyldimethylsilyl chloride (2.79 g) and the mixture was stirred for 3 hours at room temperature. To the reaction mixture was added water and was extracted with ethyl acetate. The organic layer was washed with water and a saturated aqueous solution of sodium chloride successively, dried and was concentrated. The residue was purified by column chromatography on silica gel (n-hexane:ethyl acetate=19:1) to give the title compound (3.06 g) having the following physical data.

TLC: Rf 0.51 and 0.47(hexane:ethyl acetate=5:1); NMR (CDCl₃): δ 4.64-4.40 (m, 2H), 3.82 (m, 1H), 1.80-1.40 (m, 12H), 1.05-0.84 (m, 15H), 0.25-0.10 (m, 6H).

### REFERENCE EXAMPLE 22

3-(t-butoxycarbonylamino)-2-(t-butyldimethylsilyloxy)-5-methyl-N′-hydroxyhexanimidamide

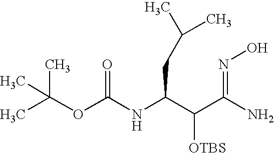

To a solution of the compound prepared in reference example 21 (4.27 g) in methanol (36 ml) were added hydroxyamine hydrochloride (2.50 g) and triethylamine (5.02 ml) at room temperature and the mixture was stirred at 50° C. The reaction mixture was concentrated, and to the residue were added water and ethyl acetate and the mixture was extracted. The organic layer was washed with a saturated aqueous solution of sodium chloride, dried over anhydrous magnesium sulfate and was concentrated. The residue was purified by column chromatography on silica gel (chloroform:methanol=20:1) to give the title compound (4.44 g) having the following physical data.

TLC: Rf 0.44(chloroform:methanol=9:1); NMR (CDCl₃): δ 6.60-6.00 and 4.86-4.42 (each m, total 3H), 4.08-4.00 (m, 1H), 3.90-3.70 (m, 1H), 1.44 (s, 9H), 1.42-1.15 (m, 3H), 1.00-0.85 (m, 15H), 0.16-0.08 (m, 6H).

### REFERENCE EXAMPLE 23

(2S)-2-(t-butoxycarbonylamino)-4-methyl-1-(5-thioxo-1,2,4-oxadiazolin)-1-(t-butyldimethylsilyloxy)-2-pentane

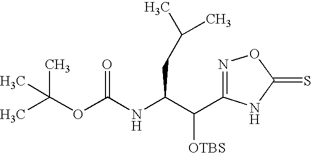

To a solution of the compound prepared in reference example 22 (4.43 g) in acetonitrile (114 ml) was added DBU (1,8-diazabicyclo[4.3.0]non-5-one) (6.81 ml) and then to the mixture was added thiocarbonyldiimidazole (TCDI) (2.23 g) at 0° C. and the mixture was stirred for 13 hours at room temperature. The reaction mixture was poured into 1N hydrochloric acid (100 ml) and was extracted with ethyl acetate and the mixture was acidified with 1N hydrochloric acid to pH 2. The solution was extracted with ethyl acetate and the organic layer was washed with a saturated aqueous solution of sodium chloride, dried over anhydrous magnesium sulfate and was concentrated. The residue was purified by column chromatography on silica gel (n-hexane:ethyl acetate=4:1) to give the title compound (4.22 g) having the following physical data.

TLC: Rf 0.44 (n-hexane:ethyl acetate=2:1); NMR (CDCl₃): δ 6.73-5.70 (m, 1H), 4.82-3.75 (m, 2H), 1.83-1.15 (m, 3H), 1.43 and 1.39 (each s, total 9H), 1.01-0.82 (m, 15H), 0.23-0.02 (m, 6H).

### REFERENCE EXAMPLE 24

N-[1-(t-butyldimethylsilyloxy)-4-methyl-1-[5-(2-morpholinoethylthio)-1,2,4-oxadiazol-3-yl]-2-pentyl]-N-t-butoxycarbonylamine

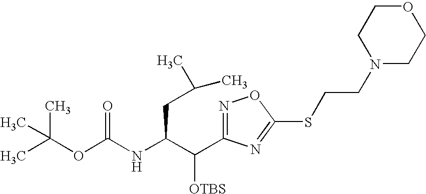

By the same procedure as described in reference example 9 using the compound prepared in reference example 23 in place of the compound prepared in reference example 8, and using 2-morpholinoethyl iodide in place of 2-chloroethylenediamine hydrochloride, the title compound having the following physical data was given.

TLC: Rf 0.54(n-hexane:ethyl acetate=1:1); NoM (CDCl₃): δ 4.89-4.78 (m, 2H), 4.13-3.92 (m, 1H), 3.71 (t, J=4.5 Hz, 4H), 3.45-3.35 (m, 2H), 2.76 (t, J=7.5 Hz, 2H), 2.52 (t, J=4.5 Hz, 4H), 1.63-1.17 (m, 12H), 0.95-0.85 (m, 15H), 0.13-0.00 (m, 6H).

### REFERENCE EXAMPLE 25

1-(t-butyldimethylsilyloxy)-4-methyl 1-[5-(2-morpholino ethylthio)-1,2,4-oxadiazol-3-yl]-2-pentylamine

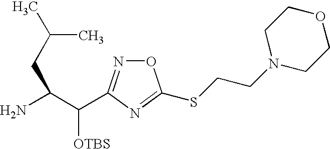

To a solution of the compound prepared in reference example 24 in methylene chloride(20 ml) was added a 90% aqueous solution of trifluoroacetic acid (6 ml) at 0° C. and the mixture was stirred for 1 hour at 0° C. and for 1 hour at room temperature. The reaction mixture was poured into ice-water and thereto was added 28% ammonia water slowly to neutralize it. The mixture was extracted with ethyl acetate. The organic layer was concentrated to give the title compound (788 mg) having the following physical data.

TLC: Rf 0.45 (chloroform:methanol=9:1); NMR (CDCl₃): δ 4.65 (d, J=3.9 Hz, 1H), 3.71 (t, J=4.5 Hz, 4H), 3.45-3.40 (m, 2H), 3.23-3.14 (m, 1H), 2.76 (t, J=6.6 Hz, 2H), 2.52 (t, J=4.5 Hz, 4H), 1.85-1.72 (m, 1H), 1.43-1.16 (m, 2H), 0.99-0.84 (m, 15H), 0.18-0.00 (m, 6H).

### REFERENCE EXAMPLE 26

1-[(1R,2S)-2-benzoylaminocyclohexyl]-N-[1-(t-butyldimethylsilyloxy)-4-methyl-1-[5-(2-morpholinoethylthio)-1,2,4-oxadiazol-3-yl]-2-pentyl]carboxamide

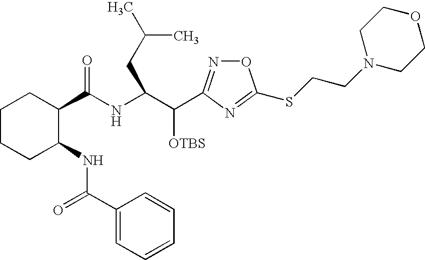

To a solution of (1R,2S)-2-benzoylaminocyclohexane carboxylic acid (439 mg) in DMF (5 ml) were added 1-ethyl-3-[3-(dimethylamino)propyl]carbodiimide hydrochloride (340 mg) and 1-hydroxybenzotriazole(272 mg) and the mixture was stirred for 30 minutes at room temperature. Thereto was added a solution of the compound prepared in reference example 25 (788 mg) in DMF (5 ml) and the mixture was stirred for 30 minutes at room temperature. The reaction mixture was poured into ice-water and was extracted with ethyl acetate. The organic layer was washed with water, a saturated aqueous solution of sodium bicarbonate and a saturated aqueous solution of sodium chloride successively, dried over anhydrous magnesium sulfate and was concentrated. The residue was purified by column chromatography on silica gel (chloroform to chloroform:methanol=49:1) to give the compound of the present invention (709 mg) having the following physical data.

TLC: Rf 0.80(ethyl acetate:methanol=9:1); NMR (CDCl₃): δ 7.78-7.75 (m, 2H), 7.49-7.38 (m, 4H), 5.99 (d, J=9.6 Hz, 1H), 4.84 (d, J=2.7 Hz, 1H), 4.43-4.15 (m, 2H), 3.70 (t, J=4.5 Hz, 4H), 3.46-3.33 (m, 2H), 2.79-2.69 (m, 2H), 2.52-2.42 (m, 1H), 2.51 (t, J=4.5 Hz, 4H), 2.05-1.28 (m, 111H), 1.01-0.71 (m, 15H), 0.20-0.00 (m, 6H).

### REFERENCE EXAMPLE 27

1-[(1R,2S)-2-benzoylaminocyclohexyl]-N-[1-hydroxy-4-methyl-1-[5-(2-morpholinoethylthio)-1,2,4-oxadiazol-3-yl]-2-pentyl]carboxamide

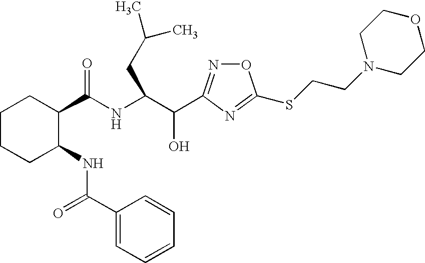

To a solution of the compound prepared in reference example 26 (709 mg) in THF (5 ml) was added tetrabutylammonium fluoride (2.1 ml; 1.0M solution in THF) and the mixture was stirred for 1 hour at room temperature. The reaction mixture was concentrated and the residue was purified by column chromatography on silica gel (ethyl acetate:methanol=39:1) to give the title compound (407 mg) having the following physical data.

TLC: Rf 0.52(ethyl acetate:methanol=9:1); NMR (CDCl₃): δ 7.76 (d, J=6.6 Hz, 2H), 7.50-7.39 (m, 3H), 7.28 (d, J=8.4 Hz, 1H), 5.98 (d, J=6.3 Hz, 1H), 4.83 (d, J=3.9 Hz, 1H), 4.51-4.40 (m, 1H), 4.34-4.19 (m, 1H), 3.70 (t, J=4.5 Hz, 4H), 3.37 (t, J=6.6 Hz, 2H), 2.74 (t, J=6.6 Hz, 2H), 2.50 (t, J=4.5 Hz, 4H), 2.14-2.00 (m, 1H), 1.92-1.17 (m, 11H), 0.83-0.80 (m, 6H).

### EXAMPLE 6

1-[(1R,2S)-2-benzoylaminocyclohexyl]-N-[4-methyl-1-[5-(2-morpholinoethylthio)-1,2,4-oxadiazol-3-yl]-1-oxo-2-pentyl]carboxamide

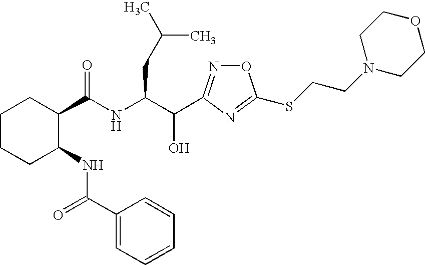

To a solution of Dess-Martin reagent ((1,1,1-triacetoxy)-1,1-dihydro-1,2-benziodoxol-3(1H)-one; 617 mg) in methylene chloride (7 ml) was added the compound prepared in reference Example 27 (407 mg) in methylene chloride (7 ml) slowly and the mixture was stirred for 30 minutes. The reaction mixture was poured into a cool saturated aqueous solution of sodium thiosulfate and the mixture was extracted with ethyl acetate. The organic layer was washed with water, a saturated aqueous solution of sodium bicarbonate and a saturated aqueous solution of sodium chloride, dried over anhydrous magnesium sulfate and was concentrated. The residue was purified by column chromatography on silica gel (ethyl acetate:methanol=49:1) to give the compound of the present invention (266 mg) having the following physical data.

TLC: Rf 0.53 (ethyl acetate:methanol=19:1); NMR (CDCl₃): δ 7.82-7.75 (m, 2H), 7.51-7.38 (m, 3H), 7.30 and 7.21 (each d, J=8.1 Hz, total 1H), 6.20 (d, J=7.5 Hz, 1H), 5.46-5.35 (m, 1H), 4.38-4.27 (m, 1H), 3.70 (t, J=4.5 Hz, 4H), 3.54-3.47 (m, 2H), 2.88-2.83 (m, 1H), 2.79-2.75 (m, 2H), 2.51 (t, J=4.5 Hz, 4H), 2.14-1.42 (m, 11H), 1.00, 0.93, 0.90, and 0.83 (each d, J=6.0 Hz, total 6H).

### EXAMPLE 6(1)

cyclohexyl-N-[4-methyl-1-[5-(2-morpholinoethylthio)-1,2,4-oxadiazol-3-yl]-1-oxo-2-pentyl]carboxamide

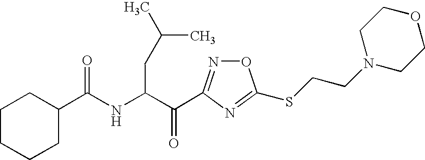

By the same procedure as described in example 6 using a corresponding compound, the compound of the present invention having the following physical data was given.

TLC: Rf 0.54(ethyl acetate:methanol=19:1); NMR (CDCl₃): δ 5.99 (d, J=7.5 Hz, 1H), 5.48-5.41 (m, 1H), 3.70 (t, J=4.5 Hz, 4H), 3.52 (t, J=6.6 Hz, 2H), 2.77 (t, J=6.6 Hz, 2H), 2.52 (t, J=4.5 Hz, 4H), 2.21-2.11 (m, 1H), 1.89-1.19 (m, 13H), 1.02 (d, J=6.0 Hz, 3H), 0.95 (d, J=6.0 Hz, 3H).

### REFERENCE EXAMPLE 28

N-(t-butoxycarbonyl)-N-[(2S)-1-[3-(2-dimethylaminoethyl)-2-thioxo-1,3,4-oxadiazolin-5-yl]-1-hydroxy-4-methyl-2-pentyl]amine

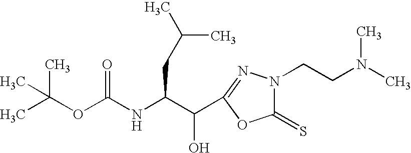

A solution of the compound prepared in reference example 8 (25.4 g), 2-chloroethyldimethylamine hydrochloride (12.7 g) and potassium carbonate (27.6 g) in DMF (240 ml) was stirred for 13 hours at 50° C. The reaction mixture was poured into water and was extracted with ethyl acetate. The organic layer was washed with a saturated aqueous solution of sodium chloride, dried over anhydrous magnesium sulfate and was concentrated. The residue was purified by column chromatography on silica gel (chloroform:methanol=100:2 to 4:1) to give the title compound (940 mg) having the following physical data.

TLC: Rf 0.42(chloroform:methanol=9:1); NMR (CDCl₃): δ 4.90-4.62 (m, 2H), 4.23-4.06 (m, 2H), 3.98-3.82 (m, 1H), 2.75 (t, J=6.6 Hz, 2H), 2.29 (s, 6H), 1.78-1.52 (m, 3H), 1.50-1.33 (m, 9H), 1.03-0.88 (m, 6H).

### REFERENCE EXAMPLE 29

[(2S)-1-[3-(2-dimethylaminoethyl)-2-thioxo-1,3,4-oxadiazolin-5-yl]-1-hydroxy-4-methyl-2-pentyl]amine bishydrochloride

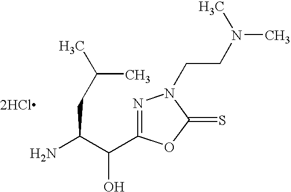

To a solution of the compound prepared in reference example 28 (932 mg) in ethyl acetate (2.4 ml) was added 4N hydrochloric acid (a solution in ethyl acetate) and the mixture was stirred for 1 hour at room temperature. The reaction mixture was concentrated to give a crude title compound having the following physical data.

TLC: Rf 0.42(chloroform:methanol:28% ammonia water=90:10:1); NMR (DMSO-d₆): δ 10.72-10.55 (br, 1H), 8.50-8.15 (br, 3H), 5.054.95 (m, 1H), 4.47 (t, J=6.0 Hz, 2H), 3.70-3.40 (m, 3H), 2.90-2.70 (m, 6H), 1.86-1.35 (m, 3H), 1.00-0.75 (m, 6H).

### REFERENCE EXAMPLE 30

(2S)-N-[(2S)-1-[3-(2-dimethylaminoethyl)-2-thioxo-1,3,4-oxadiazolin-5-yl]-1-hydroxy-4-methyl-2-pentyl]-2-benzyloxycarbonylamino-4-methylpentanamide

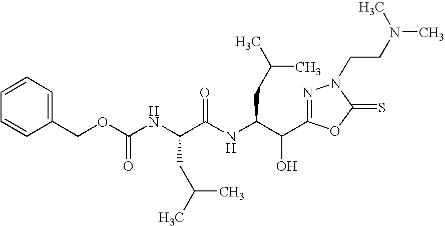

To a solution of the compound prepared in reference example 29 (158 mg) and N-benzyloxycarbonylleucine (90 mg) in DMF (1.7 ml) were added 1-hydroxybenzotriazole (62 mg) and 1-ethyl-3-[3-(dimethylamino)propyl]carbodiimide hydrochloride(78 mg) and N-methylmorpholine(93 μl) and the mixture was stirred for 17 hours at room temperature. The reaction mixture was poured into a saturated aqueous solution of sodium bicarbonate and was extracted with ethyl acetate. The organic layer was washed with a saturated aqueous solution of sodium chloride, dried over anhydrous magnesium sulfate and was concentrated. The residue was purified by column chromatography on silica gel (methylene chloride:methanol=40:1) to give the title compound (107 mg) having the following physical data.

TLC: Rf 0.48(chloroform:methanol=9:1); NMR (CDCl₃): δ 7.42-7.25 (m, 5H), 6.83-6.70 (br, 1H), 6.04-5.88 (br, 1H), 5.20-5.02 (m, 11), 5.18 (d, J=12.3 Hz, 1H), 5.07 (d, J=12.3 Hz, 1H), 4.75 (d, J=3.9 Hz, 1H), 4.26-3.92 (m, 3H), 2.85-2.64 (m, 2H), 2.27 (s, 6H), 1.90-1.35 (m, 6H), 1.07-0.83 (m, 12H).

### EXAMPLE 7

(2S)-N-[1-[3-(2-dimethylaminoethyl)-2-thioxo-1,3,4-oxadiazolin-5-yl]-4-methyl-1-oxo-2-pentyl]-2-benzyloxycarbonylamino-4-methylpentanamide

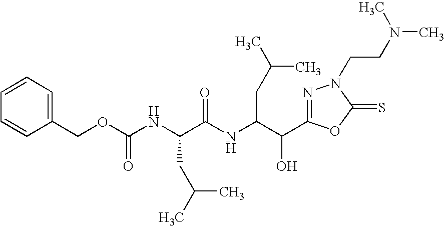

To a solution of oxalyl chloride (33 μl) in methylene chloride(0.3 ml) was added dimethylsulfoxide (0.75 ml) in methylene chloride (1 ml) at −78° C. and to the mixture were added the compound prepared in reference example. 30 (100 mg) in methylene chloride (2 ml) and N-methylmorpholine (0.16 ml) and the mixture was stirred for 1 hour and the mixture was warmed to 0° C. The reaction mixture was poured into water, and was extracted with ethyl acetate. The organic layer was washed with a saturated aqueous solution of sodium chloride, dried over anhydrous magnesium sulfate and was concentrated. The residue was purified by column chromatography (chloroform:methanol=20:1) to give the title compound (87 mg) having the following physical data.

TLC: Rf 0.63 (chloroform:methanol=9:1); NMR (CDCl₃): δ 7.42-7.26 (m, 5H), 6.75-6.65 and 6.55-6.47 (each br, total 1H), 5.37-5.25 (m, 1H), 5.25-5.03 (m, 3H), 4.35-4.05 (m, 3H), 2.78 (t, J=6.3 Hz, 2H), 2.29 and 2.28 (each s, total 6H), 1.85-1.40 (m, 6H), 1.06-0.82 (m, 12H).

### EXAMPLE 7(1)-EXAMPLE 7(5)

By the same procedure as described in example 7 using a corresponding compound, the compounds of the present invention having the following physical data were given.

### EXAMPLE 7(1)

1-[(1R,2S)-2-benzoylaminocyclohexyl]-N-[4-methyl-1-[3-(2-dimethylaminoethyl)-2-thioxo-1,3,4-oxadiazolin-5-yl]-1-oxo-2-pentyl]carboxamide

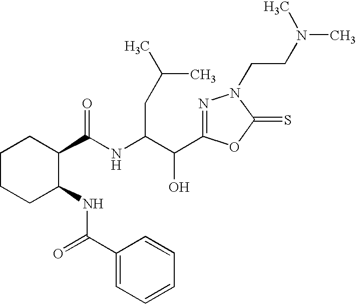

TLC: Rf 0.60 (chloroform:methanol=9:1); NMR (CDCl₃): δ 7.84-7.72 (m, 2H), 7.55-7.37 (m, 3H), 7.15-7.04 (m, 1H), 6.29 and 6.20 (each brd, J=7.5 Hz, total 1H), 5.37-5.20 (m, 1H), 4.43-4.08 (m, 3H), 2.91-2.81 (m, 1H), 2.78 and 2.77 (each t, J=6.3 Hz, total 2H), 2.28 (s, 6H), 2.13-1.40 (m, 11H), 0.99, 0.94, 0.89 and 0.84 (each d, J=6.3 Hz, total 6H).

### EXAMPLE 7(2)

cyclohexyl-N-[1-[3-(2-dimethylaminoethyl)-2-thioxo-1,3,4-oxadiazolin-5-yl]-4-methyl-1-oxo-2-pentyl]carboxamide

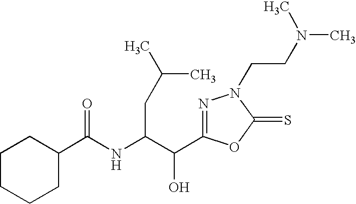

TLC: Rf 0.75 (chloroform:methanol=9:1); NMR (CDCl₃): δ 5.89 (brd, J=7.8 Hz, 1H), 5.42-5.32 (m, 1H), 4.30 (dt, J=13.8, 6.3 Hz, 1H), 4.18 (dt, J=13.8, 6.3 Hz, 1H), 2.78 (t, J=6.3 Hz, 2H), 2.29 (s, 6H), 2.22-2.09 (m, 1H), 1.93-1.17 (m, 13H), 1.01 and 0.97 (each d, J=6.0 Hz, each 3H).

### EXAMPLE 7(3)

1-benzoylaminocyclohexyl-N-[4-methyl-1-(5-thioxo-1,3,4-oxadiazolin-2-yl)-1-oxo-2-pentyl]carboxamide

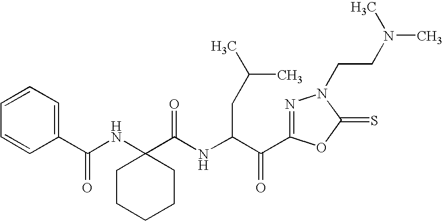

TLC: Rf 0.50 (chloroform:methanol=9:1); NMR (CDCl₃): δ 7.98 (brd, J=6.6 Hz, 1H), 7.82-7.74 (m, 2H), 7.60-7.43 (m, 3H), 6.07 (brs, 1H), 5.28-5.19 (m, 1H), 4.26 (dt, J=13.8, 6.3 Hz, 1H), 4.15 (dt, J=13.8, 6.3 Hz, 1H), 2.77 (t, J=6.3 Hz, 2H), 2.42-1.28 (m, 13H), 2.29 (s, 6H), 0.99 and 0.97 (each d, J=6.0 Hz, each 3H).

### EXAMPLE 7(4)

(2S)-N-[(2S)-1-[3-(1-methylethyl)-2-thioxo-1,3,4-oxadiazolin-5-yl]-4-methyl-1-oxo-2-pentyl]-2-benzyloxycarbonylamino-4-methylpentanamide

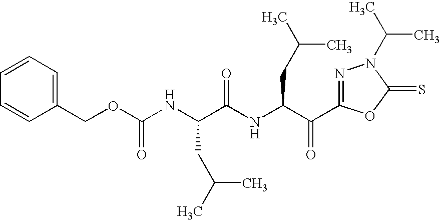

TLC: Rf 0.33 (ethyl acetate:n-hexane=1:3); NMR (CDCl₃): δ 7.45-7.27 (m, 5H), 6.55 (d, J=7.5 Hz, 1H), 5.39-5.29 (m, 1H), 5.21-5.02 (m, 3H), 5.02-4.87 (m, 1H), 4.29-4.11 (m, 1H), 1.80-1.37 (m, 6H), 1.45 and 1.44 (each d, J=6.6 Hz, each 3H), 1.08-0.84 (m, 12H).

### EXAMPLE 7(5)

1-[(1R,2S)-2-benzoylaminocyclohexyl]-N-[(2S)-4-methyl-1-oxo-1-[3-(1-methylethyl)-2-thioxo-1,3,4-oxadiazolin-5-yl]-2-pentyl]carboxamide

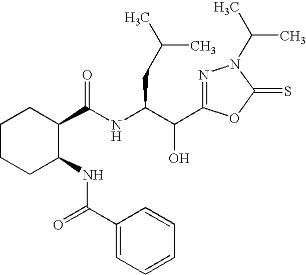

TLC: Rf 0.44 (ethyl acetate:n-hexane=1:1); NMR (CDCl₃): δ 7.82-7.71 (m, 2H), 7.55-7.36 (m, 3H), 7.10 (d, J=8.7 Hz, 1H), 6.20 (d, J=7.8 Hz, 1H), 5.40-5.28 (m, 1H), 5.03-4.86 (m, 1H), 4.40-4.28 (m, 1H), 2.90-2.79 (m, 1H), 2.17-1.38 (m, 111H), 1.45 and 1.44 (each d, J=6.6 Hz, each 3H), 0.90 and 0.84 (each d, J=6.0 Hz, each 3H).

### REFERENCE EXAMPLE 31

cyclohexyl-[(2S)-[1-hydroxy-4-methyl-1-(2-thioxo-1,3,4-oxadiazolin-5-yl)-2-pentyl]carboxamide

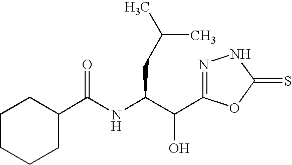

By the same procedure as described in reference example 4 using the compound prepared in reference example 8, its Boc-deprotected compound was given. Next, by the same procedure as described in reference example 10 using cyclohexanecarboxylic acid in place of cycloheptanecarboxylic acid, the title compound having the title compound was given.

TLC: Rf 0.68 (ethyl acetate:methanol=9:1); NMR (DMSO-d₆): δ 14.3 (brs, 1H), 7.55 and 7.51 (each d, J=9.0 Hz, total 1H), 6.27 and 6.15 (each d, J=6.0 Hz, total 1H), 4.61 and 4.28 (each m, total 1H), 4.10-3.94 (m, 1H), 2.12-1.95 (m, 1H), 1.70-1.05 (m, 13H), 0.86 and 0.80 (each d, J=6.3 Hz, each 3H).

### REFERENCE EXAMPLE 32

cyclohexyl-N-[(2S)-1-hydroxy-1-(5-methylthio-1,3,4-oxadiazol-2-yl)-4-methyl-2-pentyl]carboxamide

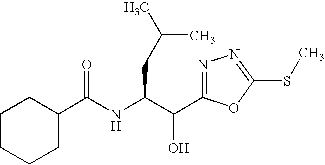

To a solution of the compound prepared in reference example 31 (1.78 g) and potassium carbonate (828 mg) in DMF (5 ml) in methyl iodide (0.31 ml) and the mixture was stirred for 1 hour at room temperature. To the reaction mixture was added water and the mixture was extracted with n-hexane/ethyl acetate. The organic layer was washed with water twice and a saturated aqueous solution of sodium chloride once successively, dried over anhydrous magnesium sulfate and concentrated to give the title compound (1.77 g) having the following physical data.

TLC: Rf 0.26(n-hexane:ethyl acetate=1:1).

### REFERENCE EXAMPLE 33

cyclohexyl-N-[(2S)-1-(t-butyldimethylsilyloxy)-1-(5-methylthio-1,3,4-oxadiazol-2-yl)-4-methyl-2-pentyl]carboxamide

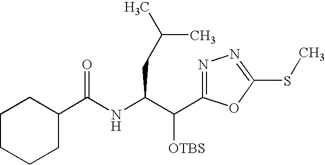

To a solution of the compound prepared in reference example 32 (1.76 g) and imidazole (408 mg) in DMF (5 ml) was added t-butyldimethylsilyl chloride(408 mg) and the mixture was stirred for 20 hours. To the reaction mixture were added ethyl acetate, n-hexane and water and the organic layer was washed with 10& citric acid, a saturated aqueous solution of sodium bicarbonate, water and a saturated aqueous solution of sodium chloride successively, dried over anhydrous magnesium sulfate and was concentrated. The residue was suspended in n-hexane and the mixture was stirred for 30 minutes and the precipitate (1.79 g) was collected. The filtrate was purified by column chromatography on silica gel (n-hexane:ethyl acetate=7:3), combined with the above precipitate, to give the title compound (2.09) having the following physical data.

TLC: Rf 0.39 and 0.42(n-hexane:ethyl acetate=8:2); NMR (CDCl₃): δ 5.79 and 5.59 (each brd, J=9.0 Hz, total 1H), 4.98 and 4.81 (each d, J=3.0, 5.4 Hz, total 1H), 4.44 and 4.31 (each m, total 1H), 2.72 and 2.71 (each s, total 3H), 2.06-2.02 (each m, total 1H), 1.90-1.20 (m, 3H), 0.95-0.88 (m, 15H), 0.15, 0.09, 0.05 and 0.00 (each s, total 6H).

### REFERENCE EXAMPLE 34

cyclohexyl-N-[(2S)-1-(t-butyldimethylsilyloxy)-1-(5-methylsulfonyl-1,3,4-oxadiazol-2-yl)-4-methyl-2-pentyl]carboxamide

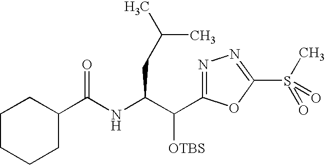

To a solution of the compound prepared in reference example 33 (2.0 g) in methylene chloride (20 ml) was added a suspension of 3-chloroperbenzoic acid (2.96 g) in methylene chloride (5 ml) and the mixture was stirred for 43 hours at room temperature. To the reaction mixture was added ethyl acetate and thereto was added 5% aqueous solution of sodium sulfite. The mixture was stirred for 10 minutes. The organic layer was extracted, and the extract was washed with a saturated aqueous solution of sodium bicarbonate twice and a saturated aqueous solution of sodium chloride once successively, dried over anhydrous magnesium sulfate and was concentrated to give the title compound having the following physical data.

TLC: Rf 0.55 and 0.58(n-hexane:ethyl acetate=7:3); NMR (CDCl₃): δ 5.62 and 5.27 (each brd, J=6.0 Hz, total 1H), 5.07 and 4.74 (each d, J=2.4, 7.2 Hz, total 1H), 4.38-4.26 (m, 1H), 3.44 and 3.43 (each s, total 3H), 2.10-1.10 (m, 14H), 1.00-0.87 (m, 15H), 0.14, 0.09, 0.05, 0.04 (each s, total 6H).

### REFERENCE EXAMPLE 35

cyclohexyl-N-[(2S)-1-(t-butyldimethylsilyloxy)-1-[5-(1-methylethyloxy)-1,3,4-oxadiazol-2-yl]-4-methyl-2-pentyl]carboxamide

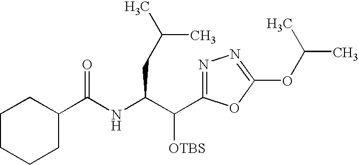

To a suspension of sodium hydride (29 mg; 60% oil) in THF (3 ml) was added isopropyl alcohol (92 μl) and the mixture was stirred 0° C. Thereto was added a solution of the compound prepared in reference example 34 (294 mg) in THF (3 ml) and the mixture was stirred for 40 minutes. To the mixture were added ethyl acetate and a saturated aqueous solution of sodium bicarbonate and the mixture was stirred for 40 minutes and was extracted. The organic layer was washed with a: saturated aqueous solution of sodium bicarbonate and water successively, dried over anhydrous magnesium sulfate and was concentrated to give the title compound (281 g) having the following physical data.

TLC: Rf 0.39 and 0.49(n-hexane:ethyl acetate=7:3); NMR (CDCl₃): δ 5.82 and 5.64 (each brd, J=9.6 Hz, total 1H), 5.18-5.02 (m, 1H), 4.84 and 4.65 (each d, J=3.3, 5.7 Hz, total 1H), 4.42 and 4.29 (each m, total 1H), 2.10-1.10 (m, 20H), 0.92-0.89 (each s, total 9H), 0.15, 0.09, 0.06 and 0.01 (each s, total 6H).

### REFERENCE EXAMPLE 36

cyclohexyl-N-[(2S)-1-hydroxy-1-[5-(1-methylethyloxy)-1,3,4-oxadiazol-2-yl]-4-methyl-2-pentyl]carboxamide

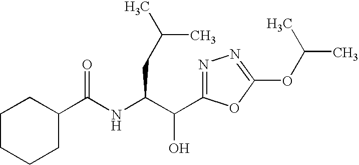

To a solution of the compound prepared in reference example 35 (278 mg) in THF (2.0 ml) was added tetrabutylammonium fluoride (1N solution in THF; 0.7 ml) and the mixture was stirred at room temperature. To the reaction mixture were added ethyl acetate and 1N hydrochloric acid and the organic layer was washed with a saturated aqueous solution of sodium bicarbonate and water successively, dried over anhydrous magnesium sulfate and concentrated to give the title compound having the following physical data. The compound was used in the next reaction without further purification.

TLC: Rf 0.13 and 0.15 (n-hexane:ethyl acetate=1:1); NMR (CDCl₃): δ 5.85 and 5.75 (each brd, J=7.8 Hz, total 1H), 5.18-5.05 (m, 1H), 4.76 and 4.22 (each d, J=3.9, 5.7 Hz, total 1H), 4.45 and 4.23 (each m, total 1H), 2.15 and 2.05 (m, 1H), 1.90-1.20 (m, 13H), 1.46 and 1.45 (each d, J=6.3 Hz, each 3H), 0.92 and 0.91 (each d, J=6.0 Hz, each 3H).

### EXAMPLE 8

cyclohexyl-N-[(2S)-1-[5-(1-methylethyloxy)-1,3,4-oxadiazol-2-yl]-4-methyl-1-oxo-2-pentyl]carboxamide

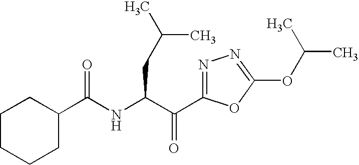

To a solution of the compound prepared in reference example 9 (250 mg) in methylene chloride (6 ml) was added Dess-Martin reagent (382 mg) and the mixture was stirred at room temperature. To the reaction mixture were added ethyl acetate and a saturated aqueous sodium thiosulfate and the mixture was stirred for 10 minutes. The extracted organic layer was washed with a saturated aqueous solution of sodium bicarbonate twice and a saturated aqueous solution of sodium chloride once, dried over anhydrous sodium sulfate and concentrated. The residue was purified by column chromatography on silica gel (n-hexane:ethyl acetate=7:3) to give the compound of the present invention (148 mg) having the following physical data.

TLC: Rf 0.70 (n-hexane:ethyl acetate=1:1); NMR (CDCl₃): δ 5.99 (brd, J=7.8 Hz, 1H), 5.44 (m, 1H), 5.27 (septet, J=6.3 Hz, 1H), 2.16 (m, 1H), 1.92-1.20 (m, 13H), 1.03 (d, J=6.3 Hz, 3H), 0.96 (d, J=6.3 Hz, 3H).

### EXAMPLE 8(1)-EXAMPLE 8(3)

By the same procedure as described in example 8 using a corresponding compound, the compounds of the present invention having the following physical data were given.

### EXAMPLE 8(1)

cyclohexyl-N-[1-[5-(2-dimethylaminoethyloxy)-1,3,4-oxadiazol-2-yl]-4-methyl]-oxo-2-pentyl]carboxamide hydrochloride

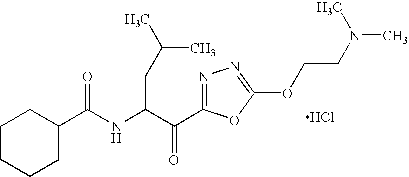

TLC: Rf 0.59 (ethyl acetate:acetic acid:water=3:1:1); NMR (DMSO-d₆): δ 10.6-10.0 (broad, 1H), 8.33 (brd, J=6.3 Hz, 1H), 5.04 (m, 1H), 4.92 (m, 2H), 3.62 (m, 2H), 2.84 (s, 6H), 2.20 (m, 1H), 1.80-1.00 (m, 13H), 0.91 (d, J=6.3 Hz, 3H), 0.90 (d, J=6.3 Hz, 3H).

### EXAMPLE 8(2)

cyclohexyl-N-[(2S)-1-[5-(2-methylpropyloxy)-1,3,4-oxadiazol-2-yl]-4-methyl-1-oxo-2-pentyl]carboxamide

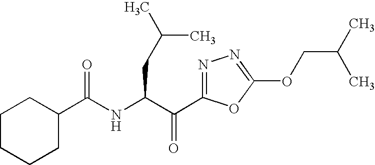

TLC: Rf 0.73 (n-hexane:ethyl acetate=1:1); NMR (CDCl₃): δ 6.01 (brd, J=7.5 Hz, 1H), 5.47-5.37 (m, 1H), 4.38 (d, J=6.6 Hz, 2H), 2.28-2.10 (m, 2H), 1.93-1.17 (m, 13H), 1.04 (d, J=6.6 Hz, 6H), 1.02 and 0.97 (each d, J=6.3 Hz, each 3H).

### EXAMPLE 8(3)

1-benzoylaminocyclohexyl-N-[1-[5-(2-dimethylaminoethyl oxy)-1,3,4-oxadiazol-2-yl]-4-methyl-1-oxo-2-pentyl]carboxamide

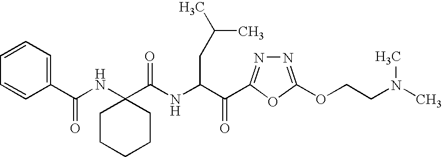

TLC: Rf 0.26 (ethyl acetate:methanol=4:1); NMR (CDCl₃): δ 7.97 (brd, J=6.9 Hz, 1H), 7.77 (d, J=6.9 Hz, 2H), 7.60-7.40 (m, 3H), 6.06 (brs, 1H), 5.42-5.33 (m, 1H), 4.65 (t, J=5.4 Hz, 2H), 2.77 (t, J=5.4 Hz, 2H), 2.34 (s, 6H), 2.35-1.28 (m, 13H), 1.00 and 0.96 (each d, J=6.0 Hz, each 3H).

### EXAMPLE 9(1)-EXAMPLE 9(69)

By the same procedure as described in example 1 using a corresponding compound, optionally subjecting to a deprotection reaction of a corresponding protective group, the compounds of the present invention having the following physical data were given.

For example, the compound of example 9(15) was given by subjecting to a deprotection reaction under alkaline condition the compound having acetyl as a protective group of hydroxy, and the compound of example 9(50) was given by subjecting to a deprotection reaction using toluene sulfonic acid the compound of example 9(15).

### EXAMPLE 9(1)

1-(2-methylbenzoylamino)cyclohexyl-N-[1-[5-(2-dimethylaminoethylthio)-1,3,4-oxadiazol-2-yl]-4-methyl-1-oxo-2-pentyl]carboxamide hydrochloride

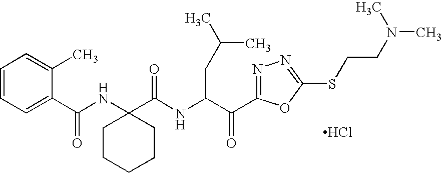

TLC: Rf 0.44 (methylene chloride:methanol:acetic acid=9:1:1); NMR (DMSO-d₆): δ 10.42-10.25 (br, 1H), 8.16 (d, J=7.2 Hz, 1H), 7.86 (s, 1H), 7.45 (d, J=7.2 Hz, 1H), 7.36-7.28 (m, 1H), 7.26-7.20 (m, 2H), 5.16-5.06 (m, 1H), 3.74-3.66 (m, 2H), 3.54-3.44 (m, 2H), 2.81 (s, 6H), 2.33 (s, 3H), 2.20-1.10 (m, 13H), 1.00-0.80 (m, 6H).

### EXAMPLE 9(2)

1-benzyloxymethylcyclohexyl-N-[1-[5-(2-diethylaminoethylthio)-1,3,4-oxadiazol-2-yl]-4-methyl-1-oxo-2-pentyl]carboxamide hydrochloride

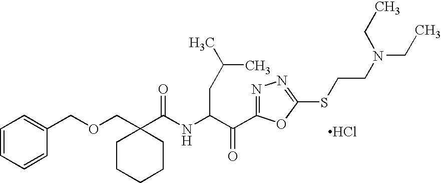

TLC: Rf 0.56 (ethyl acetate:methanol=9:1); NMR (DMSO-d₆): δ 8.07 (d, J=6.6 Hz, 1H), 7.35-7.24 (m, 5H), 5.09-5.02 (m, 1H), 4.41 (s, 2H), 3.71-3.66 (m, 2H), 3.48-3.34 (m, 4H), 3.22-3.14 (m, 4H), 2.04-1.87 (m, 2H), 1.74-1.14 (m, 11H), 1.24 (t, J=7.2 Hz, 6H), 0.90-0.84 (m, 6H).

### EXAMPLE 9(3)

1-(4-methyl-1,2,3-thiadiazol-5-ylcarbonylamino)cyclohexyl-N-[1-[5-(2-dimethylaminoethylthio)-1,3,4-oxadiazol-2-yl]-4-methyl-1-oxo-2-pentyl]carboxamide hydrochloride

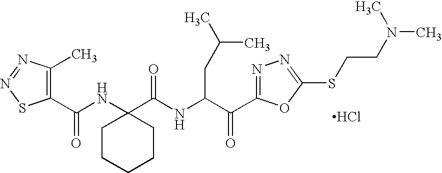

TLC: Rf 0.70 (methylene chloride:methanol:acetic acid=10:1:1); NMR (DMSO-d₆): δ 10.25-10.15 (br, 1H), 8.39 (s, 1H), 8.25 (d, J=7.5 Hz, 1H), 5.10-5.00 (m, 1H), 3.72-3.64 (m, 2H), 3.54-3.44 (m, 2H), 2.82 (s, 6H), 2.74 (s, 3H), 2.10-1.20 (m, 13H), 0.91-0.86 (m, 6H).

### EXAMPLE 9(4)

1-[5-methyl-2-trifluoromethylfuran-3-ylcarbonylamino]cyclohexyl-N-[1-[5-(2-dimethylaminoethylthio)-1,3,4-oxadiazol-2-yl]-4-methyl-1-oxo-2-pentyl]carboxamide hydrochloride

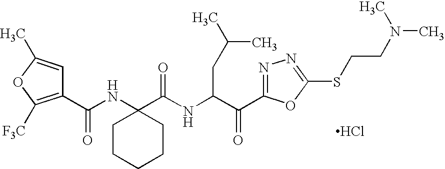

TLC: Rf 0.75 (methylene chloride:methanol:acetic acid=10:1:1); NMR (DMSO-d₆): δ 10.41-10.30 (br, 1H), 8.05 (d, J=6.9 Hz, 1H), 7.92 (s, 1H), 6.69 (s, 1H), 5.12-5.00 (m, 1H), 3.78-3.62 (m, 2H), 3.55-3.45 (m, 2H), 2.82 (s, 6H), 2.37 (s, 3H), 2.10-1.15 (m, 13H), 0.92-0.84 (m, 6H).

### EXAMPLE 9(5)

1-(isoxazole-5-ylcarbonylamino)cyclohexyl-N-[1-[5-(2-dimethylaminoethylthio)-1,3,4-oxadiazol-2-yl]-4-methyl-1-oxo-2-pentyl]carboxamide hydrochloride

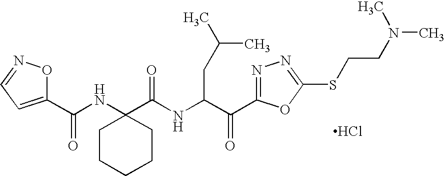

TLC: Rf 0.51 (methanol:chloroform=1:9); NMR (DMSO-d₆): δ 10.52 (br, 1H), 8.74 (d, J=1.8 Hz, 1H), 8.24 (brd, J=6.6 Hz, 1H), 8.17 (s, 1H), 7.13 (d, J=1.8 Hz, 1H), 5.00 (m, 1H), 3.69 (m, 2H), 3.49 (m, 2H), 2.82 (s, 6H), 2.20-2.03 (m, 2H), 1.82-1.17 (m, 11H), 0.86 (d, J=6.0 Hz, 3H), 0.84 (d, J=6.0 Hz, 3H).

### EXAMPLE 9(6)

1-anilinocarbonylcyclohexyl-N-[1-[5-(2-dimethylaminoethylthio)-1,3,4-oxadiazol-2-yl]-4-methyl-1-oxo-2-pentyl]carboxamide hydrochloride

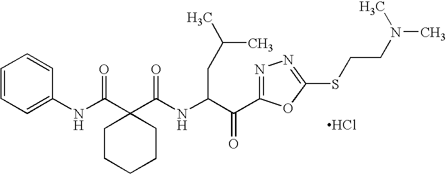

TLC: Rf 0.53 (ethyl acetate:methanol=9:1); NM (DMSO-d₆): δ 9.01 (s, 1H), 8.32 (d, J=6.6 Hz, 1H), 7.58 (d, J=8.1 Hz, 2H), 7.31-7.26 (m, 2H), 7.07-7.03 (m, 1H), 5.16-5.06 (m, 1H), 3.68 (t, J=7.8 Hz, 2H), 3.49-3.44 (m, 2H), 2.81 (s, 6H), 2.20-1.87 (m, 5H), 1.78-1.22 (m, 8H), 0.78-0.83 (m, 6H).

### EXAMPLE 9(7)

1-benzyloxycarbonylaminocyclohexyl-N-[1-[5-(2-dimethylaminoethylthio)-1,3,4-oxadiazol-2-yl]-4-methyl-1-oxo-2-pentyl]carboxamide hydrochloride

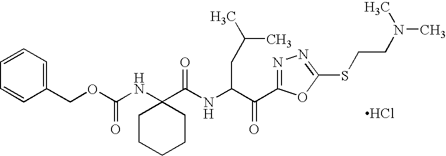

TLC: Rf 0.65 (methylene chloride:methanol:acetic acid=10:1:1); NMR (DMSO-d₆): δ 10.95-10.80 (br, 1H), 8.09 (d, J=6.6 Hz, 1H), 7.50-7.22 (m, 5H), 7.07 (s, 1H), 5.10-4.84 (m, 3H), 3.80-3.65 (m, 2H), 3.54-3.40 (m, 2H), 2.80 (s, 6H), 2.02-1.05 (m, 13H), 0.89-0.78 (m, 6H).

### EXAMPLE 9(8)

1-(4-methylphenyloxycarbonylamino)cyclohexyl-N-[1-[5-(2-dimethylaminoethylthio)-1,3,4-oxadiazol-2-yl]-4-methyl-1-oxo-2-pentyl]carboxamide hydrochloride

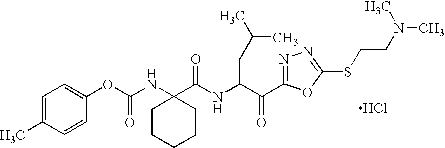

TLC: Rf 0.70 (methylene chloride:methanol:acetic acid=10:1:1); NMR (DMSO-d₆): δ 10.18-10.02 (br, 1H), 8.23 (d, J=6.6 Hz, 1H), 7.52 (s, 1H), 7.15 (d, J=8.4 Hz, 2H), 6.94 (d, J=8.4 Hz, 2H), 5.12-5.02 (m, 1H), 3.74-3.64 (m, 2H), 3.52-3.42 (m, 2H), 2.81 (s, 6H), 2.28 (s, 3H), 2.00-1.20 (m, 13H), 0.91-0.86 (m, 6H).

### EXAMPLE 9(9)

1-(4-chlorophenyloxycarbonylamino)cyclohexyl-N-[1-[5-(2-dimethylaminoethylthio)-1,3,4-oxadiazol-2-yl]-4-methyl-1-oxo-2-pentyl]carboxamide hydrochloride

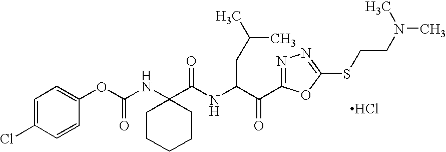

TLC: Rf 0.59 (methylene chloride:methanol:acetic acid=10:1:1); NMR (DMSO-d₆): δ 10.00-9.88 (br, 1H), 8.27 (d, J=6.6 Hz, 1H), 7.66 (s, 1H), 7.43 (d, J=8.7 Hz, 2H), 7.10 (d, J=8.7 Hz, 2H), 5.12-5.02 (m, 1H), 3.72-3.62 (m, 2H), 3.54-3.42 (m, 2H), 2.81 (s, 6H), 2.00-1.18 (m, 13H), 0.92-0.84 (m, 6H).

### EXAMPLE 9(10)

1-(4-bromophenyloxycarbonylamino)cyclohexyl-N-[1-[5-(2-dimethylaminoethylthio)-1,3,4-oxadiazol-2-yl]-4-methyl-1-oxo-2-pentyl]carboxamide hydrochloride

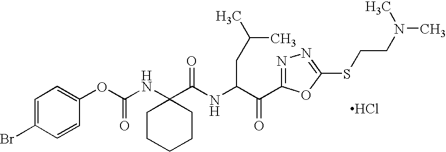

TLC: Rf 0.48 (methylene chloride:methanol:acetic acid=20:1:1); NMR (DMSO-d₆): δ 10.38-10.20 (br, 1H), 8.34-8.22 (m, 1H), 7.67 (s, 1H), 7.60-7.52 (m, 2H), 7.12-7.00 (m, 2H), 5.12-5.02 (m, 1H), 3.72-3.62 (m, 2H), 3.64-3.40 (m, 2H), 2.81 (s, 6H), 2.02-1.18 (m, 13H), 1.02-0.76 (m, 6H).

### EXAMPLE 9(11)

1-benzyloxymethylcarbonylcyclohexyl-N-[1-[5-(2-dimethylaminoethylthio)-1,3,4-oxadiazol-2-yl]-4-methyl-1-oxo-2-pentyl]carboxamide hydrochloride

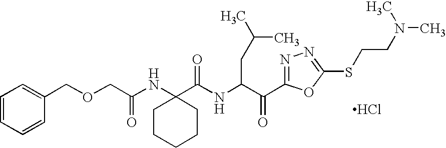

TLC: Rf 0.68 (methylene chloride:methanol:acetic acid=20:1:1); NMR (DMSO-d₆): δ 10.44-10.28 (br, 1H), 8.19 (d, J=6.6 Hz, 1H), 7.44-7.28 (m, 5H), 7.26 (s, 1H), 5.04-4.94 (m, 1H), 4.56 (s, 2H), 3.93 (s, 2H), 3.74-3.64 (m, 2H), 3.56-3.44 (m, 2H), 2.81 (s, 6H), 2.08-1.10 (m, 13H), 0.92-0.85 (m, 6H).

### EXAMPLE 9(12)

1-phenethylcyclohexyl-N-[1-[5-(2-dimethylaminoethylthio)-1,3,4-oxadiazol-2-yl]-4-methyl-1-oxo-2-pentyl]carboxamide hydrochloride

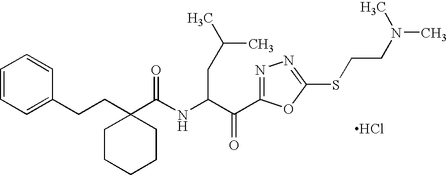

TLC: Rf 0.56 (ethyl acetate:methanol=9:1); NMR (DMSO-d₆): δ 8.19 (d, J=6.0 Hz, 1H), 7.29-7.24 (m, 2H), 7.17-7.14 (m, 3H), 5.19-5.12 (m, 1H), 3.71 (t, J=6.3 Hz, 2H), 3.48 (m, 2H), 2.81 (s, 6H), 2.35 (t, J=8.7 Hz, 2H), 2.10-2.07 (m, 2H), 1.84-1.14 (m, 13H), 0.94 (d, J=6.3 Hz, 3H), 0.93 (d, J=6.3 Hz, 3H).

### EXAMPLE 9(13)

2,2-dimethylpropyloxy-N-[1-[5-(2-dimethylaminoethylthio)-1,3,4-oxadiazol-2-yl]-4-methyl-1-oxo-2-pentyl]carboxamide hydrochloride

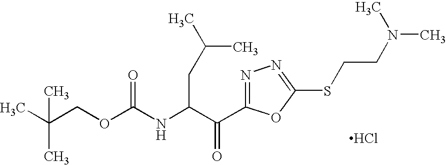

TLC: Rf 0.84 (chloroform:methanol:acetic acid=10:2:1); NMR (CDCl₃): δ 12.8 (brs, 1H), 5.34-5.20 (m, 2H), 3.95-3.87 (m, 2H), 3.77 (s, 2H), 3.64-3.52 (m, 2H), 2.96 and 2.95 (each s, total 6H), 1.90-1.50 (m, 3H), 1.05 and 0.99 (each d, J=6.3 Hz, each 3H), 0.92 (s, 9H).

### EXAMPLE 9(14)

2,2,2-trichloroethyloxy-N-[1-[5-(2-dimethylaminoethylthio)-1,3,4-oxadiazol-2-yl]-4-methyl-1-oxo-2-pentyl]carboxamide hydrochloride

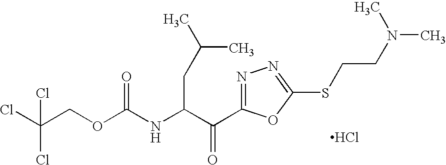

TLC: Rf 0.80 (chloroform:methanol:acetic acid=10:2:1); NMR (CDCl₃): δ 5.65 (brd, 1H), 5.31 (m, 1H), 4.76 and 4.70 (each d, J=12 Hz, total 2H), 3.96-3.87 (m, 2H), 3.60-3.52 (m, 2H), 2.95 (s, 6H), 1.90-1.50 (m, 3H), 1.07 and 0.99 (each d, J=6.3 Hz, each 3H).

### EXAMPLE 9(15)

1-(4-hydroxybenzoylamino)cyclohexyl-N-[1-[5-(2-dimethylaminoethylthio)-1,3,4-oxadiazol-2-yl]-4-methyl-1-oxo-2-pentyl]carboxamide hydrochloride

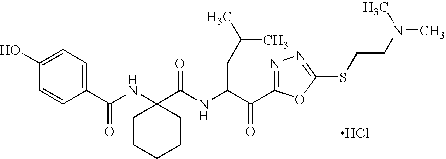

TLC: Rf 0.60 (chloroform:methanol:acetic acid=10:2:1); NMR (DMSO-d₆): δ 10.24 (brs, 1H), 9.98 (s, 1H), 8.04 (brd, J=7.5 Hz, 1H), 7.67 (d, J=8.7 Hz, 2H), 7.52 (brs, 1H), 6.80 (d, J=8.7 Hz, 2H), 5.03(m, 1H), 3.75-3.65 (m, 2H), 3.55-3.45 (m, 2H), 2.82 (s, 6H), 2.15-1.15 (m, 13H), 0.87 and 0.84 (each d, J=6.3 Hz, each 3H).

### EXAMPLE 9(16)

1-(t-butoxycarbonylamino)cyclohexyl-N-[1-[5-(2-benzylmethylaminoethylthio)-1,3,4-oxadiazol-2-yl]-4-methyl-1-oxo-2-pentyl]carboxamide hydrochloride

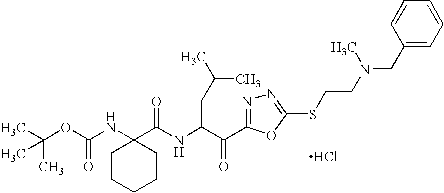

TLC: Rf 0.68 (methylene chloride:methanol:acetic acid=20:1:1); NMR (DMSO-d₆): δ 10.70-10.65 (br, 1H), 7.97 (d, J=6.6 Hz, 1H), 7.64-7.52 (m, 2H), 7.50-7.40 (m, 3H), 6.54 (s, 1H), 5.12-5.00 (m, 1H), 4.54-4.42 (m, 1H), 4.38-4.24 (m, 1H), 3.90-3.68 (m, 2H), 3.68-3.40 (m, 2H), 2.75 (s, 3H), 1.90-1.10 (m, 22H),0.92 (d, J=6.0 Hz, 6H).

### EXAMPLE 9(17)

1-benzoylaminocyclohexyl-N-[1-[5-(2-benzylmethylaminoethylthio)-1,3,4-oxadiazol-2-yl]-4-methyl-1-oxo-2-pentyl]carboxamide hydrochloride

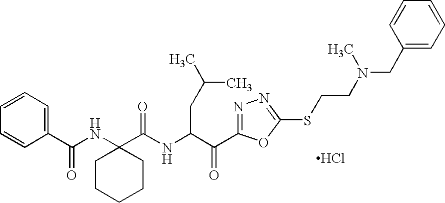

TLC: Rf 0.69 (methylene chloride:methanol:acetic acid=20:1:1); NMR (DMSO-d₆): δ 10.50-10.45 (br, 1H), 8.08 (d, J=6.0 Hz, 1H), 7.84-7.78 (m, 3H), 7.60-7.54 (m, 2H), 7.51 (s, 1H), 7.48-7.42 (m, 5H), 5.08-4.98 (m, 1H), 4.54-4.48 (m, 1H), 4.35-4.22 (m, 1H), 3.90-3.68 (m, 2H), 3.64-3.48 (m, 2H), 2.73 (s, 3H), 2.18-1.15 (m, 13H), 0.90-0.80 (m, 6H).

### EXAMPLE 9(18)

N-[1-(5-dimethylaminoethylthio-1,3,4-oxadiazol-2-yl)-4-methyl-1-oxo-2-pentyl]-4,4-dimethyl-2-pentenamide hydrochloride

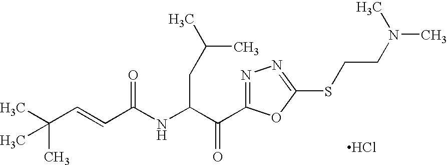

TLC: Rf 0.47 (ethyl acetate:methanol=8:2); NMR (CDCl₃): δ 13.1-12.9 (broad, 1H), 6.87 (d, J=16 Hz, 1H), 6.09 (m, 1H), 5.75 (d, J=16 Hz, 1H), 5.51 (m, 1H), 3.96-3.86 (m, 2H), 3.62-3.50 (m, 2H), 3.00-2.85 (m, 6H), 1.90-1.55 (m, 3H), 1.08 (s, 9H), 1.05 and 0.99 (each d, J=6.3 Hz, each 3H).

### EXAMPLE 9(19)

4-(t-butyl)cyclohexyl-N-[1-[5-(2-dimethylaminoethylthio)-1,3,4-oxadiazol-2-yl]-4-methyl-1-oxo-2-pentyl]carboxamide hydrochloride

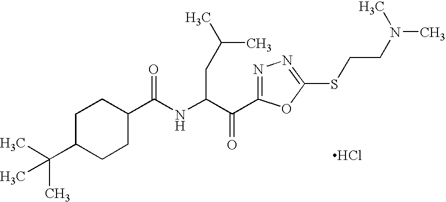

TLC: Rf 0.56 (ethyl acetate:methanol=8:2); NMR (CDCl₃): δ 13.2-12.9 (broad, 1H), 6.13 and 6.02 (each m, 1H), 5.50-5.35 (m, 1H), 3.95-3.85 (m, 2H), 3.58-3.47 (m, 2H), 3.00-2.85 (m, 6H), 2.60-0.85 (m, 19H), 0.85 and 0.82 (each s, total 9H).

### EXAMPLE 9(20)

N-[1-(5-dimethylaminoethylthio-1,3,4-oxadiazol-2-yl)-4-methyl-1-oxo-2-pentyl]-3,3-dimethyl-2-pentanamide hydrochloride

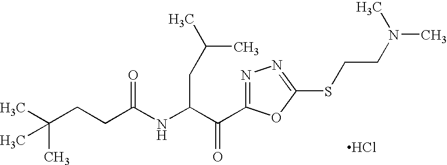

TLC: Rf 0.47 (ethyl acetate:methanol=8:2); NMR (CDCl₃): δ 13.2-12.9 (broad, 1H), 6.20-5.97 (m, 1H), 5.43 (m, 1H), 3.95-3.85 (m, 2H), 3.60-3.50 (m, 2H), 3.00-2.85 (m, 6H), 2.25-1.40 (m, 7H), 1.03 and 0.99 (each d, J=6.3 Hz, total 6H), 0.90 (s, 9H).

### EXAMPLE 9(21)

cyclohexyl-N-[1-[5-(2-dimethylaminoethylthio)-1,3,4-oxadiazol-2-yl]-1-oxo-3-phenyl-2-propyl]carboxamide hydrochloride

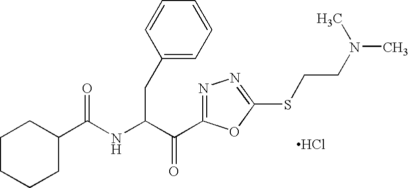

TLC: Rf 0.63 (methylene chloride:methanol=10:1); NMR (DMSO-d₆): δ 10.37-10.15 (br, 1H), 8.51 (d, J=6.6 Hz, 1H), 7.32-7.18 (m, 5H), 5.22-5.17 (m, 1H), 3.74-3.66 (m, 2H), 3.54-3.44 (m, 2H), 3.23 (dd, J=13.8, 4.5 Hz, 1H), 2.88 (dd, J=13.8, 9.6 Hz, 1H), 2.82 (s, 6H), 2.20-2.07 (m, 1H), 1.65-1.42 (m, 5H), 1.30-1.00 (m, 5H).

### EXAMPLE 9(22)

cycloheptyl-N-[1-[5-(2-dimethylaminoethylthio)-1,3,4-oxadiazol-2-yl]-1-oxo-3-phenyl-2-propyl]carboxamide hydrochloride

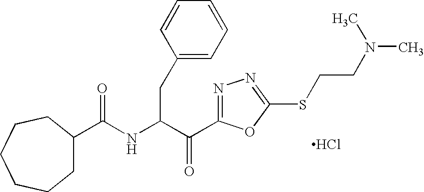

TLC: Rf 0.57 (methylene chloride:methanol=10:1); NMR (DMSO-d₆): δ 10.37-10.22 (br, 1H), 8.50 (d, J=6.6 Hz, 1H), 7.30-7.18 (m, 5H), 5.24-5.14 (m, 1H), 3.74-3.65 (m, 2H), 3.54-3.44 (m, 2H), 3.22 (dd, J=13.8, 4.2 Hz, 1H), 2.88 (dd, J=13.8, 9.6 Hz, 1H), 2.82 (s, 6H), 2.38-2.22 (m, 1H), 1.70-1.24 (m, 12H).

### EXAMPLE 9(23)

cycloheptyl-N-[(2S)-1-[5-(2-dimethylaminoethylthio)-1,3,4-oxadiazol-2-yl]-3-methyl-1-oxo-2-butyl]carboxamide hydrochloride

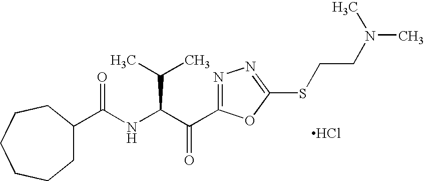

TLC: Rf 0.42 (ethyl acetate:methanol=8:2); NMR (DMSO-d₆): δ 10.15 (brs, 1H), 8.28 (d, J=5.7 Hz, 1H), 4.89 (dd, J=6.6, 5.7 Hz, 1H), 3.71 (m, 2H), 3.50 (m, 2H), 2.82 (s, 6H), 2.42 (m, 1H), 2.25 (m, 1H), 1.80-1.33 (m, 12H), 0.93 and 0.91 (each d, J=6.6 Hz, total 6H).

### EXAMPLE 9(24)

cyclohexyl-N-[1-[5-(2-dimethylaminoethylthio)-1,3,4-oxadiazol-2-yl]-4-phenyl-1-oxo-2-butyl]carboxamide hydrochloride

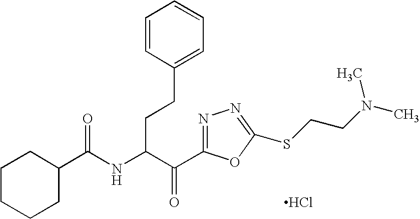

TLC: Rf 0.59 (methylene chloride:methanol=10:1); NMR (DMSO-d₆): δ 10.55-10.40 (br, 1H), 8.55 (d, J=5.7 Hz, 1H), 7.31-7.10 (m, 5H), 4.95-4.84 (m, 1H), 3.75-3.65 (m, 2H), 2.82 (s, 6H), 2.78-2.52 (m, 2H), 2.30-2.08 (m, 2H), 1.97-1.90 (m, 1H), 1.80-1.52 (m, 5H), 1.40-1.04 (m, 5H).

### EXAMPLE 9(25)

cycloheptyl-N-[1-[5-(2-dimethylaminoethylthio)-1,3,4-oxadiazol-2-yl]-1-oxo-4-phenyl-2-butyl]carboxamide hydrochloride

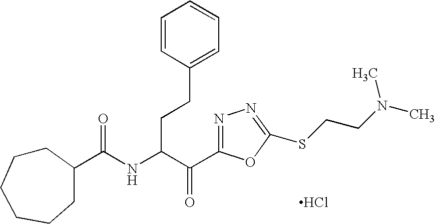

TLC: Rf 0.59 (methylene chloride:methanol=10:1); NMR (DMSO-d₆): δ 10.58-10.45 (br, 1H), 8.54 (d, J=6.0 Hz, 1H), 7.32-7.15 (m, 5H), 4.94-4.82 (m, 1H), 3.75-3.67 (m, 2H), 3.55-3.42 (m, 2H), 2.81 (s, 6H), 2.75-2.50 (m, 2H), 2.48-2.36 (m, 1H), 2.24-2.10 (m, 1H), 2.00-1.85 (m, 1H), 1.80-1.32 (m, 12H).

### EXAMPLE 9(26)

cycloheptyl-N-[(2S)-3,3-dimethyl-1-[5-(2-dimethylaminoethylthio)-1,3,4-oxadiazol-2-yl]-1-oxo-2-butyl]carboxamide hydrochloride

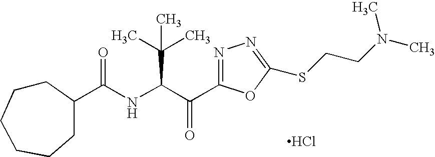

TLC: Rf 0.50 (ethyl acetate:methanol=9:1); NMR (DMSO-d₆): δ 8.22 (d, J=5.4 Hz, 1H), 4.95 (d, J=5.4 Hz, 1H), 3.73 (t, J=7.5 Hz, 2H), 3.50 (t, J=7.5 Hz, 2H), 2.82 (s, 6H), 2.60-2.40 (m, 1H), 1.75-1.32 (m, 12H), 0.99 (s, 9H).

### EXAMPLE 9(27)

cycloheptyl-N-[3-cyclohexyl-1-[5-(2-dimethylaminoethylthio)-1,3,4-oxadiazol-2-yl]-1-oxo-2-propyl]carboxamide hydrochloride

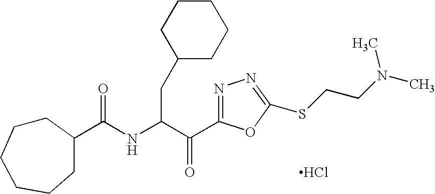

TLC: Rf 0.64 (ethyl acetate:methanol:water=40:10:1); NMR (DMSO-d₆): δ 8.36 (d, J=6.3 Hz, 1H), 5.10-5.02 (m, 1H), 3.80-3.41 (m, 4H), 2.82 (s, 3H), 2.61 (s, 3H), 2.60-2.32 (m, 1H), 1.88-0.80 (m, 25H).

### EXAMPLE 9(28)

cyclohexyl-N-[1-[5-[2-(N-t-butyl-N-methylamino)ethylthio]-1,3,4-oxadiazol-2-yl]-4-methyl-1-oxo-2-pentyl]carboxamide hydrochloride

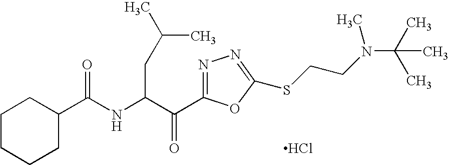

TLC: Rf 0.38 (methanol:chloroform=i:9); NMR(CDCl₃): δ 12.3 (br, 1H), 5.92 (br, 1H), 5.41 (m, 1H), 4.20-3.60 (m, 3H), 3.23 (m, 1H), 2.88 (brs, 3H), 2.23-2.05 (m, 1H), 1.90-1.10 (m, 22H), 1.01 and 0.90 (each d, J=6.0 Hz, each 3H).

### EXAMPLE 9(29)

2-cycloheptylcarbonyl-3-[5-(2-dimethylaminoethylthio)-1,3,4-oxadiazol-2-ylcarbonyl]-1,2,3,4-tetrahydro isoquinoline hydrochloride

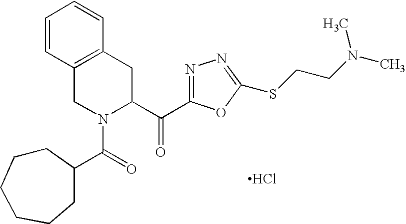

TLC: Rf 0.55 (ethyl acetate:methanol=9:1); NMR (DMSO-d₆): δ 10.79 (brs, 1H), 7.40-7.12 (m, 4H), 5.27 (dd, J=7.5, 6.3 Hz, 1H), 4.81 and 4.74 (each d, J=15.3 Hz, total 2H), 3.73 (m, 2H), 3.50 (m, 2H), 3.29 (dd, J=15.6, 6.3 Hz, 1H), 3.14 (dd, J=15.6, 7.5 Hz, 1H), 2.97 (m, 1H), 2.81 (brs, 6H), 1.80-1.20 (m, 12H).

### EXAMPLE 9(30)

cycloheptyl-N-[1-[5-(2-dimethylaminoethylthio)-1,3,4-oxadiazol-2-ylcarbonyl]cyclohexyl]carboxamide hydrochloride

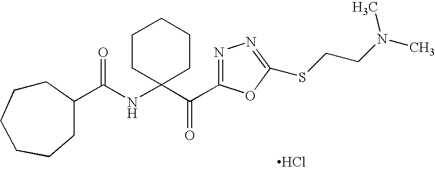

TLC: Rf 0.51 (ethyl acetate:methanol=9:1); NMR (DMSO-d₆): δ 10.89 (brs, 1H), 8.64 (s, 1H), 3.70 (m, 2H), 3.44 (m, 2H), 2.79 (s, 6H), 2.48 (m, 1H), 2.05-1.00 (m, 22H).

### EXAMPLE 9(31)

cycloheptyl-N-[2-cyclohexyl-1-[5-(2-dimethylaminoethylthio)-1,3,4-oxadiazol-2-yl]-1-oxo-2-ethyl]carboxamide hydrochloride

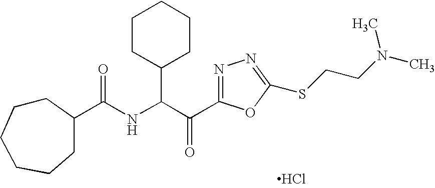

TLC: Rf 0.70 (ethyl acetate:methanol=4:1); NMR (DMSO-d₆): δ 10.64 (brs, 1H), 8.31 (d, J=6.3 Hz, 1H), 4.88 (t, J=6.3 Hz, 1H), 3.72 (t, J=7.2 Hz, 2H), 3.49 (t, J=7.2 Hz, 2H), 2.81 (s, 6H), 2.00-1.00 (m, 24H).

### EXAMPLE 9(32)

cyclohexyl-N-[4,4-dimethyl-1-[5-(2-dimethylaminoethylthio)-1,3,4-oxadiazol-2-yl]1-oxo-2-pentyl]carboxamide hydrochloride

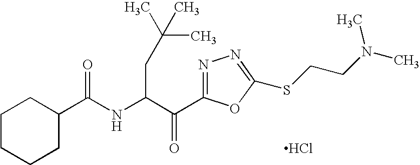

TLC: Rf 0.60 (methylene chloride:methanol=10:1); NMR (DMSO-d₆): δ 10.60-10.40 (br, 1H), 8.34 (d, J=6.9 Hz, 1H), 5.18-5.02 (m, 1H), 3.74-3.67 (m, 2H), 3.54-3.43 (m, 2H), 2.81 (s, 6H), 2.24-2.12 (m, 1H), 1.78-1.05 (m, 12H), 0.94 (s, 9H).

### EXAMPLE 9(33)

cycloheptyl-N-[4,4-dimethyl-1-[5-(2-dimethylaminoethylthio)-1,3,4-oxadiazol-2-yl-]-1-oxo-2-pentyl]carboxamide hydrochloride

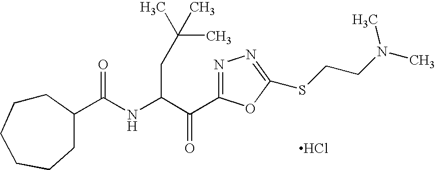

TLC: Rf 0.79 (methylene chloride:methanol=10:1); NMR (DMSO-d₆): δ 10.30-10:10 (br, 1H), 8.32 (d, J=6.6 Hz, 1H), 5.15-5.04 (m, 14), 3.73-3.65 (m, 2H), 3.53-3.42 (m, 2H), 2.80 (s, 6H), 2.42-2.30 (m, 1H), 1.82-1.20 (m, 14H), 0.94 (s, 9H).

### EXAMPLE 9(34)

cycloheptyl-N-[1-[5-(2-dimethylaminoethylthio)-1,3,4-oxadiazol-2-yl]-2-(tetrahydropyran-4-yl)-1-oxo-2-ethyl]carboxamide hydrochloride

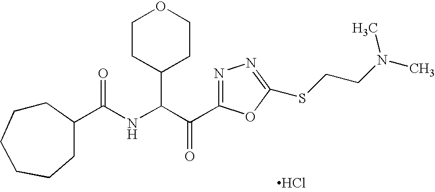

TLC: Rf 0.39 (methanol:chloroform:water=1:9:0.1); NMR (DMSO-d₆): δ 10.6 (br, 1H), 8.40 (brd, J=6.3 Hz, 1H), 4.91 (t, J=6.3 Hz, 1H), 3.90-3.70 (m, 2H), 3.69 (m, 2H), 3.42-3.20 (m, 4H), 2.73 (s, 6H), 2.42 (m, 1H), 2.20 (m, 1H), 1.70-1.30 (m, 16H).

### EXAMPLE 9(35)

cycloheptyl-N-[1-[5-(2-dimethylaminoethylthio)-1,3,4-oxadiazol-2-yl]-4-methoxy-1-oxo-2-butyl]carboxamide hydrochloride

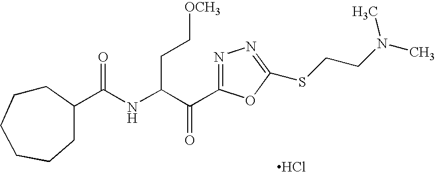

TLC: Rf 0.29 (ethyl acetate:methanol=8:2); NMR (DMSO-d₆): δ 10.88 (brs, 1H), 8.47 (d, J=5.4 Hz, 1H), 4.98 (m, 1H), 3.73 (m, 2H), 3.48 (m, 2H), 3.41 (m, 2H), 3.04 (s, 3H), 2.80 (s, 6H), 2.40 (m, 1H), 2.15-1.95 (m, 2H), 1.70-1.20 (m, 12H).

### EXAMPLE 9(36)

cyclohexyl-N-[1-[5-[2-(1,2,3,4-tetrahydroisoquinoline-2-yl)ethylthio]-1,3,4-oxadiazol-2-yl]-4-methyl-1-oxo-2-pentyl]carboxamide hydrochloride

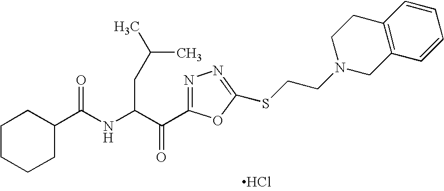

TLC: Rf 0.65 (methylene chloride:methanol=10:1); NMR (DMSO-d₆): δ 11.08-10.85 (br, 1H), 8.39 (d, J=6.3 Hz, 1H), 7.34-7.16 (m, 4H), 5.08-4.98 (m, 1H), 4.72-4.30 (m, 2H), 3.92-3.74 (m, 2H), 3.72-3.58 (m, 2H), 3.30-2.98 (m, 4H), 2.30-2.12 (m, 1H), 1.80-1.05 (m, 13H), 0.93-0.88 (m, 6H).

### EXAMPLE 9(37)

cyclohexyl-N-[(2S)-1-[5-[2-(N-benzyl-N-methylamino)ethylthio]-1,3,4-oxadiazol-2-yl]-3-methyl-1-oxo-2-butyl]carboxamide hydrochloride

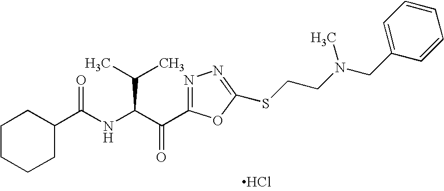

TLC: Rf 0.62 (n-hexane:ethyl acetate=1:1); NMR (DMSO-d₆): δ 8.31 (d, J=6.3 Hz, 1H), 7.59 (br-s, 2H), 7.44 (br-s, 3H), 4.90 (t, J=6.3 Hz, 1H), 4.53-4.41 (m, 1H), 4.33-4.26 (m, 1H), 3.88-3.71 (m, 2H), 3.65-3.32 (m, 2H), 2.73 and 2.72 (each s, total 3H), 2.36-2.20 (m, 2H), 1.76-1.54 (m, 5H), 1.38-1.03 (m, 5H), 0.92 (d, J=6.3 Hz, 3H), 0.90 (d, J=6.3 Hz, 3H).

### EXAMPLE 9(38)

cyclohexyl-N-[(2S)-1-[5-(2-dimethylaminoethylthio)-1,3,4-oxadiazol-2-yl]-3-methyl-1-oxo-2-butyl]carboxamide hydrochloride

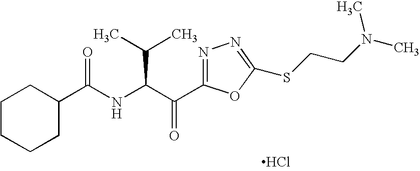

TLC: Rf 0.66 (ethyl acetate:methanol:water=40:10:1); NMR (DMSO-d₆): δ 8.31 (d, J=6.3 Hz, 1H), 4.90 (t, J=6.3 Hz, 1H), 3.73 (t, J=6.9 Hz, 2H), 3.49 (t, J=6.9 Hz, 2H), 2.81 (s, 6H), 2.18-2.01 (m, 2H), 1.77-1.53 (m, 5H), 1.36-1.06 (m, 5H), 0.92 (d, J=6.6 Hz, 3H), 0.90 (d, J=6.3 Hz, 3H).

### EXAMPLE 9(39)

1-benzoylaminocyclohexyl-N-[(2S)-1-[5-(2-dimethylaminoethylthio)-1,3,4-oxadiazol-2-yl]-3-methyl-1-oxo-2-butyl]carboxamide hydrochloride

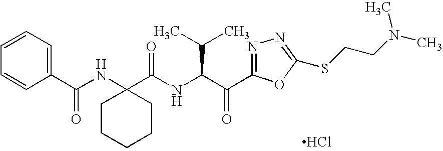

free compound: TLC: Rf 0.32 (ethyl acetate:methanol=9:1); NMR (DMSO-d₆): δ 7.95 (s, 1H), 7.87 (d, J=7.5 Hz, 1H), 7.80 (d, J=7.8 Hz, 2H), 7.57-7.45 (m, 3H), 4.92 (t-like, J=7.5 Hz, 1H), 3.48 (t, J=6.9 Hz, 2H), 2.64 (t, J=6.9 Hz, 2H), 2.33-2.12 (m, 3H), 2.19 (s, 6H), 1.76-1.40 (m, 7H), 1.32-1.14 (m, 1H), 0.89 (d, J=6.6 Hz, 3H), 0.80 (d, J=6.3 Hz, 3H).

hydrochloride: TLC: Rf 0.62 (ethyl acetate:methanol:water=40:10:1); NMR (DMSO-d₆): δ 7.96 (s, 1H), 7.95-7.81 (m, 1H), 7.80 (d, J=6.9 Hz, 2H), 7.58-7.45 (m, 3H), 4.95 (t-like, J=6.0 Hz, 1H), 3.69 (t, J=6.9 Hz, 2H), 3.45 (t, J=6.9 Hz, 2H), 2.78 (s, 6H), 2.33-2.15 (m, 3H), 1.76-1.14 (m, 8H), 0.90 (d, J=6.6 Hz, 3H), 0.80 (d, J=6.3 Hz, 3H).

### EXAMPLE 9(40)

cycloheptyl-N-[1-[5-(2-dimethylaminoethylthio)-1,3,4-oxadiazol-2-yl]-1-oxo-2-phenyl-2-ethyl]carboxamide hydrochloride

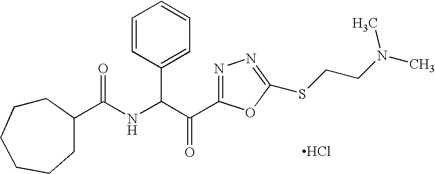

TLC: Rf 0.39 (methanol:methylene chloride=1:9); NMR (DMSO-d₆): δ 10.60 (br, 1H), 8.82 (brd, J=5.4 Hz, 1H), 7.45-7.35 (m,5H), 6.14 (d, J=5.4 Hz, 1H), 3.70 (m, 2H), 3.45 (m, 2H), 2.78 (s, 6H), 2.43 (m, 1H), 1.80-1.32 (m, 12H).

### EXAMPLE 9(41)

1-morpholinocarbonylaminocyclohexyl-N-[(2S)-1-[5-(2-dimethylaminoethylthio)-1,3,4-oxadiazol-2-yl]-3-methyl-1-oxo-2-butyl]carboxamide hydrochloride

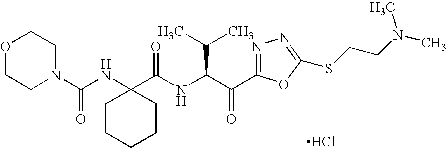

TLC: Rf 0.44 (ethyl acetate:methanol:water=40:10:1); NMR (DMSO-d₆): δ 8.00 (d, J=7.2 Hz, 1H), 6.19 (s, 1H), 4.88 (t-like, J=7.2 Hz, 1H), 3.71 (t, J=5.4 Hz, 2H), 3.54-3.47 (m, 6H), 3.27 (t, J=4.5 Hz, 4H), 2.82 (s, 6H), 2.37-2.24 (m, 1H), 2.07-1.93 (m, 2H), 1.65-1.14 (m, 8H), 0.92 (d, J=6.6 Hz, 3H), 0.85 (d, J=6.6 Hz, 3H).

### EXAMPLE 9(42)

1-(4-dimethylaminomethylbenzoylaminocyclohexyl)-N-[(2S)-3-methyl-1-[5-(1-methylethylthio)-1,3,4-oxadiazol-2-yl]-1-oxo-2-butyl]carboxamide hydrochloride

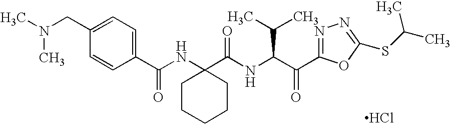

TLC: Rf 0.51 (ethyl acetate:methanol:water=40:10:1); NMR (DMSO-d₆): δ 8.04 (s, 1H), 7.92 (d, J=6.9 Hz, 1H), 7.89 (d, J=8.4 Hz, 2H), 7.66 (d, J=8.4 Hz, 2H), 4.89 (t-like, J=6.9 Hz, 1H), 4.32 (s, 2H), 4.01-3.91 (m, 1H), 2.69 (s, 6H), 2.37-2.11 (m, 3H), 1.77-1.14 (m, 8H), 1.46 (d, J=6.9 Hz, 6H), 0.89 (d, J=6.9 Hz, 3H), 0.80 (d, J=6.9 Hz, 3H).

### EXAMPLE 9(43)

1-(3-dimethylaminomethylbenzoylaminocyclohexyl)-N-[(2S)-3-methyl-1-[5-(1-methylethylthio)-1,3,4-oxadiazol-2-yl]-1-oxo-2-butyl]carboxamide hydrochloride

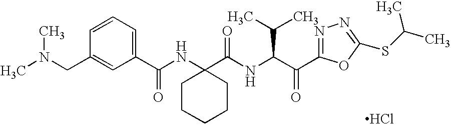

TLC: Rf 0.51 (ethyl acetate:methanol:water=40:10:1); NMR (DMSO-d₆): δ 8.04 (s, 1H), 7.99 (s, 1H), 7.95 (d, J=7.8 Hz, 1H), 7.89 (d, J=7.5 Hz, 1H), 7.60 (d, J=7.5 Hz, 1H), 7.56 (t-like, J=7.5 Hz, 1H), 4.89 (t-like, J=6.6 Hz, 1H), 4.34 (s, 2H), 4.00-3.93 (m, 1H), 2.70 (s, 6H), 2.33-2.19 (m, 3H), 1.80-1.16 (m, 8H), 1.46 (d, J=6.6 Hz, 6H), 0.89 (d, J=6.6 Hz, 3H), 0.80 (d, J=6.6 Hz, 3H).

### EXAMPLE 9(44)

cyclohexyl-N-[1-[5-(2-diisopropylaminoethylthio)-1,3,4-oxadiazol-2-yl]-4-methyl-1-oxo-2-pentyl]carboxamide hydrochloride

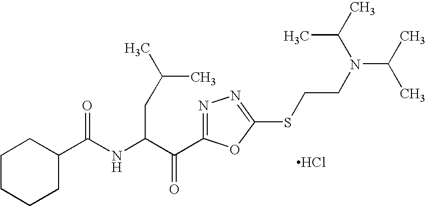

TLC: Rf 0.55 (methylene chloride:methanol=20:1); NMR (DMSO-d₆): δ 10.25-10.16 (br, 114), 8.39 (d, J=6.3 Hz, 1H), 5.06-4.96 (m, 1H), 3.95-3.55 (m, 4H), 3.50-3.40 (m, 2H), 2.30-2.14 (m, 1H), 1.80-1.14 (m, 13H), 1.37 and 1.32 (each d, J=5.7 Hz, total 12H), 0.93-0.87 (m, 6H).

### EXAMPLE 9(45)

cycloheptyl-N-[1-[5-(2-dimethylaminoethylthio)-1,3,4-oxadiazol-2-yl]-3-ethyl-1-oxo-2-pentyl]carboxaniide hydrochloride

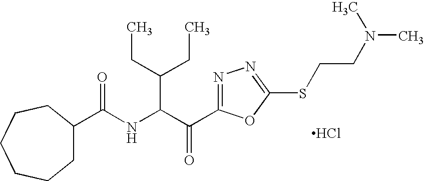

TLC: Rf 0.63 (methanol:ethyl acetate=1:4); NMR (DMSO-d₆): δ 10.41 (br, 1H), 8.14 (brd, J=6.3 Hz, 1H), 5.23 (dd, J=6.3, 5.7 Hz, 1H), 3.73 (m, 2H), 3.55 (m, 2H), 2.84 (m, 6H), 2.00-1.17 (m, 18H), 0.89 and 0.78 (each t, J=7.2 Hz, each 3H).

### EXAMPLE 9(46)

cyclohexyl-N-[1-[5-[2-(N-isopropyl-N-methylamino)ethylthio]-1,3,4-oxadiazol-2-yl]-4-methyl-1-oxo-2-pentyl]carboxamide hydrochloride

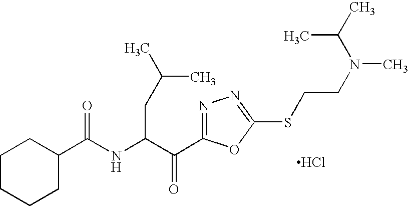

TLC: Rf 0.60 (methylene chloride:methanol=10:1); NMR (DMSO-d₆): δ 10.35-10.20 (br, 1H), 8.38 (d, J=6.3 Hz, 1H), 5.08-4.96 (m, 1H), 3.78-3.68 (m, 2H), 3.67-3.50 (m, 3H), 2.78-2.66 (m, 3H), 2.28-2.12 (m, 1H), 1.80-1.04 (m, 19H), 0.93-0.82 (m, 6H).

### EXAMPLE 9(47)

cyclohexyl-N-[1-[5-[2-(2,5-dihydropyrrol-1-yl)ethylthio]-1,3,4-oxadiazol-2-yl]-4-methyl-1-oxo-2-pentyl]carboxamide hydrochloride

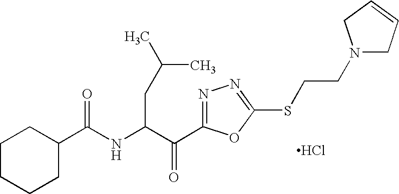

TLC: Rf 0.63 (ethyl acetate:methanol=9:1); NM (DMSO-d₆): δ 8.38 (d, J=6.3 Hz, 1H), 5.93 (s, 2H), 5.06-4.99 (m, 1H), 4.36-4.20 (m, 2H), 4.12-3.88 (m, 2H), 3.70 (s, 4H), 2.28-2.12 (m, 1H), 1.77-1.49 (m, 8H), 1.37-1.03 (m, 5H), 0.92-0.86 (m, 6H).

### EXAMPLE 9(48)

cyclohexyl-N-[1-[5-[2-[N-methyl-N-[2-(pyridin-2-yl)ethyl]amino]ethylthio]-1,3,4-oxadiazol-2-yl]-4-methyl-1-oxo-2-pentyl]carboxamide bishydrochloride

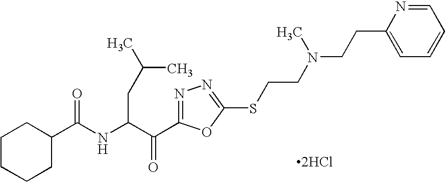

TLC: Rf 0.50 (n-hexane:ethyl acetate=1:1); NMR (DMSO-d₆): δ 8.37 (d, J=6.3 Hz, 1H), 5.03-4.96 (m, 1H), 3.89 (s, 2H), 3.55 (t, J=6.6 Hz, 2H), 2.91 (t, J=6.6 Hz, 2H), 2.38 (s, 3H), 2.26-2.02 (m, 1H), 1.80-1.48 (m, 8H), 1.34-1.08 (m, 5H), 0.91 (d, J=6.0 Hz, 3H), 0.90 (d, J=6.0 Hz, 3H).

### EXAMPLE 9(49)

cyclohexyl-N-[(2S)-1-[5-[2-(tetrahydropyran-2-yloxy)ethylthio]-1,3,4-oxadiazol-2-yl]-4-methyl-1-oxo-2-pentyl]carboxamide

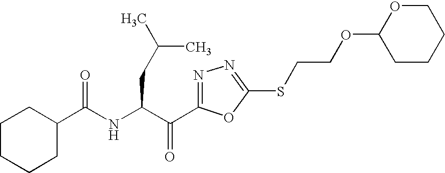

TLC: Rf 0.42 (n-hexane:ethyl acetate=2:1); NMR (DMSO-d₆): δ 8.35 (d, J=6.6 Hz, 1H), 5.04-4.95 (m, 1H), 4.64-4.59 (m, 1H), 3.97-3.86 (m, 1H), 3.78-3.66 (m, 2H), 3.58-3.50 (m, 2H), 3.46-3.36 (m, 1H), 2.24-2.12 (m, 1H), 1.79-1.03 (m, 19H), 0.91 and 0.90 (each d, J=6.0 Hz, total 6H).

### EXAMPLE 9(50)

cyclohexyl-N-[(2S)-1-[5-(2-hydroxyethylthio)-1,3,4-oxadiazol-2-yl]-4-methyl-1-oxo-2-pentyl]carboxamide

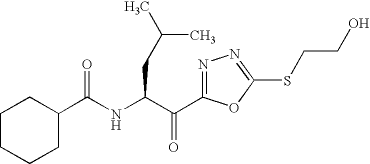

TLC: Rf 0.20 (n-hexane:ethyl acetate=1:1); NMR (DMSO-d₆): δ 8.35 (d, J=6.3 Hz,1H), 5.17 (t, J=5.4 Hz, 11), 5.05-4.95 (m, 1H), 3.78-3.68 (m, 2H), 3.45-3.37 (m, 2H), 2.25-2.10 (m, 1H), 1.80-1.03 (m, 13H), 0.91 and 0.90 (each d, J=6.3 Hz, total 6H).

### EXAMPLE 9(51)

cyclohexyl-N-[1-[5-[2-(N-benzyl-N-ethylamino)ethylthio]-1,3,4-oxadiazol-2-yl]-4-methyl-1-oxo-2-pentyl]carboxamide hydrochloride

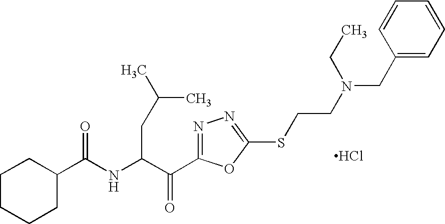

TLC: Rf 0.42 (n-hexane:ethyl acetate=2:1); NMR (DMSO-d₆): δ 8.39 (d, J=6.3 Hz, 1H), 7.63 (br-s, 2H), 7.43 (br-s, 3H), 5.07-4.96 (m, 1H), 4.47-4.32 (m, 2H), 3.88-3.65 (m, 2H), 3.53-3.31 (m, 2H), 3.23-3.04 (m, 2H), 2.27-2.12 (m, 1H), 1.80-1.48 (m, 8H), 1.32-1.14 (m, 8H), 0.91 (d, J=6.3 Hz, 3H), 0.90 (d, J=6.3 Hz, 3H).

### EXAMPLE 9(52)

cycloheptyl-N-[(3S)-1-[5-[2-dimethylaminoethylthio]-1,3,4-oxadiazol-2-yl]-3-methyl-1-oxo-2-pentyl]carboxamide hydrochloride

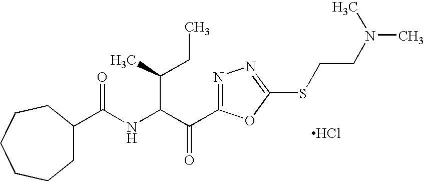

TLC: Rf 0.36 (ethyl acetate:methanol=9:1); NMR (DMSO-d₆): δ 10.92 (brs, 1H), 8.32 and 8.17 (each d, J=6.6 Hz, total 1H), 5.14 and 4.91 (each m, total 1H), 3.78-3.68 (m, 2H), 3.55-3.35 (m, 2H), 2.80 (s, 6H), 2.50-2.40 (m, 1H), 2.20-1.95 (m, 1H), 1.80-1.00 (m, 14H), 0.98-0.78 (m, 6H).

### EXAMPLE 9(53)

cyclohexyl-N-[1-[5-[2-[4-(t-butoxycarbonyl)piperazin-1-yl]ethylthio]-1,3,4-oxadiazol-yl]-3-methyl-1-oxo-2-pentyl]carboxamide hydrochloride

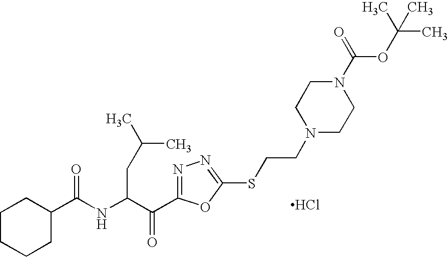

TLC: Rf 0.37 (n-hexane:ethyl acetate=3:2); NMR (DMSO-d₆): δ 11.62-11.42 (br, 1H), 8.39 (d, J=6.3 Hz, 1H), 5.08-4.98 (m, 1H), 4.10-3.82 (m, 2H), 3.80-3.75 (m, 2H), 3.75-2.90 (m, 8H), 2.25-2.15 (m, 1H), 1.80-1.00 (m, 13H), 1.41 (s, 9H), 0.91 and 0.90 (each d, J=6.3 Hz, total 6H).

### EXAMPLE 9(54)

cyclohexyl-N-[1-[5-[2-(N-methyl-N-phenethylamino)ethylthio]-1,3,4-oxadiazol-2-yl]-4-methyl-1-oxo-2-pentyl]carboxamide hydrochloride

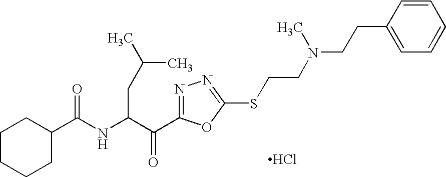

TLC: Rf 0.55 (n-hexane:ethyl acetate=1:1); NMR (DMSO-d₆): δ 11.80-10.80 (br, 1H), 8.40 (d, J=6.0 Hz, 1H), 7.40-7.18 (m, 5H), 5.30-4.95 (m, 1H), 3.84-3.70 (m, 2H), 3.69-3.20 (m, 4H), 3.06 (t, J=8.4 Hz, 2H), 2.88 (s, 3H), 2.28-2.16 (m, 1H), 1.80-1.00 (m, 13H), 0.91 and 0.90 (each d, J=6.3 Hz, total 6H).

### EXAMPLE 9(55)

cyclohexyl-N-[1-[5-[2-[N-(2-methoxyethyl)-N-methylamino]ethylthio]-1,3,4-oxadiazol-2-yl]-4-methyl-1-oxo-2-pentyl]carboxamide hydrochloride

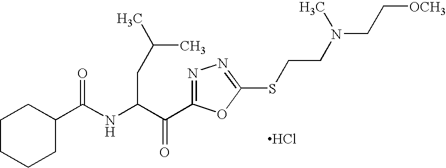

TLC: Rf 0.40 (n-hexane:ethyl acetate=1:5); NMR (DMSO-d₆): δ 10.80-10.60 (br, 1H), 8.40 (d, J=6.3 Hz, 1H), 5.30-4.98 (m, 1H), 3.84-3.66 (m, 5H), 3.65-3.27 (m, 6H), 2.83 (s, 3H), 2.28-2.12 (m, 1H), 1.80-1.00 (m, 13H), 0.91 and 0.90 (each d, J=6.3 Hz, total 6H).

### EXAMPLE 9(56)

cyclohexyl-N-[1-[5-[2-(N-ethoxycarbonylmethyl-N-methylamino)ethylthio]-1,3,4—oxadiazol-2-yl]-4-methyl-1-oxo-2-pentyl]carboxamide hydrochloride

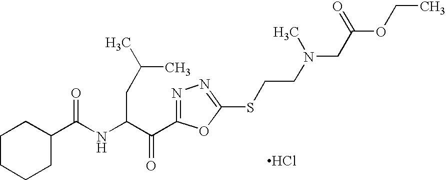

TLC: Rf 0.43 (n-hexane:ethyl acetate=1:1); NMR (DMSO-d₆): δ 11.18-10.65 (br, 1H), 8.41 (d, J=6.3 Hz, 1H), 5.10-4.98 (m, 1H), 4.40-4.18 (m, 4H), 3.80-3.70 (m, 2H), 3.70-3.55 (m, 2H), 2.90 (s, 3H), 2.30-2.16 (m, 1H), 1.80-1.20 (m, 16H), 0.91 and 0.90 (each d, J=6.3 Hz, total 6H).

### EXAMPLE 9(57)

cyclohexyl-N-[1-[5-(1,1-dimethyl-2-dimethylaminoethylthio)-1,3,4-oxadiazol-2-yl]-4-methyl-1-oxo-2-pentyl]carboxamide hydrochloride

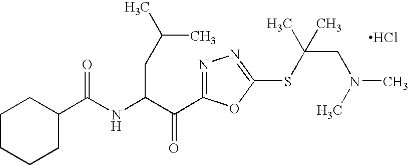

TLC: Rf 0.69 (ethyl acetate); NMR (DMSO-d₆): δ 10.14 (brs, 1H), 8.40 (d, J=6.6 Hz, 1H), 5.08-4.98 (m, 1H), 3.72-3.65 (m, 2H), 2.89 and 2.88 (each s, total 6H), 2.29-2.12 (m, 1H), 1.83-0.98 (m, 13H), 1.68 and 1.67 (each s, total 6H), 0.91 (d, J=6.0 Hz, 6H).

### EXAMPLE 9(58)

cyclohexyl-N-[1-[5-(2,2-dimethyl-2-dimethylaminoethylthio)-1,3,4-oxadiazol-2-yl]-4-methyl-1-oxo-2-pentyl]carboxamide hydrochloride

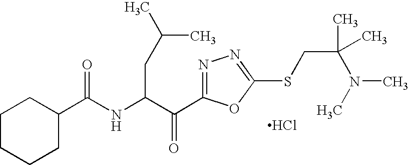

TLC: Rf 0.36 (ethyl acetate); NMR (DMSO-d₆): δ 10.94 (brs, 1H), 8.41 (d, J=6.3 Hz, 1H), 5.03-4.93 (m, 1H), 3.86 (s, 21), 2.76 and 2.74 (each s, total 6H), 2.27-2.12 (m, 1H), 1.81-1.00 (m, 13H), 1.43 (s, 6H), 0.91 and 0.90 (each d, J=6.0 Hz, total 6H).

### EXAMPLE 9(59)

cyclohexyl-N-[(2S)-1-[5-[2-(N-t-butoxycarbonyl-N-methylamino)ethylthio]-1,3,4-oxadiazol-2-yl]-4-methyl-1-oxo-2-pentyl]carboxamide

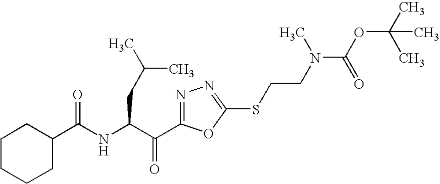

TLC: Rf 0.32 (n-hexane:ethyl acetate=7:3); NMR (DMSO-d₆): δ 8.35 (brd, J=6.3 Hz, 1H), 5.00 (m, 1H), 3.65-3.52 (m, 2H), 3.47 (brt, J=6.3 Hz, 2H), 2.80 (s, 3H), 2.19 (m, 1H), 1.80-1.05 (m, 13H), 1.32 and 1.29 (brs, 9H), 0.91 and 0.90 (each d, J=6.3 Hz, total 6H).

### EXAMPLE 9(60)

cycloheptyl-N-[2-(1-t-butoxycarbonylpiperidin-4-yl)-1-[5-(2-isopropylthio)-1,3,4-oxadiazol-2-yl]-1-oxo-2-ethyl]carboxamide

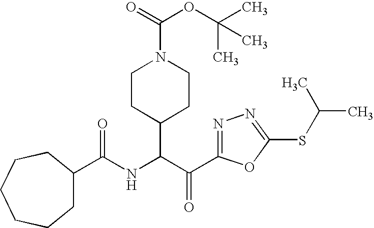

TLC: Rf 0.58 (ethyl acetate:n-hexane=1:1); NMR (CDCl₃): δ 6.14 (brd, J=8.7 Hz, 11H), 5.45 (dd, J=8.7, 5.4 Hz, 1H), 4.12 (m, 2H), 4.05 (sept, J=6.6 Hz, 1H), 2.80-2.50 (m, 2H), 2.30 (m, 1H), 1.93-1.18 (m, 32H).

### EXAMPLE 9(61) cycloheptyl-N-[2-(1-t-butoxycarbonylpiperidin-4-yl)-1-[5-[2-(2-dimethylamino)ethylthio]-1,3,4-oxadiazol-2-yl]-1-oxo-2-ethyl]carboxamide hydrochloride

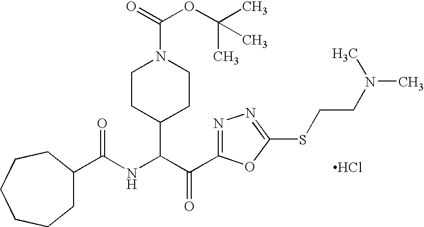

TLC: Rf 0.50 (methanol:ethyl acetate=1:4); NMR (DMSO-d₆): δ 10.48 (br, 1H), 8.38 (brd, J=6.6 Hz, 1H), 4.94 (t, J=5.7 Hz, 1H), 4.02-3.88 (m, 2H), 3.71 (t, J=8.1 Hz, 2H), 3.49 (t, J=8.1 Hz, 2H), 2.81 (m, 6H), 2.80-2.40 (m, 3H), 2.12 (m, 1H), 1.70-1.10 (m, 25H).

### EXAMPLE 9(62) cycloheptyl-N-[2-(1-acetylpiperidin-4-yl)-1-[5-(2-isopropylthio)-1,3,4-oxadiazol-2-yl]-1-oxo-2-ethyl]carboxamide

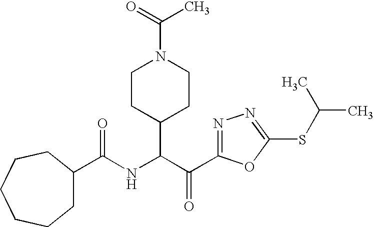

TLC: Rf 0.56 (ethyl acetate:methanol=9:1); NMR (CDCl₃): δ 6.18 (m, 1H), 5.56-5.41 (m, 1H), 4.70-4.59 (m, 1H), 4.05 (sept, J=6.9 Hz, 1H), 3.83 (m, 1H), 3.10-2.90 (m, 1H), 2.60-2.23 (m, 3H), 2.07 (s, 3H), 1.92-1.18 (m, 22H).

### EXAMPLE 9(63)

cycloheptyl-N-[2-(1-benzoylpiperidin-4-yl)-1-[5-(2-isopropylthio)-1,3,4-oxadiazol-2-yl]-1-oxo-2-ethyl]carboxamide

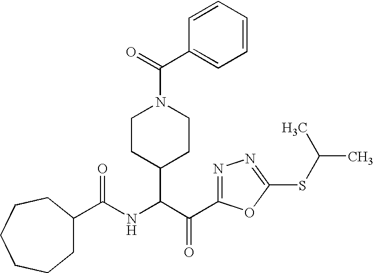

TLC: Rf 0.23 (ethyl acetate:n-hexane=1:1); NMR (CDCl₃): δ 7.45-7.30 (m, 5H), 6.21 (brd, J=8.4 Hz, 1H), 5.45 (m, 1H), 4.72 (m, 1H), 4.06 (sept, J=6.6 Hz, 1H), 3.80 (m, 1H), 3.10-2.50 (m, 2H), 2.50-2.23 (m, 2H), 1.93-1.18 (m, 22H).

### EXAMPLE 9(64)

cycloheptyl-N-[1-[5-(2-isopropylthio)-1,3,4-oxadiazol-2-yl]-2-(1-methoxycarbonylpiperidin-4-yl)-1-oxo-2-ethyl]carboxamide

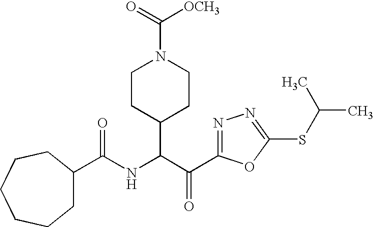

TLC: Rf 0.81 (methanol:ethyl acetate=1:9); NMR (CDCl₃): δ 6.13 (brd, J=8.4 Hz, 1H), 5.45 (dd, J=8.4, 5.4 Hz, 1H), 4.18 (m, 2H), 4.05 (sept, J=6.6 Hz, 1H), 3.67 (s, 3H), 2.80-2.60 (m, 2H), 2.40-2.20 (m, 2H), 1.93-1.18 (m, 22H).

### EXAMPLE 9(65)

cycloheptyl-N-[2-(1-benzylpiperidin-4-yl)-1-[5-(2-isopropylthio)-1,3,4-oxadiazol-2-yl]-1-oxo-2-ethyl]carboxamide hydrochloride

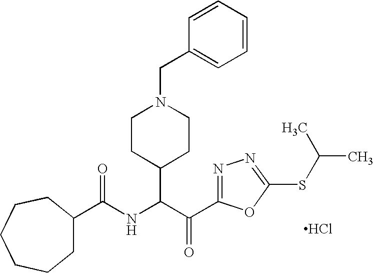

TLC: Rf 0.64 (ethyl acetate:methanol=9:1); NMR (CDCl₃): δ 10.07 (br, 1H), 8.55 (brd, J=6.6 Hz, 1H), 7.60-7.40 (m, 5H), 4.83 (t, J=6.6 Hz, 1H), 4.30-4.10 (m, 2H), 3.94 (sept, J=6.0 Hz, 1H), 3.40-2.78 (m, 4H), 2.25-2.15 (m, 2H), 1.93-1.18 (m, 22H).

### EXAMPLE 9(66)

1-benzoylaminocyclohexyl-N-[(2S)-1-[5-[2-(N-methoxycarbonyl-N-methylamino)ethylthio]-1,3,4-oxadiazol-2-yl]-4-methyl-1-oxo-2-pentyl]carboxamide

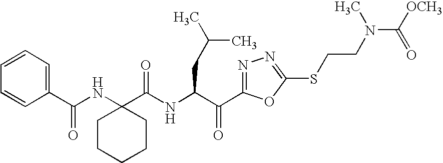

TLC: Rf 0.59 (ethyl acetate:n-hexane=2:1); NMR (CDCl₃): δ 8.08 (brd, J=6.9 Hz, 1H), 7.77 (d, J=6.9 Hz, 2H), 7.60-7.40 (m, 3H), 6.09 (brs, 1H), 5.40-5.29 (m, 1H), 3.69 (s, 3H), 3.69-3.30 (m, 4H), 2.97 (brs, 3H), 2.38-2.20 (m, 2H), 2.05-1.93 (m, 2H), 1.90-1.28 (m, 9H), 1.01 and 0.97 (each d, J=6.0 Hz, each 3H).

### EXAMPLE 9(67)

cyclohexyl-N-[(2S)-1-[5-(2-N-methoxycarbonyl-N-methylamino-2-dimethylethylthio)-1,3,4-oxadiazol-2-yl]-4-methyl-1-oxo-2-pentyl]carboxamide

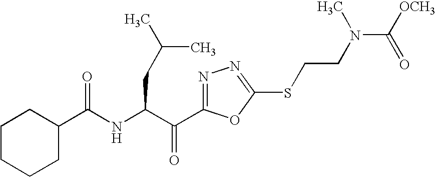

TLC: Rf 0.58 (ethyl acetate:n-hexane=2:1); NMR (CDCl₃): δ 5.98 (brd, J=7.5 Hz, 1M, 5.42 (m, 1H), 3.71 (s, 3H), 3.70-3.40 (m, 2H), 2.99 (brs, 3H), 2.18 (m, 1H), 1.90-1.20 (m, 13H), 1.03 and 0.97 (each d, J=6.0 Hz, each 3H).

### EXAMPLE 9(68)

cycloheptyl-N-[1-[5-(2-dimethylaminoethylthio)-1,3,4-oxadiazol-2-yl]-2-(1-acetylpiperidin-4-yl)-1-oxo-2-ethyl]carboxamide hydrochloride

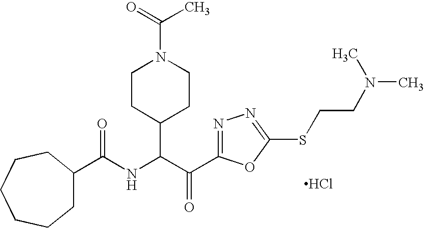

TLC: Rf 0.15 (methanol:ethyl acetate=1:4); NMR (DMSO-d₆): δ 10.58 (brs, 1H), 8.37 (m, 1H), 4.93 (m, 1H), 4.53-4.30 (m, 1H), 3.90-3.80 (m, 1H), 3.78-3.70 (m, 2H), 3.60-3.40 (m, 2H), 2.92 (m, 1H), 2.81 and 2.80 (each s, totally 6H), 2.40-2.30 (m, 1H), 2.30-2.10 (m, 1H), 1.95 (s, 3H), 1.70-1.05 (m, 17H).

### EXAMPLE 9(69)

cyclohexyl-N-[1-[5-(2-dimethylaminoethylthio)-1,3,4-oxadiazol-2-yl]-4-methoxy-4-methyl-1-oxo-2-pentyl]carboxamide hydrochloride

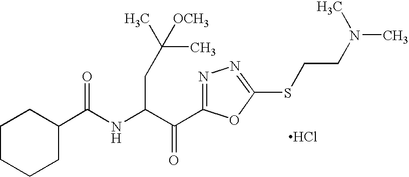

TLC: Rf 0.53 (methylene chloride:methanol=9:1); NMR (DMSO-d₆): δ 10.54 (brs, 1H), 8.38 (d, J=4.8 Hz, 1H), 5.08-4.98 (m, 1H), 3.82-3.64 (m, 2H), 3.57-3.44 (m, 2H), 2.82 (s, 6H), 2.76 (s, 3H), 2.25-2.05 and 1.92-1.78 (each m, total 3H), 1.76-0.93 (m, 10H), 1.21 and 1.08 (each s, total 6H).

### EXAMPLE 10(1)-EXAMPLE 10(4)

By the same procedure as described in example 5 using a corresponding compound, the compounds of the present invention having the following physical data were given.

### EXAMPLE 10(1)

cyclohexyl-N-[(2S)-1-(4-benzyl-5-oxo-1,3,4-oxadiazolin-2-yl)-3-methyl-1-oxo-2-butyl]carboxamide

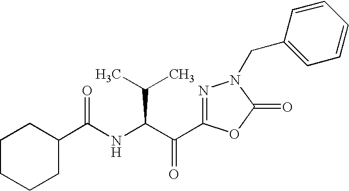

TLC: Rf 0.39 (n-hexane:ethyl acetate=2:1); NMR (DMSO-d₆): δ 8.17 (d, J=6.6 Hz, 1H), 7.41-7.31 (m, 5H), 5.07-4.95 (m, 2H), 4.72 (t-like, J=6.0 Hz, 1H), 2.29-2.11 (m, 2H), 1.72-1.48 (m, 5H), 1.32-1.02 (m, 5H), 0.87 (d, J=6.6 Hz, 3H), 0.84 (d, J=6.6 Hz, 3H).

### EXAMPLE 10(2)

cyclohexyl-N-[(2S)-1-(5-oxo-4-phenethyl-1,3,4-oxadiazolin-2-yl)-3-methyl-1-oxo-2-butyl]carboxamide

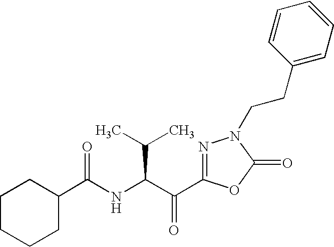

TLC: Rf 0.32 (n-hexane:ethyl acetate=2:1); NMR (CDCl₃): δ 7.34-7.19 (m, 5H), 5.97 (d, J=8.4 Hz, 1H), 5.29 (dd, J=8.4, 5.1 Hz, 1H), 4.194.02 (m, 2H), 3.12 (t, J=7.2 Hz, 2H), 2.23-2.08 (m, 2H), 1.88-1.24 (m, 10H), 0.99 (d, J=6.9 Hz, 3H), 0.85 (d, J=6.9 Hz, 3H).

### EXAMPLE 10(3)

cyclohexyl-N-[(2S)-3-methyl-1-oxo-1-[5-oxo-4-(3-phenylpropyl)-1,3,4-oxadiazolin-2-yl]-2-butyl]carboxamide

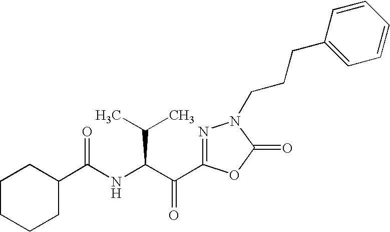

TLC: Rf 0.38 (n-hexane:ethyl acetate=2:1); NMR (CDCl₃): δ 7.32-7.26 (m, 2H), 7.22-7.18 (m, 3H), 5.99 (d, J=7.8 Hz, 1H), 5.32 (dd, J=7.8, 4.8 Hz, 1H), 3.87 (t, J=6.9 Hz, 2H), 2.71 (t, J=7.5 Hz, 2H), 2.32-2.12 (m, 4H), 1.88-1.23 (m, 10H), 1.03 (d, J=6.6 Hz, 3H), 0.88 (d, J=6.6 Hz, 3H).

### EXAMPLE 10(4)

cyclohexyl-N-[1-[4-(3-dimethylaminopropyl)-5-oxo-1,3,4-oxadiazolin-2-yl]-3-methyl-1-oxo-2-butyl]carboxamide hydrochloride

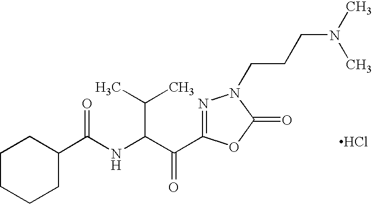

TLC: Rf 0.41 (ethyl acetate:acetic acid:water=3:1:1); NMR (DMSO-d₆): δ 8.20 (d, J=6.9 Hz, 1H), 4.87 (t-like, J=6.6 Hz, 1H), 3.88 (t, J=6.6 Hz, 2H), 3.16-3.09 (m, 2H), 2.72 (s, 3H), 2.70 (s, 3H), 2.37-2.04 (m, 4H), 1.69-1.61 (m, 5H), 1.36-1.03 (m, 5H), 0.91 (d, J=6.6 Hz, 3H), 0.87 (d, J=6.6 Hz, 3H).

### EXAMPLE 11

cyclohexyl-N-[(2S)-3-methyl-1-oxo-1-(5-oxo-4-phenyl-1,3,4-oxadiazolin-2-yl)-2-butyl]carboxamide

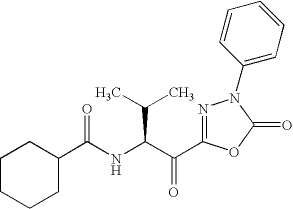

By the same procedure as described in example 4, the compound of the present invention having the following physical data was given.

TLC: Rf 0.59 (n-hexane:ethyl acetate=2:1); NMR (DMSO-d₆): δ 8.24 (d, J=6.9 Hz, 1H), 7.80 (d, J=8.7 Hz, 2H), 7.56 (t-like, J=7.8 Hz, 2H), 7.39 (t-like, J=7.8 Hz, 1H), 4.91 (t-like, J=6.3 Hz, 1H), 2.34-2.25 (m, 2H), 1.69-1.58 (m, 5H), 1.36-1.10 (m, 5H), 0.95 (d, J=6.9 Hz, 3H), 0.91 (d, J=6.9 Hz, 3H).

### EXAMPLE 12(1)-EXAMPLE 12(46)

By the same procedure as described in example 2 using a corresponding compound, the compound of the present invention having the following physical data were given.

### EXAMPLE 12(1)

cyclohexyl-N-[(2S)-1-[5-(2-methylaminoethylthio)-1,3,4-oxadiazol-2-yl]-4-methyl 1-oxo-2-pentyl]carboxamide hydrochloride

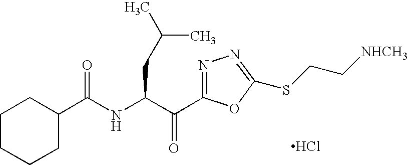

TLC: Rf 0.57 (methylene chloride:methanol=10:1); NMR (DMSO-d₆): δ 9.25-9.10 (br, 2H), 8.40 (d, J=6.3 Hz, 1H), 5.05-4.95 (m, 1H), 3.68-3.62 (m, 2H), 3.38-3.28 (m, 2H), 2.62-2.52 (m, 3H), 2.26-2.14 (m, 1H), 1.82-1.08 (m, 13H), 0.91 and 0.90 (each d, J=6.3 Hz, total 6H).

### EXAMPLE 12(2)

cycloheptyl-N-[1-[5-(2-isopropylthio)-1,3,4-oxadiazol-2-yl]-2-(piperidin-4-yl)-1-oxo-2-ethyl]carboxamide hydrochloride

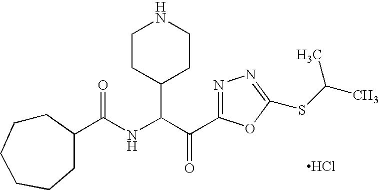

TLC: Rf 0.78 (ethyl acetate:n-hexane=1:1); NMR (DMSO-d₆): δ 8.83 (br, 1H), 8.54 (brd, J=6.9 Hz, 1H), 8.42 (br, 1H), 4.86 (t, J=6.9 Hz, 1H), 3.95 (sept, J=6.6 Hz, 1H), 3.28 (m, 2H), 2.90-2.70 (m, 2H), 2.41-2.15 (m, 2H), 1.80-1.20 (m, 16H).

### EXAMPLE 12(3)

cycloheptyl-N-[1-[5-(2-dimethylaminoethylthio)-1,3,4-oxadiazol-2-yl]-2-(piperidin-4-yl)-1-oxo-2-ethyl]carboxamide bishydrochloride

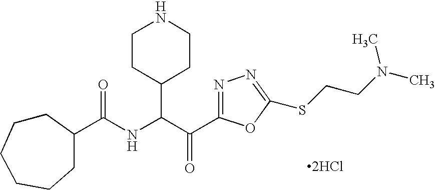

TLC: Rf 0.72 (methanol:methylene chloride:28% ammonia water=1:4:0.1);NMR (DMSO-d₆): δ 10.63 (br, 1H), 8.86 (m, 1H), 8.56 (brd, J=6.6 Hz, 1H), 8.54 (br, 1H), 4.94 (t, J=6.6 Hz, 1H), 3.71 (t, J=7.8 Hz, 2H), 3.49 (t, J=7.8 Hz, 2H), 3.23 (m, 2H), 2.81 (m, 6H), 2.90-2.10 (m, 4H), 1.83-1.30 (m, 16H).

### EXAMPLE 12(4)

cyclohexyl-N-[(2S)-3-methyl-1-[4-(2-methylaminoethyl)-5-oxo-1,3,4-oxadiazolin-2-yl]-1-oxo-2-butyl]carboxamide hydrochloride

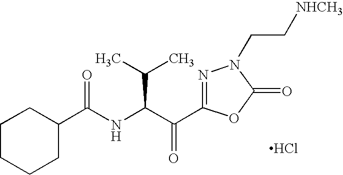

TLC: Rf 0.51 (ethyl acetate: acetic acid:water=3:1:1); NMR (DMSO-d₆): δ 9.02 (br, 2H), 8.18 (d, J=7.2 Hz, 1H), 4.88 (t-like, J=6.6 Hz, 1H), 4.13 (t, J=7.4 Hz, 2H), 3.26 (t, J=7.4 Hz, 2H), 2.58 (s, 3H), 2.37-2.20 (m, 2H), 1.69-1.61 (m, 5H), 1.37-1.03 (m, 5H), 0.92 (d, J=6.9 Hz, 3H), 0.86 (d, J=6.9 Hz, 3H).

### EXAMPLE 12(5)

cyclohexyl-N-[(2S)-3-methyl-1-[5-(2-methylaminoethylthio)-1,3,4-oxadiazol-2-yl]-1-oxo-2-butyl]carboxamide hydrochloride

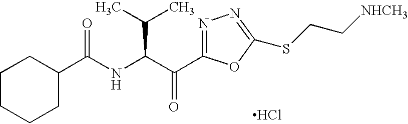

TLC: Rf 0.38 (ethyl acetate:methanol:water=40:10:1); NMR (DMSO-d₆): δ 9.08 (br, 2H), 8.32 (d, J=6.3 Hz, 1H), 4.89 (t, J=6.3 Hz, 1H), 3.65 (t, J=6.9 Hz, 2H), 3.36-3.32 (m, 2H), 2.58 (s, 3H), 2.39-2.16 (m, 2H), 1.67-1.58 (m, 5H), 1.38-1.06 (m, 5H), 0.92 (d, J=6.0 Hz, 3H), 0.90 (d, J=6.0 Hz, 3H).

### EXAMPLE 12(6)

cyclohexyl-N-[(2S)-4-methyl-1-[5-(3-methylaminopropylthio)-1,3,4-oxadiazol-2-yl]1-oxo-2-pentyl]carboxamide hydrochloride

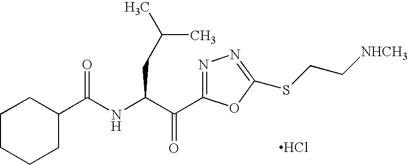

TLC: Rf 0.49 (ethyl acetate:acetic acid:water=3:1:1); NMR (CDCl₃): δ 8.66(brs, 2H), 8.37 (brd, J=6.3 Hz, 1H), 5.09 (m, 1H), 3.42 (t, J=6.9 Hz, 2H), 2.99 (t, J=6.9 Hz, 2H), 2.53 (s, 3H), 2.30-2.05 (m, 3H), 1.80-1.50 (m, 8H), 1.35-1.05 (m, 5H), 0.90 and 0.89 (each d, J=6.3 Hz, each 3H).

### EXAMPLE 12(7) cyclohexyl-N-[1-[5-(2-methylaminoethylthio)-1,3,4-oxadiazol-2-yl]-4,4-dimethyl-1-oxo-2-pentyl]carboxamide hydrochloride

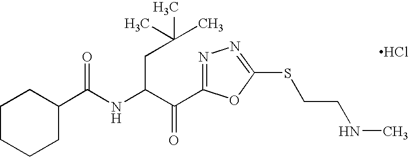

NMR (DMSO-d₆): δ 9.08 (br, 2H), 8.35 (d, J=6.9 Hz, 1H), 5.10 (t, J=7.8 Hz, 1H), 3.64 (t, J=6.6 Hz, 2H), 3.40-3.28 (m, 2H), 2.62-2.53 (m, 3H), 2.25-2.10 (m, 1H), 1.81-1.05 (m, 12H), 0.94 (s, 9H).

### EXAMPLE 12(8)

cyclohexyl-N-[1-[5-(2-methylaminoethylthio)-1,3,4-oxadiazol-2-yl]-4-methoxy-4-methyl-1-oxo-2-pentyl]carboxamide hydrochloride

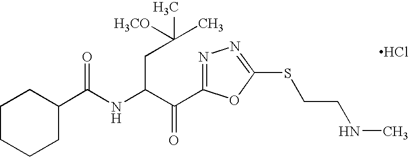

NMR (DMSO-d₆): δ 9.08 (brs, 2H), 8.38 (d, J=4.2 Hz, 1H), 5.08-4.97 (m, 1H), 3.69-3.60 (m, 2H), 3.40-3.28 (m, 2H), 2.75 (s, 3H), 2.62-2.52 (m, 3H), 2.22-2.00 and 1.87-1.77 (each m, 3H), 1.72-0.90 (m, 10H), 1.21 and 1.08 (each s, total 6H).

### EXAMPLE 12(9)

cyclohexyl-N-[1-[3-(2-methylaminoethyl)-2-oxo-1,3,4-oxadiazolin-5-yl]-4,4-dimethyl-1-oxo-2-pentyl]carboxamide hydrochloride

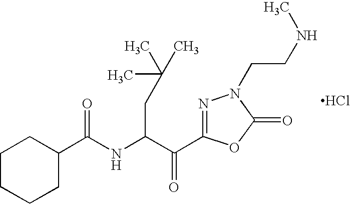

TLC: Rf 0.23 (methylene chloride:methanol=9:1); NMR (DMSO-d₆): δ 9.03 (br, 2H), 8.22 (d, J=6.9 Hz, 1H), 5.08-4.99 (m, 1H), 4.12 (t, J=5.4 Hz, 2H), 3.30-3.12 (m, 2H), 2.57 (brs, 3H), 2.25-2.12 (m, 1H), 1.83-1.05 (m, 12H), 0.93 (s, 9H).

### EXAMPLE 12(10)

cyclohexyl-N-[(2S)-1-[3-(2-methylaminoethyl)-2-oxo-1,3,4-oxadiazolin-5-yl]-4-methyl-1-oxo-2-pentyl]carboxamide hydrochloride

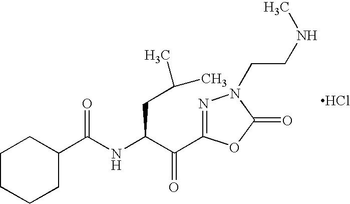

TLC: Rf 0.26 (methylene chloride:methanol=9:1); NMR (DMSO-d₆): δ 9.01 (br, 2H), 8.27 (d, J=6.6 Hz, 1H), 5.01-4.90 (m, 1H), 4.13 (t, J=5.4 Hz, 2H), 3.25 (m, 2H), 2.58 (brs, 3H), 2.28-2.14 (m, 1H), 1.80-1.05 (m, 13H), 0.90 and 0.89 (each d, J=6.0 Hz, total 6H).

### EXAMPLE 12(11)

cyclohexyl-N-[2-cyclopropyl-1-[5-(2-methylaminoethylthio)-1,3,4-oxadiazol-2-yl]-1-oxo-2-ethyl]carboxamide hydrochloride

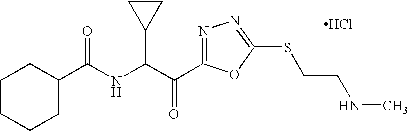

NMR (DMSO-d₆): δ 9.13 (br, 2H), 8.68 (d, J=4.8 Hz, 1H), 4.24 (dd, J=9.3, 4.8 Hz, 1H), 3.66 (t, J=6.9 Hz, 2H), 3.35 (t, J=6.0 Hz, 2H), 2.58 (t, J=5.4 Hz, 3H), 2.30-2.17 (m, 1H), 1.78-1.00 (m, 11), 0.62-0.35 (m, 4H).

### EXAMPLE 12(12)

cyclohexyl-N-[(2S)-3-methyl-1-[3-(2-methylaminoethyl)-2-oxo-1,3,4-oxadiazolin-5-yl]-1-oxo-2-butyl]carboxamide hydrochloride

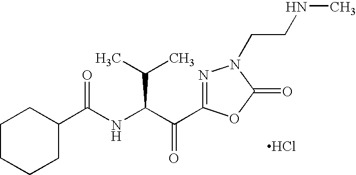

TLC: Rf 0.51 (ethyl acetate:acetic acid:water=3:1:1); NMR (DMSO-d₆): δ 8.93 (br, 1H), 8.28 (d, J=7.2 Hz, 1H), 4.88 (t-like, J=6.9 Hz, 1H), 4.13 (t, J=5.7 Hz, 2H), 3.26 (t, J=5.7 Hz, 2H), 2.59 (s, 3H), 2.38-2.19 (m, 2H), 1.69-1.61 (m, 5H), 1.37-1.08 (m, 5H), 1.03-0.85 (m, 6H).

### EXAMPLE 12(13)

cyclohexyl-N-[(2S)-1-[5-(2-methylaminoethylthio)-1,3,4-oxadiazol-2-yl]-1-oxo-3-phenyl-2-propyl]carboxamide hydrochloride

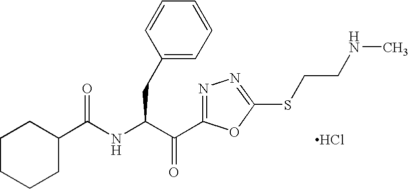

NMR (DMSO-d₆): δ 9.06 (br, 2H), 8.53 (d, J=6.6 Hz, 1H), 7.31-7.20 (m, 5H), 5.22-5.14 (m, 1H), 3.66-3.20 (m, 4H), 3.02-2.77 (m, 2H), 2.60-2.56 (m, 3H), 2.23-2.06 (m, 1H), 1.77-1.01 (m, 10H).

### EXAMPLE 12(14)

cyclohexyl-N-[(2S)-1-[3-(2-hydroxyethyl)-2-oxo-1,3,4-oxadiazolin-5-yl]-3-methyl-1-oxo-2-butyl]carboxamide

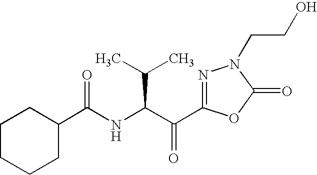

TLC: Rf 0.45 (n-hexane:ethyl acetate=1:3); NMR (DMSO-d₆): δ 8.16 (d, J=7.2 Hz, 1H), 4.94 (t, J=5.7 Hz, 1H), 4.83 (t-like, J=6.6 Hz, 1H), 3.81 (t, J=5.1 Hz, 2H), 3.69-3.63 (m, 2H), 2.34-2.13 (m, 2H), 1.69-1.61 (m, 5H), 1.36-1.05 (m, 5H), 0.90 (d, J=6.9 Hz, 31), 0.86 (d, J=6.6 Hz, 3H).

### EXAMPLE 12(15)

cyclohexyl-N-[(2S)-3,3-dimethyl-1-[5-(2-methylaminoethylthio)-1,3,4-oxadiazol-2-yl]-1-oxo-2-butyl]carboxamide hydrochloride

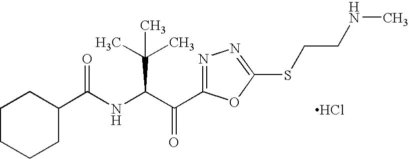

NMR (DMSO-d₆): δ 8.15 and 7.96 (each br-s, total 3H), 7.15 and 7.03 (each d, J=5.7 Hz, total 1H), 5.10 and 5.02 (d and m, J=5.7 Hz, total 1H), 3.27 (s, 11H), 1.01 and 0.96 (each s, total 9H).

### EXAMPLE 12(16)

cyclohexyl-N-[(2S)-1-[5-(2-methylaminoethylthio)-1,3,4-oxadiazol-2-yl]-1-oxo-2-hexyl]carboxamide hydrochloride

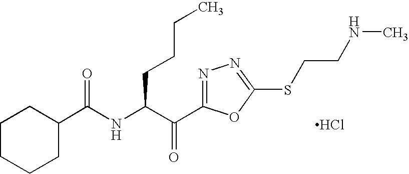

NMR (DMSO-d₆): δ 8.27 and 8.14 (each br-s, total 3H), 7.13 and 6.95 (each m, total 1H), 4.96 and 4.86 (each m, total 1H), 3.39 (br-s, 1H), 1.72-1.50 (m, 2H), 1.46-1.15 (m, 4H), 0.85 (t, J=6.6 Hz, 3H).

### EXAMPLE 12(17)

cyclohexyl-N-[2-methyl-1-[5-(2-methylaminoethylthio)-1,3,4-oxadiazol-2-yl]-1-oxo-2-propyl]carboxamide hydrochloride

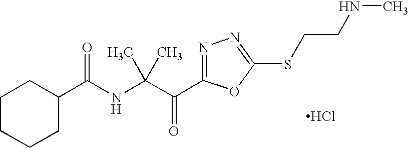

NR (DMSO-d₆): δ 9.16 (br, 2H), 8.87 (s, 1H), 3.63 (t, J=6.6 Hz, 2H), 3.36-3.25 (m, 2H), 2.58-2.54 (m, 3H), 2.17-2.05 (m, 1H), 1.64-1.36 (m, 5H), 1.42 (s, 6H), 1.25-1.01 (m, 5H).

### EXAMPLE 12(18)

cyclohexyl-N-[(2S)-1-[5-(2-ethylaminoethylthio)-1,3,4-oxadiazol-2-yl]-4-methyl-1-oxo-2-pentyl]carboxamide hydrochloride

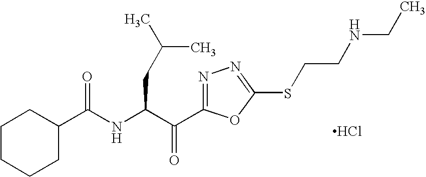

NR (DMSO-d₆): δ 9.01 (br, 2H), 8.40 (d, J=6.0 Hz, 1H), 5.05-4.98 (m, 1H), 3.64 (t, J=6.9 Hz, 2H), 3.40-3.28 (m, 2H), 3.06-2.91 (m, 2H), 2.27-2.12 (m, 1H), 1.79-1.50 (m, 8H), 1.35-1.07 (m, 8H), 0.91 (d, J=6.3 Hz, 3H), 0.90 (d, J=6.3 Hz, 3H).

### EXAMPLE 12(19)

cyclohexyl-N-[(2S)-1-[5-(2-benzylaminoethylthio)-1,3,4-oxadiazol-2-yl]-4-methyl-1-oxo-2-pentyl]carboxamide hydrochloride

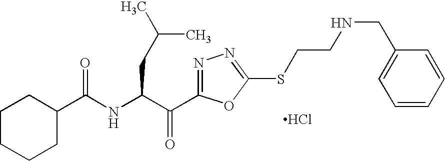

NMR (DMSO-d₆): δ 9.61 (br, 2H), 8.40 (d, J=6.3 Hz, 1H1), 7.57-7.54 (m, 2H), 7.43-7.36 (m, 3H), 5.06-4.97 (m, 1H), 4.19 (s, 2H),3.70 (t, J=6.6 Hz, 211), 3.46-3.32 (m, 2H), 2.28-2.12 (m, 1H), 1.80-1.48 (m, 8H), 1.37-1.05 (m, 5SH), 0.91 (d, J=6.3 Hz, 311), 0.90 (d, J=6.3 Hz, 3H).

### EXAMPLE 12(20)

cyclohexyl-N-[(2S)-1-[5-[2-(1-methylethylamino)ethylthio]-1,3,4-oxadiazol-2-yl]-4-methyl-1-oxo-2-pentyl]carboxamide hydrochloride

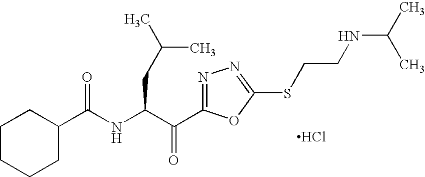

NMR (DMSO-d₆): δ 8.95 (br, 2H), 8.41 (d, J=6.3 Hz, 1H), 5.04-4.97 (m, 1H), 3.63 (t, J=6.9 Hz, 2H), 3.53 (br-m, 3H), 2.26-2.13 (m, 1H), 1.80-1.49 (m, 8H), 1.36-1.07 (m, 11H), 0.91 (d, J=6.6 Hz, 3H), 0.90 (d, J=6.6 Hz, 3H).

### EXAMPLE 12(21)

cyclohexyl-N-[1-[5-(2-methylaminoethylthio)-1,3,4-oxadiazol-2-yl]-1-oxo-3-propyl-2-hexyl]carboxamide hydrochloride

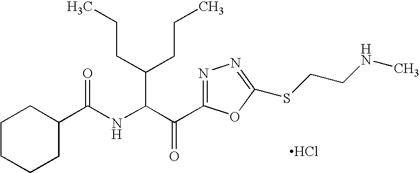

NMR (DMSO-d₆): δ 9.10 (br-m, 1H), 8.13 (d, J=7.2 Hz, 1H), 5.26-5.21 (m, 1H), 3.65 (t, J=6.6 Hz, 2H), 3.35 (t, J=6.6 Hz, 2H), 2.59-2.56 (m, 3H), 2.40-2.27 (m, 1H), 2.17-2.03 (m, 1H), 1.68-1.63 (m, 5H), 1.30-1.14 (m, 13H), 0.86 (t, J=6.3 Hz, 3H), 0.90 (t, J=6.3 Hz, 3H).

### EXAMPLE 12(22)

cyclohexyl-N-[1-[5-(2-benzylaminoethylthio)-1,3,4-oxadiazol-2-yl]-2-(tetrahydropyran-4-yl)-1-oxo-2-ethyl]carboxamide hydrochloride

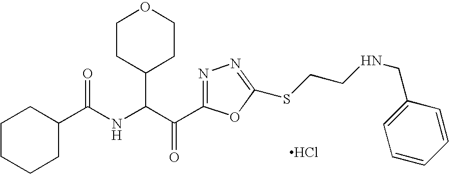

NMR (DMSO-d₆): δ 9.51 (br-m, 2H), 8.43 (d, J=6.6 Hz, 1H), 7.56-7.53 (m, 2H), 7.45-7.36 (m, 3H), 4.91 (t-like, J=6.6 Hz, 1H), 4.20 (m, 2H),3.83 (t, J=8.4 Hz, 2H), 3.70 (t, J=6.6 Hz, 2H), 3.47-3.35 (m, 2H), 3.23 (t, J=8.7 Hz, 2H), 2.37-2.08 (m, 2H), 1.67-1.14 (m, 14H).

### EXAMPLE 12(23)

cyclohexyl-N-[1-[5-(2-methylaminoethylthio)-1,3,4-oxadiazol-2-yl]-2-(4-trifluoromethylphenyl)-1-oxo-2-ethyl]carboxamide hydrochloride

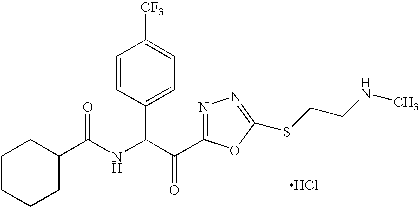

NMR (DMSO-d₆): δ 9.06 (d, J=5.7 Hz, 1H), 8.98 (br-m, 2H), 7.78 (d, J=8.4 Hz, 2H), 7.65 (d, J=8.4 Hz, 2H), 6.27 (d, J=5.7 Hz, 1H), 3.62 (t, J=6.9 Hz, 2H), 3.20 (t, J=6.9 Hz, 2H), 2.58-2.55 (m, 3H), 2.37-2.22 (m, 1H), 1.83-1.57 (m, 5H), 1.40-1.03 (m, 5H).

### EXAMPLE 12(24)

cyclohexyl-N-[1-[5-(2-methylaminoethylthio)-1,3,4-oxadiazol-2-yl]-2-(2-methylphenyl)-1-oxo-2-ethyl]carboxamide hydrochloride

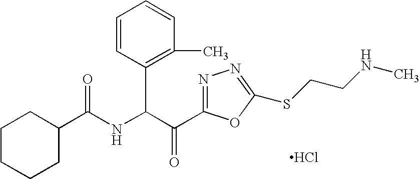

NMR (DMSO-d₆): δ 8.97 (br-m, 2H), 8.70 (d, J=6.3 Hz, 1H), 7.27-7.11 (m, 4H), 6.41 (d, J=6.3 Hz, 1H), 3.61 (t, J=7.2 Hz, 2H), 3.37-3.25 (m, 2H), 2.88-2.52 (m, 3H), 2.47 (s, 3H), 2.35-2.23 (m, 1H), 1.80-1.54 (m, 5H), 1.40-1.07 (m, 5H).

### EXAMPLE 12(25)

cyclohexyl-N-[1-[5-(2-propylaminoethylthio)-1,3,4-oxadiazol-2-yl]-2-(tetrahydropyran-4-yl)-1-oxo-2-ethyl]carboxamide hydrochloride

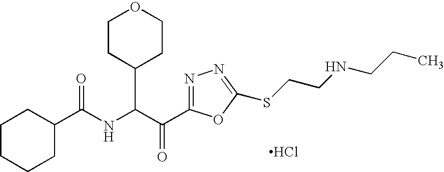

NMR (DMSO-d₆): δ 9.06 (br-m, 2H), 8.43 (d, J=6.3 Hz, 1H), 4.90 (t-like, J=6.3 Hz, 1H), 3.88-3.79 (m, 2H), 3.65-(t, J=6.9 Hz, 2H), 3.42-3.33 (m, 2H), 3.27-3.20 (m, 2H), 2.96——2.85 (m, 2H), 2.35-2.10 (m, 2H), 1.68-1.05 (m, 16H), 0.91(t, J=7.5 Hz, 3H).

### EXAMPLE 12(26)

cyclohexyl-N-[(2S)-1-[5-(2-propylaminoethylthio)-1,3,4-oxadiazol-2-yl]-3-methyl-1-oxo-2-butyl]carboxamide hydrochloride

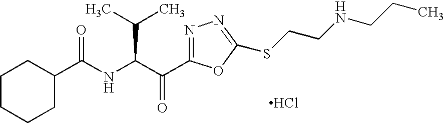

NMR (DMSO-d₆): δ 9.12 (br-m, 2H), 8.33 (d, J=6.3 Hz, 1H), 4.88 (t-like, J=6.3 Hz, 1H), 3.66 (t, J=6.9 Hz, 2H), 3.41-3.35 (m, 2H), 2.95-2.83 (m, 2H), 2.35-2.17 (m, 2H), 1.66-1.59 (m, 7H), 1.28-1.14 (m, 5H), 0.93-0.88 (m, 9H).

### EXAMPLE 12(27)

cyclohexyl-N-[(2S)-1-[5-(2-benzylaminoethylthio)-1,3,4-oxadiazol-2-yl]-3-methyl-1-oxo-2-butyl]carboxamide hydrochloride

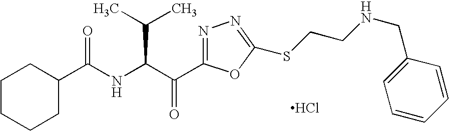

NMR (DMSO-d₆): δ 9.53 (br-m, 2H), 8.32 (d, J=6.3 Hz, 1H), 7.56-7.53 (m, 2H), 7.45-7.36 (m, 3H), 4.89 (t-like, J=6.3 Hz, 1H), 4.20 (m, 2H), 3.70 (t, J=6.9 Hz, 2H), 3.46-3.33 (m, 2H), 2.37-2.17 (m, 2H), 1.77-1.54 (m, 5H), 1.39-1.07 (m, 5H), 0.92 (d, J=6.6 Hz, 3H), 0.91 (d, J=6.6 Hz, 3H).

### EXAMPLE 12(28)

cyclohexyl-N-[(2S,3 S)-1-[5-(2-methylaminoethylthio)-1,3,4-oxadiazol-2-yl]-3-methyl-1-oxo-2-pentyl]carboxamide hydrochloride

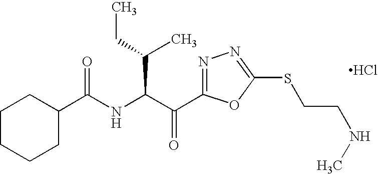

NMR(DMSO-d₆): δ 9.07 (brs, 2H), 8.34 (d, J=6.3 Hz, 1H), 4.92 (t, J=6.3 Hz, 1H), 3.65 (t, J=6.9 Hz, 2H), 3.35 (m, 2H), 2.58 (m, 3H), 2.28 (m, 1H), 2.00 (m, 1H), 1.70-1.00 m, 12H), 0.90-0.80 (m, 6H).

### EXAMPLE 12(29)

cycloheptyl-N-[1-[5-(2-methylaminoethylthio)-1,3,4-oxadiazol-2-yl]-4,4-dimethyl-1-oxo-2-pentyl]carboxamide hydrochloride

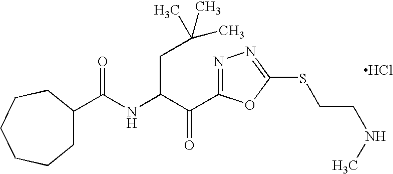

NMR(DMSO-d₆): δ 8.95 (brs, 2H), 8.33 (brd, J=6.6 Hz, 1H), 5.08 (m, 1H), 3.64 (m, 2H), 3.40 (m, 2H), 2.59 (brs, 3H), 2.35 (m, 1H), 1.80-1.30 (m, 14H), 0.94 (s, 9H).

### EXAMPLE 12(30)

cyclohexyl-N-[2-cyclopentyl-1-[5-(2-methylaminoethylthio)-1,3,4-oxadiazol-2-yl]-1-oxo-2-ethyl]carboxamide hydrochloride

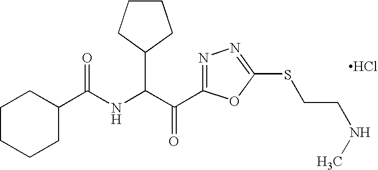

NMR (DMSO-d₆): δ 0.96-1.88 (m, 18H), 2.15-2.39 (m, 2H), 2.57 (s, 3H), 3.34 (m, 2H), 3.65 (t, J=6.9 Hz, 2H), 4.85 (m, 1H), 8.44 (d, J=6.0 Hz, 1H), 9.15 (brs, 2H).

### EXAMPLE 12(31)

cyclohexyl-N-[1-[5-(2-methylaminoethylthio)-1,3,4-oxadiazol-2-yl]-2-(piperidin-4-yl)-1-oxo-2-ethyl]carboxamide bishydrochloride

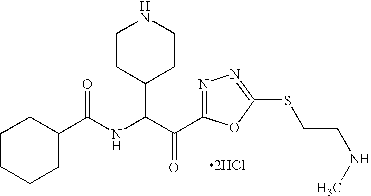

NMR (DMSO-d₆): δ 0.99-1.89 (m, 14H), 2.11-2.36 (m, 2H), 2.58 (m, 3H), 2.68-2.94 (m, 2H), 3.13-3.40 (m, 4H), 3.65 (t, J=6.7 Hz, 2H), 4.90 (m, 1H), 8.47-8.76 (broad, 2H), 8.85-9.32 (m, 3H).

### EXAMPLE 12(32)

cyclohexyl-N-[2-cyclohexyl-1-[5-(2-methylaminoethylthio)-1,3,4-oxadiazol-2-yl]-1-oxo-2-ethyl]carboxamide hydrochloride

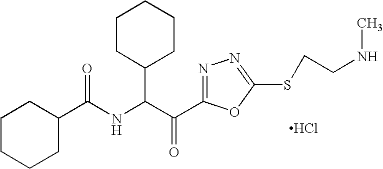

NMR (DMSO-d₆): δ 9.05 (br, 2H), 8.32 (brd, J=6.0 Hz, 1H), 4.88 (t, J=6.0 Hz, 1H), 3.63 (t, J=6.3 Hz, 2H), 3.33 (m, 2H), 2.57 (m, 3H), 2.28 (m, 1H), 2.00-1.40 (m, 10H), 1.40-1.00 (m, 11H).

### EXAMPLE 12(33)

cyclohexyl-N-[1-[5-(2-methylaminoethylthio)-1,3,4-oxadiazol-2-yl]-2-phenyl-1-oxo-2-ethyl]carboxamide hydrochloride

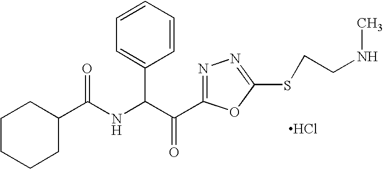

NMR (DMSO-d₆): δ 8.87 (brd, J=5.4 Hz, 1H), 8.90-8.75 (br, 2H), 7.50-7.30 (m, 5H), 6.16 (d, J=5.4 Hz, 1H), 3.60 (t, J=6.6 Hz, 2H), 3.33 (m, 2H), 2.57 (m, 3H), 2.30 (m, 1H), 1.80-1.53 (m, 5H), 1.40-1.03 (m, 5H).

### EXAMPLE 12(34)

cyclohexyl-N-[2-(4-methoxyphenyl)-1-[5-(2-methylaminoethylthio)-1,3,4-oxadiazol-2-yl]-1-oxo-2-ethyl]carboxamide hydrochloride

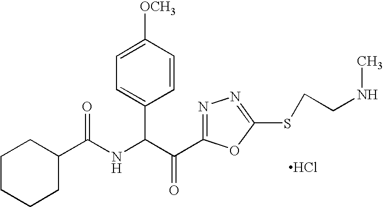

NMR (DMSO-d₆): δ 8.91 (br, 2H), 8.78 (brd, J=5.4 Hz, 1H), 7.35 (d, J=8.7 Hz, 2H), 6.94 (d, J=8.7 Hz, 2H), 6.09 (d, J=5.4 Hz, 1H), 3.71 (s, 3H), 3.60 (t, J=6.0 Hz, 2H), 3.33 (m, 2H), 2.56 (m, 3H), 2.33 (m, 1H), 1.80-1.55 (m, 5H), 1.40-1.03 (m, 5H).

### EXAMPLE 12(35)

cycloheptyl-N-[(2S)-4,4-dimethyl-1-[5-(2-methylaminoethylthio)-1,3,4-oxadiazol-2-yl]-1-oxo-2-pentyl]carboxamide hydrochloride

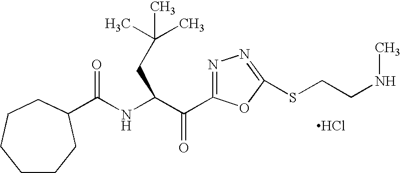

NMR(DMSO-d₆): δ 8.98 (br, 2H), 8.34 (d, J=6.9 Hz, 1H), 5.15-5.02 (m, 1H), 3.70-3.60 (m, 2H), 3.40-3.30 (m, 2H), 2.58 (s, 3H), 2.40-2.30 (m, 1H), 1.80-1.30 (m, 14H), 0.94 (s, 9H).

### EXAMPLE 12(36)

N-[(2S)-1-[5-(2-methylaminoethylthio)-1,3,4-oxadiazol-2-yl]-4-methyl-1-oxo-2-pentyl]acetamide hydrochloride

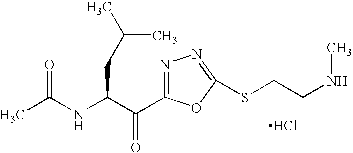

NMR (DMSO-d₆): δ 9.17 (br, 2H), 8.55 (d, J=6.0 Hz, 1H), 5.15-5.00 (m, 1H), 3.65 (t, J=6.6 Hz, 1H), 3.42-3.28 (m, 2H), 2.57 (s, 3H), 1.85 (s, 3H), 1.80-1.40 (m, 3H), 1.00-0.80 (m, 6H).

### EXAMPLE 12(37)

15 (tetrahydropyran-4-yl)-N-[(2S)-1-[5-(2-methylaminoethylthio)-1,3,4-oxadiazol-2-yl]-4-methyl-1-oxo-2-pentyl]carboxamide hydrochloride

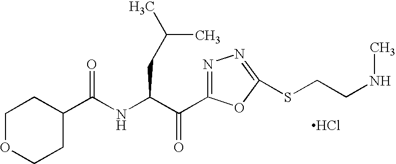

NMR (DMSO-d₆): δ 9.17 (br, 2H), 8.52 (d, J=6.0 Hz, 1H), 5.15-5.00 (m, 1H), 3.90-3.75 (m, 2H), 3.74-3.60 (m, 2H), 3.40-3.20 (m, 4H), 2.57 (s, 3H), 1.80-1.40 (m, 7H), 0.98-0.80 (m, 6H).

### EXAMPLE 12(38)

t-butyl-N-[(2S)-4-methyl-1-[5-(2-methylaminoethylthio)-1,3,4-oxadiazol-2-yl]-1-oxo-2-pentyl]carboxamide hydrochloride

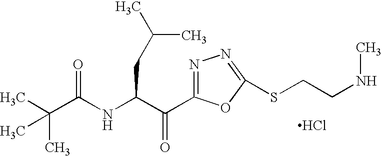

NMR(DMSO-d₆): δ 9.12 (br, 2H), 8.07 (d, J=6.3 Hz, 1H), 5.16-4.90 (m, 1H), 3.65 (t, J=6.6 Hz, 2H), 3.42-3.28 (m, 2H), 2.58 (s, 3H), 1.80-1.50 (m, 3H), 1.08 (s, 9H), 1.00-0.89 (m, 6H).

### EXAMPLE 12(39)

N-[(2S)-4-methyl-1-[5-(2-methylaminoethylthio)-1,3,4-oxadiazol-2-yl]-1-oxo-2-pentyl]benzamide hydrochloride

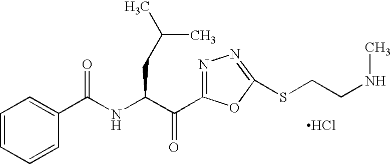

NMR (DMSO-d₆): δ 9.30-9.10 (br, 2H), 9.06 (d, J=6.6 Hz, 1H), 7.89-7.42 (m, 5H), 5.38-5.25 (m, 1H), 3.66 (t, J=6.9 Hz, 2H), 3.42-3.22 (m, 2H), 2.58 (s, 3H), 1.90-1.70 (m, 3H), 1.10-0.80 (m, 6H).

### EXAMPLE 12(40)

cycloheptyl-N-[(2S)-4-methyl-1-[5-(2-methylaminoethylthio)-1,3,4-oxadiazol-2-yl]-1-oxo-2-pentyl]carboxamide hydrochloride

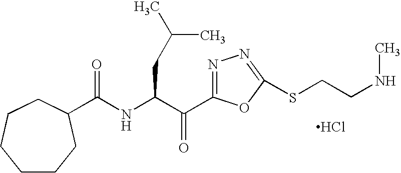

NMR (DMSO-d₆): δ 9.17 (br, 2H), 8.40 (d, J=6.3 Hz, 1H), 5.10-4.90 (m, 1H), 3.65 (t, J=6.0 Hz, 2H), 3.42-3.24 (m, 2H), 2.57 (s, 3H), 1.80-1.22 (m, 15H), 1.00-0.78 (m, 6H).

### EXAMPLE 12(41)

N-[(2S)-1-4-methyl-[5-(2-methylaminoethylthio)-1,3,4-oxadiazol-2-yl]-1-oxo-2-pentyl]phenylacetamide hydrochloride

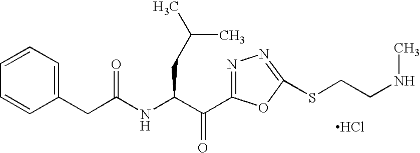

NMR (DMSO-d₆): δ 8.95 (br, 2H), 8.83 (d, J=6.6 Hz, 1H), 7.38-7.18 (m, 5H), 5.18-5.02 (m, 1H), 3.62 (t, J=6.9 Hz, 2H), 3.40-3.30 (m, 2H), 2.58 (s, 3H), 1.80-1.55 (m, 3H), 1.00-0.80 (m, 6H).

### EXAMPLE 12(42)

N-[(2S)-4-methyl-1-[5-(2-methylaminoethylthio)-1,3,4-oxadiazol-2-yl]-1-oxo-2-pentyl]-phenoxyacetamide hydrochloride

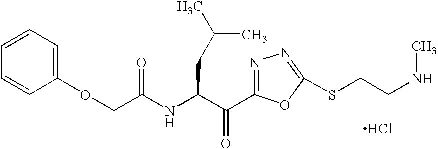

NMR (DMSO-d₆): δ 9.16-8.82 (br, 2H), 8.83 (d, J=6.6 Hz, 1H), 7.35-7.18 (m, 5H), 5.18-5.02 (m, 1H), 3.65-3.60 (m, 2H), 3.49 (s, 2H), 3.40-3.30 (m, 2H), 2.58 (s, 3H), 1.80-1.55 (m, 3H), 1.00-0.80 (m, 6H).

### EXAMPLE 12(43)

N-[(2S)-4-methyl-1-[5-(2-methylaminoethylthio)-1,3,4-oxadiazol-2-yl]-1-oxo-2-pentyl]-4,4-dimethyl-2-pentenamide hydrochloride

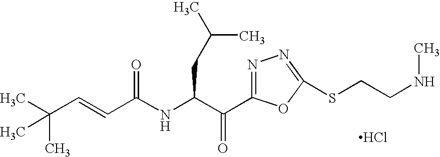

NMR (DMSO-d₆): δ 9.22-9.00(br, 2H), 8.60 (d, J=6.3 Hz, 1H), 6.62 (d, J=15.6 Hz, 1H), 6.93 (d, J=15.6 Hz, 1H), 5.20-5.10 (m, 1H), 3.70-3.60 (m, 2H), 3.40-3.23 (m, 2H), 2.58 (s, 3H), 1.80-1.10 (m, 3H), 1.02 (s, 9H), 0.90-0.80 (m, 6H).

### EXAMPLE 12(44)

N-[(2S)-4-methyl-1-[5-(2-methylaminoethylthio)-1,3,4-oxadiazol-2-yl]-1-oxo-2-pentyl]-4,4-dimethylpentanamide hydrochloride

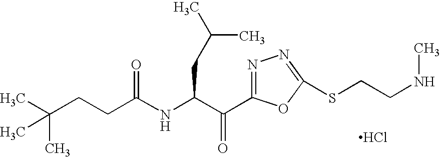

NMR (DMSO-d₆): δ 0.83 (s, 9H), 0.91 (m, 6H), 1.34 (m, 2H), 1.61 (m, 3H), 2.10 (m, 2H), 2.57 (m, 3H), 3.32 (m, 2H), 3.65 (t, J=6.87 Hz, 2H), 5.04 (m, 1H), 8.52 (d, J=6.32 Hz, 1H), 9.15 (br, 2H).

### EXAMPLE 12(45)

N-(2-methylpropyloxycarbonyl)-N-[(2S)-4-methyl-1-[5-(2-methylaminoethylthio)-1,3,4-oxadiazol-2-yl]-1-oxo-2-pentyl]amine hydrochloride

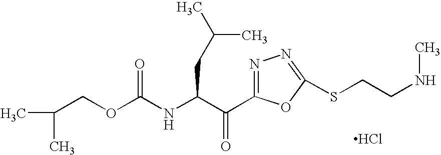

NMR (DMSO-d₆): δ 9.22-9.00(br, 2H), 7.82 (d, J=7.5 Hz, 1H), 5.02-4.90 (m, 1H), 3.72 (d, J=6.3 Hz, 2H), 3.70-3.50 (m, 4H), 2.58 (s, 3H), 1.80-1.40 (m, 4H), 0.90-0.80 (m, 12H).

### EXAMPLE 12(46)

cyclohexyl-N-[3-ethyl-1-[5-(2-methylaminoethylthio)-1,3,4-oxadiazol-2-yl]-1-oxo-2-pentyl]carboxamide hydrochloride

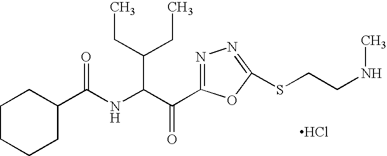

NMR (DMSO-d₆): δ 8.98 (br, 2H), 8.16 (d, J=6.6 Hz, 1H), 5.25-5.22 (m, 1H), 3.70-3.50 (m, 2H), 3.40-3.28 (m, 2H), 2.58 (s, 3H), 1.80-1.00 (m, 16H), 0.90-0.70 (m, 6H).

### FORMULATION EXAMPLE

### FORMULATION EXAMPLE 1

The following components were admixed in a conventional method, dried, and punched out to give 100 tablets each containing 50 mg of active ingredient.

| | |
|---|---|
| Cycloheptyl-N-[1-[5-(2-dimethylaminoethylthio)- | 5.0 g |
| 1,3,4-oxadiazol-2-yl]-4-methyl-1- | |
| oxo-2-pentyl]carboxamide hydrochloride | |
| Carboxymethylcellulose calcium (disintegrating agent) | 0.2 g |
| Magnesium stearate(lubricating agent) | 0.1 g |
| Microcrystalline cellulose | 4.7 g |

### FORMULATION EXAMPLE 2

The following components were admixed in a conventional method. The solution was sterilized in conventional method, placed 2 ml portions into 5 ml ampoules and freeze-dried to give 100 ampoules each containing 20 mg of the active ingredient.

| | |
|---|---|
| Cycloheptyl-N-[1-[5-(2-dimethylaminoethylthio)- | 2.0 g |
| 1,3,4-oxadiazol-2-yl]-4-methyl-1- | |
| oxo-2-pentyl]carboxamide hydrochloride | |
| Mannitol | 20 g |
| Distilled water | 500 ml |

1. An oxadiazole derivative of formula (I), 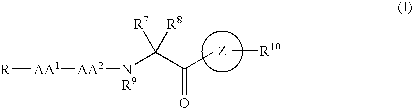
wherein R is
(i) hydrogen,
(ii) C1-8 alkyl,
(iii) CycA,
(iv) C1-8 alkyl substituted with a group selected from halo, CycA, nitro, CF₃ and cyano, 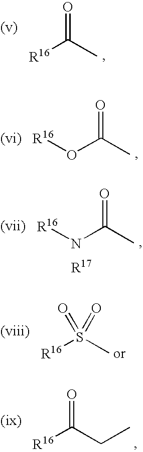
CycA is a mono-, bi- or tri-cyclic C3-15 carboring, or a mono-, bi- or tri-cyclic 3-15 membered heteroring comprising 1-4 of nitrogen, 1-2 of oxygen and/or 1 of sulfur,
R¹⁶ is
(1) C1-8 alkyl,
(2) C2-8 alkenyl,
(3) C2-8 alkynyl,
(4) CycA, or
(5) C1-8 alkyl, C2-8 alkenyl or C2-8 alkynyl substituted with 1-5 of halogen, nitro, trifluoromethyl, cyano, CycA, NR¹⁸R¹⁹, —OR¹⁸, —NHC(O) —CycA and —NHC(O)O—(C1-8 alkyl),
R¹⁷, R¹⁸ and R¹⁹ are each independently, hydrogen, C1-4 alkyl, CycA or C1-4 alkyl substituted with C1-4 alkyl,
AA¹ is
(i) a single bond, or 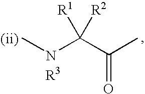
wherein R¹ and R² are the same or different to represent
(i) hydrogen,
(ii) C1-8 alkyl,
(iii) CycA or
(iv) C1-8 alkyl substituted with 1-5 of group selected from the following (1) to (8):
(1) —NR²¹R²²,
(2)—OR²³,
(3) —SR²⁴,
(4) —COR²⁵,
(5) —NR²⁶CONR²¹R²²,
(6) guanidino,
(7) CycA,
(8) NR²⁶SO₂R²¹; or
R¹ and R² are taken together to form C2-8 alkylene wherein one carbon atom may be replaced by oxygen, sulfur or —NR²⁰— and the alkylene may be substituted with —NR²¹R²² or —OR²³,
R²⁰ is hydrogen, C1-4 alkyl, —COO—(C1-4 alkyl), phenyl or C1-4 alkyl substituted with phenyl,
R²¹, R²², R²³, R²⁴ and R²⁶ are the same or different to represent hydrogen, C1-4 alkyl, phenyl or C1-4 alkyl substituted with phenyl,
R²⁵ is C1-4 alkyl, phenyl, —NR², R²² wherein all symbols have the same meaning as above, —OR²³ wherein R¹³ is the same meaning as above, or C1-4 alkyl substituted with phenyl,
R³ is hydrogen, C1-8 alkyl, phenyl or C1-8 alkyl substituted with phenyl or
R³ is taken together with R¹ to form C2-6 alkylene wherein one carbon atom may be replaced by oxygen, sulfur or —NR²⁰— and the alkylene may be substituted with —NR²¹R²² or —OR²³, or
when AA¹ is 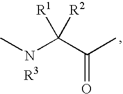
AA¹ and R may be taken together to form 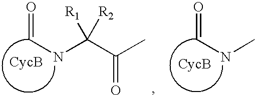
wherein
is a 5-12 membered mono- or bi-cyclic heteroring and the other symbols are the same meanings as above,
AA² is 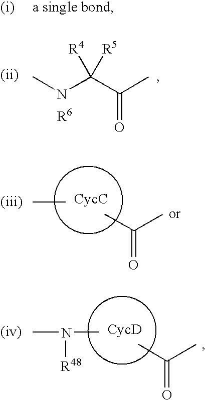
wherein R⁴ and R⁵ are the same or different to represent
(1) hydrogen,
(2) C1-8 alkyl,
(3) CycA or
(4) C1-8 alkyl substituted with 1-5 of group selected from the following (a) to (h):
(a) —NR_WR⁴²,
(b) —OR⁴³,
(c) —SR⁴⁴,
(d) —COR⁴⁵,
(e) —NR⁴⁶CONR⁴¹R⁴²,
(f) guanidino,
(g) CycA,
(h) —NR⁴⁶SO₂R⁴¹; or
R⁴ and R⁵ are taken together to form C2-8 alkylene wherein one carbon atom may be replaced by oxygen, sulfur or —NR⁴⁰— and the alkylene may be substituted with —NR⁴¹R⁴² or —OR⁴³,
R⁴⁰ is hydrogen, C1-4 alkyl, —COO—(C1-4 alkyl), phenyl or C1-4 alkyl substituted with phenyl,
R⁴¹, R⁴², R⁴³, R⁴⁴ and R⁴⁶ are the same or different to represent hydrogen, C1-4 alkyl, phenyl or C1-4 alkyl substituted with phenyl,
R⁴⁵ is C1-4 alkyl, phenyl, —NR⁴¹R⁴² wherein all symbols are the same meaning as above, —OR⁴³ wherein R⁴³ has the same meaning as above, or C1-4 alkyl substituted with phenyl,
R⁶ is hydrogen, C1-8 alkyl, phenyl or C1-8 alkyl substituted with phenyl or
R⁶ is taken together with R⁴ to form C2-6 alkylene wherein one carbon atom may be replaced by oxygen, sulfur or —NR⁴⁰— and the alkylene may be substituted with —NR⁴¹R⁴² or —OR⁴³,
R⁴⁸ is hydrogen, C1-4 alkyl, phenyl or C1-4 alkyl substituted with phenyl or when AA¹ is a single bond, R⁴⁸ and R may be taken together to form C2-6 alkylene wherein one carbon atom may be replaced by oxygen, sulfur or —NR⁴⁷ wherein R⁴⁷ is hydrogen or C1-4 alkyl,
CycC is a 3-17 membered mono- or bi-cyclic heteroring,
CycD is a C3-14 mono- or bi-cyclic carboring or a 3-14 membered mono- or bi-cyclic heteroring, or
AA² and AA¹ are taken together to form, 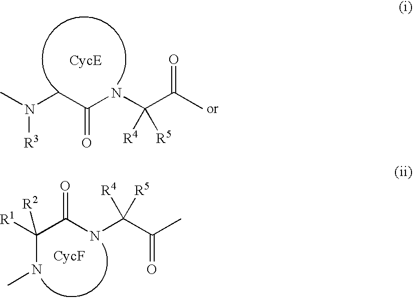
wherein CycE is a 4-18 membered mono- or bi-cyclic heteroring, CycF is a 5-8 membered monocyclic heteroring, and the other symbols have the same meanings as above,
R⁷ and R⁸ are the same or different to represent
(i) hydrogen,
(ii) C1-8 alkyl,
(iii) CycA or
(iv) C1-8 alkyl substituted with 1-5 of group selected from the following (1) to (8);
(1) —NR⁶¹R⁶²,
(2)—OR⁶³,
(3) —SR⁶⁴,
(4) —COR⁶⁵,
(5) —NR⁶⁶CONR⁶¹R⁶², (6) guanidino, (7) CycA, (8)—NR⁶⁶SO₂R⁶¹, or
R⁷ and R⁸ are taken together to form C2-8 alkylene wherein one carbon atom may be replaced by oxygen, sulfur or —NR⁶⁰— and the alkylene may be substituted with —NR⁶¹R⁶² or OR⁶³,
R⁶⁰ is hydrogen, C1-4 alkyl, —COO-(C1-4 alkyl), phenyl or C1-4 alkyl substituted with phenyl,
R⁶¹, R⁶², R⁶³, R¹ and R⁶⁶ are the same or different to represent hydrogen, C1-4 alkyl, phenyl or C1-4 alkyl substituted with phenyl, R⁶⁵ is C1-4 alkyl, phenyl, —NR⁶¹R⁶² wherein all symbols are the same meanings as above, —OR⁶³ wherein R⁶³ is the same meaning as above, or C1-4 alkyl substituted with phenyl,
R⁹ is hydrogen, C1-8 alkyl, phenyl or C1-8 alkyl substituted with phenyl or
R⁹ is taken together with R⁷ to form C2-6 alkylene wherein one carbon atom may be replaced by oxygen, sulfur or —NR⁶⁰— and the alkylene may be substituted with —NR⁶¹R⁶² or —OR⁶³, 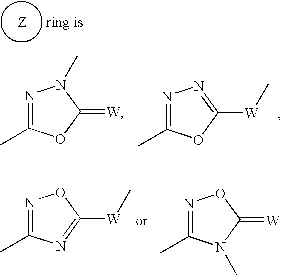
wherein W is oxygen or sulfur,
R¹⁰ is
(i) C1-8 alkyl,
(ii) C2-8 alkenyl,
(iii) CycA,
(iv) —COR⁷¹, or
(v) C1-8 alkyl substituted with 1-3 of CycA, guanidino, —COR⁷¹, —NR⁷²R⁷³, —OR⁷⁴, cyano, —P(O) (OR⁷⁸)₂ and —O—(C1-4 alkylene)-(C1-4 alkoxy)
wherein R⁷¹ is
(1) C1-4 alkyl,
(2) C1-4 alkoxy,
(3) CycA,
(4) —O-CycA,
(5) —NR⁷²R⁷³,
(6) C1-4 alkyl substituted with CycA,
(7) C1-4 alkoxy substituted with CycA or
(8) hydroxy,
R⁷² and R⁷³ are the same or different to represent
(1) hydrogen,
(2) C1-8 alkyl,
(3) C1-8 alkoxy,
(4) C2-8 acyl,
(5) C2-8 alkoxycarbonyl,
(6) CycA,
(7) —C(O)CycA,
(8) —SO₂CycA or
(9) C1-8 alkyl substituted with CycA, —C(O)CycA, —SO₂CycA, C1-8 alkoxy, C2-8 acyl or C2-8 alkoxycarbonyl,
R⁷⁴ is
(1) hydrogen,
(2) C1-8 alkyl,
(3) CycA,
(4) C1-8 alkyl substituted with —SiR⁷⁵R⁷¹R⁷⁷ wherein R⁷⁵, R⁷⁶ and R⁷⁷ are the same or different to represent C1-8 alkyl, phenyl or C1-8 alkyl substituted with phenyl, or
(5) C1-8 alkyl substituted with CycA,
R⁷⁸ is C1-8 alkyl, phenyl or C1-8 alkyl substituted with phenyl;
wherein CycA in R, R¹, R², R⁴, R⁵, R⁷, R⁸, R¹⁶ may be the same or different and CycA, CycB, CycC, CycD, CycE and CycF may be independently substituted with 1-5 of R²⁷;
R²⁷ is
(1) C1-8 alkyl,
(2) halo,
(3) —NR¹R¹²,
(4) —OR¹³,
(5)—SR¹⁴,
(6) CycG,
(7) nitro,
(8) cyano,
(9) oxo,
(10) —COR¹⁵,
(11)-SO₂R⁵, or
(12) C1-8 alkyl substituted with 1-5 of the following (a) to (j):
(a) halo,
(b) —NR¹¹R¹²,
(c) —OR¹³,
(d) —SR¹⁴,
(e) CycG,
(f) nitro,
(g) cyano,
(h) —COR¹⁵,
(j) —SO₂R¹⁵;
wherein R¹¹ and R¹² are the same or different to represent hydrogen, C1-4 alkyl, C1-4 alkoxy, —C(O)O—(C1-4 alkyl), CycG, or C1-4 alkyl substituted with CycG,
R¹³ and R¹⁴ are the same or different to represent hydrogen, C1-4 alkyl, trifluoromethyl, CycG, or C1-4 alkyl substituted with CycG,
CycG is a 5-8 membered mono- or bi-cyclic carboring or a 5-8 membered mono- or bi-cyclic heteroring,
R¹⁵ is C1-4 alkyl, CycG, —NR¹¹R¹² wherein all symbols have the same meanings as above, —OR¹³ wherein R¹³ has the same meaning as above, or C1-4 alkyl substituted with CycG, —NR¹¹R¹² wherein all symbols have the same meanings as above or —OR¹³ wherein R¹³ has the same meaning as above, or
a non-toxic salt thereof.

2. The compound according to claim 1, wherein R¹⁶ is C1-8 alkyl, C2-8 alkenyl or C2-8 alkynyl substituted with 1-5 group selected from halo, nitro, trifluoromethyl, cyano, CycA, NR¹⁸R¹⁹, —NHC(O) —CycA and —NHC(O)O(C1-8 alkyl), wherein when the substituent does not contain —NHC(O)O(C1-8 alkyl), the alkyl, alkenyl or alkynyl has more than one substituent, or a non-toxic salt thereof.

3. The compound according to claim 1, wherein R¹⁰ is C1-8 alkyl substituted with 1-3 of CycA, guanidino, —COR⁷¹, —NR⁷²R⁷³, —OR⁷⁴, cyano or —P(O)(OR⁷⁸)₂, —O-(C1-4 alkylene)-(C1-4 alkoxy), wherein at least one substituent is —O—(C1-4 alkylene)-(C1-4 alkoxy), or a non-toxic salt thereof.

4. The compound according to claim 1, wherein R²⁷ is C1-8 alkyl substituted with SCF₃ or NR¹¹R¹² wherein at least one of R¹¹ or R¹² contains CycG, —C(O)O—(C1-4 alkyl) or C1-4 alkoxy, or a non-toxic salt thereof.

5. The compound according to claim 1, wherein at least one of R⁷² or R⁷³ is C1-8 alkyl substituted with C1-8 alkoxy, C2-8 acyl, C2-8 alkoxycarbonyl, CycA wherein phenyl is excluded, —C(O) —CycA, —SO₂CycA or CycA wherein phenyl is excluded, —C(O) —CycA, —SO₂CycA, C1-8 alkoxy, C2-8 acyl or C2-8 alkoxycarbonyl, or a non-toxic salt thereof.

6. The compound according to claim 1, wherein 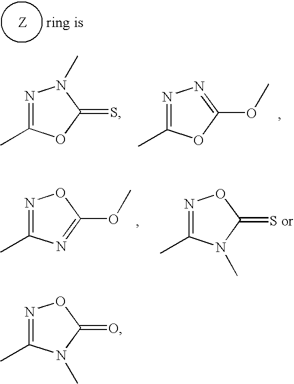
or a non-toxic salt thereof.

7. The compound according to claim 1, which is
(1) cycloheptyl-N-[1-[5-(2-dimethylaminoethylthio)-1,3,4-oxadiazol-2-yl]-4-methyl-1-oxo-2-pentyl]carboxamide,
(2) 1-benzoylaminocyclohexyl-N-[4-methyl-1-[5-(2-pyrrolidinoethylthio)-1,3,4-oxadiazol-2-yl]-1-oxo-2-pentyl]carboxamide,
(3) 1-(4-morpholino-2-butynoylamino)cyclohexyl-N-[4-methyl-1-[5-(1-methylethylthio)-1,3,4-oxadiazol-2-yl]-1-oxo-2-pentyl]carboxamide,
(4) cycloheptyl-N-[(2S)-1-[5-(1-methylethylthio)-1,3,4-oxadiazol-2-yl]-4-methyl-1-oxo-2-pentyl]carboxamide,
(5) 1-(3-morpholinomethylbenzoylamino)cyclohexyl-N-[4-methyl-1-[5-(1-methylethylthio)-1,3,4-oxadiazol-2-yl]-1-oxo-2-pentyl]carboxamide,
(6) 1-[(1R,2S)-2-(2-dimethylaminomethyl-4-fluorobenzoylamino)cyclohexyl]-N-[4-methyl-1-(5-methylthio-1,3,4-oxadiazol-2-yl)-1-oxo-2-pentyl]carboxamide,
(7) 1-[(1R,2S)-2-morpholinocyclohexyl]-N-[(2S)-4-methyl-1-[5-(1-methylethylthio)-1,3,4-oxadiazol-2-yl]-1-oxo-2-pentyl]carboxamide,
(8) (2S)-N-[4-methyl-1-[5-(N-methylpiperidin-4-ylthio)-1,3,4-oxadiazol-2-yl]-1-oxo-2-pentyl]-2-benzyloxycarbonylamino-4-methylpentanamide,
(9) cyclohexyl-N-[4-methyl-1-[5-(N-methylpiperidin-4-ylthio)-1,3,4-oxadiazol-2-yl]-1-oxo-2-pentyl]carboxamide,
(10) 1-[(1R,2S)-2-benzoylaminocyclohexyl]-N-[4-methyl-1-[5-(N-methylpiperidin-4-ylthio)-1,3,4-oxadiazol-2-yl]-1-oxo-2-pentyl]carboxamide,
(11) 1-benzoylaminocyclohexyl-N-[4-methyl-1-[5-(N-methylpiperidin-4-ylthio)-1,3,4-oxadiazol-2-yl]-1-oxo-2-pentyl]carboxamide,
(12) cyclohexyl-N-[(2S)-1-[5-(1-methylethylthio)-1,3,4-oxadiazol-2-yl]-1-oxo-2-hexyl]carboxamide,
(13) cycloheptyl-N-[(2S)-1-[5-(1-methylethylthio)-1,3,4-oxadiazol-2-yl]-1-oxo-2-hexyl]carboxamide,
(14) cyclooctyl-N-[(2S)-4-methyl-1-[5-(1-methylethylthio)-1,3,4-oxadiazol-2-yl]-1-oxo-2-pentyl]carboxamide,
(15) 1-morpholinocarbonylaminocyclohexyl-N-[1-[5-(2-dimethylaminoethylthio)-1,3,4-oxadiazol-2-yl]-4-methyl-1-oxo-2-pentyl]carboxamide,
(16) cyclohexyl-N-[1-[5-(2-dimethylaminoethylthio)-1,3,4-oxadiazol-2-yl]-1-oxo-2-hexyl]carboxamide,
(17) cycloheptyl-N-[1-[5-(2-dimethylaminoethylthio)-1,3,4-oxadiazol-2-yl]-1-oxo-2-hexyl]carboxamide,
(18) cyclohexyl-N-[1-[5-(3-pyrrolidinopropylthio)-1,3,4-oxadiazol-2-yl]-4-methyl-1-oxo-2-pentyl]carboxamide,
(19) 1-[(1R,2 S)-2-benzoylaminocyclohexyl]-N-[4-methyl-1-[5-(3-pyrrolidinopropylthio)-1,3,4-oxadiazol-2-yl]-1-oxo-2-pentyl]carboxamide,
(20) cyclohexyl-N-[1-[5-(3-morpholinopropylthio)-1,3,4-oxadiazol-2-yl]-4-methyl-1-oxo-2-pentyl]carboxamide,
(21) 1-[(1R,2S)-2-benzoylaminocyclohexyl]-N-[4-methyl-1-[5-(3-morpholinopropylthio)-1,3,4-oxadiazol-2-yl]-1-oxo-2-pentyl]carboxamide,
(22) cyclooctyl-N-[1-[5-(2-dimethylaminoethylthio)-1,3,4-oxadiazol-2-yl]-4-methyl-1-oxo-2-pentyl]carboxamide,
(23) cyclohexyl-N-[1-[5-(1-methylethylthio)-1,3,4-oxadiazol-2-yl]-4-methyl-1-oxo-2-pentyl]carboxamide,
(24) N-[(2S)-4-methyl-1-[5-(1-methylethylthio)-1,3,4-oxadiazol-2-yl]-1-oxo-2-pentyl]-(3,4-dihydro-4-oxo-2-phenylpyrimidin-3-yl)acetamide,
(25) N-cyclopentyloxycarbonyl-N-[4-methyl-1-[5-(1-methylethylthio)-1,3,4-oxadiazol-2-yl]-1-oxo-2-pentyl]amine,
(26) cyclohexyl-N-[1-[5-(3-dimethylaminopropylthio)-1,3,4-oxadiazol-2-yl]-1-oxo-2-hexyl]carboxamide,
(27) cycloheptyl-N-[1-[5-(3-dimethylaminopropylthio)-1,3,4-oxadiazol-2-yl]-1-oxo-2-hexyl]carboxamide,
(28) N-[(2S)-4-methyl-1-[5-(1-methylethylthio)-1,3,4-oxadiazol-2-yl]-1-oxo-2-pentyl]-5-methylhexanamide,
(29) N-[4-methyl-1-[5-(1-methylethylthio)-1,3,4-oxadiazol-2-yl]-1-oxo-2-pentyl]-4-cyclohexylbutanamide,
(30) (2S)-N-[(2S)-[4-methyl-1-(1-methylethylthio)-1,3,4-oxadiazol-5-yl]-1-oxo-2-pentyl]-2-(t-butoxycarbonylamino)-4-methylpentanamide,
(31) N-(2,2-dimethylpropyloxycarbonyl)-N-[4-methyl-2-(1-methylethylthio)-1,3,4-oxadiazol-5-yl-1-oxo-2-pentyl]amine,
(32) N-[4-methyl-1-[5-(1-methylethylthio)-1,3,4-oxadiazol-2-yl]-1-oxo-2-pentyl]cyclohexylacetamide, (3-3) 1-(tetrahydropyran-4-yl)-N-[4-methyl-1-[5-(1-methylethylthio)-1,3,4-oxadiazol-2-yl]-1-oxo-2-pentyl]carboxamide,
(34) 1-cyclopropylcarbonylaminocyclohexyl-N-[(2S)-4-methyl-1-[5-(1-methylethylthio)-1,3,4-oxadiazol-2-yl]-1-oxo-2-pentyl]carboxamide,
(35) N-(t-butoxycarbonyl)-N-[1-[5-(2-dimethylaminoethylthio)-1,3,4-oxadiazol-2-yl]-4-methyl-1-oxo-2-pentyl]amine,
(36) cyclohexyl-N-[(2S)-1-[5-(2-t-butoxycarbonylaminoethylthio)-1,3,4-oxadiazol-2-yl]-4-methyl-1-oxo-2-pentyl]carboxamide,
(37) cyclononyl-N-[4-methyl-1-[5-(1-methylethylthio)-1,3,4-oxadiazol-2-yl]-1-oxo-2-pentyl]carboxamide,
(38) N-[4-methyl-1-[5-(1-methylethylthio)-1,3,4-oxadiazol-2-yl]-1-oxo-2-pentyl]-2-propylpentanamide,
(39) N-[4-methyl-1-[5-(1-methylethylthio)-1,3,4-oxadiazol-2-yl]-1-oxo-2-pentyl]-6-phenylhexanamide,
(40) 1-(2,2,3,3-tetramethylcyclopropyl)-N-[4-methyl-1-[5-(1-methylethylthio)-1,3,4-oxadiazol-2-yl]-1-oxo-2-pentyl]carboxamide,
(41) N-[4-methyl-1-[5-(1-methylethylthio)-1,3,4-oxadiazol-2-yl]-1-oxo-2-pentyl]-4-(2-thienyl)butanamide,
(42) N-[4-methyl-1-[5-(1-methylethylthio)-1,3,4-oxadiazol-2-yl]-1-oxo-2-pentyl]-(tricyclo[3.3.1.1]decan-1-yl)acetamide,
(43) tricyclo[3.3.1.1]decan-1-yl-N-[4-methyl-1-[2-(1-methylethylthio)-1,3,4-oxadiazol-5-yl]-1-oxo-2-pentyl]carboxamide,
(44) cyclohexyl-N-[(2S)-1-[5-(t-butoxycarbonylmethylthio)-1,3,4-oxadiazol-2-yl]-4-methyl-1-oxo-2-pentyl]carboxaniide,
(45) cyclohexyl-N-[(2S)-1-[5-(2-methoxyethylthio)-1,3,4-oxadiazol-2-yl]-4-methyl-1-oxo-2-pentyl]carboxamide,
(46) cyclohexyl-N-[(2S)-1-[5-(1,3-dioxolan-2-ylmethylthio)-1,3,4-oxadiazol-2-yl]-4-methyl-1-oxo-2-pentyl]carboxamide,
(47) cycloheptyl-N-[(2S)-4-methyl-1-[5-(2-morpholinoethylthio)-1,3,4-oxadiazol-2-yl]-1-oxo-2-pentyl]carboxamide,
(48) cycloheptyl-N-[(2S)-1-(5-cyanomethylthio-1,3,4-oxadiazol-2-yl)-4-methyl-1-oxo-2-pentyl]carboxamide,
(49) cycloheptyl-N-[1-[5-(1-t-butoxycarbonyl-1-methylethylthio)-1,3,4-oxadiazol-2-yl]-4-methyl-1-oxo-2-pentyl]carboxamide,
(50) cycloheptyl-N-[1-[5-(2,4-dioxo-1,5,5-trimethylpyrrolidin-3-ylmethylthio)-1,3,4-oxadiazol-2-yl]-4-methyl-1-oxo-2-pentyl]carboxamide,
(51) cycloheptyl-N-[(2S)-1-[5-(3,3,3-trifluoropropylthio)-1,3,4-oxadiazol-2-yl]-4-methyl-1-oxo-2-pentyl]carboxamide,
(52) 1-(t-butoxycarbonylamino)cyclopentyl-N-[(2S)-4-methyl-1-[5-(1-methylethylthio)-1,3,4-oxadiazol-2-yl]-1-oxo-2-pentyl]carboxamide,
(53) 1-(t-butoxycarbonylamino)cyclohexyl-N-[4-methyl-1-[5-(2-dimethylaminoethylthio)-1,3,4-oxadiazol-2-yl]-1-oxo-2-pentyl]carboxamide,
(54) (indan-2-yl)-N-[1-[5-(1-methylethylthio)-1,3,4-oxadiazol-2-yl]-4-methyl-1-oxo-2-pentyl]carboxamide,
(55) 1-cyclopropylcarbonylaminocyclohexyl-N-[4-methyl-1-[5-(2-dimethylaminoethylthio)-1,3,4-oxadiazol-2-yl]-1-oxo-2-pentyl]carboxamide,
(56) N-[4-methyl-1-[5-(1-methylethylthio)-1,3,4-oxadiazol-2-yl-1-oxo]-2-pentyl]-3-diethylaminopropanamide,
(57) 1-methylpiperidin-4-yl-N-[4-methyl-1-[5-(1-methylethylthio)-1,3,4-oxadiazol-2-yl]-1-oxo-2-pentyl]carboxamide,
(58) N-[4-methyl-1-[5-(1-methylethylthio)-1,3,4-oxadiazol-2-yl]-1-oxo-2-pentyl]-1-piperidinopropanamide,
(59) N-[4-methyl-1-[5-(1-methylethylthio)-1,3,4-oxadiazol-2-yl]-1-oxo-2-pentyl]-4-dimethylaminobutanamide,
(60) N-[(2S)-4-methyl-1-[5-(1-methylethylthio)-1,3,4-oxadiazol-2-yl]-1-oxo-2-pentyl]-(3,4-dihydro-4-oxo-2-methylpyrimidin-3-yl)acetamide,
(61) N-[(2S)-4-methyl-1-[5-(1-methylethylthio)-1,3,4-oxadiazol-2-yl]-1-oxo-2-pentyl]-(3,4-dihydro-4-oxo-2-cyclohexylmethylpyrimidin-3-yl)acetamide,
(62) 1-(benzo[b]thiophen-2-ylcarbonylaminocyclohexyl)-N-[4-methyl-1-[5-(1-methylethylthio)-1,3,4-oxadiazol-2-yl]-1-oxo-2-pentyl]carboxamide,
(63) 1-(benzo[b]thiophen-2-ylcarbonylamino)cyclohexyl-N-[4-methyl-1-[5-(2-dimethylaminoethylthio)-1,3,4-oxadiazol-2-yl]-1-oxo-2-pentyl]carboxamide,
(64) cycloheptyl-N-[(2S)-1-(5-dimethylaminocarbonylmethylthio-1,3,4-oxadiazol-2-yl)-4-methyl-1-oxo-2-pentyl]carboxamide,
(65) N-[1-[5-(2-dimethylaminoethylthio)-1,3,4-oxadiazol-2-yl]-4-methyl-1-oxo-2-pentyl]acetamide,
(66) cycloheptyl-N-[(2S)-4-methyl-1-[5-(2-methylpropylthio)-1,3,4-oxadiazol-2-yl]-1-oxo-2-pentyl]carboxamide,
(67) N-benzyloxycarbonyl-N-[1-[5-(2-dimethylaminoethylthio)-1,3,4-oxadiazol-2-yl]-4-methyl-1-oxo-2-pentyl]amine,
(68) 1-(3-diethylaminopropanoylamino)cyclohexyl-N-[4-methyl-1-[5-(1-methylethylthio)-1,3,4-oxadiazol-2-yl]-1-oxo-2-pentyl]carboxamide,
(69) cycloheptyl-N-[(2S)-4-methyl-1-oxo-1-[5-(pyridin-2-ylmethylthio)-1,3,4-oxadiazol-2-yl]-2-pentyl]carboxamide,
(70) cycloheptyl-N-[(2S)-4-methyl-1-oxo-1-[5-(pyridin-3-ylmethylthio)-1,3,4-oxadiazol-2-yl]-2-pentyl]carboxamide,
(71) cycloheptyl-N-[(2S)-1-[5-(pyridin-4-ylmethylthio)-1,3,4-oxadiazol-2-yl]-4-methyl-1-oxo-2-pentyl]carboxamide,
(72) cycloheptyl-N-[(2S)-1-[5-(1-dimethylaminocarbonyl-1-methylethylthio)-1,3,4-oxadiazol-2-yl]-4-methyl-1-oxo-2-pentyl]carboxamide,
(73) 1-(4-dimethylaminomethylbenzoylamino)cyclohexyl-N-[4-methyl-1-[5-(2-dimethylaminoethylthio)-1,3,4-oxadiazol-2-yl]-1-oxo-2-pentyl]carboxaniide,
(74) 1-(3-dimethylaminomethylbenzoylamino)cyclohexyl-N-[1-[5-(2-dimethylaminoethylthio)-1,3,4-oxadiazol-2-yl]-4-methyl-1-oxo-2-pentyl]carboxamide,
(75) cycloheptyl-N-[4-methyl-1-oxo-1-[5-(2-trimethylammonioethylthio)-1,3,4-oxadiazol-2-yl]-2-pentyl]carboxamide iodide,
(76) cycloheptyl-N-[(2S)-4-methyl-1-[5-(2,4-dioxo-1-methyl-1,3-imidazolidin-3-ylmethylthio)-1,3,4-oxadiazol-2-yl]-1-oxo-2-pentyl]carboxamide,
(77) 1-benzoylaminocyclohexyl-N-[(2S)-4-methyl-1-[5-(2,4-dioxo-1-methyl-1,3-imidazolidin-3-ylmethylthio)-1,3,4-oxadiazol-2-yl]-1-oxo-2-pentyl]carboxamide,
(78) cycloheptyl-N-[(2S)-4-methyl-1-[5-(1,2,4-oxadiazol-3-ylmethylthio)-1,3,4-oxadiazol-2-yl]-1-oxo-2-pentyl]carboxamide,
(79) 1-benzoylaminocyclohexyl-N-[(2S)-4-methyl-1-[5-(2,4-dioxo-1,5,5-trimethyl-1,3-imidazolidin-3-ylmethylthio)-1,3,4-oxadiazol-2-yl]-1-oxo-2-pentyl]amide,
(80) cycloheptyl-N-[(2S)-4-methyl-1-[5-(2-pyrazolylethylthio)-1,3,4-oxadiazol-2-yl]-1-oxo-2-pentyl]carboxamide,
(81) 1-(N-methylpiperidin-4-ylcarbonylamino)cyclohexyl-N-[4-methyl-1-[5-(1-methylethylthio)-1,3,4-oxadiazol-2-yl]-1-oxo-2-pentyl]carboxamide,
(82) 1-(3-piperidinopropanoylamino)cyclohexyl-N-[4-methyl-1-[5-(1-methylethylthio)-1,3,4-oxadiazol-2-yl]-1-oxo-2-pentyl]carboxamide,
(83) 1-(4-phenylbenzoylamino)cyclohexyl-N-[4-methyl-1-[5-(2-dimethylaminoethylthio)-1,3,4-oxadiazol-2-yl]-1-oxo-2-pentyl]carboxamide,
(84) 1-(3-trifluoromethylbenzoylamino)cyclohexyl-N-[4-methyl-1-[5-(2-dimethylaminoethylthio)-1,3,4-oxadiazol-2-yl]-1-oxo-2-pentyl]carboxamide,
(85) 1-(4-trifluoromethyloxybenzoylamino)cyclohexyl-N-[4-methyl-1-[5-(2-dimethylaminoethylthio)-1,3,4-oxadiazol-2-yl]-1-oxo-2-pentyl]carboxamide,
(86) cyclohexyl-N-[1-[5-(2-azetidinoethylthio)-1,3,4-oxadiazol-2-yl]-4-methyl-1-oxo-2-pentyl]carboxamide,
(87) 1-(4-trifluoromethylbenzoylamino)cyclohexyl-N-[1-[5-(2-dimethylaminoethylthio)-1,3,4-oxadiazol-2-yl]-4-methyl-1-oxo-2-pentyl]carboxamide,
(88) 1-(2-trifluoromethylbenzoylamino)cyclohexyl-N-[1-[5-(2-dimethylaminoethylthio)-1,3,4-oxadiazol-2-yl]-4-methyl-1-oxo-2-pentyl]carboxamide,
(89) 1-(3-trifluoromethyloxybenzoylamino)cyclohexyl-N-[1-[5-(2-dimethylaminoethylthio)-1,3,4-oxadiazol-2-yl]-4-methyl-1-oxo-2-pentyl]carboxamide,
(90) 1-(4-fluorobenzoylamino)cyclohexyl-N-[1-[5-(2-dimethylaminoethylthio)-1,3,4-oxadiazol-2-yl]-4-methyl-1-oxo-2-pentyl]carboxamide,
(91) 1-(3-fluorobenzoylamino)cyclohexyl-N-[1-[5-(2-dimethylaminoethylthio)-1,3,4-oxadiazol-2-yl]-4-methyl-1-oxo-2-pentyl]carboxamide,
(92) 1-(2-fluorobenzoylamino)cyclohexyl-N-[1-[5-(2-dimethylaminoethylthio)-1,3,4-oxadiazol-2-yl]-4-methyl-1-oxo-2-pentyl]carboxamide,
(93) 1-(3-methoxybenzoylamino)cyclohexyl-N-[1-[5-(2-dimethylaminoethylthio)-1,3,4-oxadiazol-2-yl]-4-methyl-1-oxo-2-pentyl]carboxamide,
(94) 1-nicotinoylaminocyclohexyl-N-[1-[5-(2-dimethylaminoethylthio)-1,3,4-oxadiazol-2-yl]-4-methyl-1-oxo-2-pentyl]carboxamide,
(95) 1-isonicotinoylaminocyclohexyl-N-[1-[5-(2-dimethylaminoethylthio)-1,3,4-oxadiazol-2-yl]-4-methyl-1-oxo-2-pentyl]carboxamide,
(96) 1-benzyloxymethylcyclohexyl-N-[(2S)-1-[5-(1-methylethylthio)-1,3,4-oxadiazol-2-yl]-4-methyl-1-oxo-2-pentyl]carboxamide,
(97) 1-benzyloxymethylcyclohexyl-N-[(2S)-1-[5-(2,4-dioxo-1,5,5-trimethylimidazolidin-3-ylmethylthio)-1,3,4-oxadiazol-2-yl]-4-methyl-1-oxo-2-pentyl]carboxamide,
(98) 1-benzyloxymethylcyclohexyl-N-[1-[5-(2-dimethylaminoethylthio)-1,3,4-oxadiazol-2-yl]-4-methyl-1-oxo-2-pentyl]carboxamide,
(99) cyclohexyl-N-[1-[5-[2-(methylphenylamino)ethylthio]-1,3,4-oxadiazol-2-yl]-4-methyl-1-oxo-2-pentyl]carboxamide,
(100) 1-cyclohexylcarbonylaminocyclohexyl-N-[1-[5-(2-dimethylaminoethylthio)-1,3,4-oxadiazol-2-yl]-4-methyl-1-oxo-2-pentyl]carboxamide,
(101) cyclohexyl-N-[1-[5-[2-(N-benzyl-N-methylamino)ethylthio]-1,3,4-oxadiazol-2-yl]-4-methyl-1-oxo-2-pentyl]carboxamide,
(102) 1-(t-butoxycarbonylamino)cyclohexyl-N-[1-[5-(2-dimethylaminoethylthio)-1,3,4-oxadiazol-2-yl]-4-methyl-1-oxo-2-pentyl]carboxamide,
(103) 1-(3-phenylpropinoylamino)cyclohexyl-N-[1-[5-(2-dimethylaminoethylthio)-1,3,4-oxadiazol-2-yl]-4-methyl-1-oxo-2-pentyl]carboxamide,
(104) 1-benzylcarbonylaminocyclohexyl-N-[1-[5-(2-dimethylaminoethylthio)-1,3,4-oxadiazol-2-yl]-4-methyl-1-oxo-2-pentyl]carboxamide,
(105) 1-cyclopentylcarbonylaminocyclohexyl-N-[1-[5-(2-dimethylaminoethylthio)-1,3,4-oxadiazol-2-yl]-4-methyl-1-oxo-2-pentyl]carboxamide,
(106) 1-(2-thienylcarbonylamino)cyclohexyl-N-[1-[5-(2-dimethylaminoethylthio)-1,3,4-oxadiazol-2-yl]-4-methyl-1-oxo-2-pentyl]carboxamide,
(107) 1-(4-methoxybenzoylamino)cyclohexyl-N-[1-[5-(2-dimethylaminoethylthio)-1,3,4-oxadiazol-2-yl]-4-methyl-1-oxo-2-pentyl]carboxamide,
(108) 1-(2-furylcarbonylamino)cyclohexyl-N-[1-[5-(2-dimethylaminoethylthio)-1,3,4-oxadiazol-2-yl]-4-methyl-1-oxo-2-pentyl]carboxamide,
(109) 1-(4-dimethylaminomethylbenzoylamino)cyclohexyl-N-[1-[5-(2,4-dioxo-1,5,5-trimethylimidazolidin-3-ylmethylthio)-1,3,4-oxadiazol-2-yl]-4-methyl-1-oxo-2-pentyl]carboxamide,
(110) 1-(3-methylbenzoylamino)cyclohexyl-N-[1-[5-(2-dimethylaminoethylthio)-1,3,4-oxadiazol-2-yl]-4-methyl-1-oxo-2-pentyl]carboxamide,
(111) 1-(4-methylbenzoylamino)cyclohexyl-N-[1-[5-(2-dimethylaminoethylthio)-1,3,4-oxadiazol-2-yl]-4-methyl-1-oxo-2-pentyl]carboxamide,
(112) cyclohexyl-N-[(2S)-1-[5-[2-(1-methyl-2,4-dioxoimidazolidin-3-yl)ethylthio]-1,3,4-oxadiazol-2-yl]-4-methyl-1-oxo-2-pentyl]carboxamide,
(113) cyclohexyl-N-[(2S)-1-[5-(2-aminoethylthio)-1,3,4-oxadiazol-2-yl]-4-methyl-1-oxo-2-pentyl]carboxamide,
(114) cycloheptyl-N-[(2S)-1-[5-(2-aminoethylthio)-1,3,4-oxadiazol-2-yl]-4-methyl-1-oxo-2-pentyl]carboxamide,
(115) N-[(2S)-1-[5-(1-methylethylthio)-1,3,4-oxadiazol-2-yl]-4-methyl-1-oxo-2-pentyl]-4-amino-4-methylpentanamide,
(116) N-[(2S)-4-methyl-1-[5-(1-methylethylthio)-1-oxo-1,3,4-oxadiazol-2-yl]-1-oxo-2-pentyl]-(2S)-2-amino-4-methylpentanamde,
(117) cycloheptyl-N-[(2S)-1-[5-(1-carboxy-1-methylethylthio)-1,3,4-oxadiazol-2-yl]-4-methyl-1-oxo-2-pentyl]carboxamide,
(118) cyclohexyl-N-[(2S)-1-(5-carboxymethylthio-1,3,4-oxadiazol-2-yl)-4-methyl-1-oxo-2-pentyl]carboxamide,
(119) 1-[(1R,2S)-2-(4-dimethylaminomethylbenzoylamino)cyclohexyl]-N-[(2S)-4-methyl-1-oxo-1-[2-oxo-3-phenyl-1,3,4-oxadiazolidin-5-yl]-2-pentyl]carboxamide,
(120) cyclohexyl-N-[(2S)-4-methyl-1-oxo-1-[2-oxo-3-phenyl-1,3,4-oxadiazolin-5-yl]-2-pentyl]carboxamide,
(121) 1-[(1R,2S)-2-(t-butoxycarbonylamino)cyclohexyl]-N-[(2S)-1-[3-cyclopropylmethyl-2-oxo-1,3,4-oxadiazolin-5-yl]-4-methyl-1-oxo-2-pentyl]carboxamide,
(122) 1-[(1R,2S)-2-[4-dimethylamino(2-butynoul)amino]cyclohexyl]-N-[1-[3-cyclopropylmethyl-2-oxo-1,3,4-oxadiazolin-5-yl]-4-methyl-1-oxo-2-pentyl]carboxamide,
(123) 1-[(1R,2S)-2-[4-morpholino(2-butynoyl)amino]cyclohexyl]-N-[1-[3-cyclopropylmethyl-2-oxo-1,3,4-oxadiazolin-5-yl]-4-methyl-1-oxo-2-pentyl]carboxamide,
(124) 1-[(1R,2S)-2-(2-butynoylamino)cyclohexyl]-N-[(2S)-1-[3-cyclopropylmethyl-2-oxo-1,3,4-oxadiazolin-5-yl]-4-methyl-1-oxo-2-pentyl]carboxamide,
(125) cyclohexyl-N-[(2S)-1-[3-(2-dimethylaminoethyl)-2-oxo-1,3,4-oxadiazolin-5-yl]-4-methyl-1-oxo-2-pentyl]carboxamide,
(126) 1-[(1R,2S)-2-benzoylaminocyclohexyl]-N-[4-methyl-1-[5-(2-morpholinoethylthio)-1,2,4-oxadiazol-3-yl]-1-oxo-2-pentyl]carboxamide,
(127) cyclohexyl-N-[4-methyl-1-[5-(2-morpholinoethylthio)-1,2,4-oxadiazol-3-yl]-1-oxo-2-pentyl]carboxamide,
(128) 1-(t-butoxycarbonylamino)cyclohexyl-N-[4-methyl-1-[5-(2-morpholinoethylthio)-1,3,4-oxadiazol-2-yl]-1-oxo-2-pentyl]carboxamide,
(129) 1-(t-butoxycarbonylamino)cyclohexyl-N-[1-[5-(3-dimethylaminopropylthio)-1,3,4-oxadiazol-2-yl]-4-methyl-1-oxo-2-pentyl]carboxamide,
(130) cyclohexyl-N-[1-[5-(2-dimethylaminoethylthio)-1,3,4-oxadiazol-2-yl]-4-methyl-1-oxo-2-pentyl] carboxamide,
(131) 1-(4-morpholinomethylbenzoylamino)cyclohexyl-N-[1-[5-(2-dimethylaminoethylthio)-1,3,4-oxadiazol-2-yl]-4-methyl-1-oxo-2-pentyl]carboxamide,
(132) 1-(3-morpholinomethylbenzoylamino)cyclohexyl-N-[1-[5-(2-dimethylaminoethylthio)-1,3,4-oxadiazol-2-yl]-4-methyl-1-oxo-2-pentyl]carboxamide,
(133) 1-(2-butynyloxycarbonylamino)cyclohexyl-N-[1-[5-(2-dimethylaminoethylthio)-1,3,4-oxadiazol-2-yl]-4-methyl-1-oxo-2-pentyl]carboxamide,
(134) 1-(3-butynyloxycarbonylamino)cyclohexyl-N-[1-[5-(2-dimethylaminoethylthio)-1,3,4-oxadiazol-2-yl]-4-methyl-1-oxo-2-pentyl]carboxamide,
(135) 1-methoxycarbonylaminocyclohexyl-N-[1-[5-(2-dimethylaminoethylthio)-1,3,4-oxadiazol-2-yl]-4-methyl-1-oxo-2-pentyl]carboxamide,
(136) 1-benzoylaminocyclohexyl-N-[1-[5-(2-diethylaminoethylthio)-1,3,4-oxadiazol-2-yl]-4-methyl-1-oxo-2-pentyl]carboxamide,
(137) cyclohexyl-N-[(2S)-1-[5-[2-(2,4-dioxoimidazolidin-3-yl)ethylthio]-1,3,4-oxadiazol-2-yl]-4-methyl-1-oxo-2-pentyl]carboxamide,
(138) 1-(4-chlorobenzoylamino)cyclohexyl-N-[1-[5-(2-dimethylaminoethylthio)-1,3,4-oxadiazol-2-yl]-4-methyl-1-oxo-2-pentyl]carboxamide,
(139) 1-(2-trifluoromethyloxybenzoylamino)cyclohexyl-N-[1-[5-(2-dimethylaminoethylthio)-1,3,4-oxadiazol-2-yl]-4-methyl-1-oxo-2-pentyl]carboxamide,
(140) 1-(1,3-benzodioxol-5-ylcarbonylamino)cyclohexyl-N-[1-[5-(2-dimethylaminoethylthio)-1,3,4-oxadiazol-2-yl]-4-methyl-1-oxo-2-pentyl]carboxamide,
(141) 1-phenoxymethylcarbonylaminocyclohexyl-N-[1-[5-(2-dimethylaminoethylthio)-1,3,4-oxadiazol-2-yl]-4-methyl-1-oxo-2-pentyl]carboxamide,
(142) 1-(2-chlorobenzoylamino)cyclohexyl-N-[1-[5-(2-dimethylaminoethylthio)-1,3,4-oxadiazol-2-yl]-4-methyl-1-oxo-2-pentyl]carboxamide,
(143) 1-(2-thienylmethylcarbonylamino)cyclohexyl-N-[1-[5-(2-dimethylaminoethylthio)-1,3,4-oxadiazol-2-yl]-4-methyl-1-oxo-2-pentyl]carboxamide,
(144) 1-benzoylaminocyclohexyl-N-[(2S)-1-[5-[2-(2,4-dioxoimidazolidin-3-yl)ethylthio]-1,3,4-oxadiazol-2-yl]-4-methyl-1-oxo-2-pentyl]carboxamide,
(145) (2S)-N-[2-methyl-1-[5-(2-dimethylaminoethylthio)-1,3,4-oxadiazol-2-yl]-1-oxo-2-propyl]-2-benzyloxycarbonylamino-4-methylpentanamide,
(146) cyclohexyl-N-[1-[5-[2-(N-ethyl-N-methylamino)ethylthio]-1,3,4-oxadiazol-2-yl]-4-methyl-1-oxo-2-pentyl]carboxamide,
(147) 1-(4-cyanobenzoylamino)cyclohexyl-N-[1-[5-(2-dimethylaminoethylthio)-1,3,4-oxadiazol-2-yl]-4-methyl-1-oxo-2-pentyl]carboxamide or
(148) 1-phenoxycarbonylaminocyclohexyl-N-[1-[5-(2-dimethylaminoethylthio)-1,3,4-oxadiazol-2-yl]-4-methyl-1-oxo-2-pentyl]carboxamide, or a non-toxic salt thereof.

8. The compound according to claim 2, which is
(1) N-(2,2,2-trichloroethyloxycarbonyl)-N-[4-methyl-1-[2-(1-methylethylthio)-1,3,4-oxadiazol-5-yl]-1-oxo-2-pentyl]amine or
(2) N-[(2S)-4-methyl-1-[5-(1-methylethylthio)-1,3,4-oxadiazol-2-yl]-1-oxo-2-pentyl]-4-(t-butoxycarbonylamino)-4-methylpentanamide, or a non-toxic salt thereof.

9. The compound according to claim 3, which is cycloheptyl-N-[(2S)-1-[5-[2-(2-methoxyethyloxy)ethylthio]-1,3,4-oxadiazol-2-yl]-4-methyl-1-oxo-2-pentyl]carboxamide or a non-toxic salt thereof.

10. The compound according to claim 4, which is
(1) 1-[4-(N-methoxy-N-methylaminomethyl)benzoylamino]cyclohexyl-N-[1-[5-(2-dimethylaminoethylthio)-1,3,4-oxadiazol-2-yl]-4-methyl-1-oxo-2-pentyl]carboxamide or
(2) 1-[4-(N-methoxy-N-methylaminomethyl)benzoylamino]cyclohexyl-N-[1-[5-(1-methylethylthio)-1,3,4-oxadiazol-2-yl]-4-methyl-1-oxo-2-pentyl]carboxamide, or a non-toxic salt thereof.

11. The compound according to claim 5, which is
(1) cyclohexyl-N-[1-[5-[2-(N-methoxy-N-methylamino)ethylthio]-1,3,4-oxadiazol-2-yl]-4-methyl-1-oxo-2-pentyl]carboxamide,
(2) cyclohexyl-N-[(2S)-1-[5-[2-(N-benzoyl-N-methylamino)ethylthio]-1,3,4-oxadiazol-2-yl]-4-methyl-1-oxo-2-pentyl]carboxamide,
(3) cyclohexyl-N-[(2S)-1-[5-[2-[N-methyl-N-(2-pyridylsulfonyl)amino]ethylthio]-1,3,4-oxadiazol-2-yl]-4-methyl-1-oxo-2-pentyl]carboxamide or
(4) cyclohexyl-N-[(2S)-1-[5-[2-[N-methyl-N-(N-oxidopyridin-2-ylsulfonyl)amino]ethylthio]-1,3,4-oxadiazol-2-yl]-4-methyl-1-oxo-2-pentyl]carboxamide, or a non-toxic salt thereof.

12. The compound according to claim 6, which is
(1) (2S)-N-[1-[3-(2-dimethylaminoethyl)-2-thioxo-1,3,4-oxadiazolin-5-yl]-4-methyl-1-oxo-2-pentyl]-2-benzyloxycarbonylamino-4-methylpentanamide,
(2) 1-[(1R,2S)-2-benzoylaminocyclohexyl]-N-[4-methyl-1-[3-(2-dimethylaminoethyl)-2-thioxo-1,3,4-oxadiazolin-5-yl]-1-oxo-2-pentyl]carboxamide,
(3) cyclohexyl-N-[1-[3-(2-dimethylaminoethyl)-2-thioxo-1,3,4-oxadiazolin-5-yl]-4-methyl-1-oxo-2-pentyl]carboxamide,
(4) 1-benzoylaminocyclohexyl-N-[4-methyl-1-(5-thioxo-1,3,4-oxadiazolin-2-yl)-1-oxo-2-pentyl]carboxamide,
(5) (2S)-N-[(2S)-1-[3-(1-methylethyl)-2-thioxo-1,3,4-oxadiazolin-5-yl]-4-methyl-1-oxo-2-pentyl]-2-benzyloxycarbonylamino-4-methylpentanamide,
(6) 1-[(1R,2S)-2-benzoylaminocyclohexyl]-N-[(2S)-4-methyl-1-oxo-1-[3-(1-methylethyl)-2-thioxo-1,3,4-oxadiazolin-5-yl]-2-pentyl]carboxamide,
(7) cyclohexyl-N-[(2S)-1-[5-(1-methylethyloxy)-1,3,4-oxadiazol-2-yl]-4-methyl-1-oxo-2-pentyl]carboxamide,
(8) cyclohexyl-N-[1-[5-(2-dimethylaminoethyloxy)-1,3,4-oxadiazol-2-yl]-4-methyl-1-oxo-2-pentyl]carboxamide,
(9) cyclohexyl-N-[(2S)-1-[5-(2-methylpropyloxy)-1,3,4-oxadiazol-2-yl]-4-methyl-1-oxo-2-pentyl]carboxamide or
(10) 1-benzoylaminocyclohexyl-N-[1-[5-(2-dimethylaminoethyloxy)-1,3,4-oxadiazol-2-yl]-4-methyl-1-oxo-2-pentyl]carboxamide or a non-toxic salt thereof.

13. The compound according to claim 1, which is
(1) 1-(2-methylbenzoylamino)cyclohexyl-N-[1-[5-(2-dimethylaminoethylthio)-1,3,4-oxadiazol-2-yl]-4-methyl-1-oxo-2-pentyl]carboxamide,
(2) 1-benzyloxymethylcyclohexyl-N-[1-[5-(2-diethylaminoethylthio)-1,3,4-oxadiazol-2-yl]-4-methyl-1-oxo-2-pentyl]carboxamide,
(3) 1-(4-methyl-1,2,3-thiadiazol-5-ylcarbonylamino)cyclohexyl-N-[1-[5-(2-dimethylaminoethylthio)-1,3,4-oxadiazol-2-yl]-4-methyl-1-oxo-2-pentyl]carboxamide,
(4) 1-[5-methyl-2-trifluoromethylfuran-3-ylcarbonylamino]cyclohexyl-N-[1-[5-(2-dimethylaminoethylthio)-1,3,4-oxadiazol-2-yl]-4-methyl-1-oxo-2-pentyl]carboxamide,
(5) 1-(isoxazol-5-ylcarbonylamino)cyclohexyl-N-[1-[5-(2-dimethylaminoethylthio)-1,3,4-oxadiazol-2-yl]-4-methyl-1-oxo-2-pentyl]carboxamide,
(6) 1-anilinocarbonylcyclohexyl-N-[1-[5-(2-dimethylaminoethylthio)-1,3,4-oxadiazol-2-yl]-4-methyl-1-oxo-2-pentyl]carboxamide,
(7) 1-benzyloxycarbonylaminocyclohexyl-N-[1-[5-(2-dimethylaminoethylthio)-1,3,4-oxadiazol-2-yl]-4-methyl-1-oxo-2-pentyl]carboxamide,
(8) 1-(4-methylphenyloxycarbonylamino)cyclohexyl-N-[1-[5-(2-dimethylaminoethylthio)-1,3,4-oxadiazol-2-yl]-4-methyl-1-oxo-2-pentyl]carboxamide,
(9) 1-(4-chlorophenyloxycarbonylamino)cyclohexyl-N-[1-[5-(2-dimethylaminoethylthio)-1,3,4-oxadiazol-2-yl]-4-methyl-1-oxo-2-pentyl]carboxamide,
(10) 1-(4-bromophenyloxycarbonylamino)cyclohexyl-N-[1-[5-(2-dimethylaminoethylthio)-1,3,4-oxadiazol-2-yl]-4-methyl-1-oxo-2-pentyl]carboxamide,
(11) 1-benzyloxymethylcarbonylcyclohexyl-N-[1-[5-(2-dimethylaminoethylthio)-1,3,4-oxadiazol-2-yl]-4-methyl-1-oxo-2-pentyl]carboxamide,
(12) 1-phenethylcyclohexyl-N-[1-[5-(2-dimethylaminoethylthio)-1,3,4-oxadiazol-2-yl]-4-methyl-1-oxo-2-pentyl]carboxamide,
(13) 2,2-dimethylpropyloxy-N-[1-[5-(2-dimethylaminoethylthio)-1,3,4-oxadiazol-2-yl]-4-methyl-1-oxo-2-pentyl]carboxamide,
(14) 1-(4-hydroxybenzoylamino)cyclohexyl-N-[1-[5-(2-dimethylaminoethylthio)-1,3,4-oxadiazol-2-yl]-4-methyl-1-oxo-2-pentyl]carboxamide,
(15) 1-(t-butoxycarbonylamino)cyclohexyl-N-[1-[5-(2-benzylmethylaminoethylthio)-1,3,4-oxadiazol-2-yl]-4-methyl-1-oxo-2-pentyl]carboxamide,
(16) 1-benzoylaminocyclohexyl-N-[1-[5-(2-benzylmethylaminoethylthio)-1,3,4-oxadiazol-2-yl]-4-methyl-1-oxo-2-pentyl]carboxamide,
(17) N-[1-(5-dimethylaminoethylthio-1,3,4-oxadiazol-2-yl)-4-methyl-1-oxo-2-pentyl]-4,4-dimethyl-2-pentenamide,
(18) 4-(t-butyl)cyclohexyl-N-[1-[5-(2-dimethylaminoethylthio)-1,3,4-oxadiazol-2-yl]4-methyl-1-oxo-2-pentyl]carboxamide,
(19) N-[1-(5-dimethylaminoethylthio-1,3,4-oxadiazol-2-yl)-4-methyl-1-oxo-2-pentyl]-3,3-dimethyl-2-pentanamide,
(20) cyclohexyl-N-[1-[5-(2-dimethylaminoethylthio)-1,3,4-oxadiazol-2-yl]-1-oxo-3-phenyl-2-propyl]carboxamide,
(21) cycloheptyl-N-[1-[5-(2-dimethylaminoethylthio)-1,3,4-oxadiazol-2-yl]-1-oxo-3-phenyl-2-propyl]carboxamide,
(22) cycloheptyl-N-[(2S)-1-[5-(2-dimethylaminoethylthio)-1,3,4-oxadiazol-2-yl]-3-methyl-1-oxo-2-butyl]carboxamide,
(23) cyclohexyl-N-[1-[5-(2-dimethylaminoethylthio)-1,3,4-oxadiazol-2-yl]-4-phenyl-1-oxo-2-butyl]carboxamide,
(24) cycloheptyl-N-[1-[5-(2-dimethylaminoethylthio)-1,3,4-oxadiazol-2-yl]-1-oxo-4-phenyl-2-butyl]carboxamide,
(25) cycloheptyl-N-[(2S)-3,3-dimethyl-1-[5-(2-dimethylaminoethylthio)-1,3,4-oxadiazol-2-yl]-1-oxo-2-butyl]carboxamide,
(26) cycloheptyl-N-[3-cyclohexyl-1-[5-(2-dimethylaminoethylthio)-1,3,4-oxadiazol-2-yl]-1-oxo-2-propyl]carboxamide,
(27) cyclohexyl-N-[1-[5-[2-(N-t-butyl-N-methylamino)ethylthio]-1,3,4-oxadiazol-2-yl]-4-methyl-1-oxo-2-pentyl]carboxamide,
(28) 2-cycloheptylcarbonyl-3-[5-(2-dimethylaminoethylthio)-1,3,4-oxadiazol-2-ylcarbonyl]-1,2,3,4-tetrahydroisoquinoline,
(29) cycloheptyl-N-[1-[5-(2-dimethylaminoethylthio)-1,3,4-oxadiazol-2-ylcarbonyl]cyclohexyl]carboxamide,
(30) cycloheptyl-N-[2-cyclohexyl-1-[5-(2-dimethylaminoethylthio)-1,3,4-oxadiazol-2-yl]-1-oxo-2-ethyl]carboxamide,
(31) cyclohexyl-N-[4,4-dimethyl-1-[5-(2-dimethylaminoethylthio)-1,3,4-oxadiazol-2-yl]-1-oxo-2-pentyl]carboxamide,
(32) cycloheptyl-N-[4,4-dimethyl-1-[5-(2-dimethylaminoethylthio)-1,3,4-oxadiazol-2-yl]-1-oxo-2-pentyl]carboxamide,
(33) cycloheptyl-N-[1-[5-(2-dimethylaminoethylthio)-1,3,4-oxadiazol-2-yl]-2-(tetrahydropyran-4-yl)-1-oxo-2-ethyl]carboxamide,
(34) cycloheptyl-N-[1-[5-(2-dimethylaminoethylthio)-1,3,4-oxadiazol-2-yl]-4-methoxy-1-oxo-2-butyl]carboxamide.
(35) cyclohexyl-N-[1-[5-[2-(1,2,3,4-tetrahydroisoquinolin-2-yl)ethylthio]-1,3,4-oxadiazol-2-yl]-4-methyl-1-oxo-2-pentyl]carboxamide,
(36) cyclohexyl-N-[(2S)-1-[5-[2-(N-benzyl-N-methylamino)ethylthio]-1,3,4-oxadiazol-2-yl]-3-methyl-1-oxo-2-butyl]carboxamide,
(37) cyclohexyl-N-[(2S)-1-[5-(2-dimethylaminoethylthio)-1,3,4-oxadiazol-2-yl]-3-methyl-1-oxo-2-butyl]carboxamide,
(38) 1-benzoylaminocyclohexyl-N-[(2S)-1-[5-(2-dimethylaminoethylthio)-1,3,4-oxadiazol-2-yl]-3-methyl-1-oxo-2-butyl]carboxamide,
(39) cycloheptyl-N-[1-[5-(2-dimethylaminoethylthio)-1,3,4-oxadiazol-2-yl]-1-oxo-2-phenyl-2-ethyl]carboxamide,
(40) 1-morpholinocarbonylaminocyclohexyl-N-[(2S)-1-[5-(2-dimethylaminoethylthio)-1,3,4-oxadiazol-2-yl]-3-methyl-1-oxo-2-butyl]carboxamide,
(41) 1-(4-dimethylaminomethylbenzoylaminocyclohexyl)-N-[(2S)-3-methyl-1-[5-(1-methylethylthio)-1,3,4-oxadiazol-2-yl]-1-oxo-2-butyl]carboxamide,
(42) 1-(3-dimethylaminomethylbenzoylaminocyclohexyl)-N-[(2S)-3-methyl-1-[5-(1-methylethylthio)-1,3,4-oxadiazol-2-yl]-1-oxo-2-butyl]carboxamide,
(43) cyclohexyl-N-[1-[5-(2-diisopropylaminoethylthio)-1,3,4-oxadiazol-2-yl]-4-methyl-1-oxo-2-pentyl]carboxamide,
(44) cycloheptyl-N-[1-[5-(2-dimethylaminoethylthio)-1,3,4-oxadiazol-2-yl]-3-ethyl-1-oxo-2-pentyl]carboxamide,
(45) cyclohexyl-N-[1-[5-[2-(N-isopropyl-N-methylamino)ethylthio]-1,3,4-oxadiazol-2-yl]-4-methyl-1-oxo-2-pentyl]carboxamide,
(46) cyclohexyl-N-[1-[5-[2-(2,5-dihydropyrrol-1-yl)ethylthio]-1,3,4-oxadiazol-2-yl]-4-methyl-1-oxo-2-pentyl]carboxamide,
(47) cyclohexyl-N-[1-[5-[2-[N-methyl-N-[2-(pyridin-2-yl)ethyl]amino]ethylthio]-1,3,4-oxadiazol-2-yl]-4-methyl-1-oxo-2-pentyl]carboxamide,
(48) cyclohexyl-N-[(2S)-1-[5-[2-(tetrahydropyran-2-yloxy)ethylthio]-1,3,4-oxadiazol-2-yl]-4-methyl-1-oxo-2-pentyl]carboxamide,
(49) cyclohexyl-N-[(2S)-1-[5-(2-hydroxyethylthio)-1,3,4-oxadiazol-2-yl]-4-methyl-1-oxo-2-pentyl]carboxamide,
(50) cyclohexyl-N-[1-[5-[2-(N-benzyl-N-ethylamino)ethylthio]-1,3,4-oxadiazol-2-yl]-4-methyl-1-oxo-2-pentyl]carboxamide,
(51) cycloheptyl-N-[(3S)-1-[5-[2-dimethylaminoethylthio]-1,3,4-oxadiazol-2-yl]-3-methyl-1-oxo-2-pentyl]carboxamide,
(52) cyclohexyl-N-[1-[5-[2-[4-(t-butoxycarbonyl)piperazin-1-yl]ethylthio]-1,3,4-oxadiazol-2-yl]-3-methyl-1-oxo-2-pentyl]carboxaniide,
(53) cyclohexyl-N-[1-[5-[2-(N-methyl-N-phenethylamino)ethylthio]-1,3,4-oxadiazol-2-yl]-4-methyl-1-oxo-2-pentyl]carboxamide,
(54) cyclohexyl-N-[1-[5-[2-[N-(2-methoxyethyl)-N-methylamino]ethylthio]-1,3,4-oxadiazol-2-yl]-4-methyl-1-oxo-2-pentyl]carboxamide,
(55) cyclohexyl-N-[1-[5-(1,1-dimethyl-2-dimethylaminoethylthio)-1,3,4-oxadiazol-2-yl]-4-methyl-1-oxo-2-pentyl]carboxamide,
(56) cyclohexyl-N-[1-[5-(2,2-dimethyl-2-dimethylaminoethylthio)-1,3,4-oxadiazol-2-yl]-4-methyl-1-oxo-2-pentyl]carboxamide,
(57) cycloheptyl-N-[2-(1-t-butoxycarbonylpiperidin-4-yl)-1-[5-(2-isopropylthio)-1,3,4-oxadiazol-2-yl]-1-oxo-2-ethyl]carboxamide,
(58) cycloheptyl-N-[2-(1-t-butoxycarbonylpiperidin-4-yl)-1-[5-[2-(2-dimethylamino)ethylthio]-1,3,4-oxadiazol-2-yl]-1-oxo-2-ethyl]carboxamide,
(59) cycloheptyl-N-[2-(1-acetylpiperidin-4-yl)-1-[5-(2-isopropylthio)-1,3,4-oxadiazol-2-yl]-1-oxo-2-ethyl]carboxamide,
(60) cycloheptyl-N-[2-(1-benzoylpiperidin-4-yl)-1-[5-(2-isopropylhtio)-1,3,4-oxadiazol-2-yl]-1-oxo-2-ethyl]carboxamide,
(61) cycloheptyl-N-[1-[5-(2-isopropylthio)-1,3,4-oxadiazol-2-yl]-2-(1-methoxycarbonylpiperidin-4-yl)-1-oxo-2-ethyl]carboxamide,
(62) cycloheptyl-N-[2-(1-benzylpiperidin-4-yl)-1-[5-(2-isopropylthio)-1,3,4-oxadiazol-2-yl]-1-oxo-2-ethyl]carboxamide,
(63) cycloheptyl-N-[1-[5-(2-dimethylaminoethylthio)-1,3,4-oxadiazol-2-yl]-2-(1-acetylpiperidin-4-yl)-1-oxo-2-ethyl]carboxamide,
(64) cyclohexyl-N-[1-[5-(2-dimethylaminoethylthio)-1,3,4-oxadiazol-2-yl]-4-methoxy-4-methyl-1-oxo-2-pentyl]carboxamide,
(65) cyclohexyl-N-[(2S)-1-(4-benzyl-5-oxo-1,3,4-oxadiazolin-2-yl)-3-methyl-1-oxo-2-butyl]carboxamide,
(66) cyclohexyl-N-[(2S)-1-(5-oxo-4-phenethyl-1,3,4-oxadiazolin-2-yl)-3-methyl-1-oxo-2-butyl]carboxamide,
(67) cyclohexyl-N-[(2S)-3-methyl-1-oxo-1-[5-oxo-4-(3-phenylpropyl)-1,3,4-oxadiazolin-2-yl]-2-butyl]carboxamide,
(68) cyclohexyl-N-[1-[4-(3-dimethylaminopropyl)-5-oxo-1,3,4-oxadiazolin-2-yl]-3-methyl-1-oxo-2-butyl]carboxamide,
(69) cyclohexyl-N-[(2S)-3-methyl-1-oxo-1-(5-oxo-4-phenyl-1,3,4-oxadiazolin-2-yl)-2-butyl]carboxamide,
(70) cyclohexyl-N-[(2S)-1-[5-(2-methylaminoethylthio)-1,3,4-oxadiazol-2-yl]-4-methyl-1-oxo-2-pentyl]carboxamide,
(71) cycloheptyl-N-[1-[5-(2-isopropylthio)-1,3,4-oxadiazol-2-yl]-2-(piperidin-4-yl)-1-oxo-2-ethyl]carboxamide,
(72) cycloheptyl-N-[1-[5-(2-dimethylaminoethylthio)-1,3,4-oxadiazol-2-yl]-2-(piperidin-4-yl)-1-oxo-2-ethyl]carboxamide,
(73) cyclohexyl-N-[(2S)-3-methyl-1-[4-(2-methylaminoethyl)-5-oxo-1,3,4-oxadiazolin-2-yl]-1-oxo-2-butyl]carboxamide,
(74) cyclohexyl-N-[(2S)-3-methyl-1-[5-(2-methylaminoethylthio)-1,3,4-oxadiazol-2-yl]-1-oxo-2-butyl]carboxamide,
(75) cyclohexyl-N-[(2S)-4-methyl-1-[5-(3-methylaminopropylthio)-1,3,4-oxadiazol-2-yl]-1-oxo-2-pentyl]carboxamide,
(76) cycloheptyl-N-[(2S)-4,4-dimethyl-1-[5-(2-methylaminoethylthio)-1,3,4-oxadiazol-2-yl]-1-oxo-2-pentyl]carboxamide,
(77) cyclohexyl-N-[(2S)-3-methyl-1-[3-(2-methylaminoethyl)-2-oxo-1,3,4-oxadiazolin-5-yl]-1-oxo-2-butyl]carboxamide,
(78) cyclohexyl-N-[(2S)-1-[5-(2-methylaminoethylthio)-1,3,4-oxadiazol-2-yl]-1-oxo-3-phenyl-2-propyl]carboxamide,
(79) cyclohexyl-N-[(2S)-1-[3-(2-hydroxyethyl)-2-oxo-1,3,4-oxadiazolin-5-yl]-3-methyl-1-oxo-2-butyl]carboxamide,
(80) cyclohexyl-N-[(2S,3 S)-1-[5-(2-methylaminoethylthio)-1,3,4-oxadiazol-2-yl]-3-methyl-1-oxo-2-pentyl]carboxamide,
(81) cyclohexyl-N-[(2S)-3,3-dimethyl-1-[5-(2-methylaminoethylthio)-1,3,4-oxadiazol-2-yl]-1-oxo-2-butyl]carboxamide,
(82) cyclohexyl-N-[(2S)-1-[5-(2-methylaminoethylthio)-1,3,4-oxadiazol-2-yl]-1-oxo-2-hexyl]carboxamide,
(83) cyclohexyl-N-[1-[5-(2-methylaminoethylthio)-1,3,4-oxadiazol-2-yl]-4,4-dimethyl-1-oxo-2-pentyl]carboxamide,
(84) cyclohexyl-N-[1-[5-(2-methylaminoethylthio)-1,3,4-oxadiazol-2-yl]-4-methoxy-4-methyl-1-oxo-2-pentyl]carboxamide,
(85) N-[(2S)-1-[5-(2-methylaminoethylthio)-1,3,4-oxadiazol-2-yl]-4-methyl-1-oxo-2-pentyl]acetamide,
(86) (tetrahydropyran-4-yl)-N-[(2S)-1-[5-(2-methylaminoethylthio)-1,3,4-oxadiazol-2-yl]-4-methyl-1-oxo-2-pentyl]carboxamide,
(87) t-butyl-N-[(2S)-4-methyl-1-[5-(2-methylaminoethylthio)-1,3,4-oxadiazol-2-yl]-1-oxo-2-pentyl]carboxamide,
(88) N-[(2S)-4-methyl-1-[5-(2-methylaminoethylthio)-1,3,4-oxadiazol-2-yl]-1-oxo-2-pentyl]benzamide,
(89) cycloheptyl-N-[(2S)-4-methyl-1-[5-(2-methylaminoethylthio)-1,3,4-oxadiazol-2-yl]-1-oxo-2-pentyl]carboxamide,
(90) cyclohexyl-N-[2-methyl-1-[5-(2-methylaminoethylthio)-1,3,4-oxadiazol-2-yl]-1-oxo-2-propyl]carboxamide,
(91) cyclohexyl-N-[(2S)-1-[5-(2-ethylaminoethylthio)-1,3,4-oxadiazol-2-yl]-4-methyl-1-oxo-2-pentyl]carboxamide,
(92) cycloheptyl-N-[1-[5-(2-methylaminoethylthio)-1,3,4-oxadiazol-2-yl]-4,4-dimethyl-1-oxo-2-pentyl]carboxamide,
(93) N-[(2S)-1-4-methyl-[5-(2-methylaminoethylthio)-1,3,4-oxadiazol-2-yl]-1-oxo-2-pentyl]phenylacetamide,
(94) N-[(2S)-4-methyl-1-[5-(2-methylaminoethylthio)-1,3,4-oxadiazol-2-yl]-1-oxo-2-pentyl]-phenoxyacetamide,
(95) cyclohexyl-N-[1-[3-(2-methylaminoethyl)-2-oxo-1,3,4-oxadiazolin-5-yl]-4,4-dimethyl-1-oxo-2-pentyl]carboxamide,
(96) cyclohexyl-N-[(2S)-1-[3-(2-methylaminoethyl)-2-oxo-1,3,4-oxadiazolin-5-yl]-4-methyl-1-oxo-2-pentyl]carboxamide,
(97) cyclohexyl-N-[(2S)-1-[5-(2-benzylaminoethylthio)-1,3,4-oxadiazol-2-yl]-4-methyl-1-oxo-2-pentyl]carboxamide,
(98) cyclohexyl-N-[(2S)-1-[5-[2-(1-methylethylamino)ethylthio]-1,3,4-oxadiazol-2-yl]-4-methyl-1-oxo-2-pentyl]carboxamide,
(99) cyclohexyl-N-[2-cyclohexyl-1-[5-(2-methylaminoethylthio)-1,3,4-oxadiazol-2-yl]-1-oxo-2-ethyl]carboxamide,
(100) N-[(2S)-4-methyl-1-[5-(2-methylaminoethylthio)-1,3,4-oxadiazol-2-yl]-1-oxo-2-pentyl]-4,4-dimethyl-2-pentenamide,
(101) cyclohexyl-N-[2-cyclopropyl-1-[5-(2-methylaminoethylthio)-1,3,4-oxadiazol-2-yl]-1-oxo-2-ethyl]carboxamide,
(102) N-[(2S)-4-methyl-1-[5-(2-methylaminoethylthio)-1,3,4-oxadiazol-2-yl]-1-oxo-2-pentyl]-4,4-dimethylpentanamide,
(103) cyclohexyl-N-[2-cyclopentyl-1-[5-(2-methylaminoethylthio)-1,3,4-oxadiazol-2-yl]-1-oxo-2-ethyl]carboxamide,
(104) cyclohexyl-N-[1-[5-(2-methylaminoethylthio)-1,3,4-oxadiazol-2-yl]-1-oxo-3-propyl-2-hexyl]carboxamide,
(105) cyclohexyl-N-[1-[5-(2-methylaminoethylthio)-1,3,4-oxadiazol-2-yl]-2-phenyl-1-oxo-2-ethyl]carboxamide,
(106) N-(2-methylpropyloxycarbonyl)-N-[(2S)-4-methyl-1-[5-(2-methylaminoethylthio)-1,3,4-oxadiazol-2-yl]-1-oxo-2-pentyl]amine,
(107) cyclohexyl-N-[1-[5-(2-methylaminoethylthio)-1,3,4-oxadiazol-2-yl]-2-(piperidin-4-yl)-1-oxo-2-ethyl]carboxamide,
(108) cyclohexyl-N-[1-[5-(2-benzylaminoethylthio)-1,3,4-oxadiazol-2-yl]-2-(tetrahydropyran-4-yl)-1-oxo-2-ethyl]carboxamide,
(109) cyclohexyl-N-[3-ethyl-1-[5-(2-methylaminoethylthio)-1,3,4-oxadiazol-2-yl]-1-oxo-2-pentyl]carboxamide,
(110) cyclohexyl-N-[1-[5-(2-methylaminoethylthio)-1,3,4-oxadiazol-2-yl]-2-(4-trifluoromethylphenyl)-1-oxo-2-ethyl]carboxamide,
(111) cyclohexyl-N-[2-(4-methoxyphenyl)-1-[5-(2-methylaminoethylthio)-1,3,4-oxadiazol-2-yl]-1-oxo-2-ethyl]carboxamide,
(112) cyclohexyl-N-[1-[5-(2-methylaminoethylthio)-1,3,4-oxadiazol-2-yl]-2-(2-methylphenyl)-1-oxo-2-ethyl]carboxamide,
(113) cyclohexyl-N-[1-[5-(2-propylaminoethylthio)-1,3,4-oxadiazol-2-yl]-2-(tetrahydropyran-4-yl)-1-oxo-2-ethyl]carboxamide,
(114) cyclohexyl-N-[(2S)-1-[5-(2-propylaminoethylthio)-1,3,4-oxadiazol-2-yl]-3-methyl-1-oxo-2-butyl]carboxamide or (115) cyclohexyl-N-[(2S)-1-[5-(2-benzylaminoethylthio)-1,3,4-oxadiazol-2-yl]-3-methyl-1-oxo-2-butyl]carboxamide or a non-toxic salt thereof.

14. The compound according to claim 2, which is 2,2,2-trichloroethyloxy-N-[1-[5-(2-dimethylaminoethylthio)-1,3,4-oxadiazol-2-yl]-4-methyl-1-oxo-2-pentyl]carboxamide or a non-toxic salt thereof.

15. The compound according to claim 5, which is
(1) cyclohexyl-N-[1-[5-[2-(N-ethoxycarbonylmethyl-N-methylamino)ethylthio]-1,3,4-oxadiazol-2-yl]-4-methyl-1-oxo-2-pentyl]carboxamide,
(2) cyclohexyl-N-[(2S)-1-[5-[2-(N-t-butoxycarbonyl-N-methylamino)ethylthio]-1,3,4-oxadiazol-2-yl]-4-methyl-1-oxo-2-pentyl]carboxamide,
(3) 1-benzoylaminocyclohexyl-N-[(2S)-1-[5-[2-(N-methoxycarbonyl-N-methylamino)ethylthio]-1,3,4-oxadiazol-2-yl]-4-methyl-1-oxo-2-pentyl]carboxamide or
(4) cyclohexyl-N-[(2S)-1-[5-(2-N-methoxycarbonyl-N-methylamino-2-dimethylethylthio)-1,3,4-oxadiazol-2-yl]-4-methyl-1-oxo-2-pentyl]carboxamide, or a non-toxic salt thereof.

16. A pharmaceutical composition comprising, as an active ingredient, the oxadiazole derivative of formula (I) according to claim 1 or a non-toxic salt thereof.

17. A pharmaceutical composition comprising, as an active ingredient, the oxadiazole derivative of formula (I) according to claim 1 or a non-toxic salt thereof, which inhibits cysteine protease.

18. The pharmaceutical composition according to claim 17, wherein the cysteine protease is cathepsin K, cathepsin S, cathepsin L, cathepsin B, cathepsin H or caspase-1.

19. The pharmaceutical composition according to claim 18, wherein the cysteine protease is cathrpsin K.

20. The pharmaceutical composition according to claim 18, wherein the cysteine protease is cathepsin S.

21. The pharmaceutical composition, comprising, as an active ingredient, the compound of formula (I) according to claim 1 or a non-toxic salt thereof for the prophylaxis and/or treatment of inflammatory diseases, diseases induced by apoptosis, diseases induced by disorders of immune responses, autoimmune diseases, diseases induced by decomposition of proteins which compose organism, shock, circulatory system disorders, blood coagulation system(s) disorders, malignant tumors, acquired immune deficiency syndrome (AIDS) and AIDS-related complex (ARC), parasitic diseases, nerve degeneration diseases, pulmonary disorders, bone resorption diseases, endocrinesthenia, etc.
